(19) United States
(12) Patent Application Publication
(10) Pub. No.: US 2016/0002247 A1
(43) Pub. Date: Jan. 7, 2016

(54) 8-SUBSTITUTED IMIDAZOPYRIMIDINONE DERIVATIVE HAVING AUTOTAXIN INHIBITORY ACTIVITY

(71) Applicants: THE UNIVERSITY OF TOKYO, Bunkyo-ku, Tokyo (JP); TOHOKU UNIVERSITY, Sendai-shi, Miyagi (JP); Shionogi & Co., Ltd., Osaka-shi, Osaka (JP)

(72) Inventors: Tetsuo NAGANO, Tokyo (JP); Takayoshi OKABE, Tokyo (JP); Hirotatsu KOJIMA, Tokyo (JP); Mitsuyasu KAWAGUCHI, Tokyo (JP); Osamu NUREKI, Tokyo (JP); Ryuichiro ISHITANI, Tokyo (JP); Hiroshi NISHIMASU, Tokyo (JP); Junken AOKI, Miyagi (JP); Nobuyuki TANAKA, Osaka (JP); Chiaki FUJIKOSHI, Osaka (JP); Yusuke TATENO, Osaka (JP); Toshihiro WADA, Osaka (JP)

(73) Assignees: THE UNIVERSITY OF TOKYO, Bunkyo-ku, Tokyo (JP); TOHOKU UNIVERSITY, Sendai-shi, Miyagi (JP); Shionogi & Co., Ltd., Osaka-shi, Osaka (JP)

(21) Appl. No.: 14/770,959

(22) PCT Filed: Feb. 27, 2014
(86) PCT No.: PCT/JP2014/054982

§ 371 Date: Aug. 27, 2015

(30) Foreign Application Priority Data
Mar. 1, 2013 (JP) 2013-041176
Oct. 31, 2013 (JP) 2013-227215

(51) Int. Cl. C07D 487/04; C07D 519/00

(52) U.S. Cl. CPC C07D 487/04; C07D 519/00

(57) ABSTRACT

A compound of formula (I) wherein variables are as defined herein having an autotaxin inhibitory effect and a pharmaceutical composition comprising the same.

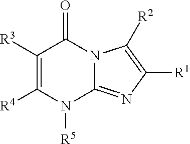

### TECHNICAL FIELD

The present invention imidazopyrimidinone derivative having autotaxin inhibitory activity, as well as a pharmaceutical comprising said imidazopyrimidinone derivatives as an active ingredient.

### BACKGROUND ART

Lysophosphatidic acid (LPA) is a lipid mediator that exhibits a variety of effects, such as cell proliferation, intracellular calcium influx, cytoskeletal changes, cell migration, via signal transduction through G protein-coupled receptor expressed on cell surface (LPA1-6). It has been reported that the lipid is involved in abnormalities of living body, such as fibrosis, pain, cancer, inflammation, arteriosclerosis (Non-Patent Document 1).

LPA can be biosynthesized by several metabolic pathways, primarily via hydrolysis of lysophosphatidylcholine by autotaxin (ENPP2, ATX). ATX is a secreted protein of ENPP (Ectonucleotide pyrophosphatase and phosphodiesterase) family (ENPP1-7) and referred to as ENPP2. ATX is the only one of this family that has a lysophospholipase D activity and thus is involved in LPA production. It has been reported that inhibiting the enzyme activity of ATX to inhibit LPA production is effective in the treatment of fibrotic diseases (Non-Patent Document 1).

Fibrosis can occur in any organ, and the mechanism of its progression is common regardless of the trigger involved.

Animal tissues and organs maintain its structure with fibers such as collagen, and injured tissues and organs are restored to the original condition through the process of wound healing with collagen production. However, in case where the tissue receives immunological, chemical, mechanical, metabolic or other injuries repeatedly or experiences a greater degree of injury, excessive accumulation of fibrous connective tissue may occur. Accumulation of such connective tissue is irreversible, and fibers abnormally increased cause fibrosis that is associated with dysfunction of tissues and organs.

Pathological feature of chronic kidney disease includes renal glomerular fibrosis and tubulointerstitial fibrosis. Dropout and fibrosis of parenchymal cells prevail in the pathology of end-stage renal failure. In chronic kidney disease patients having tubulointerstitial fibrosis, the progress to renal failure is faster as compared to chronic kidney disease patients without such fibrosis.

For preventing and treating chronic kidney disease, treatments with an antihypertensive drug, such as angiotensin receptor antagonists and calcium antagonists, have been practiced, as well as advice on daily living and dietary. However, the effect from such conventional treatments is not enough to be satisfied, and there still exists an ongoing need for new drugs to make prevention and treatment more effective.

Patent Document 1 discloses imidazopyrimidinone derivatives that inhibit gonadotropin releasing hormone. However, it is not described or suggested that such compounds inhibit autotaxin or may be a therapeutic agent for chronic kidney disease.

Patent Documents 2 to 15 describe polycyclic compounds that inhibit autotaxin, but no description or suggestion is provided for the imidazopyrimidinone derivative of the invention. Patent Documents 16-23 describe monocyclic compounds that inhibit autotaxin, but no description or suggestion is provided for the imidazopyrimidinone derivatives of the invention.

### PRIOR ART DOCUMENTS

### Patent Documents

WO2003/51885
WO2012/127885
WO2012/5227
WO2011/116867
WO2011/5669
WO2010/60532
WO2012/100018
US Patent Application Publication No. 2012/100592
WO2012/24620
WO2011/53597
WO2010/115491
WO2010/112124
WO2009/46841
WO2009/46842
WO2010/63352
WO2012/138648
WO2011/41462
WO2011/41694
WO2011/17350
WO2010/112116
WO2010/68775
US Patent Application Publication No. 2010/16258
WO2009/46804

### Non-Patent Documents

Nature, vol. 411, pp. 494-498 (2001)

### SUMMARY OF INVENTION

### Problem to be Solved by the Invention

The object of the present invention is to provide 8-substituted-imidazopyrimidinone derivatives having an excellent inhibitory activity on autotaxin.

### Means for Solving the Problem

The present invention is based on the inventor's discovery of the 8-substituted imidazopyrimidinone derivatives having an excellent inhibitory activity on autotaxin.

The present invention relates to the following.

[1] An autotaxin inhibitor comprising a compound of formula (I):

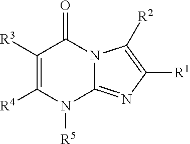

wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
R², R³ and R⁴ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino,
—N═C(R^{4a})(OR^{4b}) wherein R^{4a} is substituted or unsubstituted alkyl and R^{4b} is substituted or unsubstituted alkyl, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group, or a pharmaceutically acceptable salt thereof.
[1′] A autotaxin inhibitor comprising a compound of formula (I) wherein
R¹ is substituted or unsubstituted alkyl, substituted unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
R², R³ and R⁴ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group,
or a pharmaceutically acceptable salt thereof.
[2] A medicament comprising the autotaxin inhibitor according to [1] or [1′] for the prevention or treatment of a disease involving autotaxin.
[3] A compound of formula (I) or a pharmaceutically acceptable salt thereof:

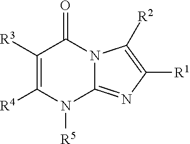

wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
R², R³ and R⁴ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino,
—N═C (R^{4a}) (OR^{4b})) wherein R^{4a} is substituted or unsubstituted alkyl and R^{4b} is substituted or unsubstituted alkyl, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group, provided that
(a) a compound
wherein R⁵ is a group of formula:

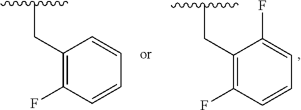 [SMILES: *Cc1ccccc1F ; *Cc1c(F)cccc1F]

and
which conforms to one of provisions (i) to (v):

(i) R² is substituted or unsubstituted amino-(C1-C2)alkyl or substituted or unsubstituted bromomethyl,

(ii) R¹ is phenyl substituted with a group other than halogen, haloalkyl or haloalkyloxy or unsubstituted phenyl, R² is methyl, and R⁴ is hydrogen or methyl,

(iii) R¹ is substituted phenyl, R² is hydrogen, R³ is substituted phenyl, and R⁴ is methyl,

(iv) R³ is bromo or alkyloxycarbonyl, and R⁴ is hydrogen, or

(v) R¹ is alkyl substituted with alkyloxycarbonyl or unsubstituted alkyl, R² is alkyl substituted with substituted or unsubstituted nitrogen-containing aromatic heterocyclic group, and R³ is substituted phenyl and R⁴ is methyl;

(b) a compound wherein R¹ is substituted or unsubstituted aromatic carbocyclic group or unsubstituted furyl, and R² is substituted or unsubstituted phenyl; and
aromatic carbocyclic group or unsubstituted furyl, and R² is substituted or unsubstituted phenyl; and
(c) the compounds of the formula:

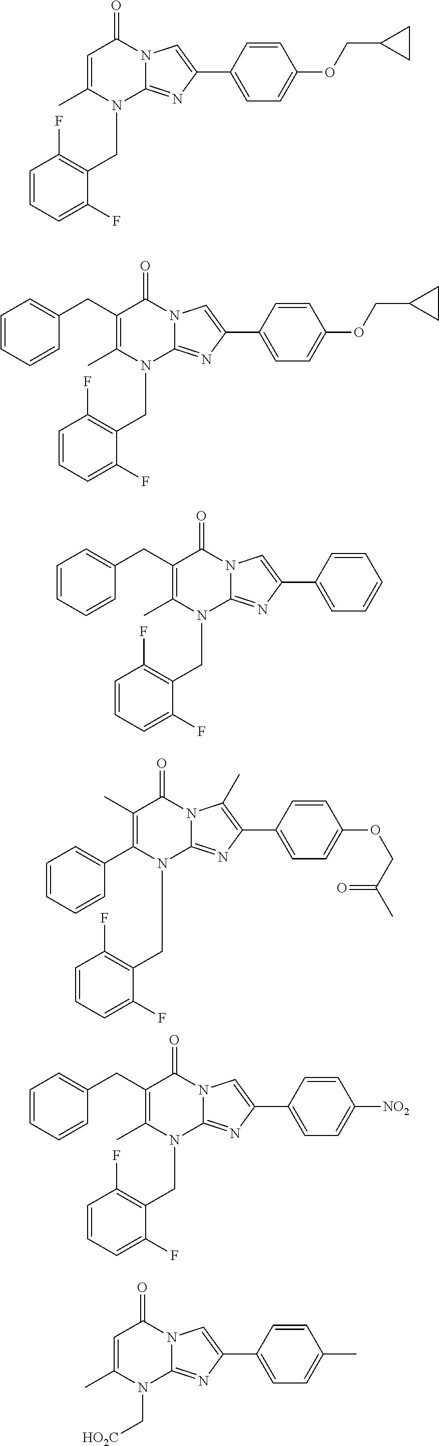

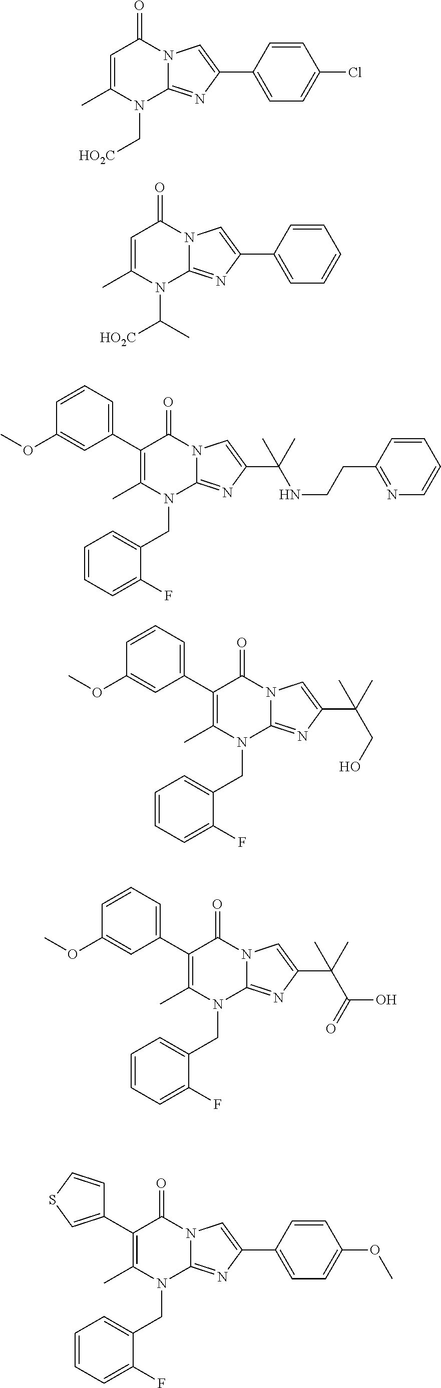

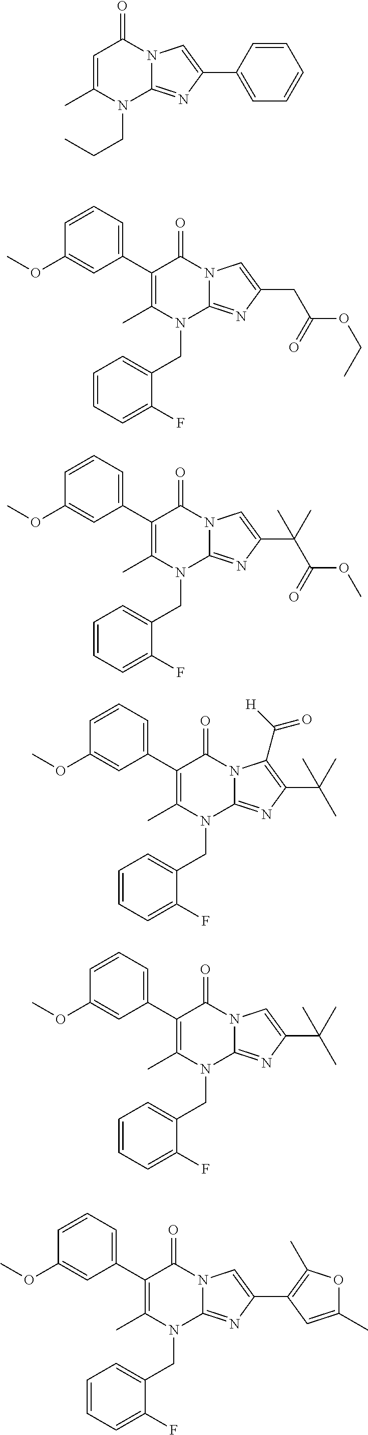

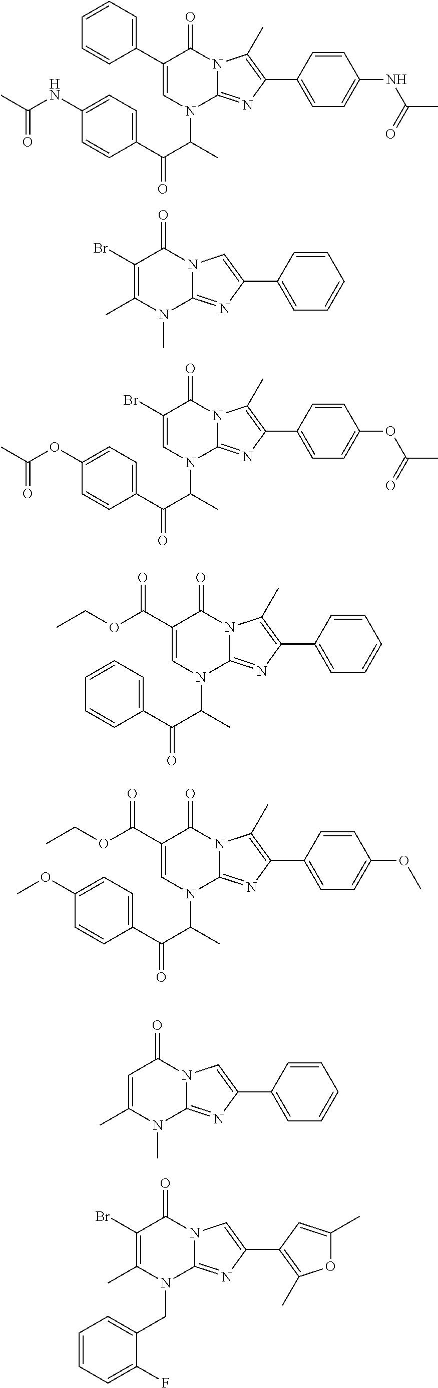

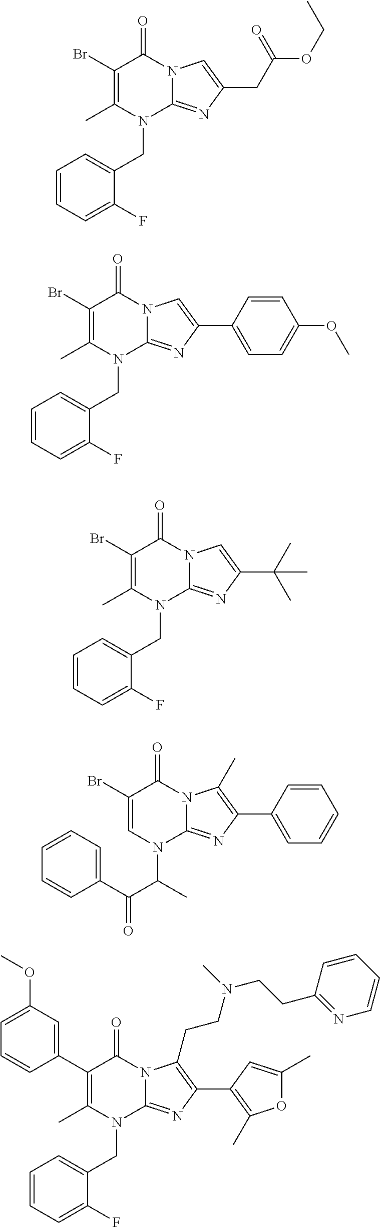

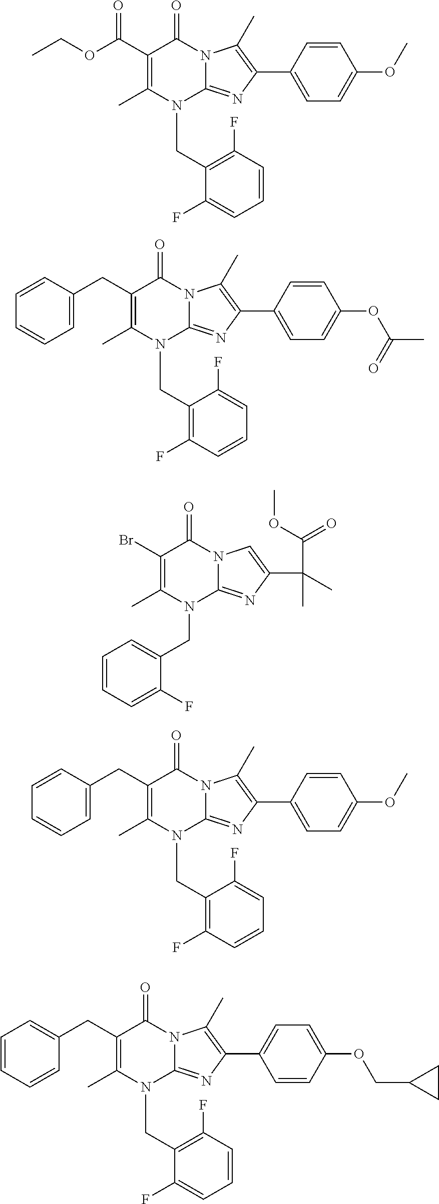

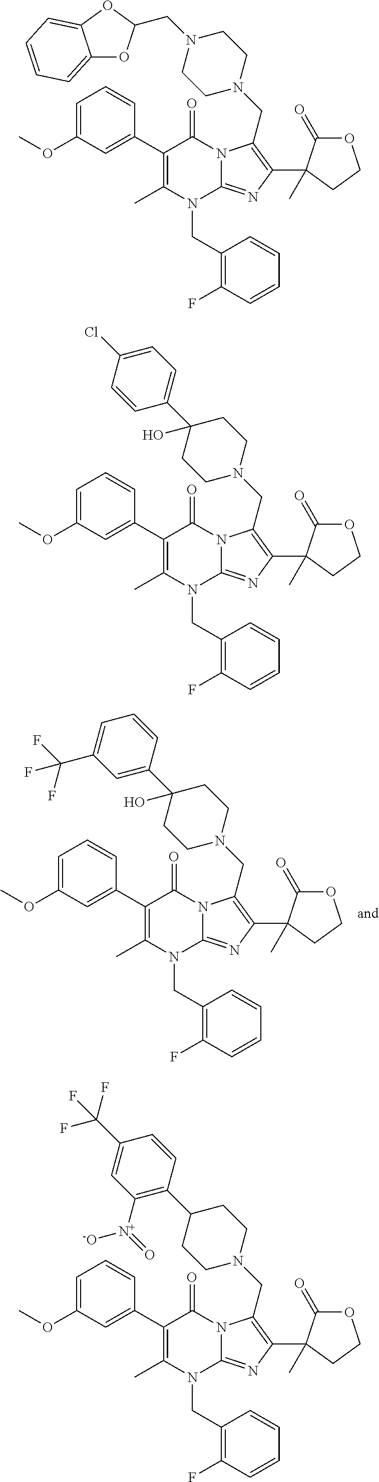

are excluded.
[3′]A compound of formula (I) or a pharmaceutically acceptable salt thereof:

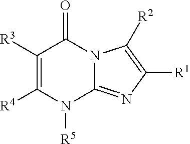

wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
R², R³ and R⁴ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino,
—N═C(R^{4a}) (OR^{4b}) wherein R^{4a} is substituted or unsubstituted alkyl and R^{4b} is substituted or unsubstituted alkyl, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group, provided that
(a) a compound
wherein R⁵ is a group of formula:

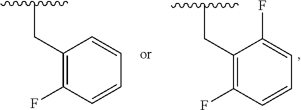 [SMILES: *Cc1ccccc1F ; *Cc1c(F)cccc1F]

which conforms to one of provisions (i) to (iv):

(i) R² is substituted or unsubstituted amino-(C1-C2)alkyl or substituted or unsubstituted bromomethyl,

(ii) R¹ is phenyl substituted with a group other than halogen, haloalkyl or haloalkyloxy or unsubstituted phenyl, R² is methyl, and R⁴ is hydrogen or methyl,

(iii) R¹ is substituted phenyl, R² is hydrogen, R³ is substituted phenyl, and R⁴ is methyl, or

(iv) R³ is bromo or alkyloxycarbonyl, and R⁴ is hydrogen;

(b) a compound wherein R¹ is substituted or unsubstituted aromatic carbocyclic group or unsubstituted furyl, and R² is substituted or unsubstituted phenyl; and
aromatic carbocyclic group or unsubstituted furyl, and R² is substituted or unsubstituted phenyl; and
(c) the compounds of the formula:

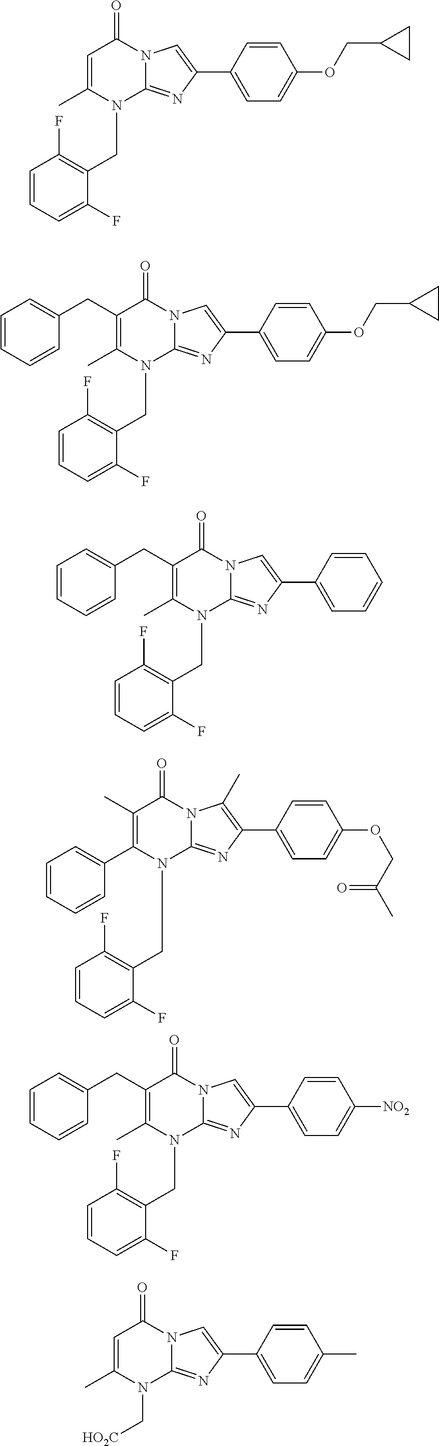

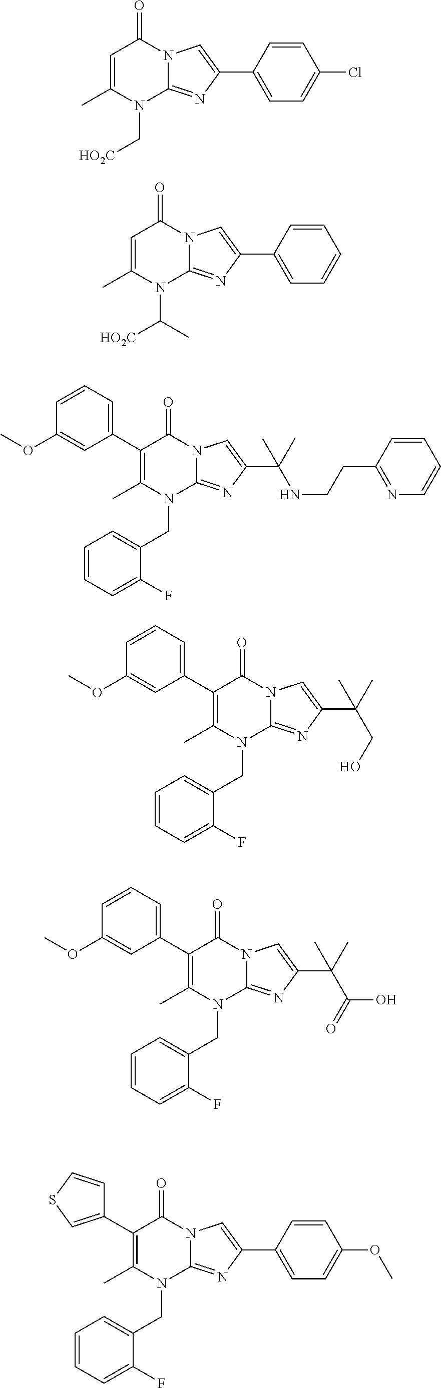

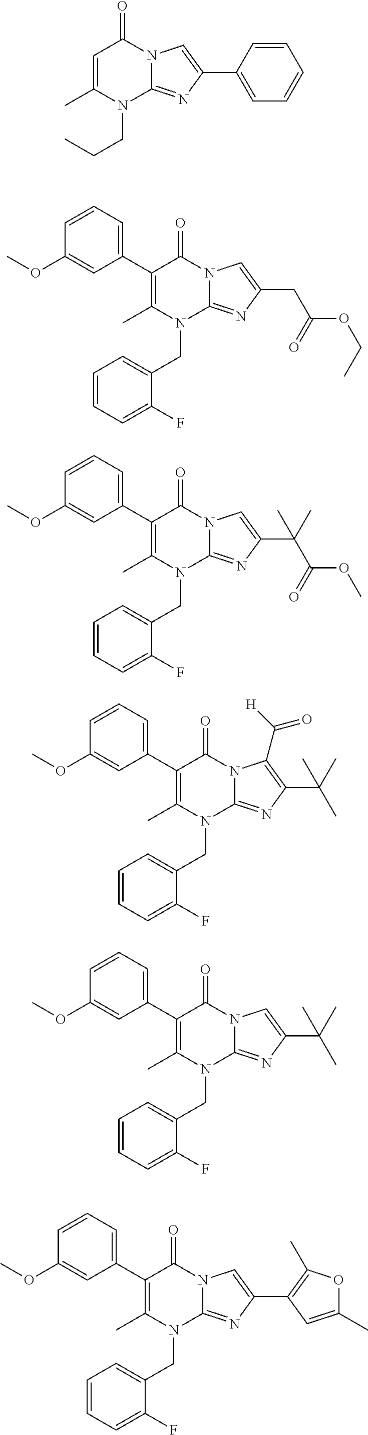

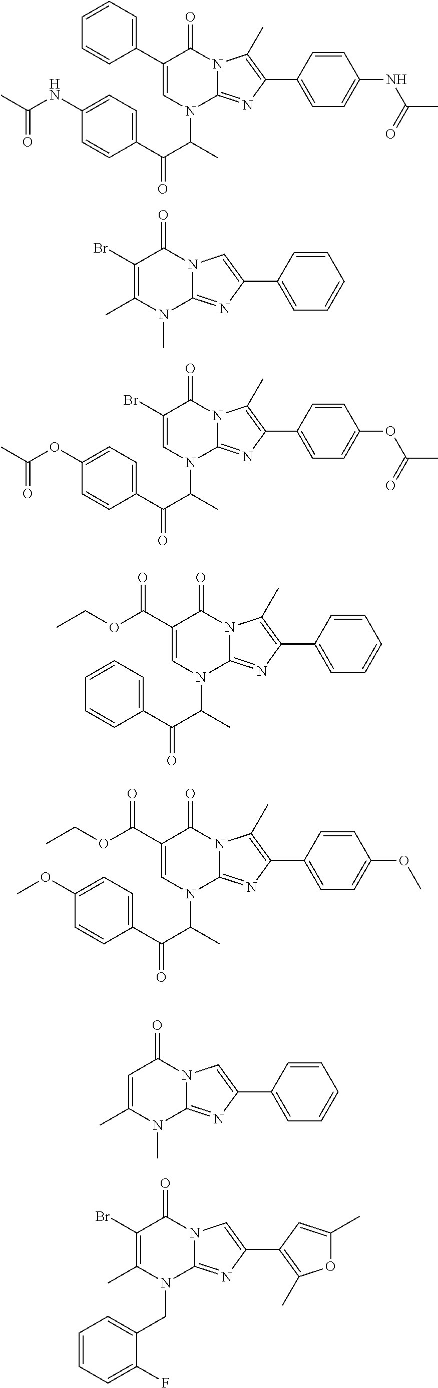

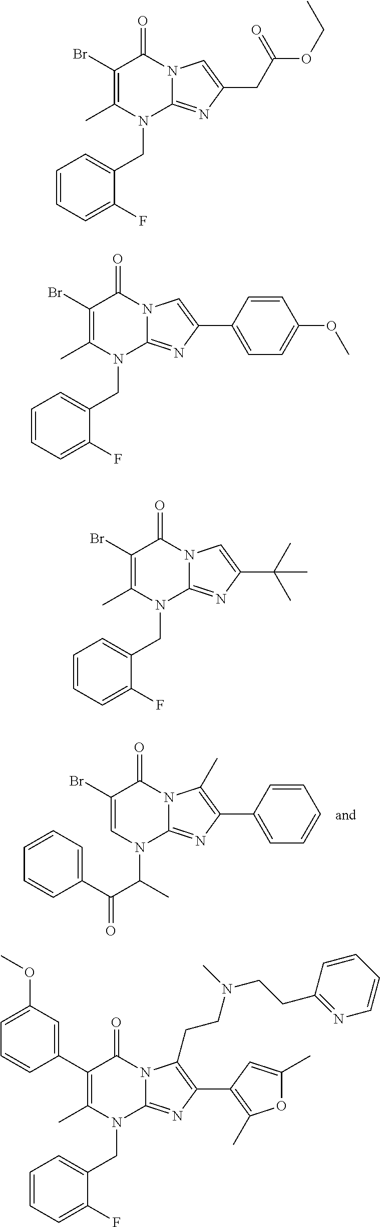

are excluded.
[4] The compound according to [3] or [3′] wherein R⁵ is substituted or unsubstituted C4-C8 alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group, or a pharmaceutically acceptable salt thereof.
[5] The compound according to [3] or [3′] wherein R⁵ is substituted or unsubstituted C4-C8 alkyl, substituted or unsubstituted alkenyl or substituted or unsubstituted alkynyl, or a pharmaceutically acceptable salt thereof.
[6] The compound according to [3] or [3′] wherein R⁵ is alkyl substituted with one or more substituents selected from the Substituent Group A consisting of halogen, cyano, hydroxy, formyl, substituted or unsubstituted non-aromatic carbocyclic group, substituted or unsubstituted non-aromatic heterocyclic group, substituted or unsubstituted aromatic heterocyclic group, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl, substituted or unsubstituted aromatic heterocyclylsulfonyl and substituted or unsubstituted amino, or a pharmaceutically acceptable salt thereof.
[7] The compound according to [3] or [3′] wherein R⁵ is a group of formula:

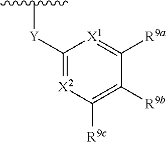

wherein
X¹ and X² are each independently N or CH,
Y is substituted or unsubstituted alkylene, substituted or unsubstituted alkenylene or substituted or unsubstituted alkynylene,
R^{9a}, R^{9b} and R^{9c} are each independently hydrogen, halogen, cyano, hydroxy, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl, substituted or unsubstituted aromatic heterocyclylsulfonyl or substituted or unsubstituted amino,
or a pharmaceutically acceptable salt thereof.
[8] The compound according to any one of [2] to [7] or [3′] wherein R² is hydrogen, halogen, formyl or substituted or unsubstituted alkyl, or a pharmaceutically acceptable salt thereof.
[9] The compound according to any one of [2] to [8] or [3′] wherein R³ is hydrogen, halogen, cyano, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group or substituted or unsubstituted amino, or a pharmaceutically acceptable salt thereof.
[10] The compound according to any one of [2] to [9] or [3′] wherein R⁴ is hydrogen, halogen, formyl, carboxy, substituted or unsubstituted alkyl, substituted or unsubstituted alkenyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted alkyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted carbamoyl or substituted or unsubstituted amino, or a pharmaceutically acceptable salt thereof.
[11] The compound according to any one of claims [2] to [9] or [3′] wherein R⁴ is halogen, formyl, substituted methyl, substituted or unsubstituted C2-C8 alkyl, substituted or unsubstituted alkenyl, substituted or unsubstituted non-aromatic carbocyclic group, substituted aromatic carbocyclic group, substituted or unsubstituted non-aromatic heterocyclic group, substituted or unsubstituted aromatic heterocyclic group, substituted or unsubstituted alkyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted carbamoyl or substituted or unsubstituted amino, or a pharmaceutically acceptable salt thereof.
[12]A pharmaceutical composition comprising the compound according to any one of [2] to [11] or [3′] or a pharmaceutically acceptable salt thereof as an active ingredient.
[13] The pharmaceutical composition according to [12] that has autotaxin inhibitory effect.
[14] The pharmaceutical composition according to [12] or [13] for the prevention or treatment of a disease involving autotaxin.
[15] Use of a compound according to any one of [2] to [11] or [3′] or a pharmaceutically acceptable salt thereof for the manufacture of a medicament for the prevention or treatment of a disease involving autotaxin.
[16]A method for the prevention or treatment of a disease involving autotaxin comprising administering a compound according to any one of [2] to [11] or [3′] or a pharmaceutically acceptable salt thereof.
[17] The compound according to any one of [2] to [11] or [3′] or a pharmaceutically acceptable salt thereof for the prevention or treatment of a disease involving autotaxin.
[18] The pharmaceutical composition according to [12] which is a chronic kidney disease therapeutic agent.
[19]A method for the prevention or treatment of a disease involving autotaxin comprising administering a compound of formula (I):

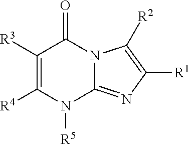

wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
R², R³ and R⁴ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino,
—N═C(R^{4a}) (OR^{4b}) wherein R^{4a} is substituted or unsubstituted alkyl and R^{4b} is substituted or unsubstituted alkyl, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group, or a pharmaceutically acceptable salt thereof.
[20]A compound of formula (I):

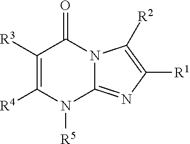

wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
R², R³ and R⁴ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino,
—N═C(R^{4a}) (OR^{4b}) wherein R^{4a} is substituted or unsubstituted alkyl and R^{4b} is substituted or unsubstituted alkyl, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group,
or a pharmaceutically acceptable salt thereof for the prevention or treatment of a disease involving autotaxin.
[21] Use of a compound of formula (I):

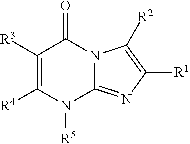

wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
R², R³ and R⁴ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino,
—N═C(R^{4a}) (OR^{4b}) wherein R^{4a} is substituted or unsubstituted alkyl and R^{4b} is substituted or unsubstituted alkyl, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group,
or a pharmaceutically acceptable salt thereof for the manufacture of a medicament for the prevention or treatment of a disease involving autotaxin.

### Effect of the Invention

The compound of the invention exhibits excellent autotaxin inhibitory activity. Also, the compound of the invention prevents fibrosis based on the autotaxin inhibitory activity.

### DESCRIPTION OF EMBODIMENTS

The definitions of the terms as used herein are as follows. Unless specified otherwise, these terms are used alone or in combination with another term in the meaning as defined.

The term “halogen” includes fluorine, chlorine, bromine and iodine. Fluorine and chlorine are particularly preferable.

Specific examples of “halogen” for R² include bromine.

Specific examples of “halogen” for R³ include fluorine.

Specific examples of “halogen” for R⁴ includes chlorine.

The term “alkyl” means a straight or branched hydrocarbon group having 1 to 10 carbon atoms, and includes alkyl of 1 to 6 carbon atoms, alkyl of 1 to 4 carbon atoms, and alkyl of 1 to 3 carbon atoms. Examples include methyl, ethyl, n-propyl, isopropyl, n-butyl, isobutyl, sec-butyl, tert-butyl, n-pentyl, isopentyl, neopentyl, hexyl, isohexyl, n-heptyl, isoheptyl, n-octyl, isooctyl, n-nonyl, and n-decyl.

Specific examples of “alkyl” for R¹ include methyl, ethyl, n-propyl, isopropyl, n-butyl, isobutyl, sec-butyl, and tert-butyl. In particular, n-propyl is preferred.

Specific examples of “alkyl” for R² include methyl, ethyl, n-propyl, isopropyl, n-butyl, isobutyl, sec-butyl, and tert-butyl. In particular, methyl is preferred.

Specific examples of “alkyl” for R³ include methyl, ethyl, n-propyl, isopropyl, n-butyl, isobutyl, sec-butyl, and tert-butyl. In particular, methyl, ethyl, n-propyl, and n-butyl are preferred.

Specific examples of “alkyl” for R⁴ include methyl, ethyl, n-propyl, isopropyl, n-butyl, isobutyl, sec-butyl, and tert-butyl. In particular, methyl and n-propyl are preferred.

Specific examples of “alkyl” for R^{4a} include methyl, ethyl, n-propyl, isopropyl, n-butyl, isobutyl, sec-butyl, and tert-butyl. In particular, methyl is preferred.

Specific examples of “alkyl” for R^{4b} include methyl, ethyl, n-propyl, isopropyl, n-butyl, isobutyl, sec-butyl, and tert-butyl. In particular, methyl, ethyl, and n-propyl are preferred.

Specific examples of “alkyl” for R⁵ include methyl, ethyl, n-propyl, isopropyl, n-butyl, isobutyl, sec-butyl, tert-butyl, n-pentyl, and isohexyl. In particular, methyl, ethyl, n-propyl, n-butyl, n-pentyl, methylbutyl, n-hexyl, isohexyl, and ethylpentyl are preferred.

The alkyl moiety of “alkyloxy”, “alkyloxycarbonyl”, “alkylcarbonyl”, “alkylsulfinyl”, “alkylsulfonyl” and “alkylthio” has the same meaning as defined above for “alkyl”.

Specific examples of the alkyl moiety of “alkyloxy” for R⁴ include methyl, ethyl, n-propyl, isopropyl, n-butyl, isobutyl, sec-butyl, tert-butyl. In particular, methyloxy, ethyloxy, n-propyloxy, isopropyloxy, tert-butyloxy, n-octyloxy, isobutylmethylhexyloxy, and n-nonyloxy are preferred.

The term “haloalkyl” and “haloalkyloxy” mean respectively alkyl and alkyloxy substituted with 1 to 5, preferably 1 to 3, “halogen” at a substitutable position.

Specific examples of “haloalkyl” for R⁵ include monohaloalkyl, dihaloalkyl, and trihaloalkyl. In particular, trifluorobutyl, fluoro-n-butyl, and fluoro-n-hexyl are preferred.

The term “alkenyl” means a linear or branched hydrocarbon group having 2 to 10 carbon atoms and one or more double bonds at any position, and includes alkenyl of 2 to 6 carbon atoms, alkenyl of 3 to 4 carbon atoms. Examples include vinyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, and decenyl.

Specific examples of “alkenyl” for R¹ include vinyl, propenyl, isopropenyl, butenyl, isobutenyl, prenyl, butadienyl, pentenyl, isopentenyl, pentadienyl, hexenyl, and isohexenyl. In particular, propenyl is preferred.

Specific examples of “alkenyl” for R⁵ include vinyl, propenyl, isopropenyl, butenyl, isobutenyl, prenyl, butadienyl, pentenyl, isopentenyl, pentadienyl, hexenyl, and isohexenyl. In particular, butenyl and pentenyl are preferred.

The alkenyl moiety of “alkenyloxy”, “alkenyloxycarbonyl”, “alkenylcarbonyl”, “alkenylsulfinyl”, “alkenylsulfonyl” and “alkenylthio” has the same meaning as defined above for “alkenyl”.

The term “alkynyl” means a linear or branched hydrocarbon group having 2 to 10 carbon atoms and one or more triple bonds at any position, and includes alkynyl of 2 to 6 carbon atoms, alkynyl of 2 to 4 carbon atoms. Examples include ethynyl, propynyl, butynyl, pentynyl, hexynyl, heptynyl, octynyl, nonynyl, and decynyl. Also, the alkynyl may further have a double bond, as well as one or more triple bonds at any position.

Specific examples of “alkynyl” for R³ include ethynyl, propynyl, butynyl, pentynyl, hexynyl, heptynyl, octynyl, nonynyl, and decynyl. In particular, propynyl is preferred.

The alkynyl moiety of “alkynyloxy”, “alkynyloxycarbonyl”, “alkynylcarbonyl”, “alkynylsulfinyl”, “alkynylsulfonyl” and “alkynylthio” has the same meaning as defined above for “alkynyl”.

Preferred examples of “alkynyloxy” for R³ include undecynyloxy.

The term “non-aromatic carbocyclic group” includes cyclic saturated hydrocarbon groups having 3 to 8 carbon atoms, groups wherein such cyclic saturated hydrocarbon ring is fused with further one or two 3- to 8-membered rings, cyclic unsaturated aliphatic hydrocarbon groups having 3 to 8 carbon atoms, and groups wherein such cyclic unsaturated aliphatic hydrocarbon ring is fused with further one or two 3- to 8-membered rings.

Specific examples of the cyclic saturated hydrocarbon group having 3 to 8 carbon atoms include cyclopropyl, cyclobutyl, cyclopentyl, cyclohexyl, cycloheptyl, cyclooctyl. In particular, a cyclic saturated hydrocarbon group having 3 to 6 carbon atoms and a cyclic saturated hydrocarbon group having 5 or 6 carbon atoms are preferred.

Specific examples of the ring to be fused with the cyclic saturated hydrocarbon group having 3 to 8 carbon atoms include non-aromatic carbocyclic rings, such as cycloalkane ring (for example: cyclohexane, cyclopentane) and cycloalkene ring (for example: cyclohexene, cyclopentene); non-aromatic heterocyclic rings, such as piperidine ring, piperazine ring and morpholine ring; aromatic carbocyclic rings, such as benzene ring and naphthalene ring; and aromatic heterocyclic rings, such as pyridine ring, pyrimidine ring, pyrrole ring and imidazole ring. The cyclic saturated hydrocarbon group having 3 to 8 carbon atoms should be involved in the linkage of such fused ring.

Specific examples of the ring to be fused with the cyclic unsaturated aliphatic hydrocarbon group having 3 to 8 carbon atoms include carbocyclic rings: such as aromatic carbocyclic rings (for example: benzene ring, naphthalene ring) and non-aromatic carbocyclic rings (for example: cycloalkane rings such as cyclohexane ring and cyclopentane ring, cycloalkene rings such as cyclohexene ring and cyclopentene ring); and heterocyclic rings: such as aromatic heterocyclic rings (for example: pyridine ring, pyrimidine ring, pyrrole ring, imidazole ring) and non-aromatic heterocyclic rings (for example: piperidine ring, piperazine ring, morpholine ring). The cyclic unsaturated aliphatic hydrocarbon group having 3 to 8 carbon atoms should be involved in the linkage of such fused ring.

Examples of the non-aromatic carbocyclic group include the following groups. These groups may have a substituent group at any substitutable position.

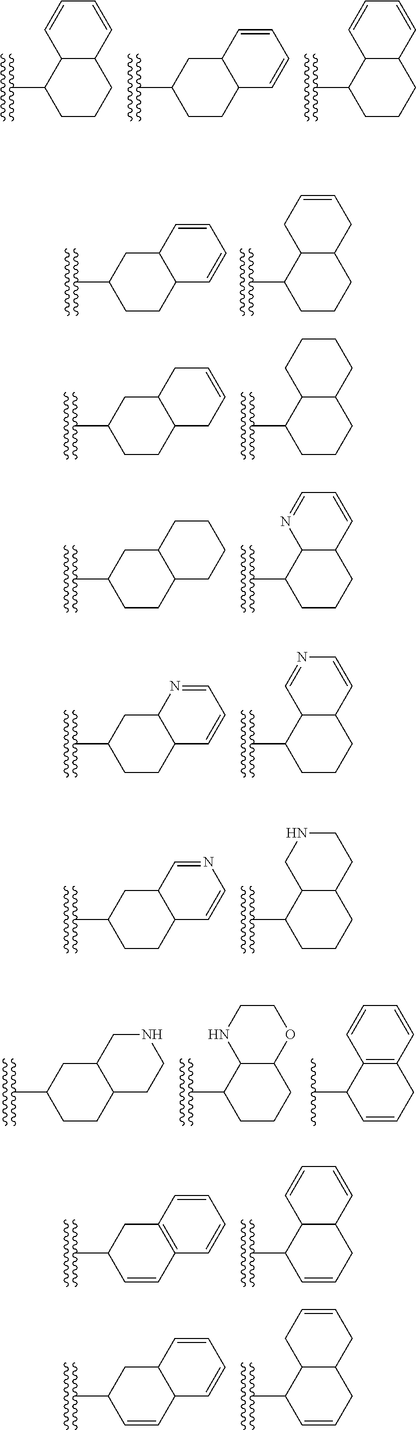

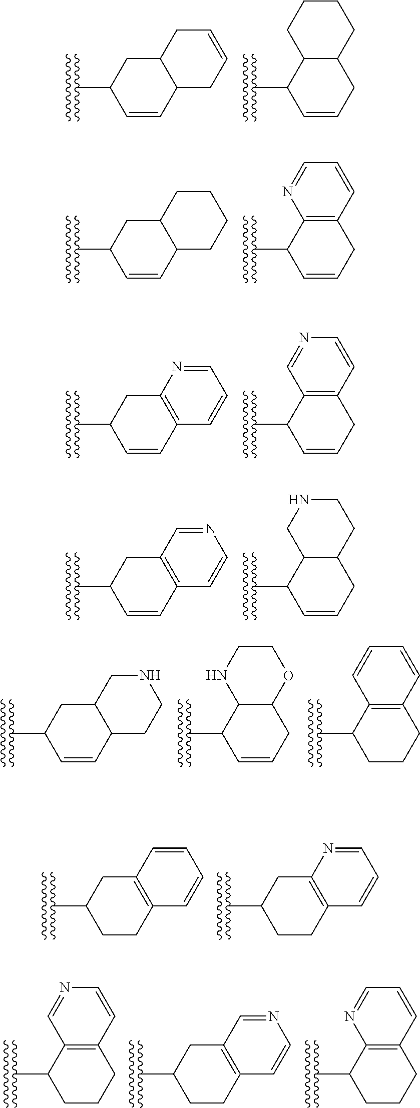

Specific examples of “non-aromatic carbocyclic group” for R¹ include cycloalkyl and cycloalkenyl. In particular, cyclopropyl, cyclobutyl, cyclopentyl and cyclohexyl are preferable.

Specific examples of “non-aromatic carbocyclic group” for R⁴ include cycloalkyl and cycloalkenyl. In particular, cyclopropyl, cyclobutyl, cyclopentyl, cyclohexyl, cyclopentenyl and cyclohexenyl are preferable.

The non-aromatic carbocyclic ring moiety of “non-aromatic carbocyclyloxy”, “non-aromatic carbocyclyloxycarbonyl”, “non-aromatic carbocyclylcarbonyl”, “non-aromatic carbocyclylsulfinyl”, “non-aromatic carbocyclylsulfonyl” and “non-aromatic carbocyclylthio” has the same meaning as defined above for “non-aromatic carbocyclic group”.

Specific examples of “non-aromatic carbon ring oxy” for R⁴ include cycloalkyloxy and cycloalkenyloxy. In particular, cyclopropyloxy, cyclobutyloxy, cyclopentyloxy and cyclohexyloxy are preferable.

The term “aromatic carbocyclic group” includes monocyclic or polycyclic aromatic carbocyclic groups and groups wherein such monocyclic or polycyclic aromatic carbocyclic ring is fused with further one or two 3- to 8-membered rings. Specific examples of the monocyclic or polycyclic aromatic carbocyclic group include phenyl, naphthyl, anthryl and phenanthryl. Particularly, phenyl is preferred.

Specific examples of the ring to be fused with the monocyclic or polycyclic aromatic carbocyclic group include non-aromatic carbocyclic rings such as cycloalkane rings (for example: cyclohexane ring, cyclopentane ring), and cycloalkene rings (for example: cyclohexene ring, cyclopentene ring); and non-aromatic heterocyclic rings such as piperidine ring, piperazine ring and morpholine ring. The monocyclic or polycyclic aromatic carbocyclic group should be involved in the linkage of such fused ring.

Examples of the aromatic carbocyclic groups include the following groups. These groups may have a substituent group at any possible position.

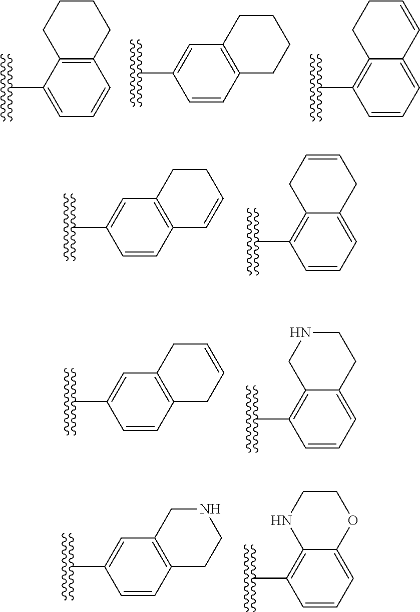

Specific examples of “aromatic carbocyclic group” for R¹ include phenyl, naphthyl, anthryl and phenanthryl. In particular, phenyl is preferred.

Specific examples of “aromatic carbocyclic group” for R³ include phenyl, naphthyl, anthryl and phenanthryl. In particular, phenyl is preferred.

The aromatic carbocyclic ring moiety of “aromatic carbocyclyloxy”, “aromatic carbocyclyloxycarbonyl”, “aromatic carbocyclylcarbonyl”, “aromatic carbocyclylcarbonyl”, “aromatic carbocyclylsulfinyl”, “aromatic carbocyclylsulfonyl” and “aromatic carbocyclylthio” has the same meaning as defined above for “aromatic carbocyclic ring”.

Preferred examples of “aromatic carbocyclyloxy” for R⁴ include phenyloxy and naphthyloxy.

The term “aromatic heterocyclic group” means monocyclic or polycyclic aromatic heterocyclic groups having one or more heteroatoms selected from O, S and N in the ring and groups wherein such monocyclic or polycyclic aromatic heterocyclic ring is fused with further one or two 3- to 8-membered rings.

Preferred examples of the monocyclic aromatic heterocyclic group include 5- or 6-membered aromatic heterocyclic groups such as pyrrolyl, imidazolyl, pyrazolyl, pyridyl, pyridazinyl, pyrimidinyl, pyrazinyl, triazolyl, triazinyl, tetrazolyl, isoxazolyl, oxazolyl, oxadiazolyl, isothiazolyl, thiazolyl, thiadiazolyl, furyl and thienyl.

Preferred examples of the polycyclic aromatic heterocyclic group include aromatic heterocyclic groups fused with a 5- or 6-membered ring, such as bicyclic aromatic heterocyclic group (for example: indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzoisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, oxazolopyridyl, thiazolopyridyl), and tricyclic aromatic heterocyclic group (for example: carbazolyl, acridinyl, xanthenyl, phenothiazinyl, phenoxathiinyl, phenoxazinyl, dibenzofuryl). Any ring of the polycyclic aromatic heterocyclic group may be involved in the linkage.

Specific examples of the ring to be fused with the monocyclic or polycyclic aromatic heterocyclic groups include non-aromatic carbocyclic rings such as cycloalkane ring (for example: cyclohexane ring, cyclopentane ring), cycloalkene rings (for example: cyclohexene ring, cyclopentene ring); non-aromatic heterocyclic rings such as piperidine ring, piperazine ring and morpholine ring. The monocyclic or polycyclic aromatic heterocyclic group should be involved in the linkage of such fused ring.

Examples of the aromatic heterocyclic groups include the following groups. These groups may have a substituent group at any possible position.

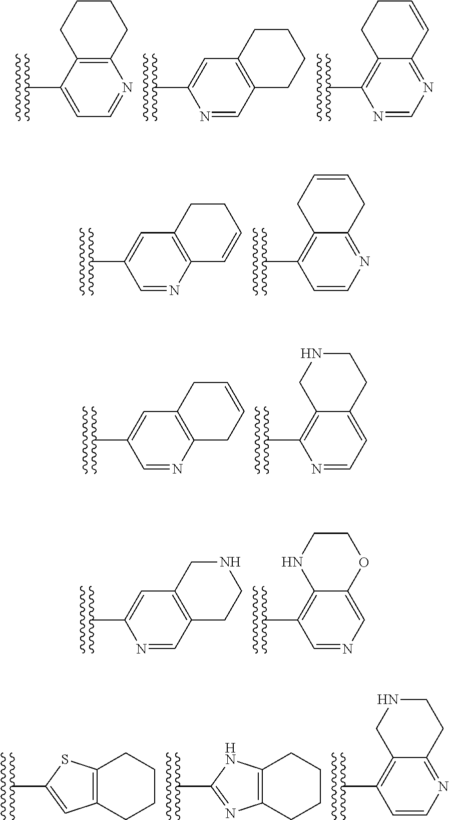

Specific examples of “aromatic heterocyclic group” for R⁴ include bicyclic heterocyclic groups aromatic ring such as pyrrolyl, imidazolyl, pyrazolyl, pyridyl, pyridazinyl, pyrimidinyl, pyrazinyl, triazolyl, triazinyl, tetrazolyl, isoxazolyl, oxazolyl, oxadiazolyl, isothiazolyl, thiazolyl, thiadiazolyl, furyl, thienyl, indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzoisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, oxazolopyridyl, thiazolopyridyl; and carbazolyl, acridinyl, xanthenyl, phenothiazinyl, phenoxathiinyl, phenoxazinyl and dibenzofuryl. In particular, furyl, thiazolyl, pyrazolyl, pyridyl, pyrimidinyl, pyrazinyl, benzofuryl and benzothiophenyl are preferable.

The aromatic heterocyclic ring moiety of “aromatic heterocyclyloxy”, “aromatic heterocyclyloxycarbonyl”, “aromatic heterocyclylcarbonyl”, “aromatic heterocyclylsulfinyl”, “aromatic heterocyclylsulfonyl” and “aromatic heterocyclylthio” has the same meaning as defined above for “aromatic heterocyclic group”.

The term “non-aromatic heterocyclic group” means monocyclic or polycyclic non-aromatic heterocyclic groups having one or more heteroatoms selected from O, S and N in the ring and groups wherein such non-aromatic heterocyclic ring is fused with further one or two 3- to 8-membered rings.

Specific examples of the monocyclic non-aromatic heterocyclic group include dioxanyl, thiiranyl, oxiranyl, oxathiolanyl, azetidinyl, thianyl, pyrrolidinyl, pyrrolinyl, imidazolidinyl, imidazolinyl, pyrazolidinyl, pyrazolinyl, piperidyl, piperidino, piperazinyl, piperazino, morpholinyl, morpholino, oxadiadinyl, dihydropyridyl, thiomorpholinyl, thiomorpholino, tetrahydrofuryl, tetrahydropyranyl, tetrahydrothiazolyl, tetrahydroisothiazolyl, oxazolidyl and thiazolidyl.

Specific examples of the polycyclic non-aromatic heterocyclic group include indolinyl, isoindolinyl, chromanyl, isochromanyl and isomannyl. Any ring of the polycyclic non-aromatic heterocyclic group may be involved in the linkage.

Examples of the non-aromatic heterocyclic groups include the following groups.

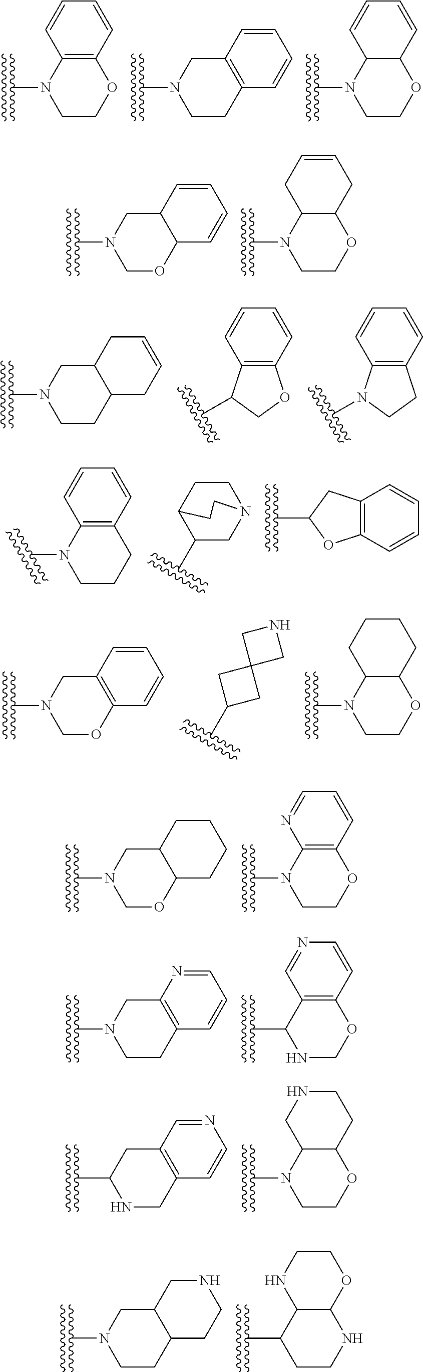

Specific examples of “non-aromatic heterocyclic group” for R⁴ include dioxanyl, thiiranyl, oxiranyl, oxathiolanyl, azetidinyl, thianyl, pyrrolidinyl, pyrrolinyl, imidazolidinyl, imidazolinyl, pyrazolidinyl, pyrazolinyl, piperidyl, piperidino, piperazinyl, piperazino, morpholinyl, morpholino, oxadiadinyl, dihydropyridyl, thiomorpholinyl, thiomorpholino, tetrahydrofuryl, tetrahydropyranyl, tetrahydrothiazolyl, tetrahydroisothiazolyl, oxazolidyl, thiazolidyl and azepanyl. In particular, azetidinyl, piperidinyl, piperazinyl, morpholinyl, morpholino and azepanyl are preferable.

The non-aromatic heterocyclic ring moiety of “non-aromatic heterocyclyloxy”, “non-aromatic heterocyclyloxycarbonyl”, “non-aromatic heterocyclylcarbonyl”, “non-aromatic heterocyclylsulfinyl”, “non-aromatic heterocyclylsulfonyl” and “non-aromatic heterocyclylthio” has the same meaning as defined above for “non-aromatic heterocyclic group”.

Preferred examples of “non-aromatic heterocyclyloxy” for R⁴ include piperidinyloxy.

The substituted or unsubstituted non-aromatic carbocyclic groups and the substituted or unsubstituted non-aromatic heterocyclic groups are optionally substituted with one or two oxo, thioxo or substituted or unsubstituted imino.

Examples of the substituent group for “substituted alkyl”, “substituted alkenyl”, “substituted alkynyl”, “substituted non-aromatic carbocyclic group”, “substituted aromatic carbocyclic group”, “substituted aromatic heterocyclic group” and “substituted non-aromatic heterocyclic group” include halogen, hydroxy, mercapto, nitro, nitroso, cyano, azido, formyl, amino, carboxy, alkyl, haloalkyl, alkenyl, alkynyl, non-aromatic carbocyclic group, aromatic carbocyclic group, aromatic heterocyclic group, non-aromatic heterocyclic group, substituted carbamoyl, substituted sulfamoyl, substituted amidino, a group of formula: —O—R^{x}, a group of formula: —O—C(═O)—R^{x}, a group of formula: —C(═O)—R^{x}, a group of formula: —C(═O)—O—R^{x}, a group of formula: —S—R^{x} or a group of formula: —SO₂—R^{x} wherein R^{x} is alkyl, haloalkyl, alkenyl, alkynyl, non-aromatic carbocyclic group, aromatic carbocyclic group, aromatic heterocyclic group, non-aromatic heterocyclic group, carbamoyl, sulfamoyl or amidino. One or more of these substituent groups may occur at any substitutable position.

Specific examples of the substituent group for “substituted alkyl” in R² include hydroxy, amino and alkylamino.

Specific examples of the substituent group for “substituted alkyl” in R³ include hydroxy, carboxy, aromatic carbocyclic group, alkylcarbonylamino, alkyloxy, alkyloxycarbonyl, alkylaminocarbonyl.

Specific examples of the substituent group for “substituted alkyl” in R⁴ include hydroxy, phenylalkyloxy and phenylcarbonyloxy.

Specific examples of the substituent group for “substituted alkyl” in R⁵ include halogen, hydroxy, cyano, alkyloxy, non-aromatic carbocyclic group, aromatic carbocyclic group, halo aromatic carbocyclic group, alkyl aromatic carbocyclic group, trihaloalkyl aromatic carbocyclic group, trihaloalkyloxy aromatic carbocyclic group, carboxy aromatic carbocyclic group, alkyloxycarbonyl aromatic carbocyclic group, alkyloxycarbonylalkyl aromatic carbocyclic group, alkylaminoalkyloxy aromatic carbocyclic group, aromatic heterocyclyl-aromatic carbocyclic group, aromatic heterocyclyloxy-aromatic carbocyclic group, alkylsulfonyl aromatic carbocyclic group, aromatic carbocyclyloxy-aromatic carbocyclic group, non-aromatic heterocyclylalkyloxy aromatic carbocyclic group, aromatic carbocyclyloxy-aromatic carbocyclic group, aromatic carbocyclyloxyalkyl aromatic carbocyclic groups, aromatic carbocyclyl-aromatic carbocyclic group, dihaloalkylsulfonyl, aromatic heterocyclic group, alkylcarbonyl, alkyloxycarbonyl, non-aromatic carbocyclylcarbamoyl, alkylaminocarbonyl, alkylcarbonyloxy, alkylamino, carboxyalkyloxy and alkylsulfonyloxy.

Specific examples of the substituent group for “substituted alkyloxy” in R⁴ include alkyloxy, aromatic carbocyclic group, alkylcarbonyl-aromatic carbocyclic group, non-aromatic carbocyclic group, halo non-aromatic carbocyclic group and alkyloxycarbonyl-non-aromatic heterocyclic group.

Specific examples of the substituent group for “substituted alkenyl” in R⁴ include aromatic carbocyclic group.

Specific examples of the substituent group for “substituted alkenyl” in R⁵ include halogen.

Specific examples of the substituent group for “substituted alkynyl” in R³ include hydroxy.

Specific examples of the substituent group for “substituted alkynyl” in R⁴ include alkyloxy.

Specific examples of the substituent group for “substituted aromatic carbocyclic group” in R¹ include halogen, cyano, carboxy, trihaloalkyl, non-aromatic carbocyclic group, alkyloxy, dihaloalkyloxy, aromatic carbocyclyloxy, alkylamino, alkyloxycarbonyl and non-aromatic heterocyclic group.

Specific examples of the substituent group for “substituted aromatic carbocyclic group” in R⁴ include cyano, halogen, hydroxy, carboxy, sulfo, amino, alkyl, hydroxyalkyl, alkyloxyalkyl, alkyloxy, hydroxyalkyloxy, halo aromatic carbocyclic group, alkyl non-aromatic heterocyclic group, alkylcarbonylaminoalkyl non-aromatic heterocyclic group, alkylthio, alkylcarbonyl, alkyloxycarbonyl, non-aromatic heterocyclylcarbonyl, alkyloxy non-aromatic heterocyclylcarbonyl, alkylcarbonyl non-aromatic heterocyclylcarbonyl, hydroxy non-aromatic heterocyclylcarbonyl, alkylsulfonyl non-aromatic heterocyclylcarbonyl, haloalkylaminocarbonyl, hydroxyalkylaminocarbonyl, alkylaminocarbonyl, aminoalkylaminocarbonyl, hydroxyalkylaminocarbonyl, aminosulfonylalkylaminocarbonyl, alkylsulfonylalkylaminocarbonyl, carbamoyl, alkylcarbamoyl, haloalkylcarbamoyl, cyanoalkylcarbamoyl, hydroxyalkylcarbamoyl, non-aromatic heterocyclylalkylcarbamoyl, alkyl non-aromatic heterocyclylalkylcarbamoyl, alkylcarbamoyl, non-aromatic carboncyclylalkylcarbamoyl, aminoalkylcarbamoyl, hydroxyalkylcycloalkylcarbamoyl, non-aromatic heterocyclylaminoalkylcarbamoyl, alkyloxyalkylcarbamoyl, alkylaminoalkylcarbamoyl, hydroxyalkylcarbamoyl, hydroxyalkyloxyalkylcarbamoyl, hydroxyalkyl(alkyl)carbamoyl, dihydroxyalkylcarbamoyl, alkylcarbonylalkylcarbamoyl, non-aromatic heterocyclylcarbonylalkylcarbamoyl, alkylcarbonylaminoalkylcarbamoyl, alkylsulfonylalkylcarbamoyl, sulfamoyl aromatic carbocyclylalkyl, alkylsulfonyl aromatic heterocyclylalkyl, aromatic heterocyclyl-aromatic heterocyclylalkyl, non-aromatic heterocyclylsulfonylalkylcarbamoyl, sulfamoyl alkylcarbamoyl, nitro aromatic carbocyclylalkyl, non-aromatic carbocyclylcarbamoyl, alkyloxy aromatic carbocyclylcarbamoyl, aromatic heterocyclylalkylcarbamoyl, alkyl non-aromatic carbocyclyl-carbamoyl, hydroxyalkyl non-aromatic carbocyclylcarbamoyl, non-aromatic heterocyclylcarbamoyl, alkylsulfonyl, aminosulfonyl, alkylaminosulfonyl, hydroxyalkylaminosulfonyl, non-aromatic heterocyclylsulfonyl, alkylamino, alkylcarbonylamino, non-aromatic heterocyclylcarbonylamino and alkylsulfonylamino.

Specific examples of the substituent group for “substituted amino”, “substituted carbamoyl”, “substituted sulfamoyl”, “substituted amidino” and “substituted imino” include hydroxy, cyano, formyl, alkyl, haloalkyl, alkenyl, alkynyl, non-aromatic carbocyclic group, aromatic carbocyclic group, aromatic heterocyclic groups, non-aromatic heterocyclic group, carbamoyl, sulfamoyl, amidino, a group of formula: —O—R, a group of formula: —C(═O)—R, a group of formula: —C(═O)—O—R and a group of formula: —SO₂—R wherein R is alkyl, haloalkyl, alkenyl, alkynyl, non-aromatic carbocyclic group, aromatic carbocyclic group, aromatic heterocyclic group or non-aromatic heterocyclic group. One or two of these substituent groups may occur at any substitutable position.

Specific examples of the substituent group for “substituted amino” in R⁴ include alkyl, hydroxyalkyl, alkyloxyalkyl, carboxyalkyl, alkylaminoalkyl, aromatic carbocyclylalkyl, alkyloxy aromatic carbocyclylalkyl, alkyloxycarbonylalkyl, carboxy aromatic carbocyclylalkyl, alkylamino aromatic carbocyclylalkyl, methylenedioxy aromatic carbocyclylalkyl, aromatic heterocyclylalkyl, alkyl aromatic heterocyclylalkyl, non-aromatic heterocyclylalkyl, alkyl non-aromatic heterocyclylamino, alkylcarbonylaminoalkyl, non-aromatic carbocyclic groups and alkylaminosulfonyl.

Preferred embodiments of the invention are described below.

Preferred embodiments of the substituent groups for R¹ to R⁵ in formula (I) are described below. Compounds having possible combination of the substituent groups in the following (Ia) to (Io) are preferable.

R¹ is, preferably, (Ia) substituted or unsubstituted alkyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group; more preferably, (Ib) substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group; particularly, (Ic) non-aromatic carbocyclic groups optionally substituted with one or more substituents selected from Substituent Group B consisting of halogen, cyano, alkyl substituted with halogen, alkyl substituted with 1-6 halogens and alkyloxy substituted with 1-6 halogens, aromatic carbocyclic groups optionally substituted with one or more substituents selected from Substituent Group B, non-aromatic heterocyclic groups optionally substituted with one or more substituents selected from Substituent Group B, or aromatic heterocyclic groups optionally substituted with one or more substituents selected from Substituent Group B.
R² is, preferably, (Id) hydrogen, halogen, hydroxy, formyl, carboxy, cyano or substituted or unsubstituted alkyl; more preferably, (Id) halogen or substituted or unsubstituted alkyl; particularly, (Ie) hydrogen.
R³ is, preferably, (If) hydrogen, halogen, cyano, carboxy, substituted or unsubstituted alkyl, substituted or unsubstituted alkenyl, substituted or unsubstituted alkynyl, or substituted or unsubstituted amino; more preferably, (Ig) hydrogen, substituted or unsubstituted alkyl, substituted or unsubstituted alkenyl or substituted or unsubstituted alkynyl; particularly, (Ih) hydrogen.
R⁴ is, preferably, (Ii) substituted or unsubstituted alkyl, substituted or unsubstituted alkyloxy, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group or substituted or unsubstituted amino; more preferably, (Ik) substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group; particularly, (Il) substituted or unsubstituted aromatic carbocyclic group or substituted or unsubstituted aromatic heterocyclic group.
R⁵ is, preferably, (Im) C1-C3 alkyl or C4-C8 alkyl substituted with one or more substituents selected from Substituent Group A consisting of halogen, cyano, hydroxy, formyl, substituted or unsubstituted non-aromatic carbocyclic group, substituted or unsubstituted non-aromatic heterocyclic group, substituted or unsubstituted aromatic heterocyclic group, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl, substituted or unsubstituted aromatic heterocyclylsulfonyl, and substituted or unsubstituted amino; more preferably (In) C1-C8 alkyl optionally substituted with one or more substituents selected from Substituent Group C consisting of halogen, cyano, substituted or unsubstituted alkylcarbonyl and substituted or unsubstituted alkyloxycarbonyl; particularly (Io) C4-C8 alkyl optionally substituted with one or more substituents selected from Substituent Group C.

The compounds of formula (I) are not limited to specific isomers and include all possible isomers (e.g., keto-enol isomers, imine-enamine isomers, diastereoisomers, enantiomers, rotamers or the like), racemates or mixtures thereof.

One or more hydrogen, carbon and/or other atoms in the compounds of formula (I) may be replaced with isotopes of hydrogen, carbon and/or other atoms respectively. Examples of the isotopes include hydrogen, carbon, nitrogen, oxygen, phosphorus, sulfur, fluorine, iodine and chlorine, such as ²H, ³H, ¹¹C, ¹³C, ¹⁴C, ¹⁵N, ¹⁸O, ¹⁷O, ³¹P, ³²P, ³⁵S, ¹⁸F, ¹²³I and ³⁶Cl respectively. The compounds of formula (I) include compounds replaced with these isotopes. The compounds replaced with the above isotopes are useful as pharmaceuticals and include all of radiolabeled compounds of the compound of formula (I). The present invention also includes a method of radiolabeling in the manufacture of the radiolabeled compounds. Such radiolabeled compounds are useful in the studies for metabolized drug pharmacokinetics and binding assay and also as a diagnostic tool.

A radiolabeled compound of the compounds of formula (I) can be prepared using methods well-known in the art. For example, a tritium-labeled compound of formula (I) can be prepared by introducing a tritium into a compound of formula (I), through a catalytic dehalogenation using a tritium. This method comprises reacting with an appropriately-halogenated precursor of the compound of formula (I) with tritium gas in the presence of an appropriate catalyst, such as Pd/C, and in the presence or absent of a base. The other appropriate methods for preparing a tritium-labeled compound can be found in “Isotopes in the Physical and Biomedical Sciences, Vol. 1, Labeled Compounds (Part A), Chapter 6 (1987)”. A ¹⁴C-labeled compound can be prepared by using a raw material having ¹⁴C carbon.

Pharmaceutically acceptable salts of the compounds of formula (I) include, for example, salts with alkaline metals such as lithium, sodium, potassium and the like; alkaline earth metals such as calcium, barium and the like; magnesium; transition metals such as zinc, iron and the like; ammonium; organic bases such as trimethylamine, triethylamine, dicyclohexylamine, ethanolamine, diethanolamine, triethanolamine, meglumine, ethylenediamine, pyridine, picoline, quinoline and the like; amino acids; inorganic acids such as hydrochloric acid, sulfuric acid, nitric acid, carbonic acid, hydrobromic acid, phosphoric acid, hydroiodic acid and the like; and organic acids such as formic acid, acetic acid, propionic acid, trifluoroacetic acid, citric acid, lactic acid, tartaric acid, oxalic acid, maleic acid, fumaric acid, mandelic acid, glutaric acid, malic acid, benzoic acid, phthalic acid, ascorbic acid, benzenesulfonic acid, p-toluenesulfonic acid, methanesulfonic acid, ethane sulfonic acid and the like, particularly salts with hydrochloric acid, sulfuric acid, phosphoric acid, tartaric acid and methanesulfonic acid. These salts can be formed according to conventional methods.

The compounds of formula (I) of the invention or salts thereof may exist in a form of solvate (e.g., hydrates or the like) and/or crystal polymorphs. The present invention encompasses those various solvates and crystal polymorphs. The “solvates” may be those wherein any numbers of solvent molecules (e.g., water molecules or the like) are coordinated with the compounds of formula (I). When the compounds of formula (I) or pharmaceutically acceptable salts thereof are allowed to stand in the atmosphere, the compounds may absorb water, resulting in attachment of adsorbed water or formation of hydrates. Recrystallization of the compounds of formula (I) or pharmaceutically acceptable salts thereof may produce crystal polymorphs.

The compounds of formula (I) of the invention may form prodrugs. Such prodrugs are encompassed by the present invention. Prodrugs are derivatives of the compounds of the invention with a chemically or metabolically degradable group(s), and the compounds are converted to a pharmaceutically active compound of the invention through solvolysis or under physiological conditions in vivo. The prodrugs include compounds that are converted to a compound of the invention through enzymatic oxidation, reduction, hydrolysis or the like under physiological conditions in vivo, compounds that are converted to a compound of the invention through hydrolysis by gastric acid, and the like. Methods for selecting and preparing suitable prodrug derivatives are described in, for example, “Design of Prodrugs, Elsevier, Amsterdam, 1985”. The prodrugs themselves may have some activity.

in case where the compound or a pharmaceutically acceptable salt thereof of the invention has hydroxyl group(s), the prodrugs may be acyloxy derivatives and sulfonyloxy derivatives that are prepared by, for example, reacting a compound having hydroxyl group(s) with suitable acyl halide, suitable acid anhydride, suitable sulfonyl chloride, suitable sulfonyl anhydride or mixed anhydride, or by reacting with a condensing agent. Examples include CH₃COO—, C₂H₅COO—, tert-BuCOO—, C₁₅H₃₁COO—, PhCOO—, (m-NaOOCPh)COO—, NaOOCCH₂CH₂COO—, CH₃CH(NH₂)COO—, CH₂N(CH₃)₂COO—, CH₃SO₃—, CH₃CH₂SO₃—, CF₃SO₃—, CH₂FSO₃—, CF₃CH₂SO₃—, p-CH₃O-PhSO₃—, PhSO₃— and p-CH₃PhSO₃—.

The term “chronic kidney disease” means a condition where either or both of

kidney disorder (urine abnormalities such as proteinuria, e.g., microalbuminuria, abnormal urinary sediment, abnormal finding of clinical imaging such as single kidney and polycystic kidney disease, decreased renal function such as increased serum creatinine, electrolyte abnormalities such as hypokalemia due to tubular damage, and abnormal finding of renal tissue biopsy) and
(2) deterioration in renal function less than 60 mL/min/1.73 m² of GFR (glomerular filtration rate)
is present for over three months.

The compounds of the invention are produced according to general procedures as described below. Also, the compounds of the invention can be prepared according to other methods based on the knowledge in Organic Chemistry.

### Preparation of Compound a4

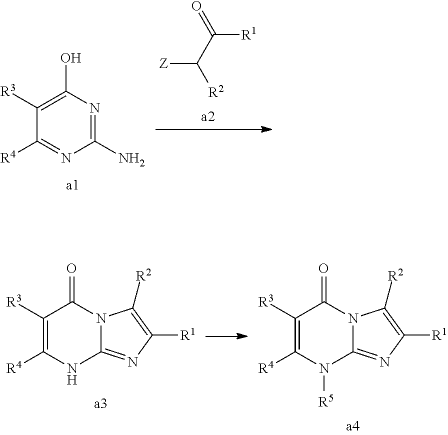

wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group; R², R³ and R⁴ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl, or substituted or unsubstituted aromatic heterocyclylsulfonyl; R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, or substituted or unsubstituted aromatic heterocyclic group; Z is a leaving group such as Cl, Br, I.

### Step 1

The compound a2 is reacted in a solution of Compound a1 in the presence or absence of a base to obtain Compound a3.

Examples of Compound a2 include halides and alkyloxysulfonyl compounds, and Compound a2 may be used in 1 to 10 equivalents, preferably 1 to 3 equivalents.

Examples of the base include sodium hydride, and the base may be used in 1 to 5 equivalents of Compound a1.

Examples of the solvent include N,N-dimethylformamide, N,N-dimethylacetamide and N-methylpyrrolidone.

The reaction temperature may be room temperature to 200° C., preferably from room temperature to reflux temperature.

The reaction time may be 0.1 to 24 hours, preferably 1 to 12 hours.

### Step 2

The compound a4 can be obtained by reacting the solution of Compound a3 with an alkylating agent in the presence of a base.

Examples of the alkylating agent include haloalkyl and alkyltriflate, and the alkylating agent may be used in 1 to 5 equivalents of Compound a3.

Examples of the base include cesium carbonate, potassium carbonate, sodium hydride and tetrabutylammonium fluoride, and the base may be used in 1 to 10 equivalents, preferably 3 to 5 equivalents of Compound a3.

Examples of the solvent include N,N-dimethylformamide, N,N-dimethylacetamide, N-methylpyrrolidone and tetrahydrofuran.

The reaction temperature may be room temperature to 200° C., preferably room temperature to reflux temperature.

The reaction time may be 0.1 to 24 hours, preferably 1 to 12 hours.

### Preparation of Compound a2

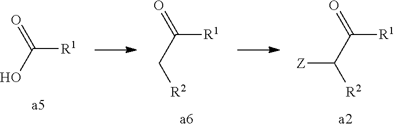

wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
R² is hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkylnylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl, or substituted or unsubstituted aromatic heterocyclylsulfonyl; Z is a leaving group such as Cl, Br, I.

### Step 1

The compound a6 can be obtained by reacting a solution of Compound a5 with an alkyl metal in the presence or absence of silane compound.

Examples of the alkyl metal include methyl lithium, and the alkyl metal may be used in 1 to 10 equivalents, preferably 3 to 5 equivalents of Compound a5.

Examples of the silane compound include trimethylsilyl chloride and trimethylsilyl bromide, and the silane compound may be used in 1 to 30 equivalents, preferably 5 to 15 equivalents of Compound a5.

Examples of the solvent include tetrahydrofuran, diethyl ether and dimethoxyethane.

The reaction temperature may be −20° C. to 50° C., preferably a temperature under ice-cooling to room temperature.

The reaction time may be 0.1 to 24 hours, preferably 1 to 5 hours.

### Step 2

The compound a2 can be obtained by reacting the solution of Compound a6 with a brominating agent.

Examples of the brominating agent include bromine, tetrabutylammonium tribromide, pyridinium tribromide, N-bromosuccinimide, and the brominating agent may be used in 1 to 10 equivalents, preferably in 1 to 5 equivalents of Compound a6.

Examples of the solvent include methanol, acetonitrile, chloroform, methylene chloride, acetic acid and tetrahydrofuran.

The reaction temperature may be −20° C. to 50° C., preferably 0° C. to room temperature.

The reaction time may be 0.1 to 24 hours, preferably 1 to 12 hours.

### Preparation of Compound a9

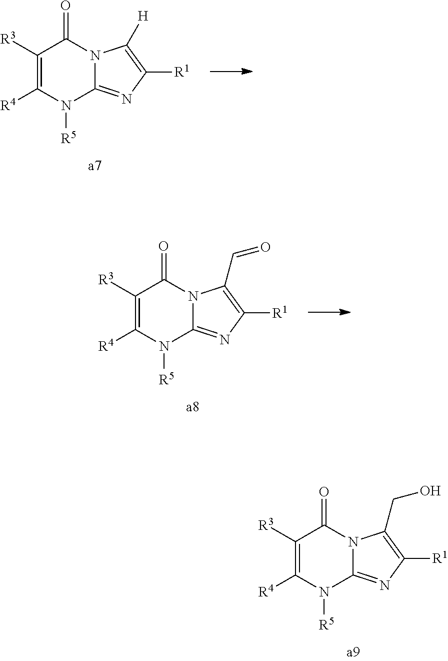

wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group; R³ and R⁴ are each independently hydrogen, halogen, hydroxy, cyano, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, a substituted or unsubstituted non-aromatic heterocyclic group, substituted or unsubstituted aromatic heterocyclic group, substituted or unsubstituted amino, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, non-aromatic substituted or unsubstituted carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group.

### Step 1

The compound 8 can be obtained by reacting a solution of Compound a7 with a formylating agent.

Examples of the solvent include N,N-dimethylformamide, N,N-dimethylacetamide, N-methylpyrrolidone and 1,2-dichloroethane.

Examples of the formylating agent include (Chloromethylene)dimethyliminium chloride, or N,N-dimethylformamide or N-methyl-N-phenylformamide in combination with phosphorous oxychloride, and the formylating agent may be used in 1 to 5 equivalents, preferably, 1 to 3 equivalents of Compound a7.

The reaction temperature may be −20° C. to 50° C., preferably 0° C. to room temperature.

The reaction time may be 0.1 to 10 hours, preferably 1 to 5 hours.

### Step 2

The compound a9 can be obtained by reacting Compounds a8 with a reducing agent.

Examples of the reducing agent include sodium borohydride, lithium borohydride, and lithium aluminum hydride, and the reducing agent may be used in 0.05 to 10 molar equivalents, preferably 0.1 to 3 equivalents of Compound a8.

Examples of the reaction solvent include methanol, ethanol, propanol, isopropanol, butanol, tetrahydrofuran, diethyl ether, dichloromethane and water, and the solvent may be used alone or in combination.

The reaction temperature may be 0° C. to reflux temperature, preferably 20° C. to room temperature.

The reaction time may be 0.2 to 24 hours, preferably 0.5 to 2 hours.

### Preparation of Compound a12

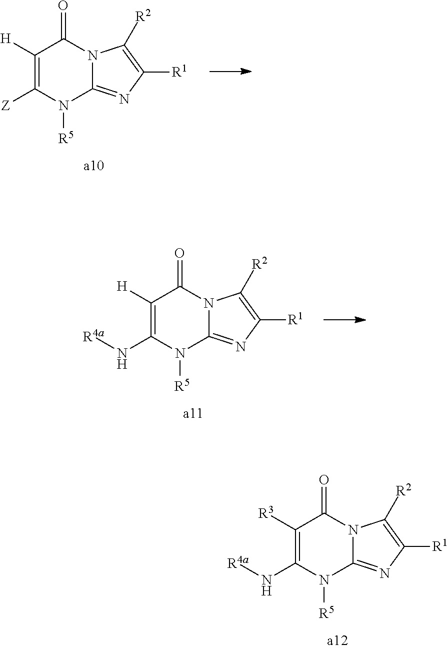

wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group; R² and R³ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R^{4a} represents substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group; Z is a leaving group.

### Step 1

A solution of compound a10 is reacted with an amine (R^{4a}NH₂ wherein R^{4a} is as defined above) in the presence of a base to obtain Compound a11.

Examples of the solvent include N,N-dimethylformamide, N,N-dimethylacetamide, N-methylpyrrolidone, ethanol and acetonitrile.

Examples of the base include 1,8-diazabicyclo[5,4,0]-7-undecene, sodium hydrogen carbonate, and the like, and the base may be used in 1 to 5 equivalents, preferably 1 to 3 equivalents, of Compound a10.

The amine (R^{4a}NH₂) may be used in 1 to 5 equivalents, preferably 1 to 3 equivalents, of Compound a10.

The reaction temperature may be 0° C. to reflux temperature, preferably room temperature to 100° C.

The reaction time may be 0.1 to 48 hours, preferably 1 to 24 hours.

### Step 2

The solution of compound a11 is reacted with an alkylating agent (R³—Y wherein Y is a leaving group such as halogen and R³ is as defined above) in the presence of a base to obtain Compound a12.

Examples of the solvent include N,N-dimethylformamide, N,N-dimethylacetamide, N-methylpyrrolidone and tetrahydrofuran, and the like.

Examples of the base include potassium carbonate, cesium carbonate, and sodium hydride, etc., and the base may be used in 1 to 5 equivalents, preferably 1 to 3 equivalents, of Compound a11.

Examples of the alkylating agent (R³—Y) include alkyl iodides alkyl bromides and the like, and the alkylating agent may be used in 1 to 5 equivalents, preferably 1 to 3 equivalents, of Compound a11.

The reaction temperature may be 0° C. to reflux temperature, preferably room temperature to 100° C.

The reaction time may be 0.1 to 48 hours, preferably 1 to 24 hours.

### Preparation of Compound a16

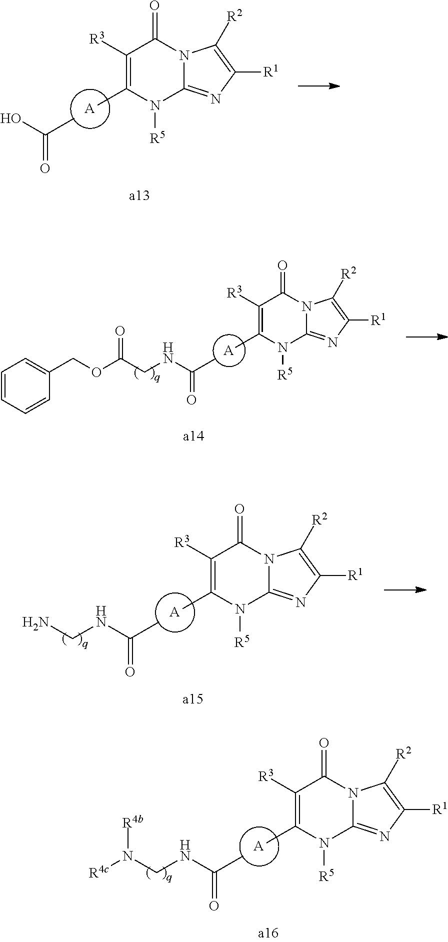

### Wherein

Ring A is substituted or unsubstituted non-aromatic carbocyclic ring, substituted or unsubstituted aromatic carbocyclic ring, substituted or unsubstituted non-aromatic heterocyclic ring, substituted or unsubstituted aromatic heterocyclic ring;
R¹ represents substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
R² and R³ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkylnylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R^{4b} and R^{4C} are independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkylnylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl, or substituted or unsubstituted aromatic heterocyclylsulfonyl;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group; q is an integer of 1 to 5.

### Step 1

A solution of compound a13 is reacted with an amine in the presence of a base, a condensing agent and additives to obtain Compound a14.

The amine may be used in 1 to 5 equivalents, preferably 1 to 3 equivalents, of Compound a13.

Examples of the solvent include methylene chloride, N,N-dimethylformamide, N,N-dimethylacetamide, N-methylpyrrolidone, tetrahydrofuran, and the like.

Examples of the bases include triethylamine, diisopropylethylamine and the like, and the base may be used in 1 to 10 equivalents, preferably 1 to 5 equivalents, of Compound a13.

Examples of the additive include 1-hydroxybenzotriazole and the like, and the additive may be used in 0.1 to 2 equivalents, preferably 0.2 to 0.5 equivalents, of Compound a13.

Examples of the condensing agent include 1-ethyl-3-(3-dimethylaminopropyl)carbodiimide hydrochloride, dicyclohexylcarbodiimide, O-(7-azabenzotriazol-1-yl)-N,N,N′,N′-tetramethyluronium hexafluorophosphate and the like, and the condensing agent may be used in 1 to 5 equivalents, preferably 1 to 3 equivalents, of Compound a13. The reaction temperature may be 0° C. to reflux temperature, preferably room temperature.

The reaction time may be 0.1 to 48 hours, preferably 1 to 24 hours.

### Step 2

The solution of Compound a14 is reacted with a deprotecting agent to obtain Compound a15.

Examples of the solvent include methylene chloride, tetrahydrofuran and the like.

Examples of the deprotecting agent include boron tribromide, boron trichloride, trimethylsilane iodide, palladium on carbon and the like, and the deprotecting agent may be used in 0.005 to 10 equivalents, preferably 0.01 to 5 equivalents, of Compound a13.

The reaction temperature may be −78° C. to room temperature, preferably −78° C. to 0° C.

The reaction time may be 0.1 to 48 hours, preferably 1 to 24 hours.

### Step 3

The solution of Compound a15 is reacted with an alkylating agent in the presence of a base to obtain Compound a16.

The alkylating agent may be used in 1 to 20 equivalents, preferably 1 to 10 equivalents, of Compound a15.

Examples of the solvents include 2-propanol and the like.

Examples of the base include sodium carbonate and the like, and the base may be used in 1 to 30 equivalents, preferably 1 to 10 equivalents, of Compound a15.

The reaction temperature may be 0° C. to reflux temperature.

The reaction time may be 0.1 to 48 hours, preferably 1 to 12 hours.

### Preparation of Compound a17

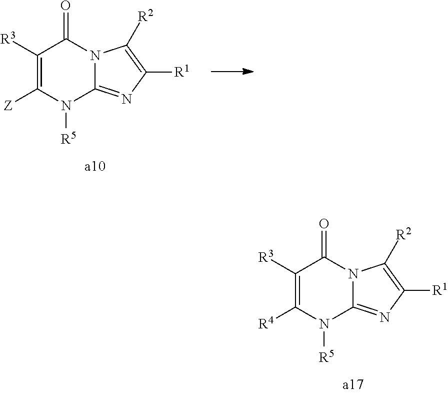

wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group; R² and R³ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R^{4a} is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
Z is a leaving group.

A solution of Compound a10 is reacted with a boronic acid or boronic acid ester in the presence of a base and a metal catalyst to obtain Compound a17.

Examples of the boronic acid include aromatic carbocyclic boronic acids, non-aromatic carbocyclic boronic acids, aromatic heterocyclic boronic acids, non-aromatic heterocyclic boronic acids and boronic acid esters thereof, and the boronic acid may be used in 1-10 equivalents, preferably 1 to 3 equivalents.

Examples of the metal catalyst include [1,1′-bis(diphenylphosphino)ferrocene]palladium(II) dichloride-dichloromethane complex, palladium acetate and the like, and the catalyst may be used in 0.01 to 0.5 equivalents, preferably 0.05 to 0.2 equivalents, of Compound a10.

Examples of the base include sodium carbonate, potassium carbonate, cesium carbonate and the like, and the base may be used in 1 to 10 equivalents, preferably 3 to 5 equivalents, of Compound a10.

Examples of the solvent include N,N-dimethylformamide, tetrahydrofuran, 1,4-dioxane, and the like.

The reaction temperature may be room temperature to reflux temperature, preferably room temperature to 100° C.

The reaction time may be 0.1 to 24 hours, preferably 1 to 12 hours.

The compound of the invention thus obtained may be purified and crystallized in a variety of solvents. Examples of the solvent to be used include alcohols (methanol, ethanol, isopropyl alcohol, n-butanol, etc.), ethers (diethyl ether, diisopropyl ether, etc.), methyl acetate, ethyl acetate, chloroform, methylene chloride, tetrahydrofuran, N,N-dimethylformamide, toluene, benzene, xylene, acetonitrile, hexane, dioxane, dimethoxyethane, water, or a mixture thereof. The compound may be dissolved in the solvent under heating, and the impurities are removed. The solution is then gradually cooled and filtered to collect the precipitated solid or crystal.

The compound of the present invention has autotaxin inhibitory activity. Accordingly, the pharmaceutical composition containing the compound of the present invention is useful as a therapeutic and/or prophylactic agent for diseases involving autotaxin. The diseases involving autotaxin include, for example, chronic kidney disease, urinary excretion failure, renal fibrosis, interstitial pneumonitis or pulmonary fibrosis, scleroderma, pain, fibromyalgia, rheumatoid arthritis, angiogenesis, cancer, formation, growth and propagation of tumor, arteriosclerosis, ocular diseases, choroidal neovascularization and diabetic retinopathy, inflammatory diseases, arthritis, neurodegeneration, restenosis, wound healing, transplant rejection and the like. The pharmaceutical composition containing the compound of the invention is useful as a therapeutic agent and/or preventive agent for these diseases.

The compounds of the invention may have a utility as a pharmaceutical, as well as autotaxin inhibitory effect, characterized by any of or all of the features as follows:

a) weak inhibitory effect on CYP enzymes (e.g., CYP1A2, CYP2C9, CYP3A4, etc.);
b) good pharmacokinetics, such as high bioavailability and appropriate clearance;
c) low toxicity (e.g., anemia-induced action);
d) high metabolic stability;
e) high water solubility;
f) high brain migration;
g) free of gastrointestinal disorders (e.g., hemorrhagic enteritis, gastrointestinal ulcers, gastrointestinal bleeding, etc.).

Also, the compound of the invention has low affinity for ENPP1, ENPP3 to 7 receptors and high selectivity for ENPP2 receptor.

The pharmaceutical composition of the invention may be administered orally or parenterally. The pharmaceutical composition may be administered orally in a formulation as conventionally used including tablets, granules, powders, capsules, pills, solutions, syrups, buccal or sublingual.

The pharmaceutical composition may be administered parenterally in a formulation as conventionally used including injections such as intramuscular or intravenous injection, suppositories, transdermal absorbents, inhalants, etc.

The pharmaceutical composition may be prepared by mixing an effective amount of the compound of the invention with various pharmaceutical additives suitable for the formulation, such as excipients, binders, moistening agents, disintegrants, lubricants, diluents and the like. For injections, an active ingredient together with a suitable carrier may be sterilized to obtain a pharmaceutical composition.

Examples of the excipients include lactose, saccharose, glucose, starch, calcium carbonate, crystalline cellulose and the like. Examples of the binders include methylcellulose, carboxymethylcellulose, hydroxypropylcellulose, gelatin, polyvinylpyrrolidone and the like. Examples of the disintegrants include carboxymethylcellulose, sodium carboxymethylcellulose, starch, sodium alginate, agar, sodium lauryl sulfate and the like. Examples of the lubricants include talc, magnesium stearate, macrogol and the like. For base materials of suppositories, cacao oil, macrogol, methylcellulose and the like may be used. Solubilizing agents, suspending agents, emulsifiers, stabilizers, preservatives, isotonic agents and the like, which are commonly used, may be added when the composition is prepared as solutions, emulsified or suspended injections. Sweetening agents, flavors and the like, which are commonly used, may be added for oral formulation.

The dosage of the pharmaceutical composition of the invention is determined in the light of the age and weight of the patient, the type and severity of the disease to be treated, and the route for administration and the like. In the case of oral administration to adults, the dosage is usually in the range of 0.05 to 100 mg/kg/day, preferably 0.1 to 10 mg/kg/day. In the case of parenteral administration, the dosage is variable depending on the administration route, but is usually 0.005 to 10 mg/kg/day, preferably in the range of 0.01 to 1 mg/kg/day. The dosage may be administered in single or divided doses.

The present invention is further explained by the following Examples and Test Examples, which are not intended to limit the scope of the present invention.

The abbreviations as used herein represent the following meanings.

Me: methyl
Et: ethyl
Bu: butyl
Ph: phenyl
PPh3, TPP: triphenylphosphine
AcOEt: ethyl acetate

### DMF: N,N-dimethylformamide

TFA: trifluoroacetic acid
DMSO: dimethyl sulfoxide
THF: tetrahydrofuran

### DIEA, Hunig's Base: N,N-diisopropylethylamine

TBAF: tetrabutylammonium fluoride
SEM: 2-(trimethylsilyl)ethoxymethyl
OAc: acetic acid group
mCPBA: meta-chloroperbenzoic acid
NMP: 1-methylpyrrolidin-2-one
LAH: lithium aluminum hydride
DBU: 1,8-diazabicyclo[5.4.0]undec-7-ene
DCM: methylene chloride
TEA: triethylamine
TMS: tetramethylsilane

NMR analysis of the compounds obtained in the Example was carried out at 400 MHz, using deuterated dimethyl sulfoxide (d6-DMSO) or deuterochloroform (CDCl₃).

LC/MS was measured under the following conditions.

### [Method A]

### Column: ACQUITY UPLC BEH C18 (1.7 μm i.d. 2.1×50 mm) (Waters)

Flow rate: 0.8 mL/min
UV detection wavelength: 254 nm

### Mobile Phase:

[A]0.1% formic acid in water
[B]0.1% formic acid in acetonitrile
Gradient: linear gradient from 10% to 100% [B] over 3.5 minutes, and then 100% [B] was maintained for 0.5 minutes.

### [Method B]

### Column: Shim-pack XR-ODS (2.2 μm, i.d. 50×3.0 mm) (Shimadzu)

Flow rate: 1.6 mL/min
UV detection wavelength: 254 nm

### Mobile Phase:

[A]0.1% formic acid in water
[B]0.1% formic acid in acetonitrile
Gradient: linear gradient from 10% to 100% [B] over 3 minutes, and then 100% [B] was maintained for 1 minute.

### Example 1

### Synthesis of 2-(4-chlorophenyl)-7-methyl-8-pentyl-imidazo[1,2-a]pyrimidin-5(8H)-one (3)

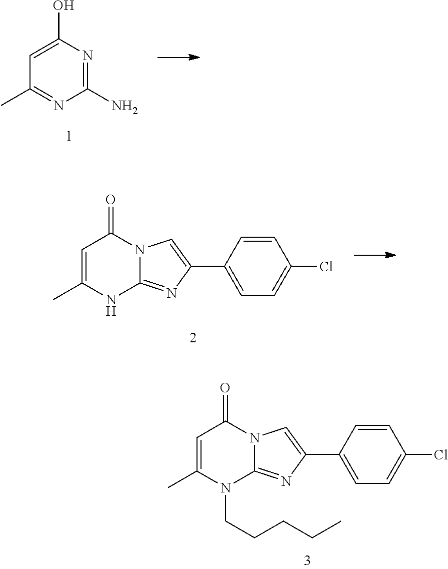

### Step 1:

To a solution of 2-amino-4-hydroxy-6-methylpyrimidine (1, 250 mg, 2.00 mmol) in N,N-dimethylformamide (10 mL) was added 2-bromo-1-(4-chlorophenyl)ethanone (467 mg, 2.00 mmol), and the solution was heated to reflux for 4 hours under argon atmosphere. The reaction was cooled to room temperature, and the precipitate was collected by filtration to yield 2-(4-chlorophenyl)-7-methyl-imidazo[1,2-a]pyrimidin-5(8H)-one (2, 301 mg, yield: 58%) as a pale yellow solid.

1H-NMR (δ ppm TMS/DMSO-d6) 8.13 (s, 1H), 7.94 (d, 2H, J=8.1 Hz), 7.48 (d, 2H, J=8.1 Hz), 5.65 (s, 1H), 2.30 (s, 3H).

### Step 2:

To a solution of the compound (2, 130 mg, 0.500 mmol) in N,N-dimethylformamide (5 mL) was added cesium carbonate (652 mg, 2.00 mmol) and 1-bromopentane (151 mg, 1.00 mmol), and the solution was stirred at room temperature for 24 hours. The reaction mixture was concentrated. The residue was dissolved in methylene chloride, and washed with water and brine. The organic layer was dried with anhydrous sodium sulfate and concentrated under reduced pressure. The obtained residue was purified by silica gel chromatography (methylene chloride) to yield 2-(4-chlorophenyl)-7-methyl-8-pentylimidazo[1,2-a]pyrimidin-5(8H)-one (3, 132 mg, yield: 80%) as a colorless solid.

1H-NMR (δ ppm TMS/DMSO-d6) 8.17 (s, 1H), 7.97 (d, 2H, J=8.1 Hz), 7.48 (d, 2H, J=8.1 Hz), 5.77 (s, 1H), 4.25 (t, 2H, J=7.1 Hz), 2.46 (s, 3H), 1.85-1.72 (m, 2H), 1.42-1.33 (m, 4H), 0.90 (t, 3H, J=6.6 Hz).

Compounds (4) to (19) were prepared in a similar manner.

TABLE 1

| Compound | Structure | 1H-NMR |
|---|---|---|
| 4 | 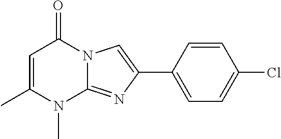 [SMILES: Cc1cc(=O)n2cc(-c3ccc(Cl)cc3)nc2n1C] | (δ ppm TMS/DMSO-d6) 8.19 (s, 1H), 7.98 (d, 2H, J = 8.1 Hz), 7.47 (d, 2H, J = 8.1 Hz), 5.78 (s, 1H), 3.81 (s, 3H), 2.44 (s, 3H). |
| 5 | 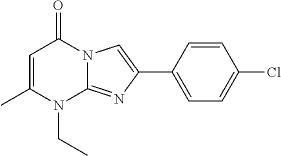 [SMILES: CCn1c(C)cc(=O)n2cc(-c3ccc(Cl)cc3)nc12] | (δ ppm TMS/DMSO-d6) 8.22 (s, 1H), 7.98 (d, 2H, J = 8.7 Hz), 7.47 (d, 2H, J = 8.7 Hz), 5.77 (s, 1H), 4.33 (q, 2H, J = 7.2 Hz), 2.47 (s, 3H), 1.37 (t, 3H, J = 7.2 Hz). |
| 6 | 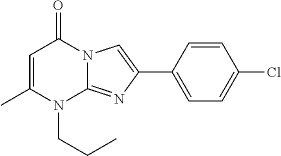 [SMILES: CCCn1c(C)cc(=O)n2cc(-c3ccc(Cl)cc3)nc12] | (δ ppm TMS/DMSO-d6) 8.17 (s, 1H), 7.97 (d, 2H, J = 8.1 Hz), 7.47 (d, 2H, J = 8.1 Hz), 5.77 (s, 1H), 4.23 (t, 2H, J = 7.2 Hz), 2.47 (s, 3H), 1.89-1.75 (m, 2H), 0.97 (t, 3H, J = 7.2 Hz). |
| 7 | 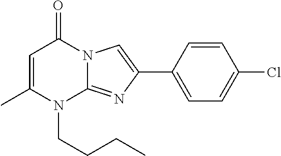 [SMILES: CCCCn1c(C)cc(=O)n2cc(-c3ccc(Cl)cc3)nc12] | (δ ppm TMS/DMSO-d6) 8.18 (s, 1H), 7.98 (d, 2H, J = 8.1 Hz), 7.48 (d, 2H, J = 8.1 Hz), 5.78 (s, 1H), 4.28 (t, 2H, J = 7.3 Hz), 2.47 (s, 3H), 1.83-1.70 (m, 2H), 1.48-1.34 (m, 2H), 0.96 (t, 3H, J = 7.3 Hz). |
| 8 | 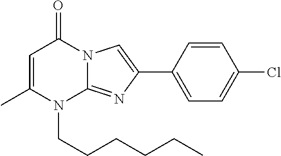 [SMILES: CCCCCCn1c(C)cc(=O)n2cc(-c3ccc(Cl)cc3)nc12] | (δ ppm TMS/DMSO-d6) 8.14 (s, 1H), 7.94 (d, 2H, J = 8.1 Hz), 7.45 (d, 2H, J = 8.1 Hz), 5.75 (s, 1H), 4.24 (t, 2H, J = 7.3 Hz), 2.44 (s, 3H), 1.83-1.68 (m, 2H), 1.42-1.23 (m, 6H), 0.85 (t, 3H, J = 6.6 Hz). |

TABLE 2

| Compound | Structure | 1H-NMR |
|---|---|---|
| 9 | 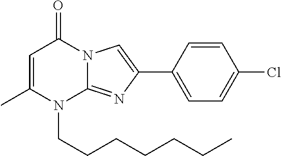 [SMILES: CCCCCCCn1c(C)cc(=O)n2cc(-c3ccc(Cl)cc3)nc12] | (δ ppm TMS/DMSO-d6) 8.17 (s, 1H), 7.97 (d, 2H, J = 8.1 Hz), 7.47 (d, 2H, J = 8.1 Hz), 5.77 (s, 1H), 4.25 (t, 2H, J = 7.2 Hz), 2.46 (s, 3H), 1.85-1.70 (m, 2H), 1.44-1.19 (m, 8H), 0.86 (t, 3H, J = 5.4 Hz). |
| 10 | 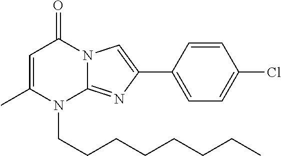 [SMILES: CCCCCCCCn1c(C)cc(=O)n2cc(-c3ccc(Cl)cc3)nc12] | (δ ppm TMS/DMSO-d6) 8.15 (s, 1H), 7.95 (d, 2H, J = 8.8 Hz), 7.44 (d, 2H, J = 8.8 Hz), 5.75 (s, 1H), 4.24 (t, 2H, J = 8.0 Hz), 2.44 (s, 3H), 1.83-1.68 (m, 2H), 1.44-1.25 (m, 10H), 0.83 (t, 3H, J = 6.6 Hz). |
| 11 | 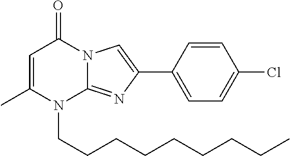 [SMILES: CCCCCCCCCn1c(C)cc(=O)n2cc(-c3ccc(Cl)cc3)nc12] | 8.13 (s, 1H), 7.94 (d, 2H, J = 8.1 Hz), 7.43 (d, 2H, J = 8.1 Hz), 5.74 (s, 1H), 4.22 (t, 2H, J = 8.0 Hz), 2.43 (s, 3H), 1.83-1.65 (m, 2H), 1.40-1.12 (m, 12H), 0.81 (t, 3H, J = 7.3 Hz). |
| 12 | 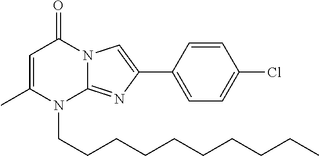 [SMILES: CCCCCCCCCCn1c(C)cc(=O)n2cc(-c3ccc(Cl)cc3)nc12] | (δ ppm TMS/DMSO-d6) 8.15 (s, 1H), 7.95 (d, 2H, J = 8.0 Hz), 7.45 (d, 2H, J = 8.0 Hz), 5.75 (s, 1H), 4.25 (t, 2H, J = 7.3 Hz), 2.44 (s, 3H), 1.85-1.67 (m, 2H), 1.42-1.12 (m, 14H), 0.82 (m, 3H). |
| 13 | 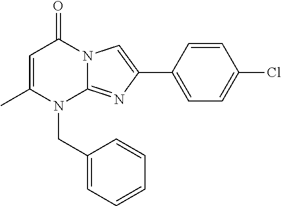 [SMILES: Cc1cc(=O)n2cc(-c3ccc(Cl)cc3)nc2n1Cc1ccccc1] | (δ ppm TMS/DMSO-d6) 8.24 (s, 1H), 7.95 (d, 2H, J = 8.1 Hz), 7.45 (d, 2H, J = 8.1 Hz), 7.40-7.22 (m, 5H), 5.85 (s, 1H), 5.62 (s, 2H), 2.35 (s, 3H). |

TABLE 3

| Compound | Structure | 1H-NMR |
|---|---|---|
| 14 | 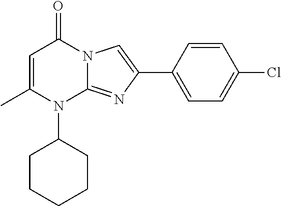 [SMILES: Cc1cc(=O)n2cc(-c3ccc(Cl)cc3)nc2n1C1CCCCC1] | (δ ppm TMS/DMSO-d6) 7.84 (s, 1H), 7.81 (d, 2H, J = 8.1 Hz), 7.38 (d, 2H, J = 8.1 Hz), 5.67 (s, 1H), 5.82 (s, 1H), 4.34- 4.05 (m, 1H), 3.28-3.00 (m, 2H), 2.45 (s, 3H), 2.07-1.90 (m, 2H), 1.88-1.68 (m, 3H), 1.53-1.27 (m, 3H). |
| 15 | 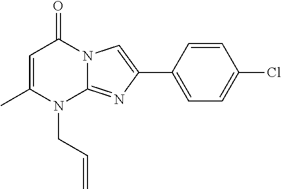 [SMILES: C=CCn1c(C)cc(=O)n2cc(-c3ccc(Cl)cc3)nc12] | (δ ppm TMS/DMSO-d6) 8.20 (s, 1H), 7.96 (d, 2H, J = 8.8 Hz), 7.47 (d, 2H, J = 8.8 Hz), 6.14-5.98 (m, 1H), 5.81 (s, 1H), 5.27-5.06 (m, 2H), 4.97 (s, 2H), 2.42 (s, 3H). |
| 16 | 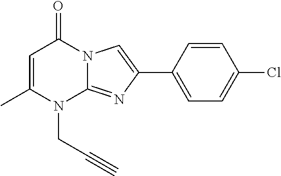 [SMILES: C#CCn1c(C)cc(=O)n2cc(-c3ccc(Cl)cc3)nc12] | (δ ppm TMS/DMSO-d6) 8.22 (s, 1H), 7.99 (d, 2H, J = 8.8 Hz), 7.48 (d, 2H, J = 8.8 Hz), 5.86 (s, 1H), 5.20 (s, 2H), 3.53 (s, 1H), 2.55 (s, 3H). |
| 17 | 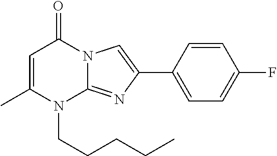 [SMILES: CCCCCn1c(C)cc(=O)n2cc(-c3ccc(F)cc3)nc12] | (δ ppm TMS/DMSO-d6) 8.11 (s, 1H), 8.03-7.95 (m, 2H), 7.29- 7.22 (m, 2H), 5.76 (s, 1H), 4.25 (t, 2H, J = 7.5 Hz), 2.46 (s, 3H), 1.85-1.72 (m, 2H), 1.43-1.32 (m, 4H), 0.90 (t, 3H, J = 6.6 Hz). |
| 18 | 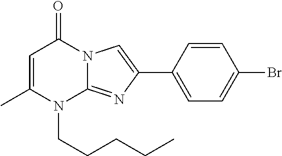 [SMILES: CCCCCn1c(C)cc(=O)n2cc(-c3ccc(Br)cc3)nc12] | (δ ppm TMS/DMSO-d6) 8.18 (s, 1H), 7.91 (d, 2H, J = 8.1 Hz), 7.61 (d, 2H, J = 8.1 Hz), 5.78 (s, 1H), 4.27 (t, 2H, J = 7.2 Hz), 2.47 (s, 3H), 1.85-1.74 (m, 2H), 1.41-1.33 (m, 4H), 0.90 (t, 3H, J = 6.6 Hz). |

TABLE 4

| Compound | Structure | 1H-NMR |
|---|---|---|
| 19 | 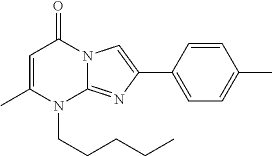 | (δ ppm TMS/DMSO-d6) 8.10 (s, 1H), 7.84 (d, 2H, J = 8.1 Hz), 7.23 (d, 2H, J = 8.1 Hz), 5.76 (s, 1H), 4.30-4.19 (m, 2H), 2.46 (s, 3H), 2.33 (s, 3H), 1.92-1.73 (m, 2H), 1.43-1.29 (m, 4H), 0.90 (t, 3H, J = 6.6 Hz). |

### Example 2

### 2-(4-chlorophenyl)-7-methyl-5-oxoimidazo[1,2-a]pyrimidin-8(5H)-yl)acetic acid ethyl ester (20)

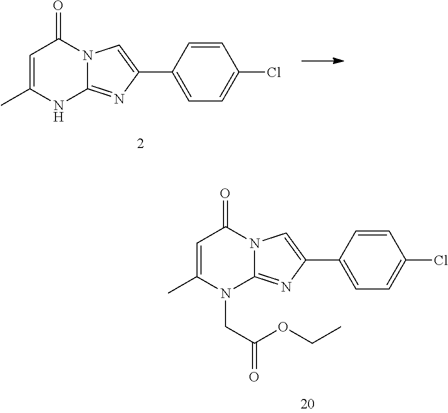 [SMILES: Cc1cc(=O)n2cc(-c3ccc(Cl)cc3)nc2[nH]1 ; CCOC(=O)Cn1c(C)cc(=O)n2cc(-c3ccc(Cl)cc3)nc12]

### Step 1:

To a solution of the compound (2, 130 mg, 0.500 mmol) in N,N-dimethylformamide (5 mL) was added cesium carbonate (652 mg, 2.00 mmol) and bromoacetic acid ethyl ester (167 mg, 1.00 mmol), and the solution was stirred for 12 hours at room temperature. The reaction mixture was concentrated. The residue was dissolved in methylene chloride, and washed with water and brine. The organic layer was dried with anhydrous sodium sulfate and concentrated under reduced pressure. The residue was purified by silica gel chromatography (methylene chloride) to yield 2-(4-chlorophenyl)-7-methyl-5-oxoimidazo[1,2-a]pyrimidin-8(5H)-yl)acetic acid ethyl ester (20, 143 mg, yield: 83%) as a colorless solid.

1H-NMR (δ ppm TMS/DMSO-d6) 8.23 (s, 1H), 7.94 (d, 2H, J=8.8 Hz), 7.47 (d, 2H, J=8.8 Hz), 5.87 (s, 1H), 5.19 (s, 2H), 4.22 (q, 2H, J=7.3 Hz), 2.39 (s, 3H), 1.23 (t, 3H, J=7.3 Hz).

Compounds (21) to (124) were prepared in a similar manner.

TABLE 5

| Compound | Structure | 1H-NMR |
|---|---|---|
| 21 | 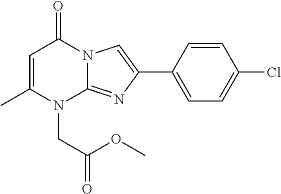 [SMILES: COC(=O)Cn1c(C)cc(=O)n2cc(-c3ccc(Cl)cc3)nc12] | (δppm TMS/DMSO-d6) 8.22 (s, 1H), 7.94 (d, 2H, J = 8.1 Hz), 7.47 (d, 2H, J = 8.1 Hz), 5.87 (s, 1H), 5.25 (s, 2H), 3.75 (s, 3H), 2.39 (s, 3H). |
| 22 | 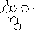 [SMILES: Cc1cc(=O)n2cc(-c3ccc(Cl)cc3)nc2n1CC(=O)OCc1ccccc1] | (δppm TMS/DMSO-d6) 8.21 (s, 1H), 7.92 (d, 2H, J = 8.1 Hz), 7.47 (d, 2H, J = 8.1 Hz), 7.42-7.32 (m, 5H), 5.86 (s, 1H), 5.26 (m, 4H), 2.39 (s, 3H). |
| 23 | 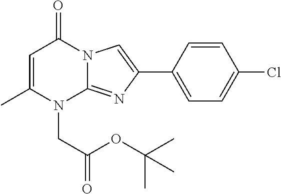 [SMILES: Cc1cc(=O)n2cc(-c3ccc(Cl)cc3)nc2n1CC(=O)OC(C)(C)C] | (δppm TMS/DMSO-d6) 8.21 (s, 1H), 7.94 (d, 2H, J = 8.8 Hz), 7.47 (d, 2H, J = 8.8 Hz), 5.85 (s, 1H), 2.37 (s, 3H), 1.44 (s, 9H). |
| 24 | 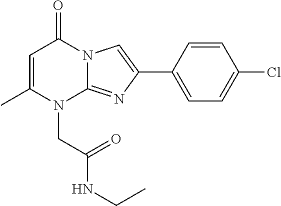 [SMILES: CCNC(=O)Cn1c(C)cc(=O)n2cc(-c3ccc(Cl)cc3)nc12] | (δppm TMS/DMSO-d6) 8.37 (m, 1H), 8.19 (s, 1H), 7.95 (d, 2H, J = 8.1 Hz), 7.47 (d, 2H, J = 8.1 Hz), 5.82 (s, 1H), 4.96 (s, 2H), 3.19-3.08 (m, 2H), 2.34 (s, 3H), 1.05 (t, 3H, J = 6.6 Hz). |
| 25 | 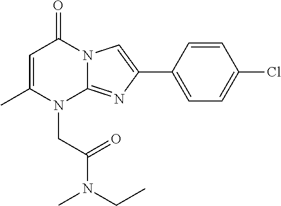 [SMILES: CCN(C)C(=O)Cn1c(C)cc(=O)n2cc(-c3ccc(Cl)cc3)nc12] | (δppm TMS/DMSO-d6) 8.19 (d, 1H, J = 1.4 Hz), 7.97-7.89 (m, 2H), 7.47 (d, 2H, J = 8.1 Hz), 5.82 (s, 1H), 5.27 (d, 2H, J = 9.5 Hz), 3.56-3.44 (q, 1H, J = 6.6 Hz), 3.14 (s, 1.5 H), 2.32 (d, 3H, J = 4.4 Hz), 1.32 (t, 1.5H, J = 6.6 Hz), 1.04 (t, 1.5H, J = 6.6 Hz). |

TABLE 6

| Compound | Structure | 1H-NMR |
|---|---|---|
| 26 | 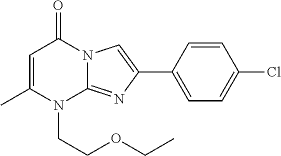 [SMILES: CCOCCn1c(C)cc(=O)n2cc(-c3ccc(Cl)cc3)nc12] | (δppm TMS/DMSO-d6) 8.18 (s, 1H), 7.96 (d, 2H, J = 8.1 Hz), 7.47 (d, 2H, J = 8.1 Hz), 5.77 (s, 1H), 4.45 (t, 2H, J = 5.1 Hz), 3.80 (t, 2H, J = 5.1 Hz), 3.44 (q, 2H, J = 7.2 Hz), 1.04 (t, 3H, J = 6.6 Hz). |
| 27 | 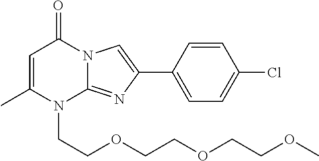 | (δppm TMS/CDCl3) 7.85 (s, 1H), 7.79 (d, 2H, J = 8.8 Hz), 7.37 (d, 2H, J = 8.8 Hz), 5.69 (s, 1H), 4.50 (t, 2H, J = 5.1 Hz), 4.45-4.10 (m, 4H), 3.97 (t, 2H, J = 5.1 Hz), 3.80-3.52 (m, 7H), 2.51 (s, 3H). |
| 28 | 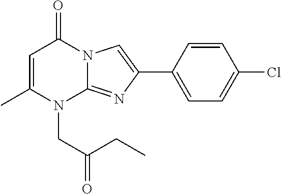 [SMILES: CCC(=O)Cn1c(C)cc(=O)n2cc(-c3ccc(Cl)cc3)nc12] | (δppm TMS/DMSO-d6) 8.19 (s, 1H), 7.93 (d, 2H, J = 8.1 Hz), 7.46 (d, 2H, J = 8.1 Hz), 5.83 (s, 1H), 2.71 (q, 2H, J = 7.2 Hz), 2.28 (s, 3H), 1.04 (t, 3H, J = 7.2 Hz). |
| 29 | 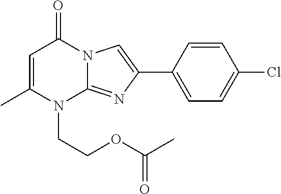 [SMILES: CC(=O)OCCn1c(C)cc(=O)n2cc(-c3ccc(Cl)cc3)nc12] | (δppm TMS/DMSO-d6) 8.18 (s, 1H), 7.97 (d, 2H, J = 8.1 Hz), 7.48 (d, 2H, J = 8.1 Hz), 5.80 (s, 1H), 4.59-4.43 (m, 4H), 2.48 (s, 3H), 1.91 (s, 3H). |
| 30 | 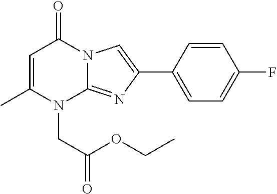 [SMILES: CCOC(=O)Cn1c(C)cc(=O)n2cc(-c3ccc(F)cc3)nc12] | (δppm TMS/DMSO-d6) 8.17 (s, 1H), 8.00-7.93 (m, 2H), 7.30- 7.19 (m, 2H), 5.86 (s, 1H), 5.19 (s, 2H), 4.22 (q, 2H, J = 6.6 Hz), 2.38 (s, 3H), 1.24 (t, 3H, J = 6.6 Hz). |

TABLE 7

| Compound | Structure | 1H-NMR |
|---|---|---|
| 31 | 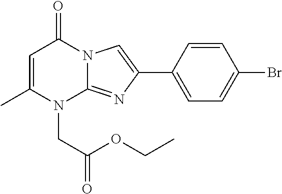 [SMILES: CCOC(=O)Cn1c(C)cc(=O)n2cc(-c3ccc(Br)cc3)nc12] | (δppm TMS/DMSO-d6) 8.24 (s, 1H), 7.88 (d, 2H, J = 8.7 Hz), 7.61 (d, 2H, J = 8.7 Hz), 5.87 (s, 1H), 5.19 (s, 2H), 4.22 (q, 2H, J = 7.2 Hz), 2.38 (s, 3H), 1.23 (t, 3H, J = 7.2 Hz). |
| 32 | 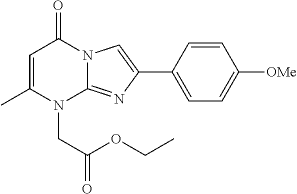 [SMILES: CCOC(=O)Cn1c(C)cc(=O)n2cc(-c3ccc(OC)cc3)nc12] | (δppm TMS/DMSO-d6) 8.03 (s, 1H), 7.84 (d, 2H, J = 8.8 Hz), 6.97 (d, 2H, J = 8.8 Hz), 5.84 (s, 1H), 5.18 (s, 2H), 4.22 (q, 2H, J = 6.6 Hz), 3.78 (s, 3H), 2.38 (s, 3H), 1.23 (t, 3H, J = 6.6 Hz). |
| 33 | 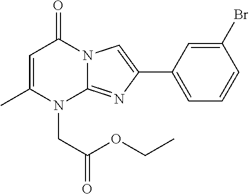 [SMILES: CCOC(=O)Cn1c(C)cc(=O)n2cc(-c3cccc(Br)c3)nc12] | (δppm TMS/DMSO-d6) 8.28 (s, 1H), 8.11 (s, 1H), 7.93 (d, 1H, J = 8.1 Hz), 7.54-7.30 (m, 2H), 5.87 (s, 1H), 5.19 (s, 2H), 4.23 (q, 2H, J = 7.3 Hz), 2.39 (s, 3H), 1.24 (t, 3H, J = 7.3 Hz). |
| 34 | 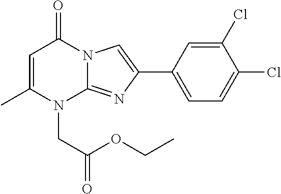 [SMILES: CCOC(=O)Cn1c(C)cc(=O)n2cc(-c3ccc(Cl)c(Cl)c3)nc12] | (δppm TMS/DMSO-d6) 8.34 (s, 1H), 8.15 (s, 1H), 7.90 (d, 1H, J = 8.1 Hz), 7.66 (d, 1H, J = 8.1 Hz), 5.87 (s, 1H), 5.19 (s, 2H), 4.22 (q, 2H, J = 6.6 Hz), 2.38 (s, 3H), 1.24 (t, 3H, J = 6.6 Hz). |
| 35 | 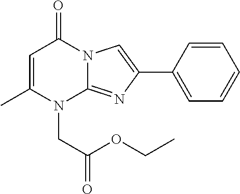 [SMILES: CCOC(=O)Cn1c(C)cc(=O)n2cc(-c3ccccc3)nc12] | (δppm TMS/DMSO-d6) 7.92 (d, 2H, J = 7.3 Hz), 7.46-7.37 (m, 2H), 7.35-7.27 (m, 1H), 5.86 (s, 1H), 5.19 (s, 2H), 4.22 (q, 2H, J = 6.6 Hz), 2.39 (s, 3H), 1.24 (t, 3H, J = 6.6 Hz). |

TABLE 8

| Compound | Structure | 1H-NMR |
|---|---|---|
| 36 | 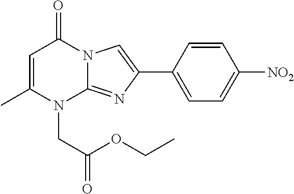 [SMILES: CCOC(=O)Cn1c(C)cc(=O)n2cc(-c3ccc([N+](=O)[O-])cc3)nc12] | (δppm TMS/CDCl3) 8.25 (d, 2H, J = 8.8 Hz), 8.02-7.97 (m, 3H), 6.71 (d, 2H, J = 8.8 Hz), 5.76 (s, 1H), 5.07 (s, 2H), 4.31 (q, 2H, J = 7.3 Hz), 2.38 (s, 3H), 1.33 (t, 3H, J = 7.3 Hz). |
| 37 | 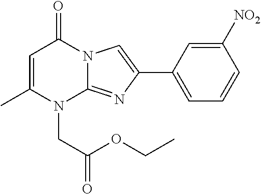 [SMILES: CCOC(=O)Cn1c(C)cc(=O)n2cc(-c3cccc([N+](=O)[O-])c3)nc12] | (δppm TMS/CDCl3) 8.74 (s, 1H), 8.17-8.07 (m, 2H), 7.97 (s, 1H), 7.61-7.52 (m, 1H), 5.80 (s, 1H), 5.11 (s, 2H), 4.32 (q, 2H, J = 7.3 Hz), 2.38 (s, 3H), 1.35 (t, 3H, J = 7.3 Hz) . |
| 38 | 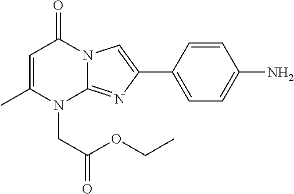 [SMILES: CCOC(=O)Cn1c(C)cc(=O)n2cc(-c3ccc(N)cc3)nc12] | (δppm TMS/CDCl3) 7.71 (s, 1H), 7.64 (d, 2H, J = 8.8 Hz), 6.71 (d, 2H, J = 8.8 Hz), 5.73 (s, 1H), 5.07 (s, 2H), 4.28 (q, 2H, J = 7.3 Hz), 3.75 (brs, 2H), 2.33 (s, 3H), 1.31 (t, 3H, J = 7.3 Hz). |
| 39 | 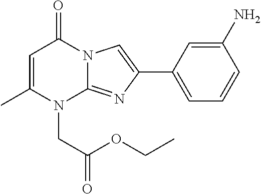 [SMILES: CCOC(=O)Cn1c(C)cc(=O)n2cc(-c3cccc(N)c3)nc12] | (δppm TMS/CDCl3) 7.82 (s, 1H), 7.24-7.16 (m, 3H), 6.67- 6.61 (m, 1H), 5.76 (s, 1H), 5.10 (s, 2H), 4.29 (q, 2H, J = 7.3 Hz), 3.73 (brs, 2H), 2.36 (s, 3H), 1.32 (t, 3H, J = 7.3 Hz). |
| 40 | 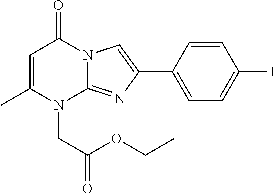 [SMILES: CCOC(=O)Cn1c(C)cc(=O)n2cc(-c3ccc(I)cc3)nc12] | (δppm TMS/DMSO-d6) 8.22 (s, 1H), 7.80-7.70 (m, 4H), 7.35- 7.27 (m, 1H), 5.87 (s, 1H), 5.18 (s, 2H), 4.21 (qd, 2H, J = 7.3, 2.2 Hz), 2.38 (s, 3H), 1.23 (td, 3H, J = 7.3, 2.2 Hz). |

TABLE 9

| Compound | Structure | 1H-NMR |
|---|---|---|
| 41 | 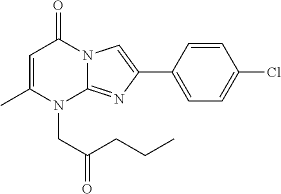 [SMILES: CCCC(=O)Cn1c(C)cc(=O)n2cc(-c3ccc(Cl)cc3)nc12] | (δppm TMS/CDCl3) 7.79 (s, 1H), 7.71 (d, 2H, J = 7.8 Hz), 7.33 (d, 2H, J = 8.0 Hz), 5.72 (s, 1H), 5.10 (s, 2H), 2.60 (t, 2H, J = 7.2 Hz), 2.24 (s, 3H), 1.76-1.73 (m, 2H), 1.00 (t, 3H, J = 7.3 Hz). |
| 42 | 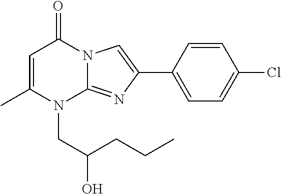 [SMILES: CCCC(O)Cn1c(C)cc(=O)n2cc(-c3ccc(Cl)cc3)nc12] | (δppm TMS/CDCl3) 7.60 (d, 2H, J = 8.0 Hz), 7.48 (s, 1H), 7.27 (d, 2H, J = 8.0 Hz), 5.42 (s, 1H), 4.43 (d, 1H, J = 3.0 Hz), 4.33-4.28 (m, 2H), 4.01 (dd, 1H, J = 14.1, 9.3 Hz), 2.41 (s, 3H), 1.63-1.56 (m, 4H), 1.03 (t, 3H, J = 6.7 Hz). |
| 43 | 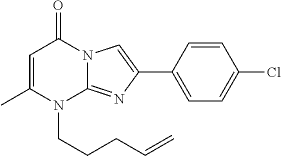 [SMILES: C=CCCCn1c(C)cc(=O)n2cc(-c3ccc(Cl)cc3)nc12] | (δppm TMS/CDCl3) 7.84-7.81 (m, 3H), 7.38 (d, 2H, J = 8.3 Hz), 5.87-5.85 (m, 1H), 5.70 (s, 1H), 5.14-5.08 (m, 2H), 4.30 (t, 2H, J = 7.8 Hz), 2.43 (s, 3H), 2.24 (q, 2H, J = 6.9 Hz), 2.04-1.96 (m, 2H). |
| 44 | 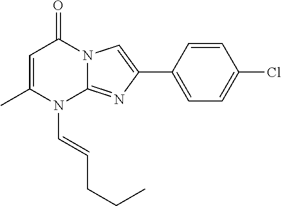 [SMILES: CCC/C=C/n1c(C)cc(=O)n2cc(-c3ccc(Cl)cc3)nc12] | (δppm TMS/CDCl3) 7.86 (s, 1H), 7.79 (d, 2H, J = 8.0 Hz), 7.37 (d, 2H, J = 7.9 Hz), 6.49 (d, 1H, J = 13.9 Hz), 6.33- 6.31 (m, 1H), 5.76 (s, 1H), 2.39 (s, 3H), 2.33 (q, 2H, J = 7.2 Hz), 1.63-1.60 (m, 2H), 1.06 (t, 3H, J = 7.3 Hz). |
| 45 | 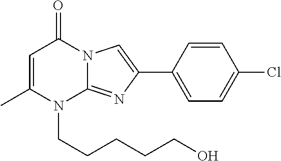 [SMILES: Cc1cc(=O)n2cc(-c3ccc(Cl)cc3)nc2n1CCCCCO] | (δppm TMS/CDCl3) 7.85 (s, 1H), 7.81 (d, 2H, J = 7.8 Hz), 7.38 (d, 2H, J = 7.5 Hz), 5.70 (s, 1H), 4.31 (t, 2H, J = 7.8 Hz), 3.70 (t, 2H, J = 6.3 Hz), 2.44 (s, 3H), 1.96-1.88 (m, 2H), 1.73-1.66 (m, 2H), 1.59- 1.55 (m, 4H). |

TABLE 10

| Compound | Structure | 1H-NMR |
|---|---|---|
| 46 | 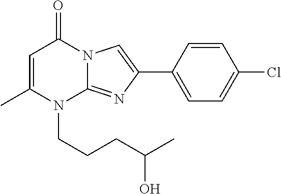 [SMILES: Cc1cc(=O)n2cc(-c3ccc(Cl)cc3)nc2n1CCCC(C)O] | (δppm TMS/CDCl3) 7.81 (s, 1H), 7.75 (d, 2H, J = 7.9 Hz), 7.35 (d, 2H, J = 7.9 Hz), 5.69 (s, 1H), 4.32-4.31 (m, 2H), 3.99 (m, 1H), 2.42 (s, 3H), 1.97-1.95 (m, 2H), 1.58-1.54 (m, 2H), 1.25 (s, 3H). |
| 47 | 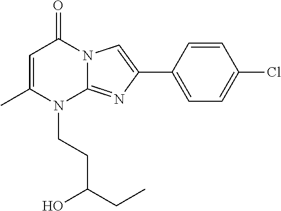 [SMILES: CCC(O)CCn1c(C)cc(=O)n2cc(-c3ccc(Cl)cc3)nc12] | (δppm TMS/CDCl3) 7.84 (s, 1H), 7.72 (d, 2H, J = 8.2 Hz), 7.37 (d, 2H, J = 8.2 Hz), 5.76 (s, 1H), 4.79 (t, 1H, J = 11.6 Hz), 4.16 (d, 1H, J = 11.6 Hz), 3.37-3.34 (m, 1H), 2.47 (s, 3H), 2.31 (t, 1H, J = 8.0 Hz), 1.97-1.94 (m, 1H), 1.82 (d, 1H, J = 8.0 Hz), 1.62-1.48 (m, 2H), 0.91 (t, 3H, J = 8.0 Hz) . |
| 48 | 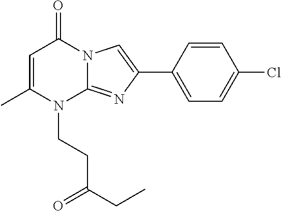 [SMILES: CCC(=O)CCn1c(C)cc(=O)n2cc(-c3ccc(Cl)cc3)nc12] | (δppm TMS/CDCl3) 7.85 (s, 1H), 7.80 (d, 2H, J = 8.4 Hz), 7.38 (d, 2H, J = 8.3 Hz), 5.72 (s, 1H), 4.53 (t, 2H, J = 8.0 Hz), 3.16 (t, 2H, J = 8.0 Hz), 2.51 (s, 3H), 2.34 (t, 3H, J = 8.0 Hz), 0.87 (t, 3H, J = 8.0 Hz) . |
| 49 | 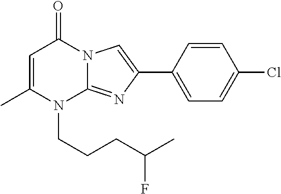 [SMILES: Cc1cc(=O)n2cc(-c3ccc(Cl)cc3)nc2n1CCCC(C)F] | (δppm TMS/CDCl3) 7.85 (s, 1H), 7.80 (d, 2H, J = 7.5 Hz), 7.38 (d, 2H, J = 7.8 Hz), 5.71 (s, 1H), 4.82-4.76 (m, 1H), 4.35 (t, 2H, J = 7.8 Hz), 2.45 (s, 3H), 2.06-1.98 (m, 2H), 1.82-1.72 (m, 2H), 1.41-1.35 (m, 3H) . |

TABLE 11

| | | | Retention | |
|---|---|---|---|---|
| | | | Time | Mass |
| Compound | Structure | LC/MS | [min] | [M + H] |
| 50 | 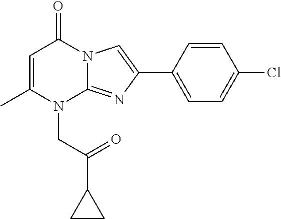 [SMILES: Cc1cc(=O)n2cc(-c3ccc(Cl)cc3)nc2n1CC(=O)C1CC1] | Method A | 1.83 | 342 |
| 51 | 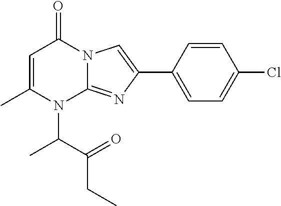 [SMILES: CCC(=O)C(C)n1c(C)cc(=O)n2cc(-c3ccc(Cl)cc3)nc12] | Method A | 2.00 | 344 |
| 52 | 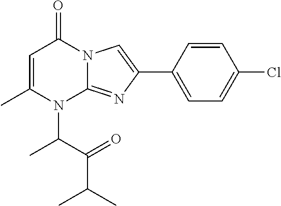 [SMILES: Cc1cc(=O)n2cc(-c3ccc(Cl)cc3)nc2n1C(C)C(=O)C(C)C] | Method A | 1.92 | 344 |
| 53 | 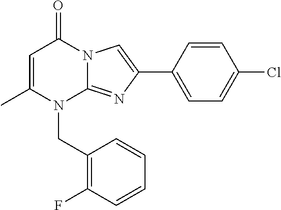 [SMILES: Cc1cc(=O)n2cc(-c3ccc(Cl)cc3)nc2n1Cc1ccccc1F] | Method A | 2.57 | 368 |
| 54 | 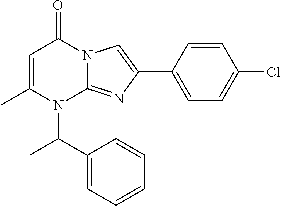 [SMILES: Cc1cc(=O)n2cc(-c3ccc(Cl)cc3)nc2n1C(C)c1ccccc1] | Method A | 2.68 | 364 |

TABLE 12

| | | | Retention | |
|---|---|---|---|---|
| | | | Time | Mass |
| Compound | Structure | LC/MS | [min] | [M + H] |
| 55 | 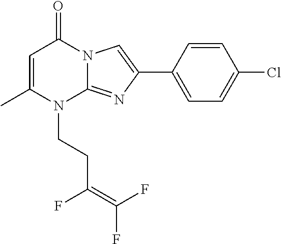 [SMILES: Cc1cc(=O)n2cc(-c3ccc(Cl)cc3)nc2n1CCC(F)=C(F)F] | Method A | 2.46 | 368 |
| 56 | 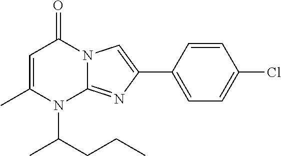 [SMILES: CCCC(C)n1c(C)cc(=O)n2cc(-c3ccc(Cl)cc3)nc12] | Method A | 2.81 | 330 |
| 57 | 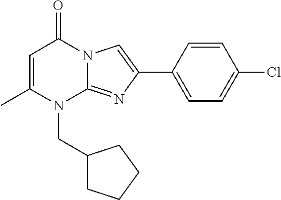 [SMILES: Cc1cc(=O)n2cc(-c3ccc(Cl)cc3)nc2n1CC1CCCC1] | Method A | 2.80 | 342 |
| 58 | 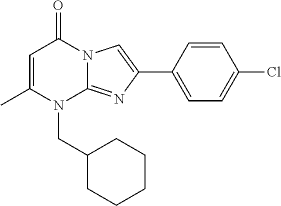 [SMILES: Cc1cc(=O)n2cc(-c3ccc(Cl)cc3)nc2n1CC1CCCCC1] | Method A | 2.94 | 356 |
| 59 | 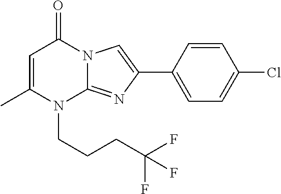 [SMILES: Cc1cc(=O)n2cc(-c3ccc(Cl)cc3)nc2n1CCCC(F)(F)F] | Method A | 2.46 | 370 |

TABLE 13

| | | | Retention | |
|---|---|---|---|---|
| | | | Time | Mass |
| Compound | Structure | LC/MS | [min] | [M + H] |
| 60 | 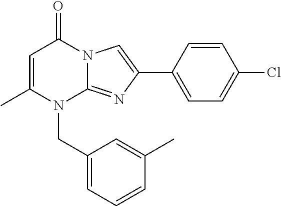 [SMILES: Cc1cccc(Cn2c(C)cc(=O)n3cc(-c4ccc(Cl)cc4)nc23)c1] | Method A | 2.69 | 364 |
| 61 | 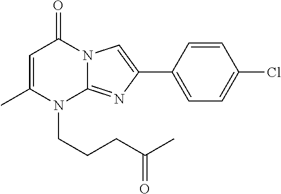 [SMILES: CC(=O)CCCn1c(C)cc(=O)n2cc(-c3ccc(Cl)cc3)nc12] | Method A | 1.89 | 344 |
| 62 | 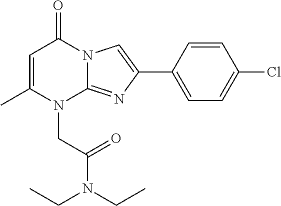 [SMILES: CCN(CC)C(=O)Cn1c(C)cc(=O)n2cc(-c3ccc(Cl)cc3)nc12] | Method A | 2.09 | 373 |
| 63 | 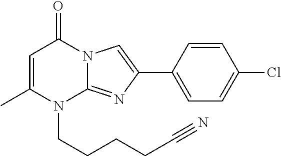 [SMILES: Cc1cc(=O)n2cc(-c3ccc(Cl)cc3)nc2n1CCCCC#N] | Method A | 2.05 | 341 |
| 64 | 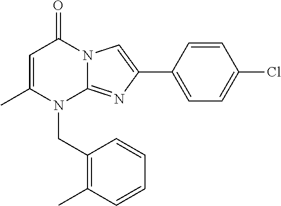 [SMILES: Cc1ccccc1Cn1c(C)cc(=O)n2cc(-c3ccc(Cl)cc3)nc12] | Method A | 2.63 | 364 |

TABLE 14

| | | | Retention | |
|---|---|---|---|---|
| | | | Time | Mass |
| Compound | Structure | LC/MS | [min] | [M + H] |
| 65 | 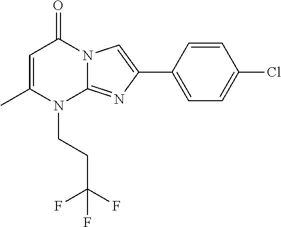 [SMILES: Cc1cc(=O)n2cc(-c3ccc(Cl)cc3)nc2n1CCC(F)(F)F] | Method A | 2.42 | 356 |
| 66 | 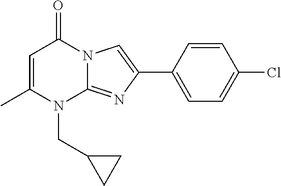 [SMILES: Cc1cc(=O)n2cc(-c3ccc(Cl)cc3)nc2n1CC1CC1] | Method A | 2.43 | 314 |
| 67 | 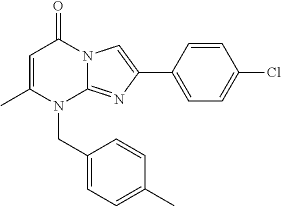 [SMILES: Cc1ccc(Cn2c(C)cc(=O)n3cc(-c4ccc(Cl)cc4)nc23)cc1] | Method A | 2.70 | 364 |
| 68 | 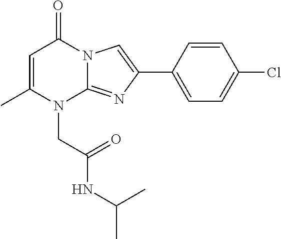 [SMILES: Cc1cc(=O)n2cc(-c3ccc(Cl)cc3)nc2n1CC(=O)NC(C)C] | Method A | 1.89 | 359 |
| 69 | 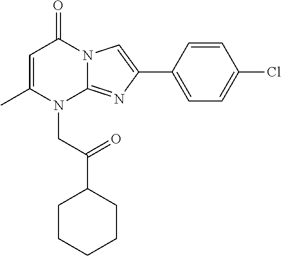 [SMILES: Cc1cc(=O)n2cc(-c3ccc(Cl)cc3)nc2n1CC(=O)C1CCCCC1] | Method A | 2.31 | 384 |

TABLE 15

| | | | Retention | |
|---|---|---|---|---|
| | | | Time | Mass |
| Compound | Structure | LC/MS | [min] | [M + H] |
| 70 | 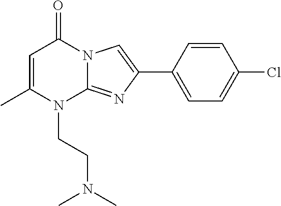 [SMILES: Cc1cc(=O)n2cc(-c3ccc(Cl)cc3)nc2n1CCN(C)C] | Method A | 1.29 | 331 |
| 71 | 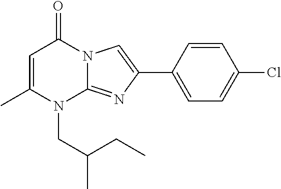 [SMILES: CCC(C)Cn1c(C)cc(=O)n2cc(-c3ccc(Cl)cc3)nc12] | Method A | 2.73 | 330 |
| 72 | 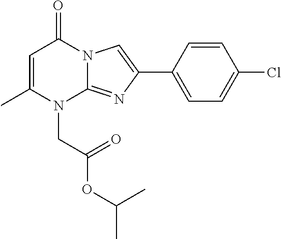 [SMILES: Cc1cc(=O)n2cc(-c3ccc(Cl)cc3)nc2n1CC(=O)OC(C)C] | Method A | 2.37 | 360 |
| 73 | 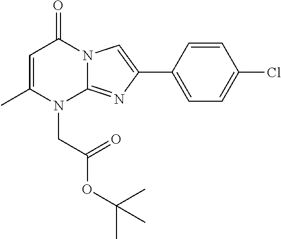 [SMILES: Cc1cc(=O)n2cc(-c3ccc(Cl)cc3)nc2n1CC(=O)OC(C)(C)C] | Method A | 2.51 | 374 |
| 74 | 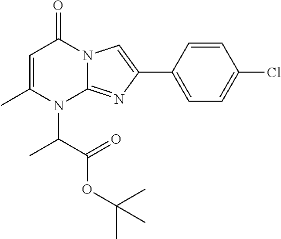 [SMILES: Cc1cc(=O)n2cc(-c3ccc(Cl)cc3)nc2n1C(C)C(=O)OC(C)(C)C] | Method A | 2.62 | 388 |

TABLE 16

| | | | Retention | |
|---|---|---|---|---|
| | | | Time | |
| Compound | Structure | LC/MS | [min] | Mass [M + H] |
| 75 | 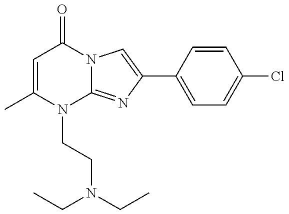 [SMILES: CCN(CC)CCn1c(C)cc(=O)n2cc(-c3ccc(Cl)cc3)nc12] | Method A | 1.43 | 359 |
| 76 | 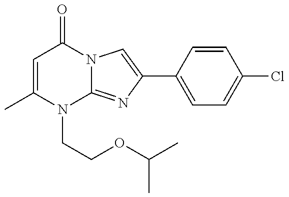 [SMILES: Cc1cc(=O)n2cc(-c3ccc(Cl)cc3)nc2n1CCOC(C)C] | Method A | 2.49 | 346 |
| 77 | 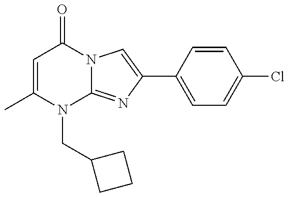 [SMILES: Cc1cc(=O)n2cc(-c3ccc(Cl)cc3)nc2n1CC1CCC1] | Method A | 2.64 | 328 |
| 78 | 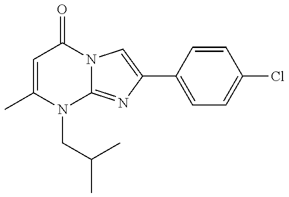 [SMILES: Cc1cc(=O)n2cc(-c3ccc(Cl)cc3)nc2n1CC(C)C] | Method A | 2.54 | 316 |
| 79 | 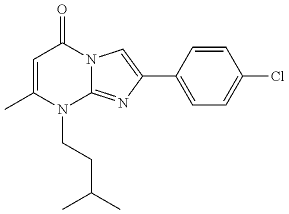 [SMILES: Cc1cc(=O)n2cc(-c3ccc(Cl)cc3)nc2n1CCC(C)C] | Method A | 2.75 | 330 |

TABLE 17

| | | | Retention | |
|---|---|---|---|---|
| | | | Time | |
| Compound | Structure | LC/MS | [min] | Mass [M + H] |
| 80 | 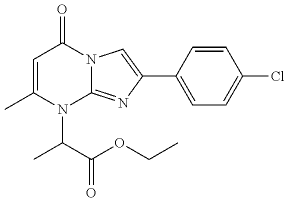 [SMILES: CCOC(=O)C(C)n1c(C)cc(=O)n2cc(-c3ccc(Cl)cc3)nc12] | Method A | 2.34 | 360 |
| 81 | 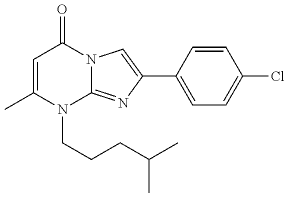 [SMILES: Cc1cc(=O)n2cc(-c3ccc(Cl)cc3)nc2n1CCCC(C)C] | Method A | 2.92 | 344 |
| 82 | 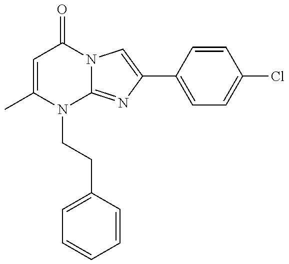 [SMILES: Cc1cc(=O)n2cc(-c3ccc(Cl)cc3)nc2n1CCc1ccccc1] | Method A | 2.67 | 364 |
| 83 | 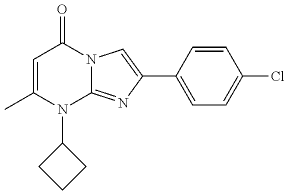 [SMILES: Cc1cc(=O)n2cc(-c3ccc(Cl)cc3)nc2n1C1CCC1] | Method A | 2.43 | 314 |
| 84 | 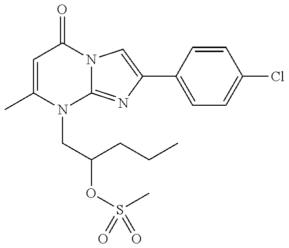 [SMILES: CCCC(Cn1c(C)cc(=O)n2cc(-c3ccc(Cl)cc3)nc12)OS(C)(=O)=O] | Method B | 2.30 | 424 |

TABLE 18

| | | | Retention | |
|---|---|---|---|---|
| | | | Time | |
| Compound | Structure | LC/MS | [min] | Mass [M + H] |
| 85 | 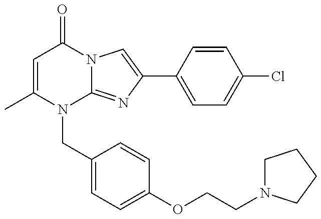 [SMILES: Cc1cc(=O)n2cc(-c3ccc(Cl)cc3)nc2n1Cc1ccc(OCCN2CCCC2)cc1] | Method A | 1.76 | 463 |
| 86 | 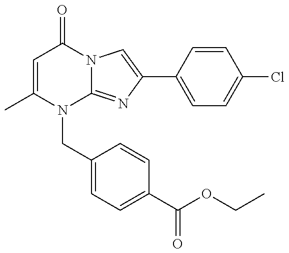 [SMILES: CCOC(=O)c1ccc(Cn2c(C)cc(=O)n3cc(-c4ccc(Cl)cc4)nc23)cc1] | Method A | 2.62 | 422 |
| 87 | 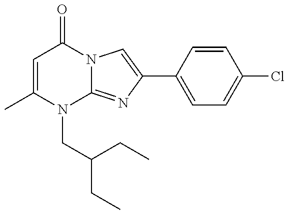 [SMILES: CCC(CC)Cn1c(C)cc(=O)n2cc(-c3ccc(Cl)cc3)nc12] | Method A | 2.92 | 344 |
| 88 | 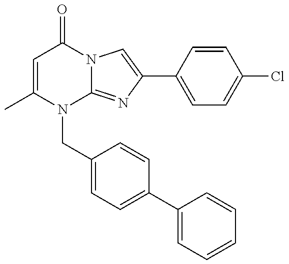 [SMILES: Cc1cc(=O)n2cc(-c3ccc(Cl)cc3)nc2n1Cc1ccc(-c2ccccc2)cc1] | Method A | 2.68 | 426 |
| 89 | 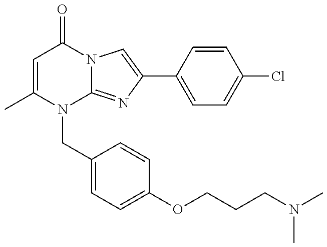 | Method A | 1.77 | 451 |

TABLE 19

| | | | Retention | |
|---|---|---|---|---|
| | | | Time | |
| Compound | Structure | LC/MS | [min] | Mass [M + H] |
| 90 | 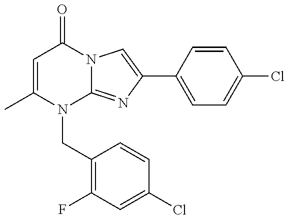 [SMILES: Cc1cc(=O)n2cc(-c3ccc(Cl)cc3)nc2n1Cc1ccc(Cl)cc1F] | Method A | 2.81 | 402 |
| 91 | 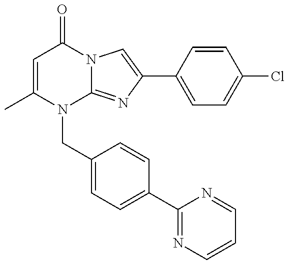 [SMILES: Cc1cc(=O)n2cc(-c3ccc(Cl)cc3)nc2n1Cc1ccc(-c2ncccn2)cc1] | Method A | 2.08 | 428 |
| 92 | 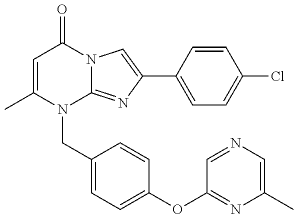 | Method A | 2.48 | 458 |
| 93 | 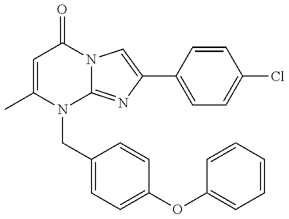 [SMILES: Cc1cc(=O)n2cc(-c3ccc(Cl)cc3)nc2n1Cc1ccc(Oc2ccccc2)cc1] | Method A | 2.96 | 442 |
| 94 | 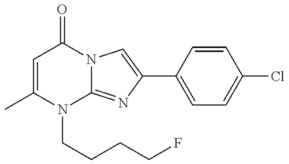 [SMILES: Cc1cc(=O)n2cc(-c3ccc(Cl)cc3)nc2n1CCCCF] | Method A | 2.28 | 334 |

TABLE 20

| | | | Retention | |
|---|---|---|---|---|
| | | | Time | |
| Compound | Structure | LC/MS | [min] | Mass [M + H] |
| 95 | 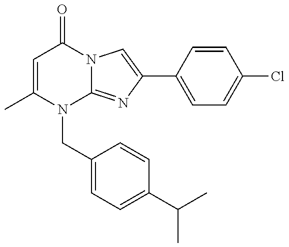 [SMILES: Cc1cc(=O)n2cc(-c3ccc(Cl)cc3)nc2n1Cc1ccc(C(C)C)cc1] | Method A | 2.99 | 392 |
| 96 | 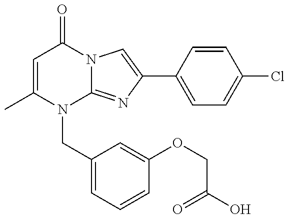 [SMILES: Cc1cc(=O)n2cc(-c3ccc(Cl)cc3)nc2n1Cc1cccc(OCC(=O)O)c1] | Method A | 2.08 | 424 |
| 97 | 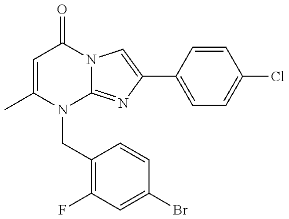 [SMILES: Cc1cc(=O)n2cc(-c3ccc(Cl)cc3)nc2n1Cc1ccc(Br)cc1F] | Method A | 2.85 | 446 |
| 98 | 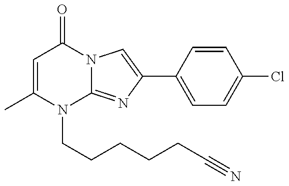 [SMILES: Cc1cc(=O)n2cc(-c3ccc(Cl)cc3)nc2n1CCCCCC#N] | Method A | 2.18 | 355 |
| 99 | 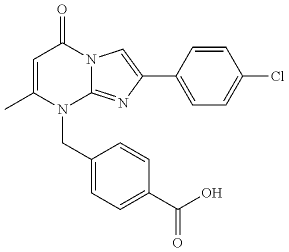 [SMILES: Cc1cc(=O)n2cc(-c3ccc(Cl)cc3)nc2n1Cc1ccc(C(=O)O)cc1] | Method A | 2.04 | 394 |

TABLE 21

| | | | Retention | |
|---|---|---|---|---|
| | | | Time | |
| Compound | Structure | LC/MS | [min] | Mass [M + H] |
| 100 | 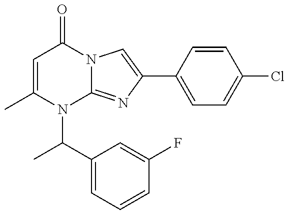 [SMILES: Cc1cc(=O)n2cc(-c3ccc(Cl)cc3)nc2n1C(C)c1cccc(F)c1] | Method A | 2.13 | 382 |
| 101 | 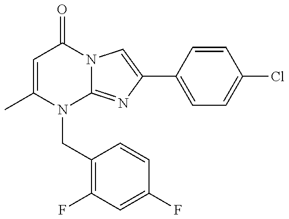 [SMILES: Cc1cc(=O)n2cc(-c3ccc(Cl)cc3)nc2n1Cc1ccc(F)cc1F] | Method A | 2.63 | 386 |
| 102 | 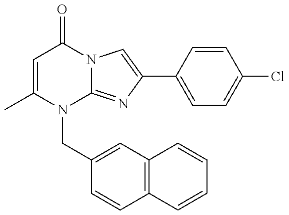 [SMILES: Cc1cc(=O)n2cc(-c3ccc(Cl)cc3)nc2n1Cc1ccc2ccccc2c1] | Method A | 2.81 | 400 |
| 103 | 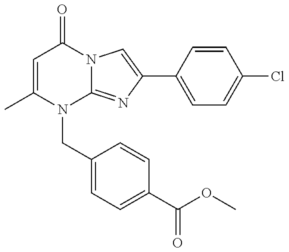 [SMILES: COC(=O)c1ccc(Cn2c(C)cc(=O)n3cc(-c4ccc(Cl)cc4)nc23)cc1] | Method A | 2.45 | 408 |
| 104 | 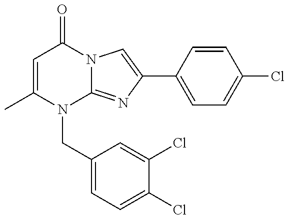 [SMILES: Cc1cc(=O)n2cc(-c3ccc(Cl)cc3)nc2n1Cc1ccc(Cl)c(Cl)c1] | Method A | 2.89 | 418 |

TABLE 22

| | | | Retention | |
|---|---|---|---|---|
| | | | Time | |
| Compound | Structure | LC/MS | [min] | Mass [M + H] |
| 105 | 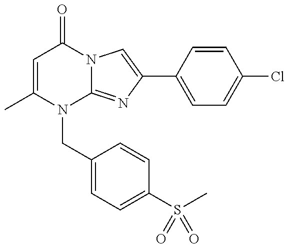 [SMILES: Cc1cc(=O)n2cc(-c3ccc(Cl)cc3)nc2n1Cc1ccc(S(C)(=O)=O)cc1] | Method A | 2.08 | 428 |
| 106 | 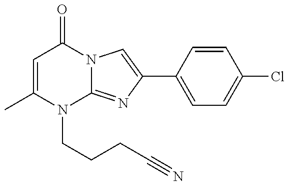 [SMILES: Cc1cc(=O)n2cc(-c3ccc(Cl)cc3)nc2n1CCCC#N] | Method A | 1.97 | 327 |
| 107 | 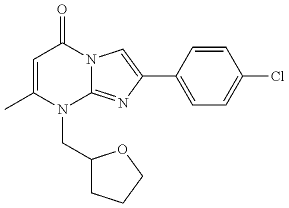 [SMILES: Cc1cc(=O)n2cc(-c3ccc(Cl)cc3)nc2n1CC1CCCO1] | Method A | 2.26 | 344 |
| 108 | 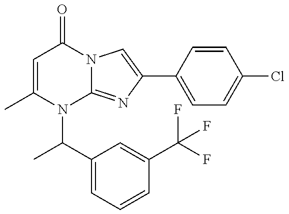 [SMILES: Cc1cc(=O)n2cc(-c3ccc(Cl)cc3)nc2n1C(C)c1cccc(C(F)(F)F)c1] | Method A | 2.33 | 432 |
| 109 | 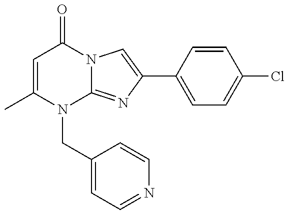 [SMILES: Cc1cc(=O)n2cc(-c3ccc(Cl)cc3)nc2n1Cc1ccncc1] | Method A | 1.49 | 351 |

TABLE 23

| | | | Retention | |
|---|---|---|---|---|
| | | | Time | |
| Compound | Structure | LC/MS | [min] | Mass [M + H] |
| 110 | 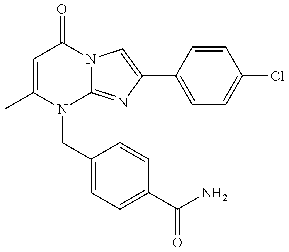 [SMILES: Cc1cc(=O)n2cc(-c3ccc(Cl)cc3)nc2n1Cc1ccc(C(N)=O)cc1] | Method A | 1.82 | 393 |
| 111 | 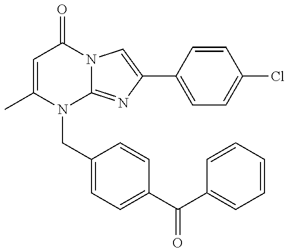 [SMILES: Cc1cc(=O)n2cc(-c3ccc(Cl)cc3)nc2n1Cc1ccc(C(=O)c2ccccc2)cc1] | Method A | 2.71 | 454 |
| 112 | 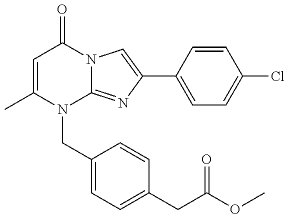 [SMILES: COC(=O)Cc1ccc(Cn2c(C)cc(=O)n3cc(-c4ccc(Cl)cc4)nc23)cc1] | Method A | 2.42 | 422 |
| 113 | 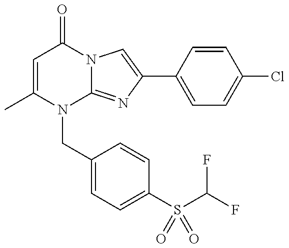 [SMILES: Cc1cc(=O)n2cc(-c3ccc(Cl)cc3)nc2n1Cc1ccc(S(=O)(=O)C(F)F)cc1] | Method A | 2.38 | 464 |
| 114 | 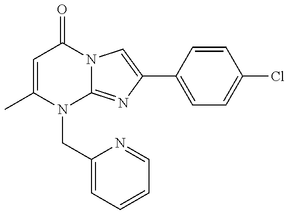 [SMILES: Cc1cc(=O)n2cc(-c3ccc(Cl)cc3)nc2n1Cc1ccccn1] | Method A | 2.07 | 351 |

TABLE 24

| | | | Retention | |
|---|---|---|---|---|
| | | | Time | |
| Compound | Structure | LC/MS | [min] | Mass [M + H] |
| 115 | 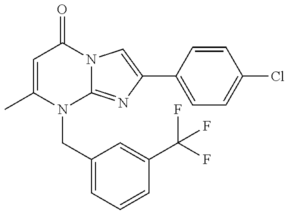 [SMILES: Cc1cc(=O)n2cc(-c3ccc(Cl)cc3)nc2n1Cc1cccc(C(F)(F)F)c1] | Method A | 2.73 | 418 |
| 116 | 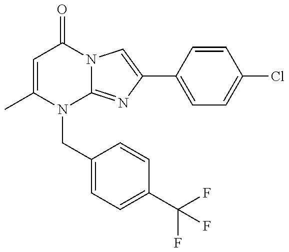 [SMILES: Cc1cc(=O)n2cc(-c3ccc(Cl)cc3)nc2n1Cc1ccc(C(F)(F)F)cc1] | Method A | 2.75 | 418 |
| 117 | 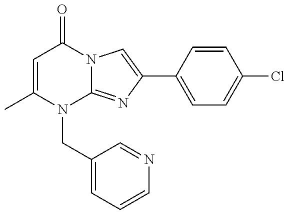 [SMILES: Cc1cc(=O)n2cc(-c3ccc(Cl)cc3)nc2n1Cc1cccnc1] | Method A | 1.61 | 351 |
| 118 | 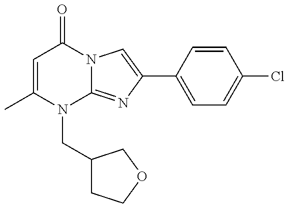 [SMILES: Cc1cc(=O)n2cc(-c3ccc(Cl)cc3)nc2n1CC1CCOC1] | Method A | 2.05 | 344 |
| 119 | 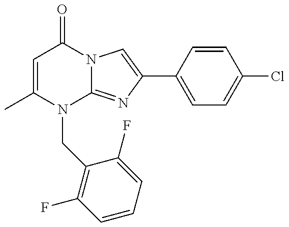 [SMILES: Cc1cc(=O)n2cc(-c3ccc(Cl)cc3)nc2n1Cc1c(F)cccc1F] | Method A | 2.52 | 386 |

TABLE 25

| | | | Retention | |
|---|---|---|---|---|
| | | | Time | |
| Compound | Structure | LC/MS | [min] | Mass [M + H] |
| 120 | 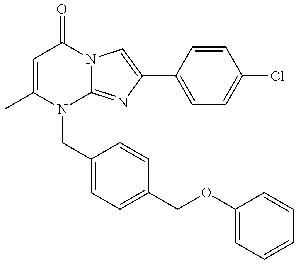 [SMILES: Cc1cc(=O)n2cc(-c3ccc(Cl)cc3)nc2n1Cc1ccc(COc2ccccc2)cc1] | Method A | 2.90 | 456 |
| 121 | 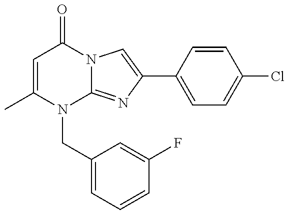 [SMILES: Cc1cc(=O)n2cc(-c3ccc(Cl)cc3)nc2n1Cc1cccc(F)c1] | Method A | 2.55 | 368 |
| 122 | 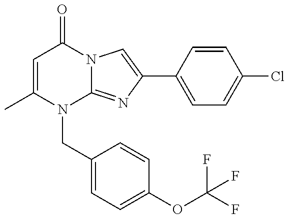 [SMILES: Cc1cc(=O)n2cc(-c3ccc(Cl)cc3)nc2n1Cc1ccc(OC(F)(F)F)cc1] | Method A | 2.82 | 434 |
| 123 | 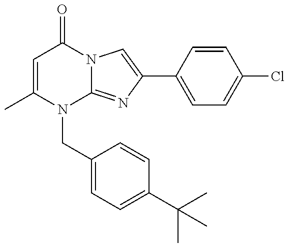 [SMILES: Cc1cc(=O)n2cc(-c3ccc(Cl)cc3)nc2n1Cc1ccc(C(C)(C)C)cc1] | Method A | 3.09 | 406 |
| 124 | 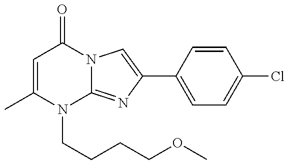 [SMILES: COCCCCn1c(C)cc(=O)n2cc(-c3ccc(Cl)cc3)nc12] | Method A | 2.24 | 346 |

### Example 3

### 8-(4-chlorophenyl)-2-propylimidazo[1,2-a]pyrimidin-5(8H)-one (127)

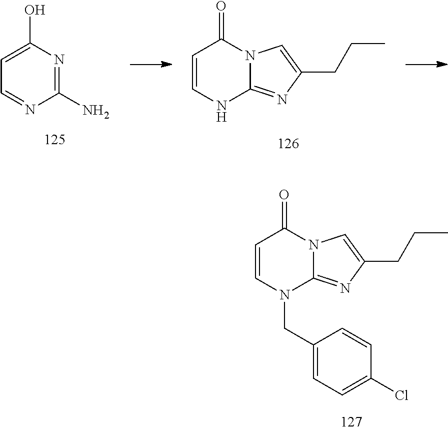 [SMILES: Nc1nccc(O)n1 ; CCCc1cn2c(=O)cc[nH]c2n1 ; CCCc1cn2c(=O)ccn(Cc3ccc(Cl)cc3)c2n1]

### Step 1:

To a solution of 2-aminopyrimidin-4-ol (125, 333 mg, 3.00 mmol) in N,N-dimethylformamide (5 mL) was added sodium hydride under ice-cooling (60 wt %, 132 mg, 3.30 mmol), and the mixture was stirred at room temperature for 30 minutes. A solution of 1-bromopentan-2-one (495 mg, 3.00 mmol, prepared according to Bioorg. Med. Chem. 15 (2007) 3225-3234) in N,N-dimethylformamide (4 mL) was added under ice-cooling, and the mixture was stirred for 1 hour. To the reaction mixture was added sodium hydroxide solution (2 mol/L, 1 mL), and the mixture was stirred at room temperature for 30 minutes. Hydrochloric acid (2 mol/L, 1.1 mL) was added, and the mixture was extracted four times with chloroform/methanol (9:1). The organic layer was dried with anhydrous sodium sulfate and concentrated under reduced pressure. The obtained residue was purified by silica gel chromatography (chloroform/methanol) to yield 2-propylimidazo[1,2-a]pyrimidin-5(8H)-one (126, 275 mg, yield: 52%) as a colorless solid.

1H-NMR (δ ppm TMS/DMSO-d6) 7.94 (s, 1H), 7.85 (d, LH, J=6.5 Hz), 7.34 (s, 1H), 5.76 (d, 1H, J=6.3 Hz), 2.57 (t, 2H, J=7.4 Hz), 1.67-1.64 (m, 2H), 0.91 (t, 3H, J=7.3 Hz).

### Step 2:

To a solution of the compound (126, 25 mg, 0.14 mmol) in N,N-dimethylformamide (0.8 mL) was added potassium carbonate (23 mg, 0.17 mmol) and 4-chlorobenzyl bromide (44 mg, 0.21 mmol), and the mixture was stirred at 50° C. for 6 hours. After cooling to room temperature, water was added, and the reaction mixture was extracted with ethyl acetate. The organic layer was washed with brine, dried with anhydrous sodium sulfate and concentrated under reduced pressure. The residue was purified by silica gel chromatography (hexane/ethyl acetate) to yield 8-(4-chlorophenyl)-2-propylimidazo[1,2-a]pyrimidin-5(8H)-one (127, 30 mg, yield: 70%) as a colorless solid.

1H-NMR (δ ppm TMS/CDCl3) 7.34-7.30 (m, 6H), 5.78 (d, 1H, J=7.5 Hz), 5.31 (s, 2H), 2.64 (t, 2H, J=7.5 Hz), 1.78-1.69 (m, 2H), 1.00 (t, 3H, J=7.3 Hz).

Compounds (128) and (129) were prepared in a similar manner.

TABLE 26

| Com- | | |
|---|---|---|
| pound | Structure | 1H-NMR |
| 128 | 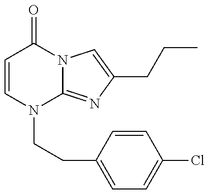 [SMILES: CCCc1cn2c(=O)ccn(CCc3ccc(Cl)cc3)c2n1] | (δppm TMS/CDCl3) 7.38 (s, 1H), 7.25 (d, 2H, J = 7.5 Hz), 7.02 (d, 2H, J = 7.5 Hz), 6.95 (d, 1H, J = 7.8 Hz), 5.61 (d, 1H, J = 7.5 Hz), 4.36 (t, 2H, J = 6.7 Hz), 3.17 (t, 2H, J = 6.7 Hz), 2.65 (t, 2H, J = 7.5 Hz), 1.80 (s, 1H), 1.76-1.72 (m, 2H), 1.01 (t, 3H, J = 7.3 Hz). |
| 129 | 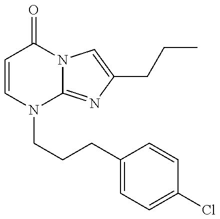 [SMILES: CCCc1cn2c(=O)ccn(CCCc3ccc(Cl)cc3)c2n1] | (δppm TMS/CDCl3) 7.37 (s, 1H), 7.29-7.26 (m, 4H), 7.10 (d, 2H, J = 8.0 Hz), 5.76 (d, 1H, J = 7.8 Hz), 4.18 (t, 2H, J = 7.2 Hz), 2.70-2.62 (m, 4H), 2.27-2.19 (m, 2H), 1.78- 1.70 (m, 2H), 1.01 (t, 3H, J = 7.4 Hz). |

### Example 4

### 4-(5-oxo-8-(4,4,4-trifluorobutyl)-2-((1r,4r)-4-(trifluoromethyl)cyclohexyl)-5,8-dihydroimidazo[1,2-a]pyrimidine-7-yl)benzamide (136)

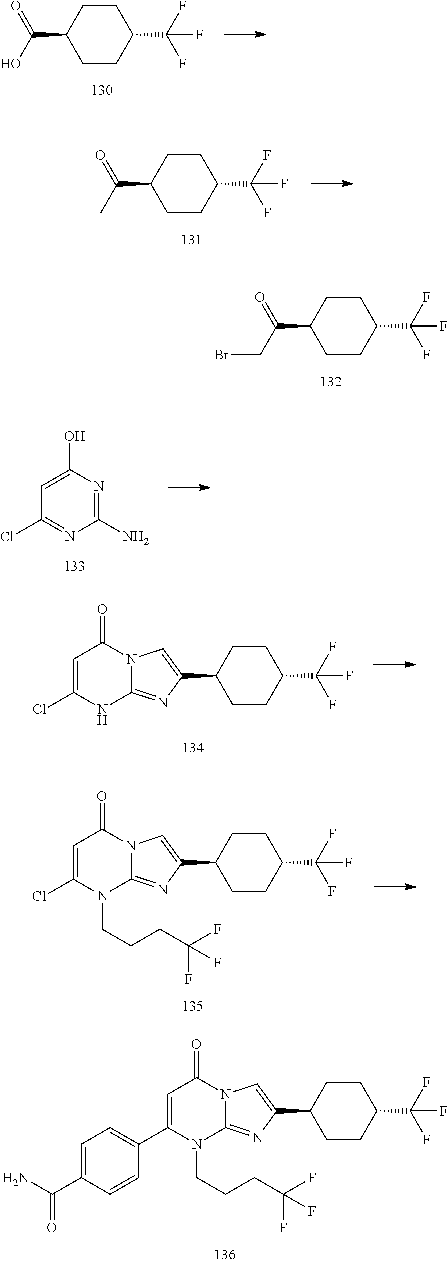

### Step 1:

To a solution of (1r,4r)-4-(trifluoromethyl)cyclohexanecarboxylic acid (130, 2.00 g, 10.2 mmol) in tetrahydrofuran (75 mL) was added methyl lithium in tetrahydrofuran (1.14 mol/L, 36 mL, 41 mmol) under ice-cooling, and the mixture was stirred for 2 hours under cooling. Chlorotrimethylsilane (26 mL, 204 mmol) was added, and the reaction mixture was warmed to room temperature. Hydrochloric acid (1 mol/L, 75 mL) was added, and the mixture was stirred at room temperature for 30 minutes. The reaction mixture was extracted twice with diethyl ether. The organic layer was dried over anhydrous magnesium sulfate and concentrated under reduced pressure to yield the crude product of 1-((1r,4r)-4-(trifluoromethyl)cyclohexyl)ethanone (131) (1.97 g).

### Step 2:

To a solution of the crude product of the compound (131) (1.10 g) in methanol (7 mL) was added bromine (0.29 mL, 5.7 mmol) in methanol (3 mL) under ice-cooling, and the mixture was stirred at room temperature for 6 hours. Water (50 mL) was added to the reaction mixture, and the mixture was extracted twice with diethyl ether. The organic layer was washed with saturated aqueous sodium bicarbonate, dried over anhydrous magnesium sulfate and concentrated under reduced pressure to yield the crude product of 2-bromo-1-((1r,4r)-4-(trifluoromethyl)cyclohexyl)ethanone (132) (2.3 g).

### Step 3:

To a solution of 2-amino-6-chloropyrimidin-4-ol (133, 200 mg, 1.37 mmol) in N,N-dimethylformamide (4 mL) was added sodium hydride (60 wt %, 55 mg, 1.4 mmol) under ice-cooling, and the mixture was stirred at room temperature for 30 minutes. A solution of the crude product of the compound (132) (670 mg) in N,N-dimethylformamide (2 mL) was added under ice-cooling, and the mixture was stirred at room temperature for 5 hours. Sodium hydroxide solution (2 mol/L, 1.27 mL) was added to the reaction mixture, and the mixture was stirred at room temperature for 2 hours. HCl (2 mol/L, 1.4 mL) and water (50 mL) were added, and the reaction mixture was extracted twice with ethyl acetate. The organic layer was dried with anhydrous magnesium sulfate and concentrated under reduced pressure to yield the crude product of 7-chloro-2-((1r,4r)-4-(trifluoromethyl)cyclohexyl)imidazo[1,2-a]pyrimidin-5(8H)-one (134)(615 mg).

### Step 4:

To a solution of the crude product of the compound (134) (300 mg) in N,N-dimethylformamide (4.5 mL) was added sodium hydride (60 wt %, 32 mg, 0.80 mmol) under ice-cooling, and the mixture was stirred at room temperature for 15 minutes. To the reaction mixture was added 4-bromo-1,1,1-trifluorobutane (0.25 mL, 2.0 mmol), and the mixture was stirred at 100° C. for 3 hours. After cooling to room temperature, water was added, and the reaction mixture was extracted with ethyl acetate. The organic layer was washed with water and brine, dried over anhydrous magnesium sulfate and concentrated under reduced pressure to give the crude product of 7-chloro-2-((1r,4r)-4-(trifluoromethyl)cyclohexyl)imidazo[1,2-a]pyrimidin-5(8H)-one (135) (101 mg), and then about half of which (50 mg) was purified by silica gel chromatography (hexane/ethyl acetate) to yield the compound (135, 11 mg, yield from the compound (133): 8%) as a pale brown solid.

1H-NMR (δ ppm TMS/DMSO-d6) 7.34 (s, 1H), 6.11 (s, 1H), 4.44 (t, 2H, J=7.3 Hz), 2.44-2.53 (m, 3H), 2.28-2.30 (br m, 1H), 1.99-2.07 (m, 6H), 1.41-1.43 (m, 4H).

### Step 5:

To a solution of the crude product of the compound (135) (50 mg),4-carbamoylphenylboronic acid (29 mg, 0.18 mmol) and [1,1′-bis(diphenylphosphino)ferrocene]palladium(II) dichloride-dichloromethane complex (9.5 mg, 0.012 mmol) in N,N-dimethylformamide (1 mL) was added aqueous sodium carbonate (2 mol/L, 0.23 mL), and the mixture was stirred at 100° C. for 20 minutes. After cooling the reaction mixture to room temperature, water was added, and the mixture was extracted with ethyl acetate. The organic layer was dried with anhydrous magnesium sulfate and concentrated under reduced pressure. The residue was purified by silica gel chromatography (hexane/ethyl acetate) to give 4-(5-oxo-8-(4,4,4-trifluorobutyl)-2-((1r,4r)-4-(trifluoromethyl)cyclohexyl)-5,8-dihydroimidazo[1,2-a]pyrimidin-7-yl)benzamide (136, 2.4 mg, yield from the compound (133): 1%) as a yellow oil.

LC/MS (Method B) Retention Time=2.19 min, Found Mass [M+H]=515.

Compounds (137) to (166) were prepared in a similar manner.

TABLE 27

| Compound | Structure | 1H-NMR |
|---|---|---|
| 137 | 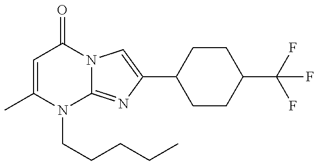 [SMILES: CCCCCn1c(C)cc(=O)n2cc(C3CCC(C(F)(F)F)CC3)nc12] | (δppm TMS/DMSO-d6) 7.26 (s, 1H), 5.71 (s, 1H), 4.16-4.19 (m, 2H), 2.50-2.52 (br m, 1H), 2.43 (s, 3H), 2.31 (br s, 1H), 2.10-2.13 (br m, 2H), 1.94- 1.97 (br m, 2H), 1.70-1.73 (br m, 2H), 1.32-1.42 (m, 8H), 0.88 (t, 3H, J = 10.0 Hz). |
| 138 | 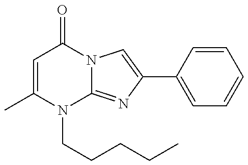 [SMILES: CCCCCn1c(C)cc(=O)n2cc(-c3ccccc3)nc12] | (δppm TMS/DMSO-d6) 8.12 (s, 1H), 7.95 (d, 2H, J = 7.8 Hz), 7.42 (t, 2H, J = 7.5 Hz), 7.31-7.33 (m, 1H), 5.77 (s, 1H), 4.27 (t, 2H, J = 7.0 Hz), 2.47 (s, 2H), 1.79-1.81 (m, 2H), 1.37-1.39 (m, 4H), 0.91 (t, 3H, J = 7.0 Hz). |

TABLE 28

| | | | Retention | |
|---|---|---|---|---|
| | | | Time | |
| Compound | Structure | LC/MS | [min] | Mass [M + H] |
| 139 | 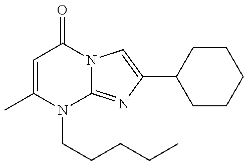 [SMILES: CCCCCn1c(C)cc(=O)n2cc(C3CCCCC3)nc12] | Method A | 2.44 | 302 |
| 140 | 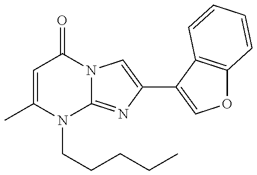 [SMILES: CCCCCn1c(C)cc(=O)n2cc(-c3coc4ccccc34)nc12] | Method A | 2.34 | 336 |
| 141 | 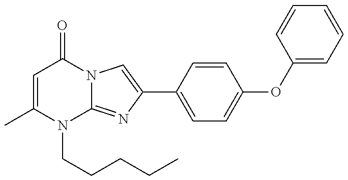 [SMILES: CCCCCn1c(C)cc(=O)n2cc(-c3ccc(Oc4ccccc4)cc3)nc12] | Method A | 2.71 | 388 |
| 142 | 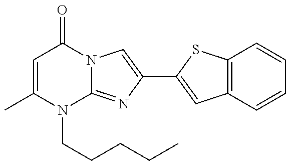 [SMILES: CCCCCn1c(C)cc(=O)n2cc(-c3cc4ccccc4s3)nc12] | Method A | 2.58 | 352 |
| 143 | 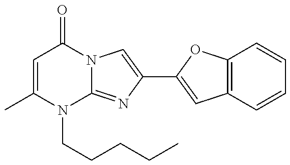 [SMILES: CCCCCn1c(C)cc(=O)n2cc(-c3cc4ccccc4o3)nc12] | Method A | 2.52 | 336 |

TABLE 29

| | | | Retention | |
|---|---|---|---|---|
| | | | Time | |
| Compound | Structure | LC/MS | [min] | Mass [M + H] |
| 144 | 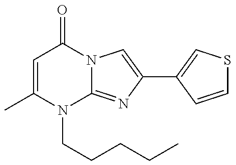 [SMILES: CCCCCn1c(C)cc(=O)n2cc(-c3ccsc3)nc12] | Method A | 2.13 | 302 |
| 145 | 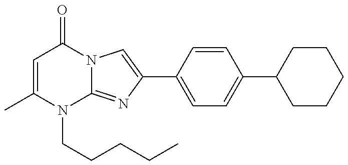 [SMILES: CCCCCn1c(C)cc(=O)n2cc(-c3ccc(C4CCCCC4)cc3)nc12] | Method A | 3.17 | 378 |
| 146 | 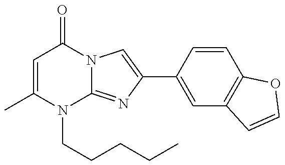 [SMILES: CCCCCn1c(C)cc(=O)n2cc(-c3ccc4occc4c3)nc12] | Method A | 2.29 | 336 |
| 147 | 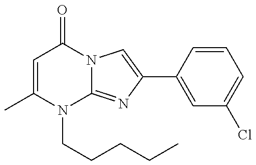 [SMILES: CCCCCn1c(C)cc(=O)n2cc(-c3cccc(Cl)c3)nc12] | Method A | 2.40 | 330 |
| 148 | 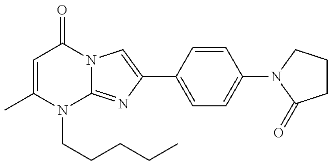 [SMILES: CCCCCn1c(C)cc(=O)n2cc(-c3ccc(N4CCCC4=O)cc3)nc12] | Method A | 1.92 | 379 |

TABLE 30

| | | | Retention | |
|---|---|---|---|---|
| | | | Time | |
| Compound | Structure | LC/MS | [min] | Mass [M + H] |
| 149 | 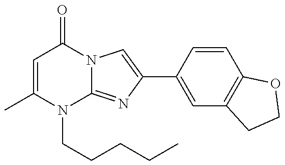 [SMILES: CCCCCn1c(C)cc(=O)n2cc(-c3ccc4c(c3)CCO4)nc12] | Method A | 2.19 | 338 |
| 150 | 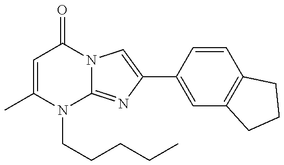 [SMILES: CCCCCn1c(C)cc(=O)n2cc(-c3ccc4c(c3)CCC4)nc12] | Method A | 2.64 | 336 |
| 151 | 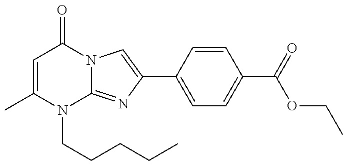 [SMILES: CCCCCn1c(C)cc(=O)n2cc(-c3ccc(C(=O)OCC)cc3)nc12] | Method A | 2.38 | 368 |
| 152 | 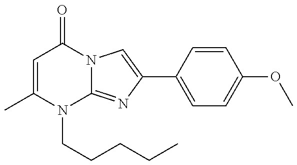 | Method A | 2.21 | 326 |
| 153 | 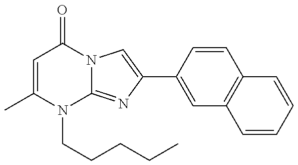 [SMILES: CCCCCn1c(C)cc(=O)n2cc(-c3ccc4ccccc4c3)nc12] | Method A | 2.53 | 346 |

TABLE 31

| | | | Retention | |
|---|---|---|---|---|
| | | | Time | |
| Compound | Structure | LC/MS | [min] | Mass [M + H] |
| 154 | 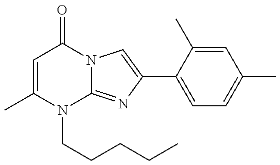 | Method A | 2.51 | 324 |
| 155 | 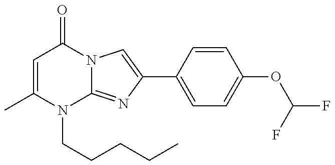 [SMILES: CCCCCn1c(C)cc(=O)n2cc(-c3ccc(OC(F)F)cc3)nc12] | Method A | 2.30 | 362 |
| 156 | 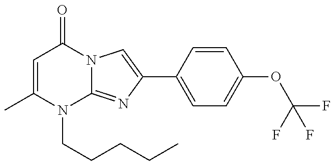 [SMILES: CCCCCn1c(C)cc(=O)n2cc(-c3ccc(OC(F)(F)F)cc3)nc12] | Method A | 2.54 | 380 |
| 157 | 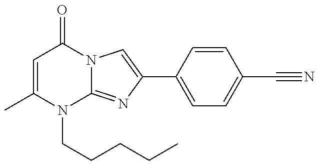 [SMILES: CCCCCn1c(C)cc(=O)n2cc(-c3ccc(C#N)cc3)nc12] | Method A | 2.05 | 321 |
| 158 | 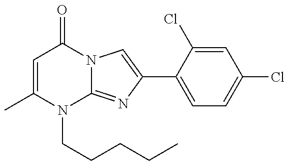 [SMILES: CCCCCn1c(C)cc(=O)n2cc(-c3ccc(Cl)cc3Cl)nc12] | Method A | 2.58 | 364 |

TABLE 32

| | | | Retention | |
|---|---|---|---|---|
| | | | Time | |
| Compound | Structure | LC/MS | [min] | Mass [M + H] |
| 159 | 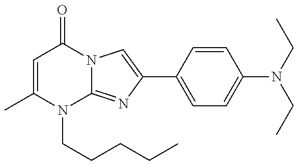 [SMILES: CCCCCn1c(C)cc(=O)n2cc(-c3ccc(N(CC)CC)cc3)nc12] | Method A | 1.67 | 367 |
| 160 | 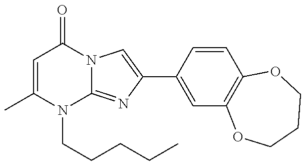 [SMILES: CCCCCn1c(C)cc(=O)n2cc(-c3ccc4c(c3)OCCCO4)nc12] | Method A | 2.24 | 368 |
| 161 | 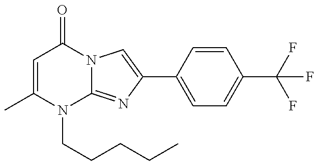 [SMILES: CCCCCn1c(C)cc(=O)n2cc(-c3ccc(C(F)(F)F)cc3)nc12] | Method A | 2.48 | 364 |
| 162 | 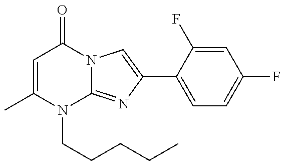 [SMILES: CCCCCn1c(C)cc(=O)n2cc(-c3ccc(F)cc3F)nc12] | Method A | 2.25 | 332 |
| 163 | 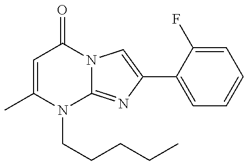 [SMILES: CCCCCn1c(C)cc(=O)n2cc(-c3ccccc3F)nc12] | Method A | 2.20 | 314 |

TABLE 33

| | | | Reten- | |
|---|---|---|---|---|
| | | | tion | |
| Com- | | | Time | Mass |
| pound | Structure | LC/MS | [min] | [M + H] |
| 164 | 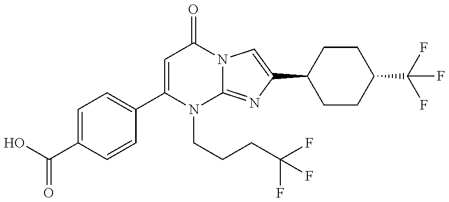 [SMILES: O=C(O)c1ccc(-c2cc(=O)n3cc([C@H]4CC[C@H](C(F)(F)F)CC4)nc3n2CCCC(F)(F)F)cc1] | Method B | 2.37 | 517 |
| 165 | 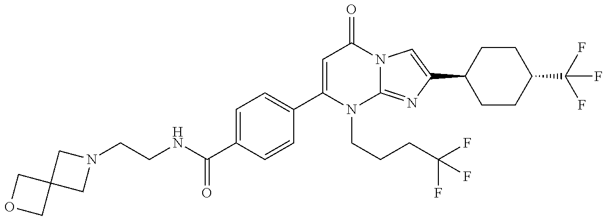 [SMILES: O=C(NCCN1CC2(COC2)C1)c1ccc(-c2cc(=O)n3cc([C@H]4CC[C@H](C(F)(F)F)CC4)nc3n2CCCC(F)(F)F)cc1] | Method B | 1.81 | 640 |
| 166 | 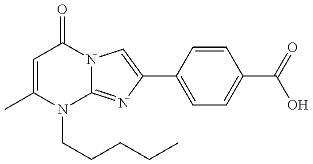 [SMILES: CCCCCn1c(C)cc(=O)n2cc(-c3ccc(C(=O)O)cc3)nc12] | Method A | 1.95 | 340 |

### Example 5

### 2-(4-chlorophenyl)-3-hydroxymethyl-7-methyl-8-pentylimidazo[1,2-a]pyrimidin-5(8H)-one (168)

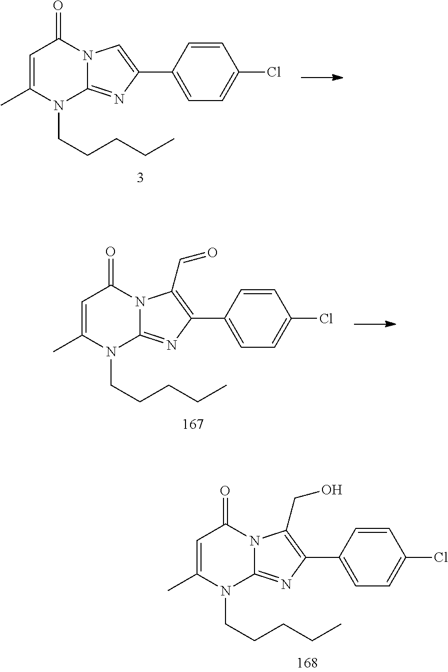 [SMILES: CCCCCn1c(C)cc(=O)n2cc(-c3ccc(Cl)cc3)nc12 ; CCCCCn1c(C)cc(=O)n2c(C=O)c(-c3ccc(Cl)cc3)nc12 ; CCCCCn1c(C)cc(=O)n2c(CO)c(-c3ccc(Cl)cc3)nc12]

To a solution of the compound (3, 150 mg, 0.455 mmol) in N,N-dimethylformamide (1.5 mL) was added (chloromethylene)dimethylammoniumiminium chloride (146 mg, 1.14 mmol), and the solution was stirred at room temperature for 90 minutes. Saturated aqueous sodium bicarbonate (30 mL) was added, and the mixture was extracted with ethyl acetate (30 mL). The organic layer was washed twice with water, dried with anhydrous magnesium sulfate and concentrated under reduced pressure. The residue was purified by silica gel chromatography (hexane/ethyl acetate) to yield 2-(4-chlorophenyl)-7-methyl-5-oxo-8-pentyl-5,8-dihydroimidazo[1,2-a]pyrimidine-3-carbaldehyde (167, 151 mg, yield: 93%) as a yellow solid.

1H-NMR (δ ppm TMS/CDCl3) 11.00 (s, 1H), 8.16 (d, 2H, J=8.4 Hz), 7.43 (d, 2H, J=8.3 Hz), 5.88 (s, 1H), 4.32 (t, 2H, J=7.8 Hz), 2.48 (s, 3H), 1.85-1.87 (m, 2H), 1.42-1.44 (m, 4H), 0.95 (t, 3H, J=10.0 Hz).

### Step 2:

To a solution of the compound (167, 20 mg, 0.056 mmol) in methanol (0.5 mL) was added sodium borohydride (4.2 mg, 0.11 mmol), and the solution was stirred at room temperature for 1 hour. Water (20 mL) was added to the reaction mixture, and the mixture was extracted with ethyl acetate (20 mL). The organic layer was dried over anhydrous magnesium sulfate and concentrated under reduced pressure. The residue was triturated in diisopropyl ether to yield 2-(4-chlorophenyl)-3-hydroxymethyl-7-methyl-8-pentylimidazo[1,2-a]pyrimidin-5(8H)-one (168, 13 mg, yield: 66%) as a colorless solid.

1H-NMR (δ ppm TMS/CDCl3) 7.62 (d, 2H, J=8.0 Hz), 7.42 (d, 2H, J=8.0 Hz), 5.71 (s, 1H), 4.93 (d, 2H, J=7.5 Hz), 4.27 (t, 2H, J=7.8 Hz), 4.14 (t, 1H, J=7.4 Hz), 2.44 (s, 3H), 1.86-1.84 (m, 2H), 1.43-1.40 (m, 4H), 0.95-0.93 (m, 3H).

Compounds (169) to (173) were prepared in a similar manner.

TABLE 34

| Compound | Structure | 1H-NMR |
|---|---|---|
| 169 | 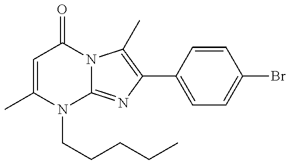 [SMILES: CCCCCn1c(C)cc(=O)n2c(C)c(-c3ccc(Br)cc3)nc12] | (δppm TMS/DMSO-d6) 7.64 (dd, 4H, J = 15.2, 8.6 Hz), 5.62 (s, 1H), 4.18 (t, 2H, J = 7.7 Hz), 2.78 (s, 3H), 2.41 (s, 3H), 1.76-1.74 (m, 2H), 1.35- 1.33 (m, 4H), 0.89 (t, 3H, J = 6.8 Hz). |
| 170 | 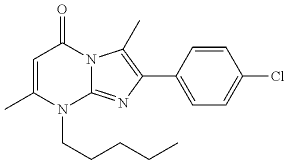 [SMILES: CCCCCn1c(C)cc(=O)n2c(C)c(-c3ccc(Cl)cc3)nc12] | (δppm TMS/DMSO-d6) 7.68 (d, 2H, J = 8.0 Hz), 7.51 (d, 2H, J = 7.8 Hz), 5.61 (s, 1H), 4.18 (t, 2H, J = 7.7 Hz), 2.78 (s, 3H), 2.40 (s, 3H), 1.74- 1.76 (m, 2H), 1.33-1.35 (m, 4H), 0.87 (t, 3H, J = 6.8 Hz). |
| 171 | 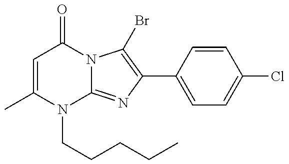 [SMILES: CCCCCn1c(C)cc(=O)n2c(Br)c(-c3ccc(Cl)cc3)nc12] | (δppm TMS/CDCl3) 8.00 (d, 2H, J = 8.5 Hz), 7.41 (d, 2H, J = 8.5 Hz), 5.61 (s, 1H), 4.22 (t, 2H, J = 7.8 Hz), 2.39 (s, 3H), 1.84-1.82 (m, 2H), 1.42- 1.41 (m, 4H), 0.94 (t, 3H, J = 6.8 Hz). |
| 172 | 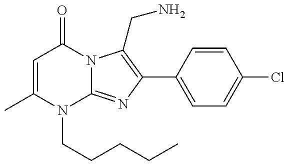 [SMILES: CCCCCn1c(C)cc(=O)n2c(CN)c(-c3ccc(Cl)cc3)nc12] | (δppm TMS/CDCl3) 7.70 (d, 2H, J = 8.3 Hz), 7.37 (d, 2H, J = 8.2 Hz), 5.64 (s, 1H), 4.21- 4.25 (m, 4H), 2.38 (s, 3H), 1.80-1.82 (br m, 4H), 1.37- 1.40 (br m, 4H), 0.92 (s, 3H). |
| 173 | 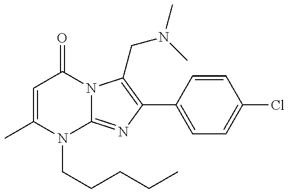 [SMILES: CCCCCn1c(C)cc(=O)n2c(CN(C)C)c(-c3ccc(Cl)cc3)nc12] | (δppm TMS/CDCl3) 7.82 (d, 2H, J = 8.4 Hz), 7.41 (d, 2H, J = 8.2 Hz), 5.62 (s, 1H), 4.24 (t, 2H, J = 7.8 Hz), 4.13 (s, 2H), 2.39 (s, 3H), 2.25 (s, 6H), 1.83-1.85 (m, 2H), 1.39- 1.42 (m, 4H), 0.94 (t, 3H, J = 10.0 Hz). |

### Example 7

### Synthesis of methyl 4-((2-(4-chlorophenyl)-6-methyl-5-oxo-8-pentyl-5,8-dihydroimidazo[1,2-a]pyrimidin-7-ylamino)methyl)benzoate (178)

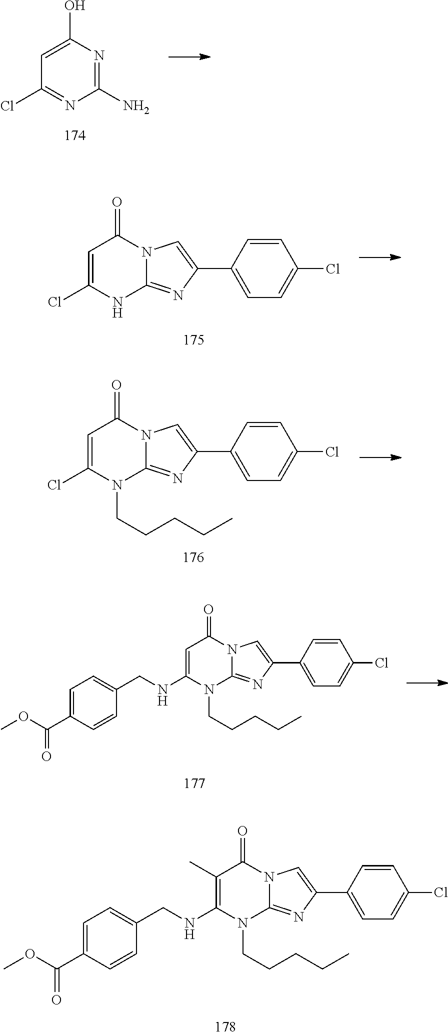 [SMILES: Nc1nc(O)cc(Cl)n1 ; O=c1cc(Cl)[nH]c2nc(-c3ccc(Cl)cc3)cn12 ; CCCCCn1c(Cl)cc(=O)n2cc(-c3ccc(Cl)cc3)nc12 ; CCCCCn1c(NCc2ccc(C(=O)OC)cc2)cc(=O)n2cc(-c3ccc(Cl)cc3)nc12 ; CCCCCn1c(NCc2ccc(C(=O)OC)cc2)c(C)c(=O)n2cc(-c3ccc(Cl)cc3)nc12]

To a solution of 2-amino-6-chloro-pyrimidin-4-ol (174, 25 g, 172 mmol) in N,N-dimethylformamide (250 mL) was added sodium hydride (60 wt %, 7.56 g, 189 mmol) under ice-cooling, and the mixture was stirred at room temperature for 30 minutes. A solution of 2-bromo-1-(4-chlorophenyl)ethanone (40 g, 172 mmol) in N,N-dimethylformamide (100 mL) was added to the solution in the iced bath, and the mixture was stirred at room temperature for 2 hours. Sodium hydroxide solution (2 mol/L, 125 mL) was added to the reaction mixture, and the mixture was stirred at room temperature for 45 minutes. Hydrochloric acid (2 mol/L, 138 mL) and water (250 mL) were added, and the precipitated solid was collected by filtration to yield a crude product of 7-chloro-2-(4-chlorophenyl)imidazo[1,2-a]pyrimidin-5(8H)-one (175) (20 g).

### Step 2

To crude product of the compound (175) (20 g) in N,N-dimethylformamide (300 mL) were added sodium hydride (60 wt %, 3.43 g, 86.0 mmol) and 1-bromopentane (32.4 g, 214 mmol), and the solution was stirred at 100° C. for 6 hours. After cooling to room temperature, the addition of water was added, and the mixture was extracted with ethyl acetate. The organic layer was washed with brine, dried with anhydrous sodium sulfate and concentrated under reduced pressure. The obtained residue was purified by silica gel chromatography (hexane/ethyl acetate) to yield 7-chloro-2-(4-chlorophenyl)-8-pentylimidazo[1,2-a]pyrimidin-5(8H)-one (176, 8.4 g, yield from the compound (174): 14%) as a colorless solid.

1H-NMR (δ ppm TMS/CDCl3) 7.85 (s, 1H), 7.80 (d, 2H, J=8.5 Hz), 7.39 (d, 2H, J=8.5 Hz), 5.99 (s, 1H), 4.51 (t, 2H, J=7.8 Hz), 1.91 (t, 2H, J=7.4 Hz), 1.44-1.43 (m, 4H), 0.95 (t, 3H, J=6.9 Hz).

### Step 3

To a solution of the compound (176, 500 mg, 1.43 mmol) in N,N-dimethylformamide (10 mL) were added 1,8-diazabicyclo[5,4,0]-7-undecene (435 mg, 2.86 mmol) and methyl 4-aminomethyl benzoate (472 mg, 2.86 mmol), and the mixture was stirred at 80° C. for 24 hours. After cooling to room temperature, water was added, and the reaction mixture was extracted with ethyl acetate. The organic layer was washed with brine, dried with anhydrous sodium sulfate and concentrated under reduced pressure. The residue was purified by silica gel chromatography (hexane/ethyl acetate) to yield methyl 4-((2-(4-chlorophenyl)-5-oxo-8-pentyl-5,8-dihydroimidazo[1,2-a]pyrimidin-7-ylamino)methyl)benzoate (177, 554 mg, yield: 81%) as a colorless solid.

1H-NMR (δ ppm TMS/CDCl3) 8.05 (d, 2H, J=7.8 Hz), 7.76-7.73 (m, 3H), 7.39-7.36 (m, 4H), 5.12 (t, 1H, J=5.1 Hz), 4.95 (s, 1H), 4.46 (d, 2H, J=4.9 Hz), 4.32 (t, 2H, J=7.6 Hz), 3.92 (s, 3H), 1.90-1.82 (m, 2H), 1.43-1.41 (m, 4H), 0.92 (t, 3H, J=6.0 Hz).

### Step 4

To a solution of the compound (177, 160 mg, 0.334 mmol) in N,N-dimethylformamide (3 mL) were added potassium carbonate (69 mg, 0.50 mmol) and methyl iodide (52 mg, 0.37 mmol), and the mixture was stirred at room temperature for 24 hours. Water was added to the reaction mixture, and the mixture was extracted with ethyl acetate. The organic layer was washed with brine, dried with anhydrous sodium sulfate and concentrated under reduced pressure. The residue was purified by silica gel chromatography (chloroform/methanol) to yield methyl 4-((2-(4-chlorophenyl)-6-methyl-5-oxo-8-pentyl-5,8-dihydroimidazo[1,2-a]pyrimidin-7-ylamino)methyl)benzoate (178, 75 mg, yield: 46%) as a colorless solid.

1H-NMR (δ ppm TMS/CDCl3) 8.06 (d, 2H, J=7.7 Hz), 7.81-7.80 (m, 3H), 7.41-7.37 (m, 4H), 4.40 (d, 2H, J=6.7 Hz), 4.28 (t, 2H, J=7.6 Hz), 3.93-3.91 (m, 4H), 2.02 (s, 3H), 1.88-1.81 (m, 2H), 1.37-1.29 (m, 4H), 0.89 (t, 3H, J=6.7 Hz).

Compounds (179) to (361) were prepared in a similar manner.

TABLE 35

| Compound | Structure | 1H-NMR |
|---|---|---|
| 179 | 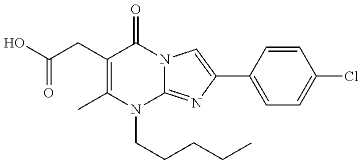 [SMILES: CCCCCn1c(C)c(CC(=O)O)c(=O)n2cc(-c3ccc(Cl)cc3)nc12] | (δppm TMS/DMSO-d6) 8.11 (s, 1H), 7.98 (d, 2H, J = 8.3 Hz), 7.46 (d, 2H, J = 8.0 Hz), 4.34 (s, 2H), 3.15 (s, 2H), 2.37 (s, 3H), 1.76 (s, 2H), 1.38 (s, 4H), 0.91 (s, 3H). |
| 180 | 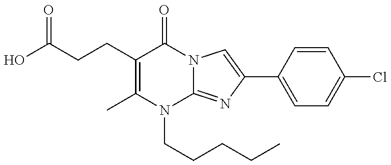 [SMILES: CCCCCn1c(C)c(CCC(=O)O)c(=O)n2cc(-c3ccc(Cl)cc3)nc12] | (δppm TMS/CDCl3) 7.82 (d, 3H, J = 9.5 Hz), 7.38 (d, 2H, J = 8.3 Hz), 4.36 (t, 2H, J = 7.8 Hz), 2.96 (t, 2H, J = 7.4 Hz), 2.67 (t, 2H, J = 7.4 Hz), 2.52 (s, 3H), 1.84 (s, 2H), 1.44 (d, 4H, J = 3.5 Hz), 0.95 (t, 3H, J = 6.4 Hz). |
| 181 | 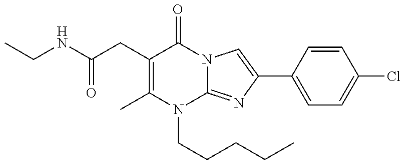 [SMILES: CCCCCn1c(C)c(CC(=O)NCC)c(=O)n2cc(-c3ccc(Cl)cc3)nc12] | (δppm TMS/DMSO-d6) 8.16 (s, 1H), 7.99 (d, 2H, J = 7.8 Hz), 7.78 (s, 1H), 7.48 (d, 2H, J = 7.8 Hz), 4.37 (s, 2H), 3.43 (s, 5H), 3.06 (d, 3H, J = 6.3 Hz), 2.40 (s, 3H), 1.78 (s, 2H), 1.39 (s, 4H), 0.99 (t, 3H, J = 7.2 Hz), 0.91 (s, 3H). |
| 182 | 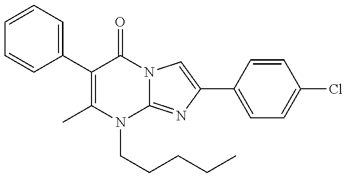 [SMILES: CCCCCn1c(C)c(-c2ccccc2)c(=O)n2cc(-c3ccc(Cl)cc3)nc12] | (δppm TMS/DMSO-d6) 8.22 (s, 1H), 8.01 (d, 2H, J = 8.0 Hz), 7.47 (dd, 4H, J = 19.4, 7.7 Hz), 7.39 (d, 1H, J = 7.0 Hz), 7.30 (d, 2H, J = 7.5 Hz), 4.38 (t, 2H, J = 7.5 Hz), 2.31 (s, 3H), 1.85 (s, 2H), 1.42 (s, 4H), 0.92 (s, 3H). |
| 183 | 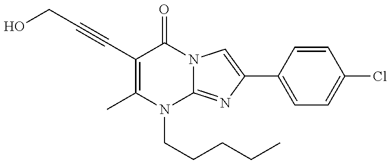 [SMILES: CCCCCn1c(C)c(C#CCO)c(=O)n2cc(-c3ccc(Cl)cc3)nc12] | (δppm TMS/DMSO-d6) 8.25 (s, 1H), 7.98 (d, 2H, J = 8.0 Hz), 7.49 (d, 2H, J = 8.3 Hz), 5.33 (s, 1H), 4.34 (s, 4H), 2.69 (s, 3H), 1.81 (s, 2H), 1.38 (s, 4H), 0.90 (s, 3H). |

TABLE 36

| Compound | Structure | 1H-NMR |
|---|---|---|
| 184 | 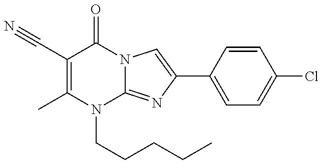 [SMILES: CCCCCn1c(C)c(C#N)c(=O)n2cc(-c3ccc(Cl)cc3)nc12] | (δppm TMS/DMSO-d6) 8.36 (s, 1H), 7.99 (d, 2H, J = 7.8 Hz), 7.51 (d, 2H, J = 7.8 Hz), 4.39 (t, 2H, J = 7.8 Hz), 2.73 (s, 3H), 1.83 (s, 2H), 1.40 (s, 4H), 0.91 (s, 3H). |
| 185 | 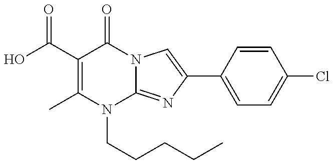 [SMILES: CCCCCn1c(C)c(C(=O)O)c(=O)n2cc(-c3ccc(Cl)cc3)nc12] | (δppm TMS/DMSO-d6) 8.10 (s, 1H), 7.96 (d, 2H, J = 8.3 Hz), 7.46 (d, 2H, J = 8.3 Hz), 4.26 (t, 2H, J = 7.7 Hz), 2.43 (s, 3H), 1.76 (s, 2H), 1.38 (s, 4H), 0.90 (t, 3H, J = 6.7 Hz). |
| 186 | 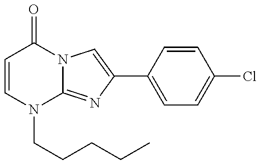 [SMILES: CCCCCn1ccc(=O)n2cc(-c3ccc(Cl)cc3)nc12] | (δppm TMS/CDCl3) 7.90 (s, 1H), 7.82 (d, 2H, J = 8.3 Hz), 7.40-7.38 (m, 3H), 5.82 (d, 1H, J = 7.5 Hz), 4.23 (t, 2H, J = 7.3 Hz), 1.95 (t, 2H, J = 7.2 Hz), 1.40-1.39 (m, 4H), 0.93 (t, 3H, J = 6.7 Hz). |
| 187 | 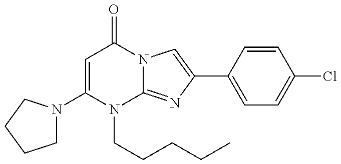 [SMILES: CCCCCn1c(N2CCCC2)cc(=O)n2cc(-c3ccc(Cl)cc3)nc12] | (δppm TMS/CDCl3) 7.80-7.77 (m, 3H), 7.36 (d, 2H, J = 8.0 Hz), 5.29 (s, 1H), 4.32 (t, 2H, J = 7.7 Hz), 3.38 (t, 4H, J = 6.0 Hz), 2.03 (t, 4H, J = 6.0 Hz), 1.87-1.85 (m, 2H), 1.39-1.29 (m, 4H), 0.90 (t, 3H, J = 7.0 Hz). |
| 188 | 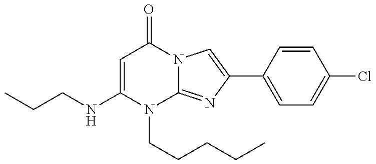 [SMILES: CCCCCn1c(NCCC)cc(=O)n2cc(-c3ccc(Cl)cc3)nc12] | (δppm TMS/CDCl3) 7.78-7.76 (m, 3H), 7.35 (d, 2H, J = 8.3 Hz), 5.01 (s, 1H), 4.49 (t, 1H, J = 5.0 Hz), 4.25 (t, 2H, J = 7.7 Hz), 3.19 (q, 2H, J = 6.4 Hz), 1.84-1.72 (m, 5H), 1.04 (t, 3H, J = 7.4 Hz), 0.94 (t, 3H, J = 6.4 Hz). |

TABLE 37

| Compound | Structure | 1H-NMR |
|---|---|---|
| 189 | 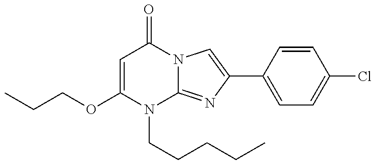 [SMILES: CCCCCn1c(OCCC)cc(=O)n2cc(-c3ccc(Cl)cc3)nc12] | (δppm TMS/CDCl3) 7.80-7.79 (m, 3H), 7.37 (d, 2H, J = 8.0 Hz), 5.24 (s, 1H), 4.30 (t, 2H, J = 7.4 Hz), 4.10 (t, 2H, J = 6.3 Hz), 1.91-1.86 (m, 4H), 1.40- 1.39 (m, 4H), 1.10 (t, 3H, J = 7.4 Hz), 0.93 (t, 3H, J = 6.7 Hz). |
| 190 | 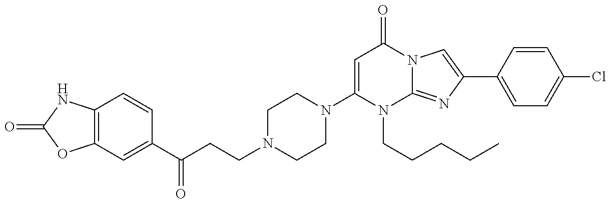 [SMILES: CCCCCn1c(N2CCN(CCC(=O)c3ccc4[nH]c(=O)oc4c3)CC2)cc(=O)n2cc(-c3ccc(Cl)cc3)nc12] | (δppm TMS/DMSO-d6) 8.11 (s, 1H), 7.94 (d, 2H, J = 8.3 Hz), 7.88 (d, 2H, J = 8.3 Hz), 7.46 (d, 2H, J = 8.0 Hz), 7.20 (d, 1H, J = 8.0 Hz), 5.44 (s, 1H), 4.20 (t, 2H, J = 7.0 Hz), 3.23 (t, 2H, J = 6.7 Hz), 3.03 (br s, 3H), 2.78 (t, 2H, J = 7.0 Hz), 2.62 (br s, 2H), 1.79-1.77 (m, 2H), 1.33-1.22 (m, 4H), 0.84 (t, 3H, J = 7.0 Hz). |
| 191 | 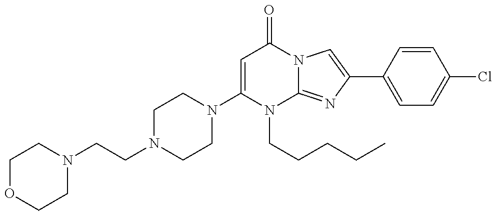 [SMILES: CCCCCn1c(N2CCN(CCN3CCOCC3)CC2)cc(=O)n2cc(-c3ccc(Cl)cc3)nc12] | (δppm TMS/CDCl3) 7.79 (d, 3H, J = 7.8 Hz), 7.37 (d, 2H, J = 8.0 Hz), 5.44 (s, 1H), 4.25 (t, 2H, J = 7.3 Hz), 3.73 (s, 4H), 3.06 (s, 4H), 2.69 (s, 3H), 2.61 (d, 2H, J = 6.8 Hz), 2.56 (d, 2H, J = 7.0 Hz), 2.51 (s, 4H), 1.84 (s, 2H), 1.39-1.29 (m, 4H), 0.91 (t, 3H, J = 7.0 Hz). |

TABLE 38

| Com- | | |
|---|---|---|
| pound | Structure | 1H-NMR |
| 192 | 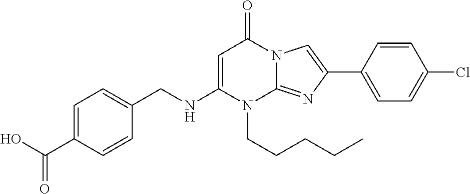 [SMILES: CCCCCn1c(NCc2ccc(C(=O)O)cc2)cc(=O)n2cc(-c3ccc(Cl)cc3)nc12] | (δ ppm TMS/DMSO-d6) 12.94 (s, 1H), 7.94-7.90 (m, 5H), 7.84 (t, 1H, J = 5.7 Hz), 7.49 (d, 2H, J = 7.7 Hz), 7.44 (d, 2H, J = 7.3 Hz), 4.69 (s, 1H), 4.55 (d, 2H, J = 5.0 Hz), 4.34 (t, 2H, J = 7.4 Hz), 1.79-1.77 (m, 2H), 1.40-1.37 (m, 4H), 0.90 (t, 3H, J = 6.0 Hz). |
| 193 | 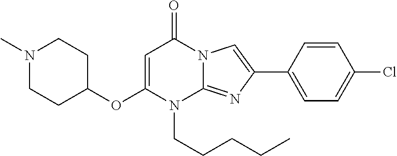 [SMILES: CCCCCn1c(OC2CCN(C)CC2)cc(=O)n2cc(-c3ccc(Cl)cc3)nc12] | (δ ppm TMS/CDC13) 7.79-7.78 (m, 3H), 7.37 (d, 2H, J = 8.3 Hz), 5.25 (s, 1H), 4.51-4.49 (m, 1H), 4.30 (t, 2H, J = 7.5 Hz), 2.67-2.65 (m, 2H), 2.38- 2.35 (m, 5H), 2.14-2.09 (m, 2H), 1.98-1.94 (m, 2H), 1.86- 1.82 (m, 2H), 1.41-1.38 (m, 4H), 0.93 (t, 3H, J = 6.5 Hz). |
| 194 | 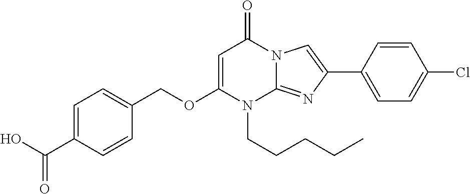 [SMILES: CCCCCn1c(OCc2ccc(C(=O)O)cc2)cc(=O)n2cc(-c3ccc(Cl)cc3)nc12] | 13.08 (s, 1H), 8.14 (d, 1H, J = 2.1 Hz), 8.01 (d, 2H, J = 7.0 Hz), 7.92 (d, 2H, J = 7.0 Hz), 7.63 (d, 2H, J = 6.8 Hz), 7.45 (d, 2H, J = 7.0 Hz), 5.57 (s, 1H), 5.45 (s, 2H), 4.25 (t, 2H, J = 6.3 Hz), 1.78-1.75 (m, 2H), 1.27-1.19 (m, 4H), 0.81 (t, 3H, J = 5.8 Hz). |

TABLE 39

| Com- | | |
|---|---|---|
| pound | Structure | 1H-NMR |
| 195 | 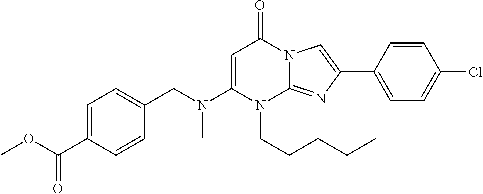 [SMILES: CCCCCn1c(N(C)Cc2ccc(C(=O)OC)cc2)cc(=O)n2cc(-c3ccc(Cl)cc3)nc12] | (δ ppm TMS/CDC13) 8.07 (d, 2H, J = 7.5 Hz), 7.82-7.79 (m, 3H), 7.39-7.37 (m, 4H), 5.48 (s, 1H), 4.31 (t, 2H, J = 7.5 Hz), 4.25 (s, 2H), 3.94 (s, 3H), 2.72 (s, 3H), 1.89-1.85 (m, 2H), 1.38-1.32 (m, 4H), 0.91 (t, 3H, J = 6.8 Hz). |
| 196 | 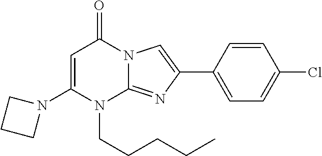 [SMILES: CCCCCn1c(N2CCC2)cc(=O)n2cc(-c3ccc(Cl)cc3)nc12] | (δ ppm TMS/CDC13) 7.78- 7.76 (m, 3H), 7.35 (d, 2H, J = 8.0 Hz), 4.83 (s, 1H), 4.19-4.13 (m, 6H), 2.47-2.40 (m, 2H), 1.90-1.82 (m, 2H), 1.42-1.36 (m, 4H), 0.94 (t, 3H, J = 6.9 Hz). |
| 197 | 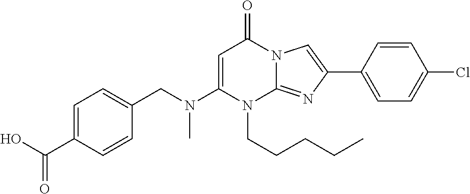 [SMILES: CCCCCn1c(N(C)Cc2ccc(C(=O)O)cc2)cc(=O)n2cc(-c3ccc(Cl)cc3)nc12] | (δ ppm TMS/DMSO-d6) 13.01 (s, 1H), 8.14 (s, 1H), 7.97-7.95 (m, 4H), 7.49-7.47 (m, 4H), 5.48 (s, 1H), 4.38 (s, 2H), 4.24 (t, 2H, J = 7.5 Hz), 2.73 (s, 3H), 1.81-1.78 (m, 2H), 1.32-1.16 (m, 4H), 0.83 (t, 3H, J = 7.2 Hz). |

TABLE 40

| Com- | | |
|---|---|---|
| pound | Structure | 1H-NMR |
| 198 | 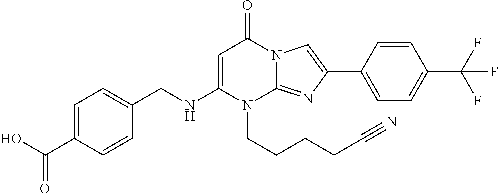 [SMILES: N#CCCCCn1c(NCc2ccc(C(=O)O)cc2)cc(=O)n2cc(-c3ccc(C(F)(F)F)cc3)nc12] | (δ ppm TMS/DMSO-d6) 8.11 (d, 3H, J = 8.3 Hz), 7.94 (d, 2H, J = 7.8 Hz), 7.87-7.89 (m, 1H), 7.73 (d, 2H, J = 8.3 Hz), 7.51 (d, 2H, J = 7.8 Hz), 4.73 (s, 1H), 4.54-4.57 (m, 2H), 4.41 (t, 2H, J = 6.9 Hz), 2.65 (t, 2H, J = 6.9 Hz), 1.90- 1.92 (m, 2H), 1.69-1.71 (m, 2H). |

TABLE 41

| Com- | | | Retention | Mass |
|---|---|---|---|---|
| pound | Structure | LC/MS | Time [min] | [M + H] |
| 199 | 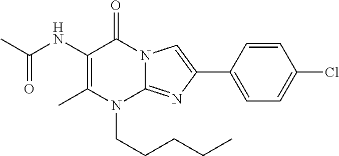 [SMILES: CCCCCn1c(C)c(NC(C)=O)c(=O)n2cc(-c3ccc(Cl)cc3)nc12] | Method A | 2.30 | 387 |
| 200 | 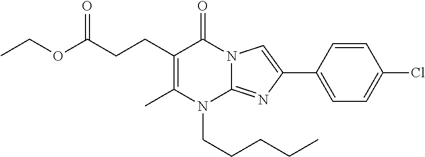 [SMILES: CCCCCn1c(C)c(CCC(=O)OCC)c(=O)n2cc(-c3ccc(Cl)cc3)nc12] | Method A | 3.10 | 430 |
| 201 | 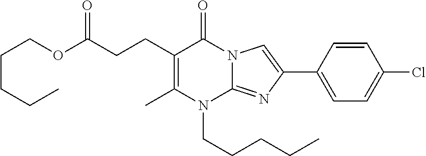 [SMILES: CCCCCOC(=O)CCc1c(C)n(CCCCC)c2nc(-c3ccc(Cl)cc3)cn2c1=O] | Method A | 3.46 | 472 |
| 202 | 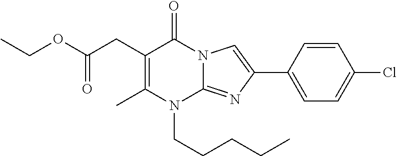 [SMILES: CCCCCn1c(C)c(CC(=O)OCC)c(=O)n2cc(-c3ccc(Cl)cc3)nc12] | Method A | 2.99 | 416 |
| 203 | 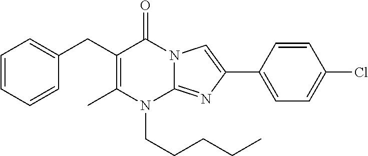 [SMILES: CCCCCn1c(C)c(Cc2ccccc2)c(=O)n2cc(-c3ccc(Cl)cc3)nc12] | Method A | 3.31 | 420 |

TABLE 42

| Com- | | | Retention | Mass |
|---|---|---|---|---|
| pound | Structure | LC/MS | Time [min] | [M + H] |
| 204 | 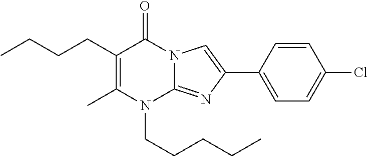 [SMILES: CCCCCn1c(C)c(CCCC)c(=O)n2cc(-c3ccc(Cl)cc3)nc12] | Method A | 3.41 | 386 |
| 205 | 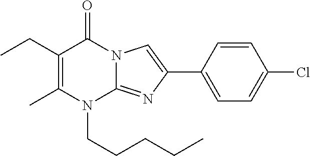 [SMILES: CCCCCn1c(C)c(CC)c(=O)n2cc(-c3ccc(Cl)cc3)nc12] | Method A | 3.10 | 358 |
| 206 | 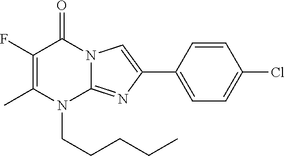 [SMILES: CCCCCn1c(C)c(F)c(=O)n2cc(-c3ccc(Cl)cc3)nc12] | Method A | 2.89 | 348 |
| 207 | 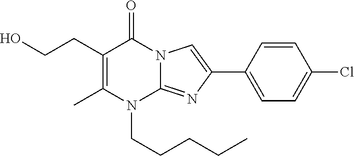 [SMILES: CCCCCn1c(C)c(CCO)c(=O)n2cc(-c3ccc(Cl)cc3)nc12] | Method A | 2.57 | 374 |
| 208 | 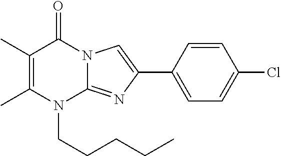 [SMILES: CCCCCn1c(C)c(C)c(=O)n2cc(-c3ccc(Cl)cc3)nc12] | Method A | 2.97 | 344 |

TABLE 43

| Com- | | | Retention | Mass |
|---|---|---|---|---|
| pound | Structure | LC/MS | Time [min] | [M + H] |
| 209 | 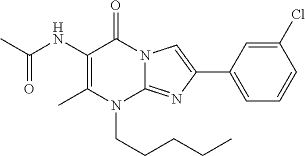 [SMILES: CCCCCn1c(C)c(NC(C)=O)c(=O)n2cc(-c3cccc(Cl)c3)nc12] | Method A | 2.29 | 387 |
| 210 | 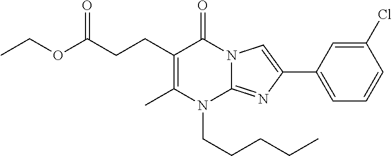 [SMILES: CCCCCn1c(C)c(CCC(=O)OCC)c(=O)n2cc(-c3cccc(Cl)c3)nc12] | Method A | 3.10 | 430 |
| 211 | 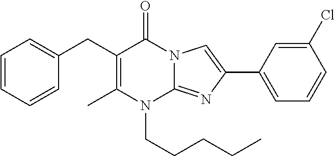 [SMILES: CCCCCn1c(C)c(Cc2ccccc2)c(=O)n2cc(-c3cccc(Cl)c3)nc12] | Method A | 3.31 | 420 |
| 212 | 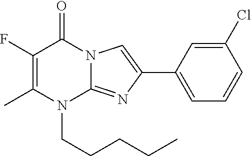 [SMILES: CCCCCn1c(C)c(F)c(=O)n2cc(-c3cccc(Cl)c3)nc12] | Method A | 2.89 | 348 |
| 213 | 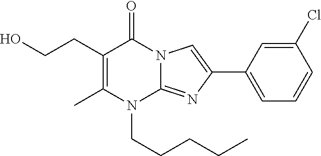 [SMILES: CCCCCn1c(C)c(CCO)c(=O)n2cc(-c3cccc(Cl)c3)nc12] | Method A | 2.58 | 374 |

TABLE 44

| Com- | | | Retention | Mass |
|---|---|---|---|---|
| pound | Structure | LC/MS | Time [min] | [M + H] |
| 214 | 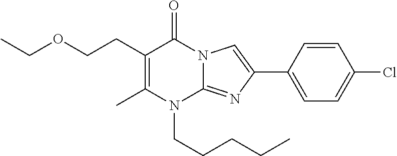 [SMILES: CCCCCn1c(C)c(CCOCC)c(=O)n2cc(-c3ccc(Cl)cc3)nc12] | Method A | 2.89 | 402 |
| 215 | 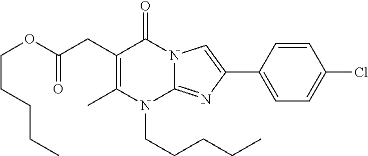 [SMILES: CCCCCOC(=O)Cc1c(C)n(CCCCC)c2nc(-c3ccc(Cl)cc3)cn2c1=O] | Method A | 3.36 | 458 |
| 216 | 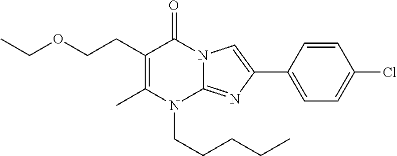 [SMILES: CCCCCn1c(C)c(CCOCC)c(=O)n2cc(-c3ccc(Cl)cc3)nc12] | Method A | 2.89 | 402 |
| 217 | 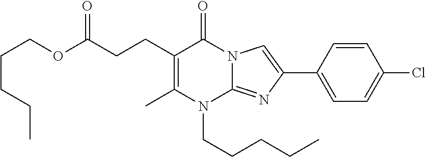 [SMILES: CCCCCOC(=O)CCc1c(C)n(CCCCC)c2nc(-c3ccc(Cl)cc3)cn2c1=O] | Method A | 3.47 | 472 |
| 218 | 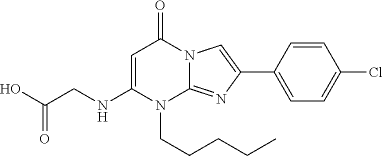 [SMILES: CCCCCn1c(NCC(=O)O)cc(=O)n2cc(-c3ccc(Cl)cc3)nc12] | Method A | 1.72 | 389 |

TABLE 45

| Com- | | | Retention | Mass |
|---|---|---|---|---|
| pound | Structure | LC/MS | Time [min] | [M + H] |
| 219 | 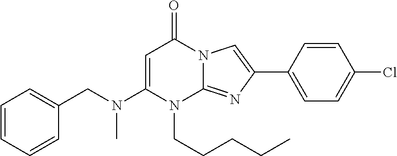 [SMILES: CCCCCn1c(N(C)Cc2ccccc2)cc(=O)n2cc(-c3ccc(Cl)cc3)nc12] | Method A | 3.26 | 435 |
| 220 | 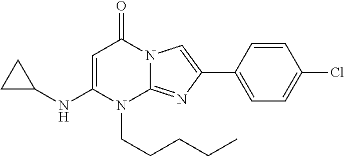 [SMILES: CCCCCn1c(NC2CC2)cc(=O)n2cc(-c3ccc(Cl)cc3)nc12] | Method A | 3.36 | 552 |
| 221 | 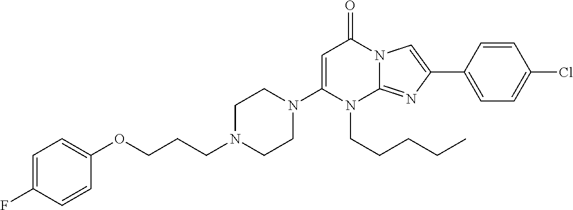 [SMILES: CCCCCn1c(N2CCN(CCCOc3ccc(F)cc3)CC2)cc(=O)n2cc(-c3ccc(Cl)cc3)nc12] | Method A | 2.66 | 371 |
| 222 | 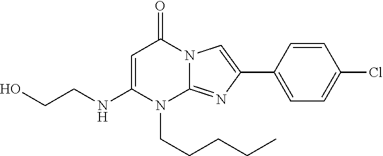 [SMILES: CCCCCn1c(NCCO)cc(=O)n2cc(-c3ccc(Cl)cc3)nc12] | Method A | 2.23 | 375 |

TABLE 46

| Com- | | | Retention | Mass |
|---|---|---|---|---|
| pound | Structure | LC/MS | Time [min] | [M + H] |
| 223 | 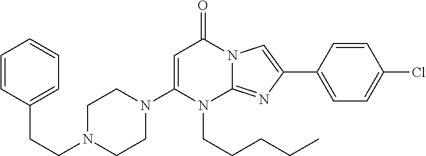 [SMILES: CCCCCn1c(N2CCN(CCc3ccccc3)CC2)cc(=O)n2cc(-c3ccc(Cl)cc3)nc12] | Method A | 3.33 | 504 |
| 224 | 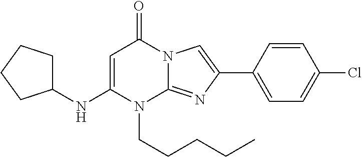 [SMILES: CCCCCn1c(NC2CCCC2)cc(=O)n2cc(-c3ccc(Cl)cc3)nc12] | Method A | 2.91 | 399 |
| 225 | 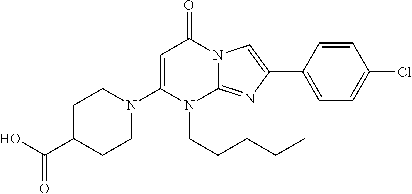 [SMILES: CCCCCn1c(N2CCC(C(=O)O)CC2)cc(=O)n2cc(-c3ccc(Cl)cc3)nc12] | Method A | 1.80 | 443 |
| 226 | 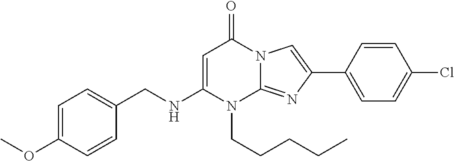 [SMILES: CCCCCn1c(NCc2ccc(OC)cc2)cc(=O)n2cc(-c3ccc(Cl)cc3)nc12] | Method A | 2.81 | 451 |
| 227 | 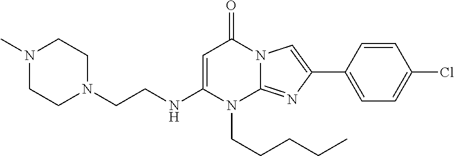 [SMILES: CCCCCn1c(NCCN2CCN(C)CC2)cc(=O)n2cc(-c3ccc(Cl)cc3)nc12] | Method A | 2.44 | 457 |

TABLE 47

| Com- | | | Retention | Mass |
|---|---|---|---|---|
| pound | Structure | LC/MS | Time [min] | [M + H] |
| 228 | 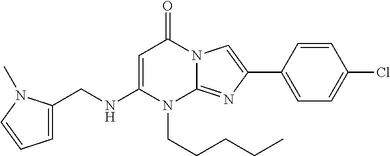 [SMILES: CCCCCn1c(NCc2cccn2C)cc(=O)n2cc(-c3ccc(Cl)cc3)nc12] | Method A | 2.74 | 424 |
| 229 | 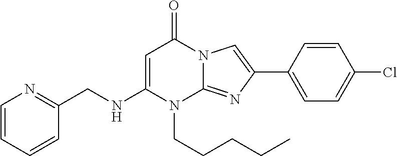 [SMILES: CCCCCn1c(NCc2ccccn2)cc(=O)n2cc(-c3ccc(Cl)cc3)nc12] | Method A | 2.61 | 422 |
| 230 | 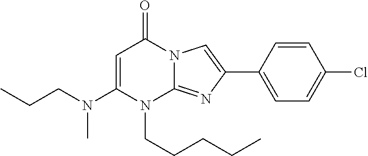 [SMILES: CCCCCn1c(N(C)CCC)cc(=O)n2cc(-c3ccc(Cl)cc3)nc12] | Method A | 3.18 | 387 |
| 231 | 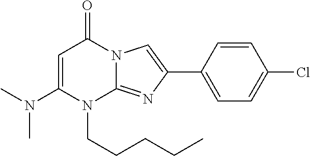 [SMILES: CCCCCn1c(N(C)C)cc(=O)n2cc(-c3ccc(Cl)cc3)nc12] | Method A | 2.84 | 359 |
| 232 | 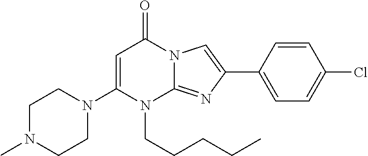 [SMILES: CCCCCn1c(N2CCN(C)CC2)cc(=O)n2cc(-c3ccc(Cl)cc3)nc12] | Method A | 2.75 | 414 |

TABLE 48

| Com- | | | Retention | Mass |
|---|---|---|---|---|
| pound | Structure | LC/MS | Time [min] | [M + H] |
| 233 | 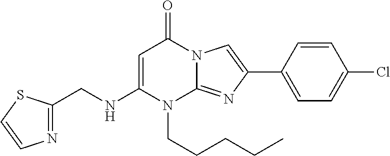 [SMILES: CCCCCn1c(NCc2nccs2)cc(=O)n2cc(-c3ccc(Cl)cc3)nc12] | Method A | 2.53 | 428 |
| 234 | 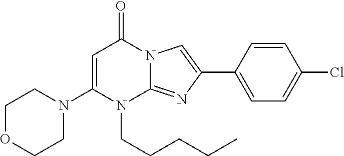 [SMILES: CCCCCn1c(N2CCOCC2)cc(=O)n2cc(-c3ccc(Cl)cc3)nc12] | Method A | 2.76 | 401 |
| 235 | 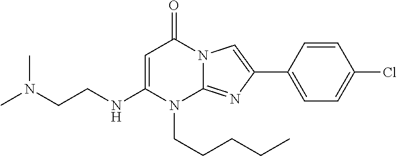 [SMILES: CCCCCn1c(NCCN(C)C)cc(=O)n2cc(-c3ccc(Cl)cc3)nc12] | Method A | 2.62 | 402 |
| 236 | 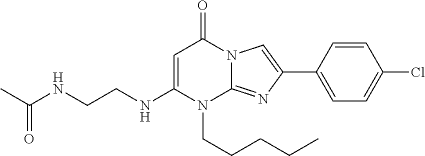 [SMILES: CCCCCn1c(NCCNC(C)=O)cc(=O)n2cc(-c3ccc(Cl)cc3)nc12] | Method A | 2.26 | 416 |
| 237 | 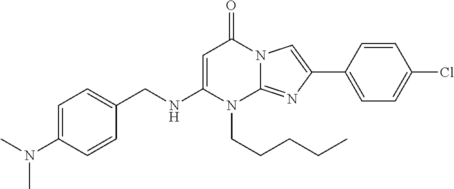 [SMILES: CCCCCn1c(NCc2ccc(N(C)C)cc2)cc(=O)n2cc(-c3ccc(Cl)cc3)nc12] | Method A | 2.93 | 464 |

TABLE 49

| | | | Retention | |
|---|---|---|---|---|
| | | | Time | Mass |
| Compound | Structure | LC/MS | [min] | [M + H] |
| 238 | 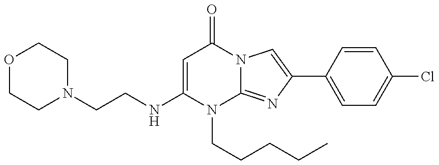 [SMILES: CCCCCn1c(NCCN2CCOCC2)cc(=O)n2cc(-c3ccc(Cl)cc3)nc12] | Method A | 2.51 | 444 |
| 239 | 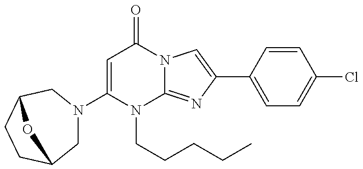 [SMILES: CCCCCn1c(N2C[C@H]3CC[C@@H](C2)O3)cc(=O)n2cc(-c3ccc(Cl)cc3)nc12] | Method A | 2.87 | 427 |
| 240 | 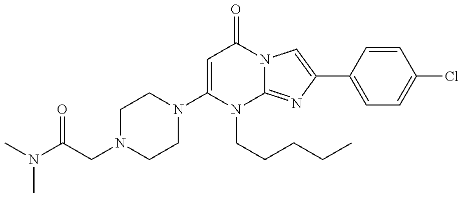 [SMILES: CCCCCn1c(N2CCN(CC(=O)N(C)C)CC2)cc(=O)n2cc(-c3ccc(Cl)cc3)nc12] | Method A | 2.54 | 485 |
| 241 | 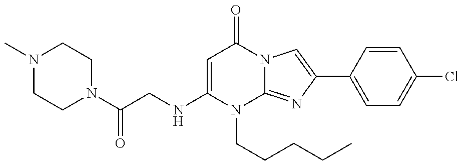 [SMILES: CCCCCn1c(NCC(=O)N2CCN(C)CC2)cc(=O)n2cc(-c3ccc(Cl)cc3)nc12] | Method A | 2.39 | 471 |
| 242 | 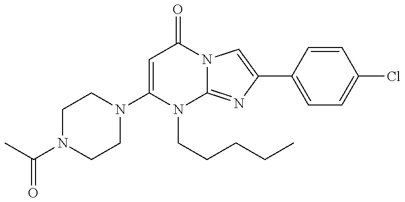 [SMILES: CCCCCn1c(N2CCN(C(C)=O)CC2)cc(=O)n2cc(-c3ccc(Cl)cc3)nc12] | Method A | 2.49 | 442 |

TABLE 50

| | | | Retention | |
|---|---|---|---|---|
| | | | Time | |
| Compound | Structure | LC/MS | [min] | Mass [M + H] |
| 243 | 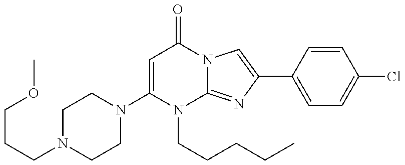 [SMILES: CCCCCn1c(N2CCN(CCCOC)CC2)cc(=O)n2cc(-c3ccc(Cl)cc3)nc12] | Method A | 2.88 | 472 |
| 244 | 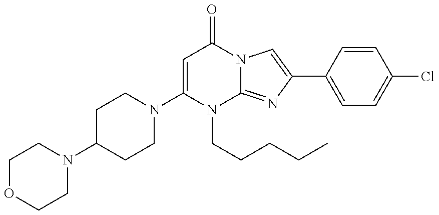 [SMILES: CCCCCn1c(N2CCC(N3CCOCC3)CC2)cc(=O)n2cc(-c3ccc(Cl)cc3)nc12] | Method A | 2.74 | 484 |
| 245 | 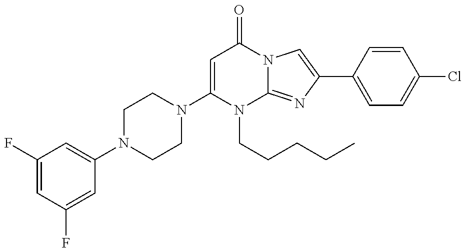 [SMILES: CCCCCn1c(N2CCN(c3cc(F)cc(F)c3)CC2)cc(=O)n2cc(-c3ccc(Cl)cc3)nc12] | Method A | 3.35 | 512 |
| 246 | 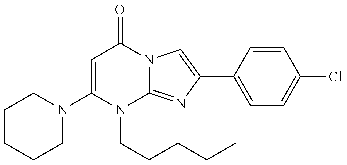 [SMILES: CCCCCn1c(N2CCCCC2)cc(=O)n2cc(-c3ccc(Cl)cc3)nc12] | Method A | 3.27 | 399 |
| 247 | 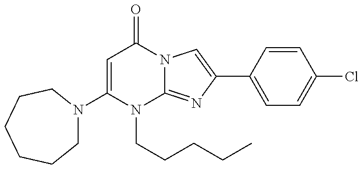 [SMILES: CCCCCn1c(N2CCCCCC2)cc(=O)n2cc(-c3ccc(Cl)cc3)nc12] | Method A | 3.34 | 413 |

TABLE 51

| | | | Retention | |
|---|---|---|---|---|
| | | | Time | |
| Compound | Structure | LC/MS | [min] | Mass [M + H] |
| 248 | 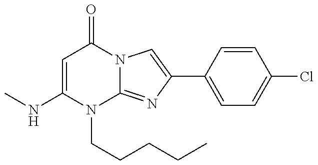 [SMILES: CCCCCn1c(NC)cc(=O)n2cc(-c3ccc(Cl)cc3)nc12] | Method A | 2.47 | 345 |
| 249 | 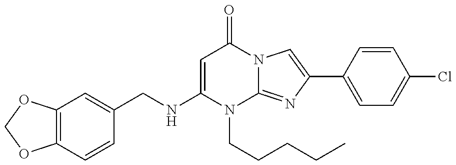 [SMILES: CCCCCn1c(NCc2ccc3c(c2)OCO3)cc(=O)n2cc(-c3ccc(Cl)cc3)nc12] | Method A | 2.77 | 465 |
| 250 | 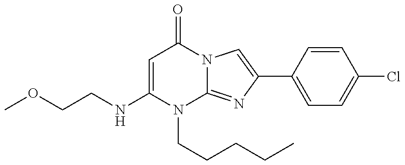 [SMILES: CCCCCn1c(NCCOC)cc(=O)n2cc(-c3ccc(Cl)cc3)nc12] | Method A | 2.53 | 389 |
| 251 | 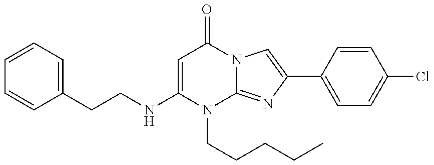 [SMILES: CCCCCn1c(NCCc2ccccc2)cc(=O)n2cc(-c3ccc(Cl)cc3)nc12] | Method A | 2.95 | 435 |
| 252 | 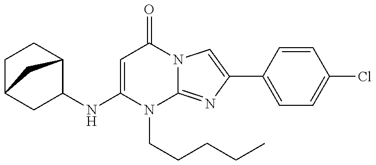 [SMILES: CCCCCn1c(NC2C[C@@H]3CC[C@H]2C3)cc(=O)n2cc(-c3ccc(Cl)cc3)nc12] | Method A | 3.08 | 425 |

TABLE 52

| | | | Retention | |
|---|---|---|---|---|
| | | | Time | |
| Compound | Structure | LC/MS | [min] | Mass [M + H] |
| 253 | 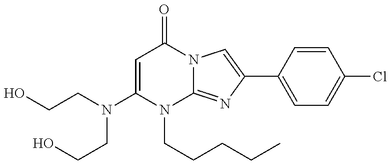 [SMILES: CCCCCn1c(N(CCO)CCO)cc(=O)n2cc(-c3ccc(Cl)cc3)nc12] | Method A | 2.17 | 419 |
| 254 | 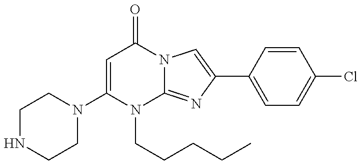 [SMILES: CCCCCn1c(N2CCNCC2)cc(=O)n2cc(-c3ccc(Cl)cc3)nc12] | Method A | 2.45 | 400 |
| 255 | 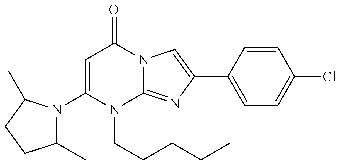 [SMILES: CCCCCn1c(N2C(C)CCC2C)cc(=O)n2cc(-c3ccc(Cl)cc3)nc12] | Method A | 3.46 | 413 |
| 256 | 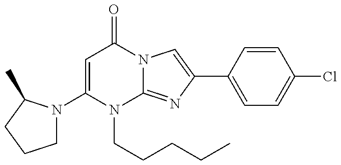 [SMILES: CCCCCn1c(N2CCC[C@H]2C)cc(=O)n2cc(-c3ccc(Cl)cc3)nc12] | Method A | 3.08 | 399 |
| 257 | 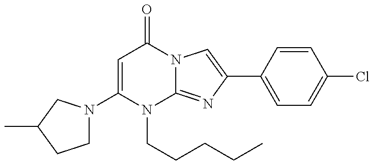 [SMILES: CCCCCn1c(N2CCC(C)C2)cc(=O)n2cc(-c3ccc(Cl)cc3)nc12] | Method A | 3.48 | 399 |

TABLE 53

| | | | Retention | |
|---|---|---|---|---|
| | | | Time | |
| Compound | Structure | LC/MS | [min] | Mass [M + H] |
| 258 | 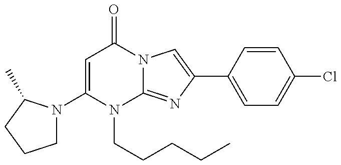 [SMILES: CCCCCn1c(N2CCC[C@@H]2C)cc(=O)n2cc(-c3ccc(Cl)cc3)nc12] | Method A | 3.08 | 399 |
| 259 | 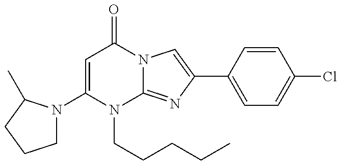 [SMILES: CCCCCn1c(N2CCCC2C)cc(=O)n2cc(-c3ccc(Cl)cc3)nc12] | Method A | 3.08 | 399 |
| 260 | 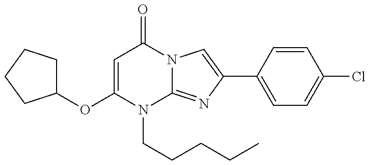 [SMILES: CCCCCn1c(OC2CCCC2)cc(=O)n2cc(-c3ccc(Cl)cc3)nc12] | Method A | 3.34 | 400 |
| 261 | 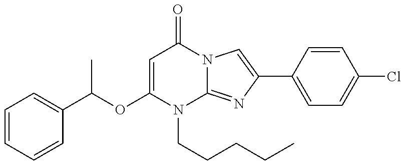 [SMILES: CCCCCn1c(OC(C)c2ccccc2)cc(=O)n2cc(-c3ccc(Cl)cc3)nc12] | Method A | 3.33 | 436 |
| 262 | 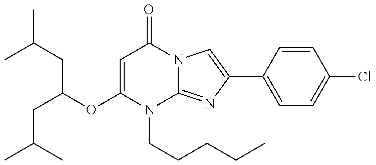 [SMILES: CCCCCn1c(OC(CC(C)C)CC(C)C)cc(=O)n2cc(-c3ccc(Cl)cc3)nc12] | Method A | 3.84 | 458 |

TABLE 54

| | | | Retention | |
|---|---|---|---|---|
| | | | Time | |
| Compound | Structure | LC/MS | [min] | Mass [M + H] |
| 263 | 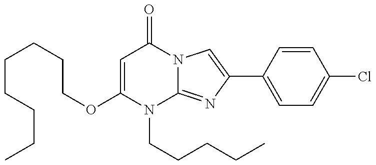 [SMILES: CCCCCCCCOc1cc(=O)n2cc(-c3ccc(Cl)cc3)nc2n1CCCCC] | Method A | 3.87 | 444 |
| 264 | 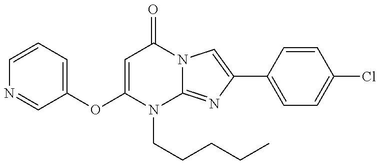 [SMILES: CCCCCn1c(Oc2cccnc2)cc(=O)n2cc(-c3ccc(Cl)cc3)nc12] | Method A | 2.81 | 409 |
| 265 | 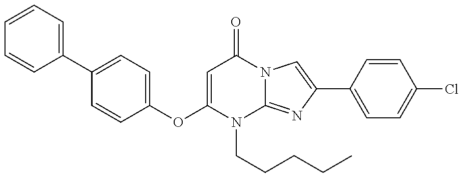 [SMILES: CCCCCn1c(Oc2ccc(-c3ccccc3)cc2)cc(=O)n2cc(-c3ccc(Cl)cc3)nc12] | Method A | 3.58 | 484 |
| 266 | 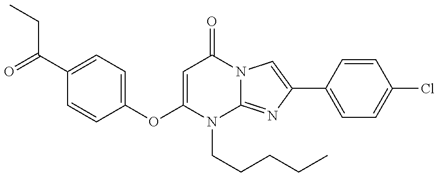 [SMILES: CCCCCn1c(Oc2ccc(C(=O)CC)cc2)cc(=O)n2cc(-c3ccc(Cl)cc3)nc12] | Method A | 3.25 | 464 |
| 267 | 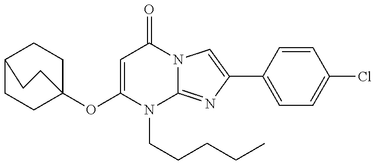 [SMILES: CCCCCn1c(OC23CCC(CC2)CC3)cc(=O)n2cc(-c3ccc(Cl)cc3)nc12] | Method A | 3.60 | 440 |

TABLE 55

| | | | Retention | |
|---|---|---|---|---|
| | | | Time | |
| Compound | Structure | LC/MS | [min] | Mass [M + H] |
| 268 | 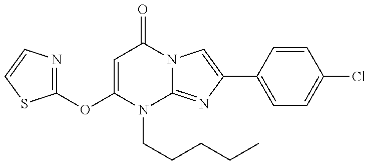 [SMILES: CCCCCn1c(Oc2nccs2)cc(=O)n2cc(-c3ccc(Cl)cc3)nc12] | Method A | 2.80 | 415 |
| 269 | 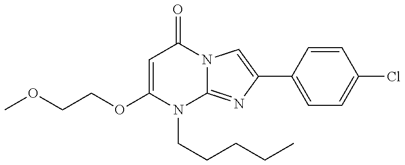 [SMILES: CCCCCn1c(OCCOC)cc(=O)n2cc(-c3ccc(Cl)cc3)nc12] | Method A | 2.84 | 390 |
| 270 | 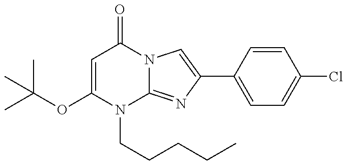 [SMILES: CCCCCn1c(OC(C)(C)C)cc(=O)n2cc(-c3ccc(Cl)cc3)nc12] | Method A | 2.85 | 388 |
| 271 | 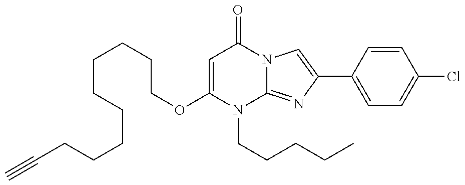 [SMILES: C#CCCCCCCCCCOc1cc(=O)n2cc(-c3ccc(Cl)cc3)nc2n1CCCCC] | Method A | 3.72 | 482 |
| 272 | 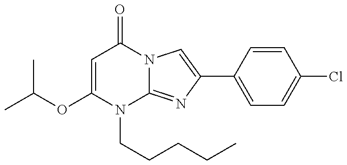 [SMILES: CCCCCn1c(OC(C)C)cc(=O)n2cc(-c3ccc(Cl)cc3)nc12] | Method A | 3.14 | 374 |

TABLE 56

| | | | Retention | |
|---|---|---|---|---|
| | | | Time | |
| Compound | Structure | LC/MS | [min] | Mass [M + H] |
| 273 | 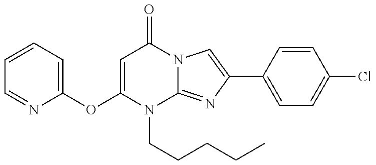 [SMILES: CCCCCn1c(Oc2ccccn2)cc(=O)n2cc(-c3ccc(Cl)cc3)nc12] | Method A | 2.97 | 409 |
| 274 | 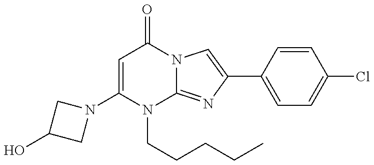 [SMILES: CCCCCn1c(N2CC(O)C2)cc(=O)n2cc(-c3ccc(Cl)cc3)nc12] | Method A | 2.36 | 387 |
| 275 | 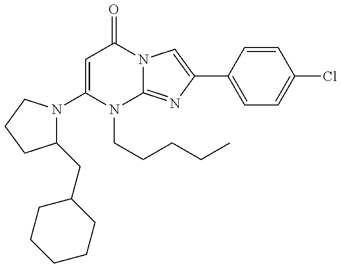 [SMILES: CCCCCn1c(N2CCCC2CC2CCCCC2)cc(=O)n2cc(-c3ccc(Cl)cc3)nc12] | Method A | 3.74 | 470 |
| 276 | 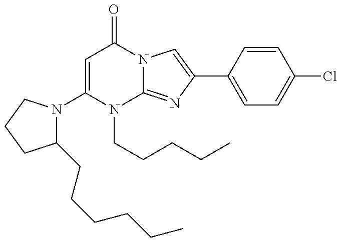 [SMILES: CCCCCCC1CCCN1c1cc(=O)n2cc(-c3ccc(Cl)cc3)nc2n1CCCCC] | Method A | 3.72 | 481 |
| 277 | 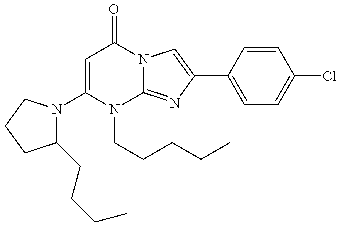 [SMILES: CCCCCn1c(N2CCCC2CCCC)cc(=O)n2cc(-c3ccc(Cl)cc3)nc12] | Method A | 3.48 | 442 |

TABLE 57

| | | | Retention | |
|---|---|---|---|---|
| | | | Time | |
| Compound | Structure | LC/MS | [min] | Mass [M + H] |
| 278 | 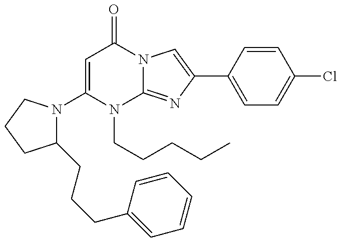 [SMILES: CCCCCn1c(N2CCCC2CCCc2ccccc2)cc(=O)n2cc(-c3ccc(Cl)cc3)nc12] | Method A | 3.51 | 503 |
| 279 | 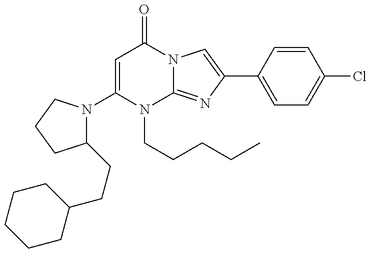 [SMILES: CCCCCn1c(N2CCCC2CCC2CCCCC2)cc(=O)n2cc(-c3ccc(Cl)cc3)nc12] | Method A | 3.85 | 496 |
| 280 | 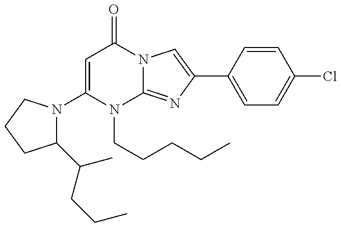 [SMILES: CCCCCn1c(N2CCCC2C(C)CCC)cc(=O)n2cc(-c3ccc(Cl)cc3)nc12] | Method A | 3.62 | 455 |
| 281 | 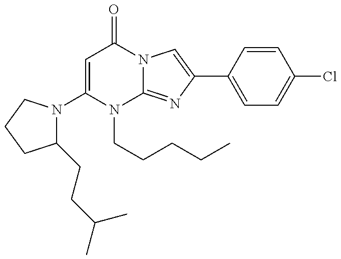 [SMILES: CCCCCn1c(N2CCCC2CCC(C)C)cc(=O)n2cc(-c3ccc(Cl)cc3)nc12] | Method A | 3.55 | 456 |
| 282 | 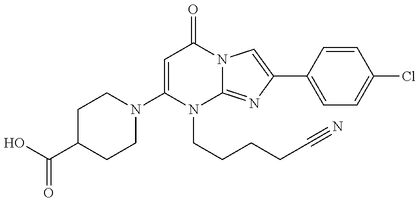 [SMILES: N#CCCCCn1c(N2CCC(C(=O)O)CC2)cc(=O)n2cc(-c3ccc(Cl)cc3)nc12] | Method A | 2.07 | 455 |

TABLE 58

| | | | Retention | |
|---|---|---|---|---|
| | | | Time | Mass |
| Compound | Structure | LC/MS | [min] | [M + H] |
| 283 | 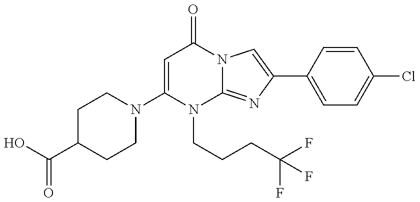 [SMILES: O=C(O)C1CCN(c2cc(=O)n3cc(-c4ccc(Cl)cc4)nc3n2CCCC(F)(F)F)CC1] | Method A | 2.40 | 483 |
| 284 | 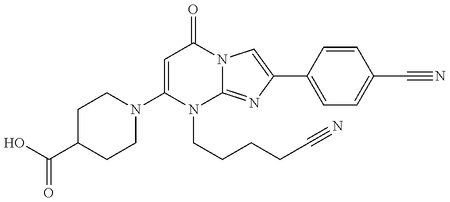 [SMILES: N#CCCCCn1c(N2CCC(C(=O)O)CC2)cc(=O)n2cc(-c3ccc(C#N)cc3)nc12] | Method A | 1.73 | 445 |
| 285 | 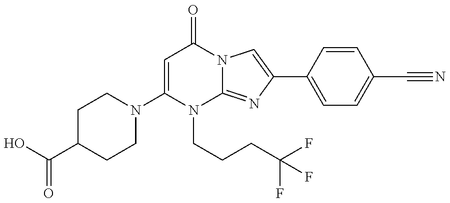 [SMILES: N#Cc1ccc(-c2cn3c(=O)cc(N4CCC(C(=O)O)CC4)n(CCCC(F)(F)F)c3n2)cc1] | Method A | 2.04 | 474 |
| 286 | 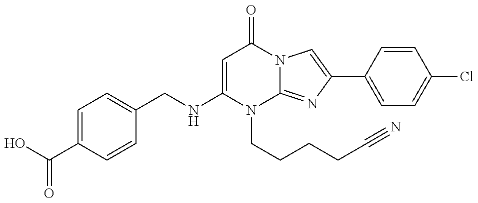 [SMILES: N#CCCCCn1c(NCc2ccc(C(=O)O)cc2)cc(=O)n2cc(-c3ccc(Cl)cc3)nc12] | Method A | 2.02 | 477 |
| 287 | 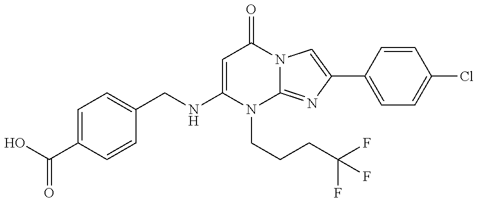 [SMILES: O=C(O)c1ccc(CNc2cc(=O)n3cc(-c4ccc(Cl)cc4)nc3n2CCCC(F)(F)F)cc1] | Method A | 2.35 | 506 |

TABLE 59

| | | | Retention | |
|---|---|---|---|---|
| | | | Time | Mass |
| Compound | Structure | LC/MS | [min] | [M + H] |
| 288 | 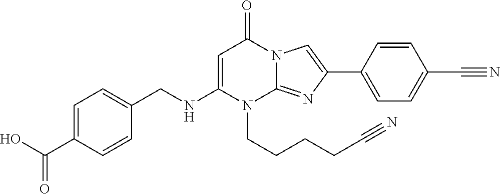 [SMILES: N#CCCCCn1c(NCc2ccc(C(=O)O)cc2)cc(=O)n2cc(-c3ccc(C#N)cc3)nc12] | | | |
| | | Method | 1.72 | 467 |
| | | A | | |
| 289 | 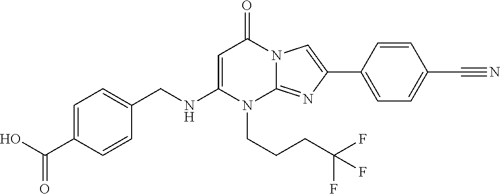 [SMILES: N#Cc1ccc(-c2cn3c(=O)cc(NCc4ccc(C(=O)O)cc4)n(CCCC(F)(F)F)c3n2)cc1] | | | |
| | | Method | 2.01 | 496 |
| | | A | | |
| 290 | 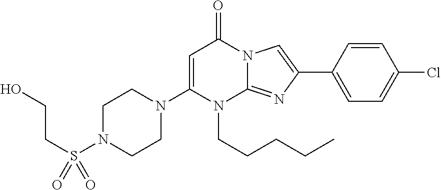 [SMILES: CCCCCn1c(N2CCN(S(=O)(=O)CCO)CC2)cc(=O)n2cc(-c3ccc(Cl)cc3)nc12] | Method A | 2.46 | 523 |

TABLE 60

| | | | Retention | |
|---|---|---|---|---|
| | | | Time | Mass |
| Compound | Structure | LC/MS | [min] | [M + H] |
| 291 | 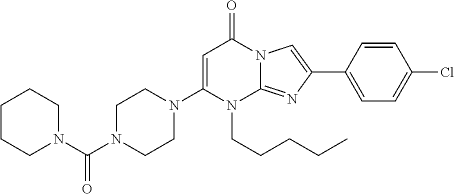 [SMILES: CCCCCn1c(N2CCN(C(=O)N3CCCCC3)CC2)cc(=O)n2cc(-c3ccc(Cl)cc3)nc12] | Method A | 2.99 | 511 |
| 292 | 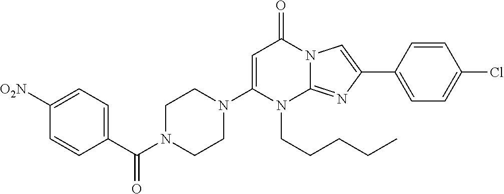 [SMILES: CCCCCn1c(N2CCN(C(=O)c3ccc([N+](=O)[O-])cc3)CC2)cc(=O)n2cc(-c3ccc(Cl)cc3)nc12] | | | |
| | | Method | 2.88 | 549 |
| | | A | | |
| 293 | 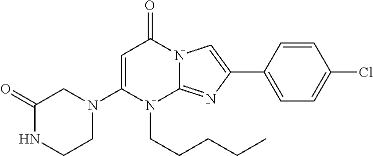 [SMILES: CCCCCn1c(N2CCNC(=O)C2)cc(=O)n2cc(-c3ccc(Cl)cc3)nc12] | Method A | 2.31 | 414 |
| 294 | 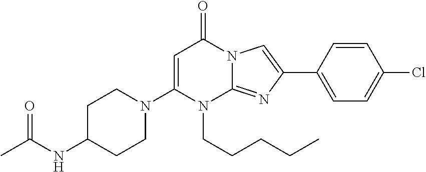 [SMILES: CCCCCn1c(N2CCC(NC(C)=O)CC2)cc(=O)n2cc(-c3ccc(Cl)cc3)nc12] | Method A | 2.48 | 456 |

TABLE 61

| | | | Retention | |
|---|---|---|---|---|
| | | | Time | Mass |
| Compound | Structure | LC/MS | [min] | [M + H] |
| 295 | 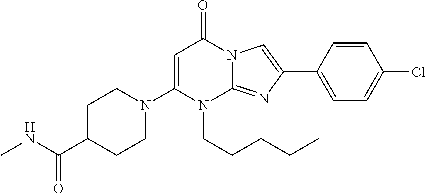 [SMILES: CCCCCn1c(N2CCC(C(=O)NC)CC2)cc(=O)n2cc(-c3ccc(Cl)cc3)nc12] | Method A | 2.47 | 456 |
| 296 | 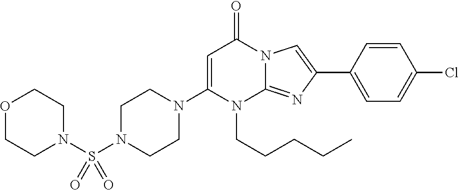 [SMILES: CCCCCn1c(N2CCN(S(=O)(=O)N3CCOCC3)CC2)cc(=O)n2cc(-c3ccc(Cl)cc3)nc12] | Method A | 2.81 | 549 |
| 297 | 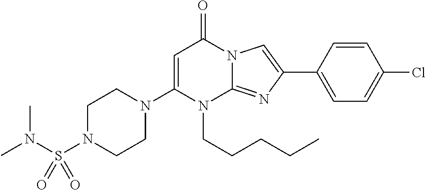 [SMILES: CCCCCn1c(N2CCN(S(=O)(=O)N(C)C)CC2)cc(=O)n2cc(-c3ccc(Cl)cc3)nc12] | Method A | 2.86 | 507 |
| 298 | 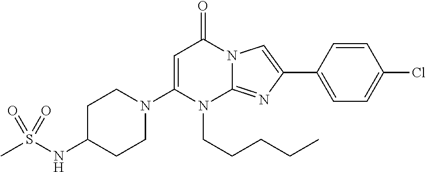 [SMILES: CCCCCn1c(N2CCC(NS(C)(=O)=O)CC2)cc(=O)n2cc(-c3ccc(Cl)cc3)nc12] | Method A | 2.61 | 492 |
| 299 | 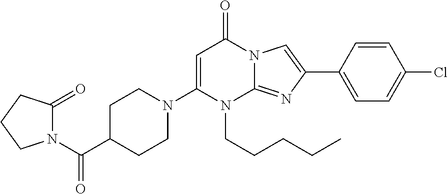 [SMILES: CCCCCn1c(N2CCC(C(=O)N3CCCC3=O)CC2)cc(=O)n2cc(-c3ccc(Cl)cc3)nc12] | Method A | 2.58 | 511 |

TABLE 62

| | | | Retention | |
|---|---|---|---|---|
| | | | Time | Mass |
| Compound | Structure | LC/MS | [min] | [M + H] |
| 300 | 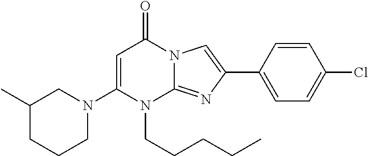 [SMILES: CCCCCn1c(N2CCCC(C)C2)cc(=O)n2cc(-c3ccc(Cl)cc3)nc12] | Method A | 3.43 | 412 |
| 301 | 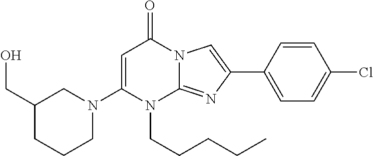 [SMILES: CCCCCn1c(N2CCCC(CO)C2)cc(=O)n2cc(-c3ccc(Cl)cc3)nc12] | Method A | 2.73 | 428 |
| 302 | 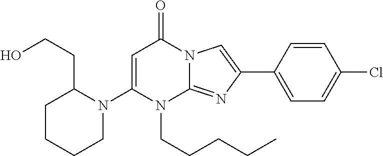 [SMILES: CCCCCn1c(N2CCCCC2CCO)cc(=O)n2cc(-c3ccc(Cl)cc3)nc12] | Method A | 2.06 | 442 |
| 303 | 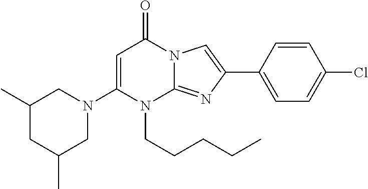 [SMILES: CCCCCn1c(N2CC(C)CC(C)C2)cc(=O)n2cc(-c3ccc(Cl)cc3)nc12] | Method A | 3.57 | 426 |
| 304 | 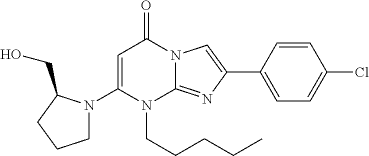 [SMILES: CCCCCn1c(N2CCC[C@H]2CO)cc(=O)n2cc(-c3ccc(Cl)cc3)nc12] | Method A | 2 .60 | 414 |

TABLE 63

| | | | Retention | |
|---|---|---|---|---|
| | | | Time | Mass |
| Compound | Structure | LC/MS | [min] | [M + H] |
| 305 | 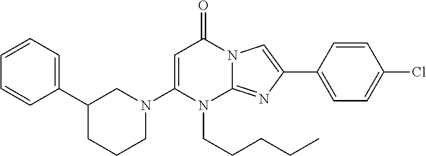 [SMILES: CCCCCn1c(N2CCCC(c3ccccc3)C2)cc(=O)n2cc(-c3ccc(Cl)cc3)nc12] | Method A | 3.53 | 474 |
| 306 | 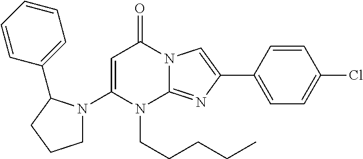 [SMILES: CCCCCn1c(N2CCCC2c2ccccc2)cc(=O)n2cc(-c3ccc(Cl)cc3)nc12] | Method A | 3.20 | 460 |
| 307 | 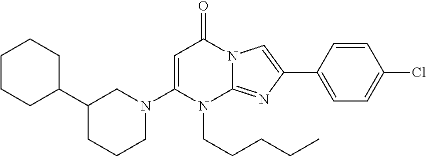 [SMILES: CCCCCn1c(N2CCCC(C3CCCCC3)C2)cc(=O)n2cc(-c3ccc(Cl)cc3)nc12] | Method A | 3.94 | 480 |
| 308 | 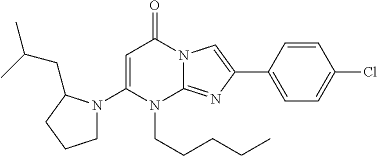 [SMILES: CCCCCn1c(N2CCCC2CC(C)C)cc(=O)n2cc(-c3ccc(Cl)cc3)nc12] | Method A | 3.41 | 440 |
| 309 | 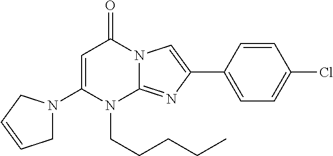 [SMILES: CCCCCn1c(N2CC=CC2)cc(=O)n2cc(-c3ccc(Cl)cc3)nc12] | Method A | 2.85 | 382 |

TABLE 64

| | | | Retention | |
|---|---|---|---|---|
| | | | Time | Mass |
| Compound | Structure | LC/MS | [min] | [M + H] |
| 310 | 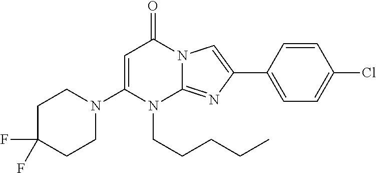 [SMILES: CCCCCn1c(N2CCC(F)(F)CC2)cc(=O)n2cc(-c3ccc(Cl)cc3)nc12] | Method A | 3.03 | 434 |
| 311 | 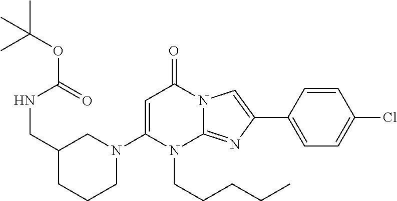 [SMILES: CCCCCn1c(N2CCCC(CNC(=O)OC(C)(C)C)C2)cc(=O)n2cc(-c3ccc(Cl)cc3)nc12] | Method A | 3.23 | 527 |
| 312 | 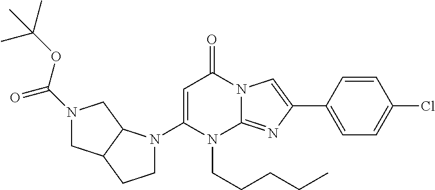 [SMILES: CCCCCn1c(N2CCC3CN(C(=O)OC(C)(C)C)CC32)cc(=O)n2cc(-c3ccc(Cl)cc3)nc12] | Method A | 3.06 | 525 |
| 313 | 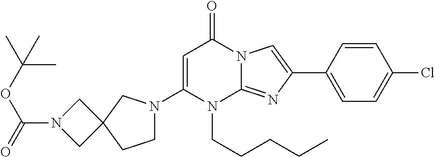 [SMILES: CCCCCn1c(N2CCC3(CN(C(=O)OC(C)(C)C)C3)C2)cc(=O)n2cc(-c3ccc(Cl)cc3)nc12] | Method A | 3.08 | 525 |
| 314 | 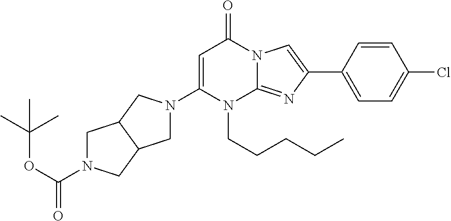 [SMILES: CCCCCn1c(N2CC3CN(C(=O)OC(C)(C)C)CC3C2)cc(=O)n2cc(-c3ccc(Cl)cc3)nc12] | Method A | 3.10 | 525 |

TABLE 65

| | | | Retention | |
|---|---|---|---|---|
| | | | Time | Mass |
| Compound | Structure | LC/MS | [min] | [M + H] |
| 315 | 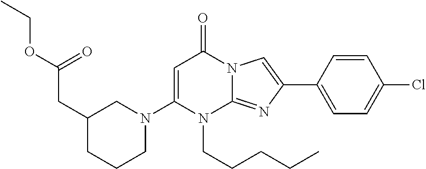 [SMILES: CCCCCn1c(N2CCCC(CC(=O)OCC)C2)cc(=O)n2cc(-c3ccc(Cl)cc3)nc12] | Method A | 3.26 | 484 |
| 316 | 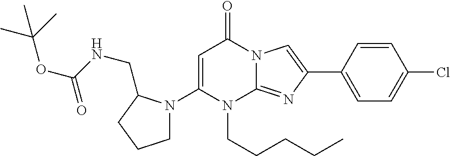 [SMILES: CCCCCn1c(N2CCCC2CNC(=O)OC(C)(C)C)cc(=O)n2cc(-c3ccc(Cl)cc3)nc12] | Method A | 3.10 | 513 |
| 317 | 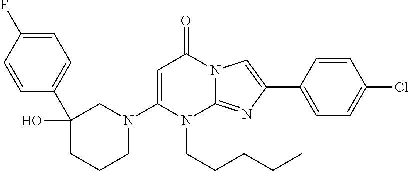 [SMILES: CCCCCn1c(N2CCCC(O)(c3ccc(F)cc3)C2)cc(=O)n2cc(-c3ccc(Cl)cc3)nc12] | Method A | 3.10 | 508 |
| 318 | 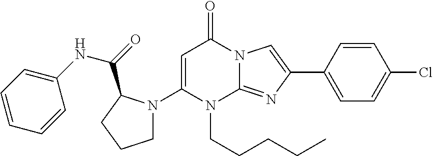 [SMILES: CCCCCn1c(N2CCC[C@H]2C(=O)Nc2ccccc2)cc(=O)n2cc(-c3ccc(Cl)cc3)nc12] | Method A | 2.91 | 503 |
| 319 | 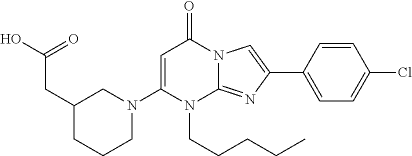 [SMILES: CCCCCn1c(N2CCCC(CC(=O)O)C2)cc(=O)n2cc(-c3ccc(Cl)cc3)nc12] | Method A | 2.71 | 456 |

TABLE 66

| | | | Retention | |
|---|---|---|---|---|
| | | | Time | Mass |
| Compound | Structure | LC/MS | [min] | [M + H] |
| 320 | 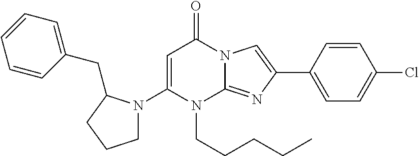 [SMILES: CCCCCn1c(N2CCCC2Cc2ccccc2)cc(=O)n2cc(-c3ccc(Cl)cc3)nc12] | Method A | 3.39 | 474 |
| 321 | 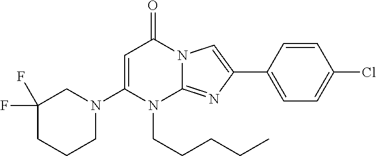 [SMILES: CCCCCn1c(N2CCCC(F)(F)C2)cc(=O)n2cc(-c3ccc(Cl)cc3)nc12] | Method A | 3.09 | 434 |
| 322 | 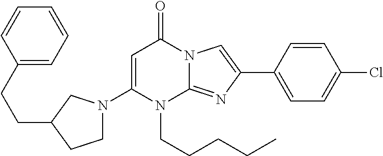 [SMILES: CCCCCn1c(N2CCC(CCc3ccccc3)C2)cc(=O)n2cc(-c3ccc(Cl)cc3)nc12] | Method A | 3.44 | 488 |
| 323 | 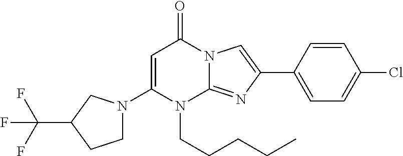 [SMILES: CCCCCn1c(N2CCC(C(F)(F)F)C2)cc(=O)n2cc(-c3ccc(Cl)cc3)nc12] | Method A | 3.09 | 452 |
| 324 | 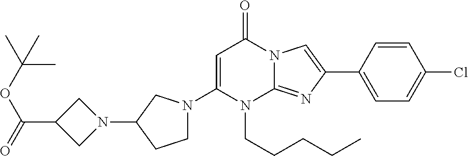 [SMILES: CCCCCn1c(N2CCC(N3CC(C(=O)OC(C)(C)C)C3)C2)cc(=O)n2cc(-c3ccc(Cl)cc3)nc12] | Method A | 3.05 | 540 |

TABLE 67

| | | | Retention | |
|---|---|---|---|---|
| | | | Time | Mass |
| Compound | Structure | LC/MS | [min] | [M + H] |
| 325 | 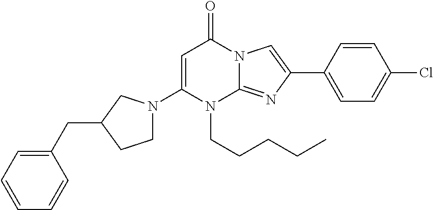 [SMILES: CCCCCn1c(N2CCC(Cc3ccccc3)C2)cc(=O)n2cc(-c3ccc(Cl)cc3)nc12] | Method A | 3.28 | 474 |
| 326 | 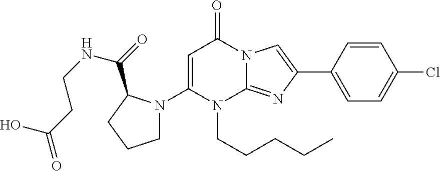 [SMILES: CCCCCn1c(N2CCC[C@H]2C(=O)NCCC(=O)O)cc(=O)n2cc(-c3ccc(Cl)cc3)nc12] | Method A | 2.31 | 499 |
| 327 | 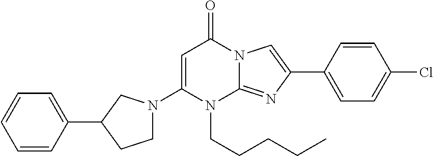 [SMILES: CCCCCn1c(N2CCC(c3ccccc3)C2)cc(=O)n2cc(-c3ccc(Cl)cc3)nc12] | Method A | 3.19 | 461 |
| 328 | 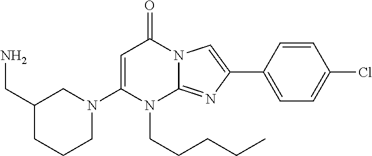 [SMILES: CCCCCn1c(N2CCCC(CN)C2)cc(=O)n2cc(-c3ccc(Cl)cc3)nc12] | Method A | 2.30 | 428 |
| 329 | 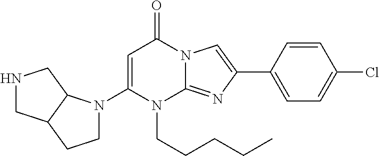 [SMILES: CCCCCn1c(N2CCC3CNCC32)cc(=O)n2cc(-c3ccc(Cl)cc3)nc12] | Method A | 2.28 | 426 |

TABLE 68

| | | | Retention | |
|---|---|---|---|---|
| | | | Time | Mass |
| Compound | Structure | LC/MS | [min] | [M + H] |
| 330 | 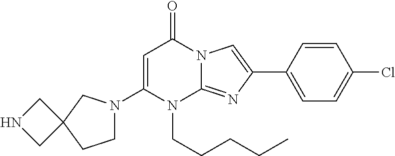 [SMILES: CCCCCn1c(N2CCC3(CNC3)C2)cc(=O)n2cc(-c3ccc(Cl)cc3)nc12] | Method A | 2.21 | 426 |
| 331 | 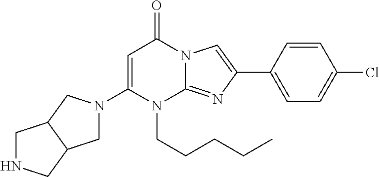 [SMILES: CCCCCn1c(N2CC3CNCC3C2)cc(=O)n2cc(-c3ccc(Cl)cc3)nc12] | Method A | 2.23 | 426 |
| 332 | 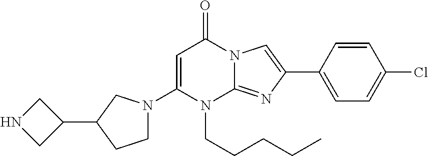 [SMILES: CCCCCn1c(N2CCC(C3CNC3)C2)cc(=O)n2cc(-c3ccc(Cl)cc3)nc12] | Method A | 2.21 | 440 |
| 333 | 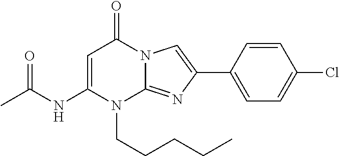 [SMILES: CCCCCn1c(NC(C)=O)cc(=O)n2cc(-c3ccc(Cl)cc3)nc12] | Method B | 2.31 | 373 |
| 334 | 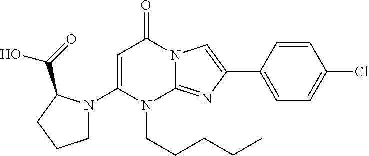 [SMILES: CCCCCn1c(N2CCC[C@H]2C(=O)O)cc(=O)n2cc(-c3ccc(Cl)cc3)nc12] | Method A | 2.49 | 429 |

TABLE 69

| | | | Retention | |
|---|---|---|---|---|
| | | | Time | Mass |
| Compound | Structure | LC/MS | [min] | [M + H] |
| 335 | 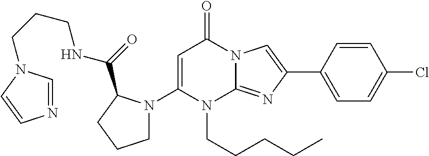 [SMILES: CCCCCn1c(N2CCC[C@H]2C(=O)NCCCn2ccnc2)cc(=O)n2cc(-c3ccc(Cl)cc3)nc12] | Method A | 1.90 | 536 |
| 336 | 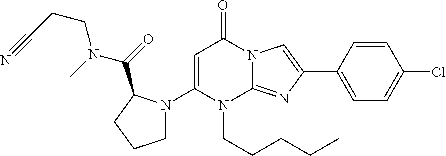 [SMILES: CCCCCn1c(N2CCC[C@H]2C(=O)N(C)CCC#N)cc(=O)n2cc(-c3ccc(Cl)cc3)nc12] | Method A | 2.51 | 495 |
| 337 | 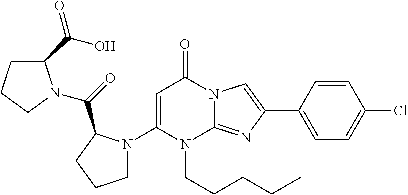 [SMILES: CCCCCn1c(N2CCC[C@H]2C(=O)N2CCC[C@H]2C(=O)O)cc(=O)n2cc(-c3ccc(Cl)cc3)nc12] | Method A | 2.37 | 526 |
| 338 | 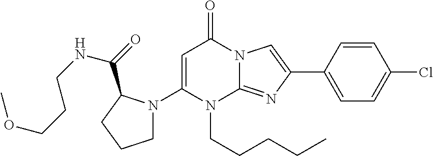 [SMILES: CCCCCn1c(N2CCC[C@H]2C(=O)NCCCOC)cc(=O)n2cc(-c3ccc(Cl)cc3)nc12] | Method A | 2.51 | 500 |
| 339 | 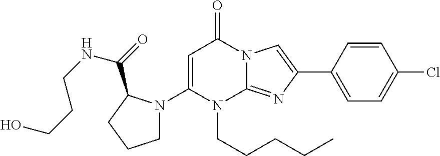 [SMILES: CCCCCn1c(N2CCC[C@H]2C(=O)NCCCO)cc(=O)n2cc(-c3ccc(Cl)cc3)nc12] | Method A | 2.25 | 486 |

TABLE 70

| | | | Retention | |
|---|---|---|---|---|
| | | | Time | Mass |
| Compound | Structure | LC/MS | [min] | [M + H] |
| 340 | 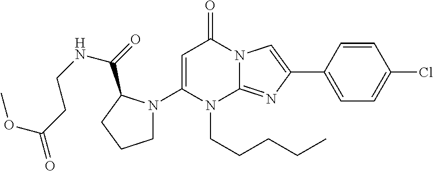 [SMILES: CCCCCn1c(N2CCC[C@H]2C(=O)NCCC(=O)OC)cc(=O)n2cc(-c3ccc(Cl)cc3)nc12] | Method A | 2.51 | 514 |
| 341 | 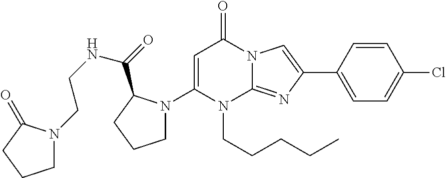 [SMILES: CCCCCn1c(N2CCC[C@H]2C(=O)NCCN2CCCC2=O)cc(=O)n2cc(-c3ccc(Cl)cc3)nc12] | Method A | 2.30 | 539 |
| 342 | 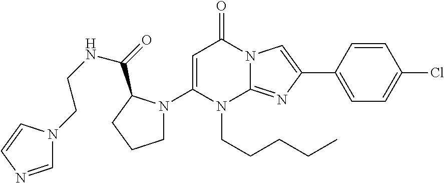 [SMILES: CCCCCn1c(N2CCC[C@H]2C(=O)NCCn2ccnc2)cc(=O)n2cc(-c3ccc(Cl)cc3)nc12] | Method A | 1.90 | 522 |
| 343 | 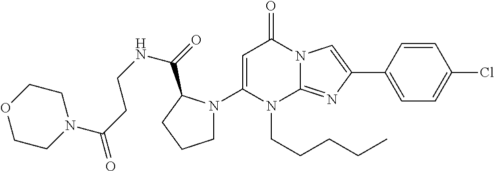 [SMILES: CCCCCn1c(N2CCC[C@H]2C(=O)NCCC(=O)N2CCOCC2)cc(=O)n2cc(-c3ccc(Cl)cc3)nc12] | Method A | 2.32 | 569 |

TABLE 71

| | | | Retention | |
|---|---|---|---|---|
| | | | Time | Mass |
| Compound | Structure | LC/MS | [min] | [M + H] |
| 344 | 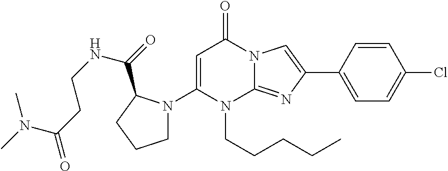 [SMILES: CCCCCn1c(N2CCC[C@H]2C(=O)NCCC(=O)N(C)C)cc(=O)n2cc(-c3ccc(Cl)cc3)nc12] | Method A | 2.35 | 527 |
| 345 | 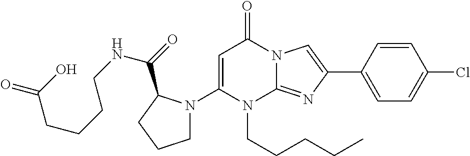 [SMILES: CCCCCn1c(N2CCC[C@H]2C(=O)NCCCCC(=O)O)cc(=O)n2cc(-c3ccc(Cl)cc3)nc12] | Method A | 2.32 | 528 |
| 346 | 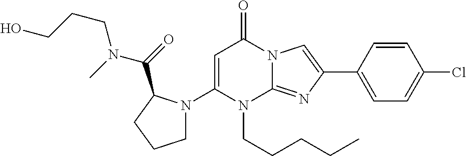 [SMILES: CCCCCn1c(N2CCC[C@H]2C(=O)N(C)CCCO)cc(=O)n2cc(-c3ccc(Cl)cc3)nc12] | Method A | 2.34 | 500 |
| 347 | 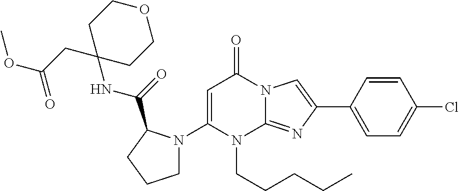 [SMILES: CCCCCn1c(N2CCC[C@H]2C(=O)NC2(CC(=O)OC)CCOCC2)cc(=O)n2cc(-c3ccc(Cl)cc3)nc12] | Method A | 2.58 | 584 |
| 348 | 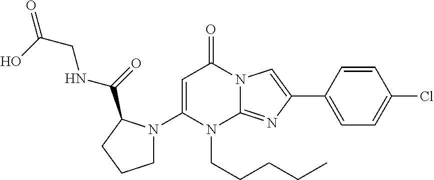 [SMILES: CCCCCn1c(N2CCC[C@H]2C(=O)NCC(=O)O)cc(=O)n2cc(-c3ccc(Cl)cc3)nc12] | Method A | 2.28 | 486 |

TABLE 72

| | | | Retention | |
|---|---|---|---|---|
| | | | Time | Mass |
| Compound | Structure | LC/MS | [min] | [M + H] |
| 349 | 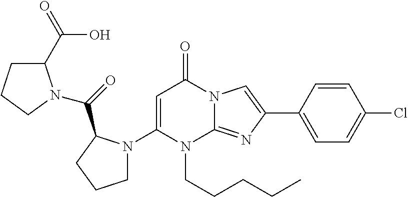 [SMILES: CCCCCn1c(N2CCC[C@H]2C(=O)N2CCCC2C(=O)O)cc(=O)n2cc(-c3ccc(Cl)cc3)nc12] | Method A | 2.37 | 526 |
| 350 | 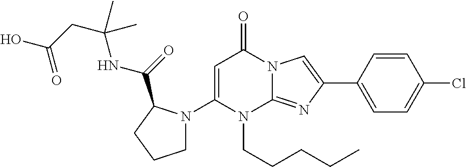 [SMILES: CCCCCn1c(N2CCC[C@H]2C(=O)NC(C)(C)CC(=O)O)cc(=O)n2cc(-c3ccc(Cl)cc3)nc12] | Method A | 2.48 | 528 |
| 351 | 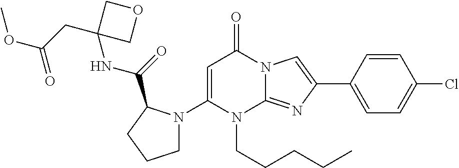 [SMILES: CCCCCn1c(N2CCC[C@H]2C(=O)NC2(CC(=O)OC)COC2)cc(=O)n2cc(-c3ccc(Cl)cc3)nc12] | Method A | 2.62 | 570 |
| 352 | 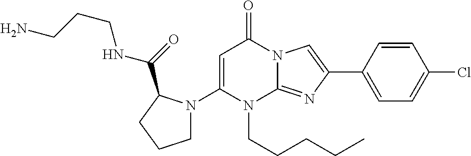 [SMILES: CCCCCn1c(N2CCC[C@H]2C(=O)NCCCN)cc(=O)n2cc(-c3ccc(Cl)cc3)nc12] | Method A | 1.90 | 485 |
| 353 | 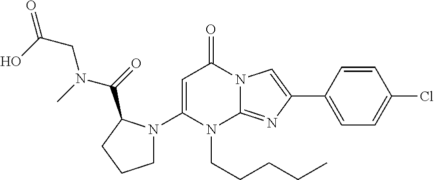 [SMILES: CCCCCn1c(N2CCC[C@H]2C(=O)N(C)CC(=O)O)cc(=O)n2cc(-c3ccc(Cl)cc3)nc12] | Method A | 2.34 | 500 |

TABLE 73

| | | | Retention | |
|---|---|---|---|---|
| | | | Time | Mass |
| Compound | Structure | LC/MS | [min] | [M + H] |
| 354 | 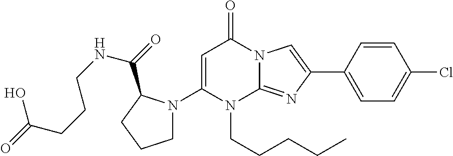 [SMILES: CCCCCn1c(N2CCC[C@H]2C(=O)NCCCC(=O)O)cc(=O)n2cc(-c3ccc(Cl)cc3)nc12] | Method A | 2.28 | 514 |
| 355 | 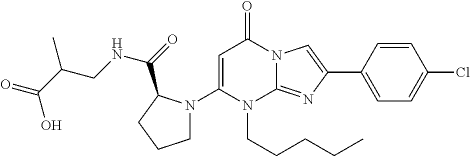 [SMILES: CCCCCn1c(N2CCC[C@H]2C(=O)NCC(C)C(=O)O)cc(=O)n2cc(-c3ccc(Cl)cc3)nc12] | Method A | 2.35 | 514 |
| 356 | 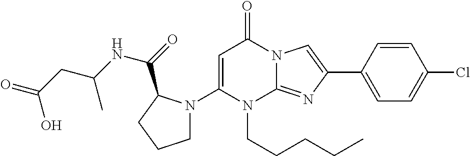 [SMILES: CCCCCn1c(N2CCC[C@H]2C(=O)NC(C)CC(=O)O)cc(=O)n2cc(-c3ccc(Cl)cc3)nc12] | Method A | 2.34 | 514 |
| 357 | 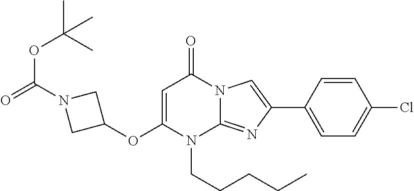 [SMILES: CCCCCn1c(OC2CN(C(=O)OC(C)(C)C)C2)cc(=O)n2cc(-c3ccc(Cl)cc3)nc12] | Method B | 2.61 | 435 |
| 358 | 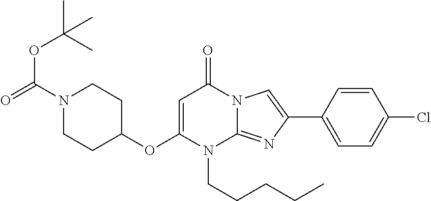 [SMILES: CCCCCn1c(OC2CCN(C(=O)OC(C)(C)C)CC2)cc(=O)n2cc(-c3ccc(Cl)cc3)nc12] | Method B | 2.46 | 387 |

TABLE 74

| | | | Retention | |
|---|---|---|---|---|
| | | | Time | Mass |
| Compound | Structure | LC/MS | [min] | [M + H] |
| 359 | 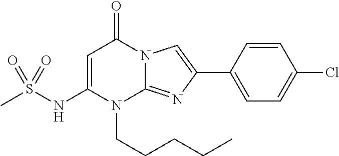 [SMILES: CCCCCn1c(NS(C)(=O)=O)cc(=O)n2cc(-c3ccc(Cl)cc3)nc12] | Method B | 2.98 | 487 |
| 360 | 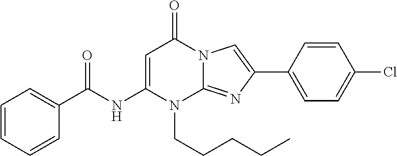 [SMILES: CCCCCn1c(NC(=O)c2ccccc2)cc(=O)n2cc(-c3ccc(Cl)cc3)nc12] | Method B | 3.12 | 515 |
| 361 | 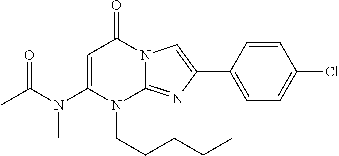 [SMILES: CCCCCn1c(N(C)C(C)=O)cc(=O)n2cc(-c3ccc(Cl)cc3)nc12] | Method B | 2.46 | 387 |

### Example 8

### N-(2-(2-oxa-6-azaspiro[3.3]heptan-6-yl)-4-(2-(4-chlorophenyl)-5-oxo-8-pentyl-5,8-dihydroimidazo[1,2-a]pyrimidin-7-yl)benzamide (365)

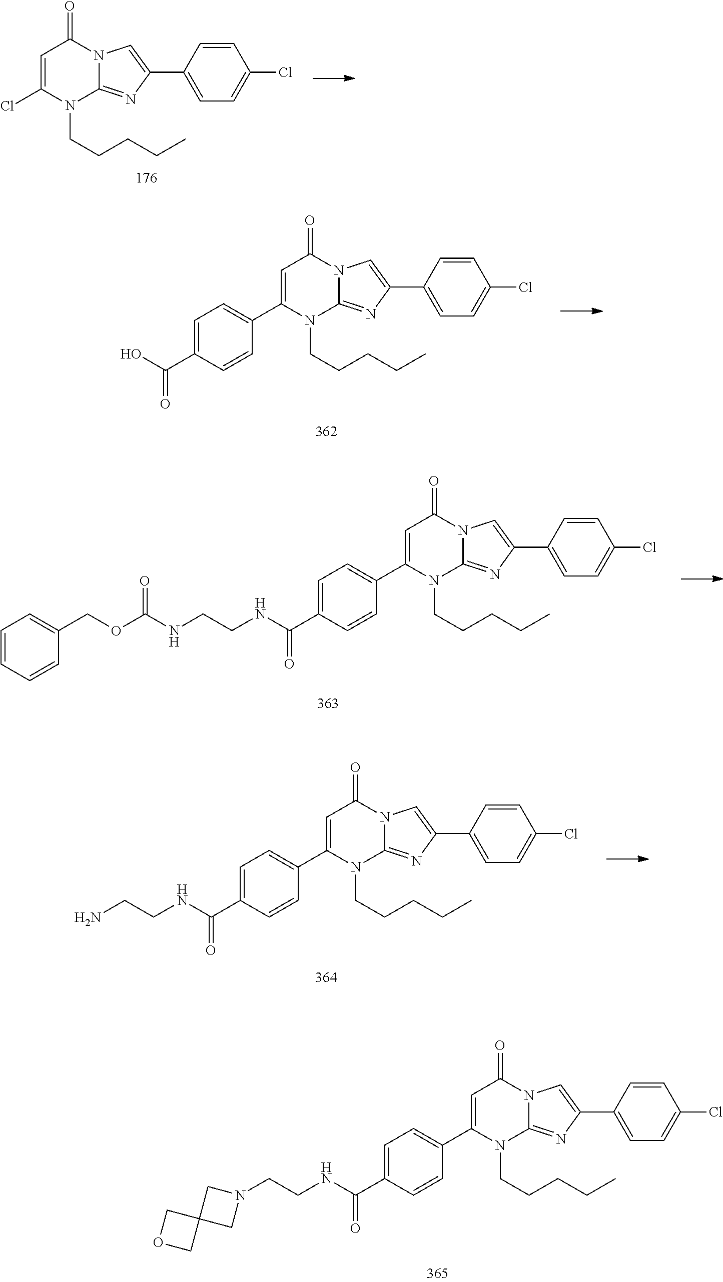 [SMILES: CCCCCn1c(Cl)cc(=O)n2cc(-c3ccc(Cl)cc3)nc12 ; CCCCCn1c(-c2ccc(C(=O)O)cc2)cc(=O)n2cc(-c3ccc(Cl)cc3)nc12 ; CCCCCn1c(-c2ccc(C(=O)NCCNC(=O)OCc3ccccc3)cc2)cc(=O)n2cc(-c3ccc(Cl)cc3)nc12 ; CCCCCn1c(-c2ccc(C(=O)NCCN)cc2)cc(=O)n2cc(-c3ccc(Cl)cc3)nc12 ; CCCCCn1c(-c2ccc(C(=O)NCCN3CC4(COC4)C3)cc2)cc(=O)n2cc(-c3ccc(Cl)cc3)nc12]

### Step 1:

To a solution of compound (176, 1.82 g, 5.20 mmol) in N,N-dimethylformamide (55 mL) were added 4-carboxyphenylboronic acid (1.29 g, 7.79 mmol), [1,1′-bis(diphenylphosphino)ferrocene]palladium(II) dichloride-dichloromethane complex (424 mg, 0.520 mmol) and aqueous sodium carbonate (2 mol/L, 15.6 mL), and the mixture was stirred at 100° C. for 2 hours. After cooling to room temperature, water was added, and the reaction mixture was extracted with ethyl acetate. The organic layer was washed with water, dried with anhydrous sodium sulfate and concentrated under reduced pressure. The residue was purified by silica gel chromatography (chloroform/methanol) to yield 4-(2-(4-chlorophenyl)-5-oxo-8-pentyl-5,8-dihydroimidazo[1,2-a]pyrimidin-7-yl)benzoic acid (362, 1.65 g, yield: 73%) as a colorless solid.

LC/MS (Method A) Retention Time=2.75 min, Found Mass [M+H]=436.

### Step 2:

To a solution of the compound (362, 400 mg, 0.918 mmol) in methylene chloride (6 mL) were added benzyl 2-aminoethyl carbamate (267 mg, 1.38 mmol), 1-hydroxybenzotriazole (25 mg, 0.18 mmol), 1-ethyl-3-(3-dimethylaminopropyl)carbodiimide hydrochloride (367 mg, 1.38 mmol) and triethylamine (0.636 mL, 4.59 mmol), and the mixture was stirred at room temperature for 18 hours. Water was added to the reaction mixture, and the mixture was extracted with ethyl acetate. The organic layer was washed with brine, dried with anhydrous sodium sulfate and concentrated under reduced pressure. The residue was purified by silica gel chromatography (hexane/ethyl acetate) to yield benzyl 2-(4-(2-(4-chlorophenyl)-5-oxo-8-pentyl-5,8-dihydroimidazo[1,2-a]pyrimidin-7-yl)benzamide)ethylcarbamate (363, 422 mg, yield: 75%) as a yellow solid.

LC/MS (Method B) Retention Time=2.74 min, Found Mass [M+H]=612.

### Step 3

To a solution of the compound (363, 152 mg, 0.248 mmol) in methylene chloride (5 mL) was added a solution of boron tribromide in methylene chloride (1 mol/L, 0.50 mL, 0.50 mmol) was added at −78° C., and the mixture was stirred for at 0° C. 4 hours. Methanol in water was added to the reaction mixture, and the mixture was extracted with ethyl acetate. The organic layer was washed with brine, dried with anhydrous sodium sulfate and concentrated under reduced pressure. The residue was purified by silica gel chromatography (chloroform/methanol) to yield N-(2-aminoethyl)-4-(2-(4-chlorophenyl)-5-oxo-8-pentyl-5,8-dihydroimidazo[1,2-a]pyrimidin-7-yl)benzamide (364, 90 mg, yield: 76%) as a yellow solid.

1H-NMR (δ ppm TMS/DMSO-d6) 8.78 (t, 1H, J=5.3 Hz), 8.30 (s, 1H), 8.04-8.01 (m, 4H), 7.78-7.74 (m, 6H), 7.50 (d, 2H, J=7.5 Hz), 5.74 (s, 1H), 4.11 (t, 2H, J=7.2 Hz), 3.04-3.01 (m, 2H), 1.64-1.62 (m, 2H), 1.13-1.05 (m, 4H), 0.74 (t, 3H, J=6.8 Hz).

### Step 4

To a solution of the compound (364, 30 mg, 0.063 mmol) in 2-propanol (1.5 mL) were added sodium carbonate (67 mg, 0.63 mmol) and 3,3-bis(chloromethyl) oxetane (97 mg, and 0.63 mmol), and the mixture was stirred at 140° C. for 2 hours. After cooling the mixture to room temperature, hydrochloric acid (1 mol/L) was added, and the mixture was extracted with ethyl acetate. The organic layer was washed with brine, dried with anhydrous sodium sulfate and concentrated under reduced pressure. The obtained residue was purified by silica gel chromatography (chloroform/methanol) to yield N-(2-(2-oxa-6-azaspiro[3.3]heptan-6-yl)-4-(2-(4-chlorophenyl)-5-oxo-8-pentyl-5,8-dihydro-imidazo[1,2-a]pyrimidine-7-yl)benzamide (365, 13 mg, yield: 37%) of a colorless solid.

1H-NMR (δ ppm TMS/CDCl3) 7.99 (d, 2H, J=7.5 Hz), 7.93 (s, 1H), 7.85 (d, 2H, J=7.3 Hz), 7.52 (d, 2H, J=7.3 Hz), 7.40 (d, 2H, J=7.5 Hz), 5.75 (s, 1H), 4.76 (s, 4H), 4.15 (t, 2H, J=7.8 Hz), 3.60 (s, 4H), 3.52 (q, 2H, J=5.3 Hz), 2.78 (t, 2H, J=5.3 Hz), 1.73-1.70 (m, 2H), 1.23-1.16 (m, 4H), 0.81 (t, 3H, J=6.8 Hz).

Compound (366) to (867) were prepared in a similar manner.

TABLE 75

| Compound | Structure | 1H-NMR |
|---|---|---|
| 366 | 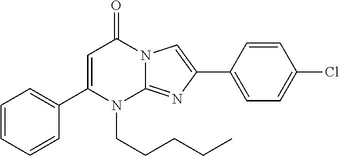 [SMILES: CCCCCn1c(-c2ccccc2)cc(=O)n2cc(-c3ccc(Cl)cc3)nc12] | (δ ppm TMS/CDCl3) 7.93 (s, 1H), 7.85 (d, 2H, J = 8.5 Hz), 7.54 (d, 3H, J = 6.3 Hz), 7.41 (t, 4H, J = 8.5 Hz), 5.77 (s, 1H), 4.17 (t, 2H, J = 7.8 Hz), 1.74-1.72 (m, 2H), 1.23-1.16 (m, 5H), 0.80 (t, 3H, J = 6.9 Hz). |
| 367 | 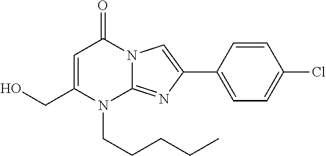 [SMILES: CCCCCn1c(CO)cc(=O)n2cc(-c3ccc(Cl)cc3)nc12] | (δ ppm TMS/CDCl3) 7.81-7.79 (m, 3H), 7.38 (d, 2H, J = 7.8 Hz), 5.92 (s, 1H), 4.67 (d, 2H, J = 6.3 Hz), 4.32 (t, 2H, J = 7.8 Hz), 1.89-1.86 (m, 2H), 1.43-1.40 (m, 4H), 0.94 (t, 3H, J = 6.4 Hz). |
| 368 | 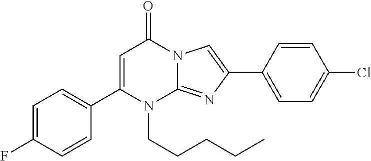 [SMILES: CCCCCn1c(-c2ccc(F)cc2)cc(=O)n2cc(-c3ccc(Cl)cc3)nc12] | (δ ppm TMS/CDCl3) 7.93 (s, 1H), 7.84 (d, 2H, J = 8.3 Hz), 7.44-7.39 (m, 4H), 7.23 (d, 1H, J = 8.3 Hz), 5.74 (s, 1H), 4.16 (t, 2H, J = 7.8 Hz), 1.73-1.71 (m, 2H), 1.22-1.15 (m, 4H), 0.82 (t, 3H, J = 6.9 Hz). |
| 369 | 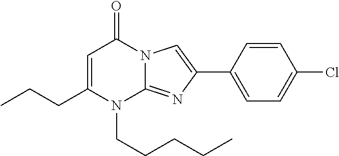 [SMILES: CCCCCn1c(CCC)cc(=O)n2cc(-c3ccc(Cl)cc3)nc12] | (δ ppm TMS/CDCl3) 7.86 (s, 1H), 7.82 (d, 2H, J = 8.0 Hz), 7.38 (d, 2H, J = 8.0 Hz), 5.71 (s, 1H), 4.29 (t, 2H, J = 7.8 Hz), 2.64 (t, 2H, J = 7.7 Hz), 1.88-1.86 (m, 2H), 1.77-1.73 (m, 2H), 1.44-1.43 (m, 4H), 1.09 (t, 3H, J = 7.3 Hz), 0.96 (t, 3H, J = 6.3 Hz). |
| 370 | 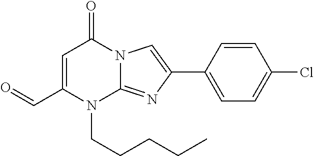 [SMILES: CCCCCn1c(C=O)cc(=O)n2cc(-c3ccc(Cl)cc3)nc12] | (δ ppm TMS/CDCl3) 9.78 (s, 1H), 7.95 (s, 1H), 7.85 (d, 2H, J = 7.8 Hz), 7.41 (d, 2H, J = 7.8 Hz), 6.37 (s, 1H), 4.78 (t, 2H, J = 7.8 Hz), 1.86-1.83 (m, 2H), 1.43-1.42 (m, 4H), 0.94 (t, 3H, J = 6.4 Hz). |

TABLE 76

| Compound | Structure | 1H-NMR |
|---|---|---|
| 371 | 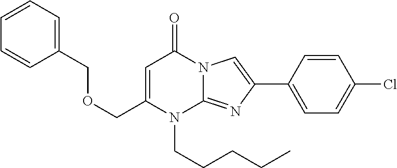 [SMILES: CCCCCn1c(COCc2ccccc2)cc(=O)n2cc(-c3ccc(Cl)cc3)nc12] | (δ ppm TMS/CDCl3) 7.87 (s, 1H), 7.82 (d, 2H, J = 7.3 Hz), 7.38-7.36 (m, 7H), 5.91 (s, 1H), 4.63 (s, 2H), 4.49 (s, 2H), 4.31 (t, 2H, J = 7.7 Hz), 1.87-1.84 (m, 2H), 1.37-1.35 (m, 4H), 0.92 (t, 3H, J = 5.6 Hz). |
| 372 | 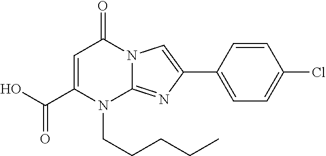 [SMILES: CCCCCn1c(C(=O)O)cc(=O)n2cc(-c3ccc(Cl)cc3)nc12] | (δ ppm TMS/DMSO-d6) 8.25 (s, 1H), 7.99 (d, 2H, J = 8.3 Hz), 7.49 (d, 2H, J = 8.0 Hz), 6.01 (s, 1H), 4.47 (t, 2H, J = 7.2 Hz), 1.85-1.82 (m, 2H), 1.33- 1.31 (m, 4H), 0.88 (t, 3H, J = 6.4 Hz). |
| 373 | 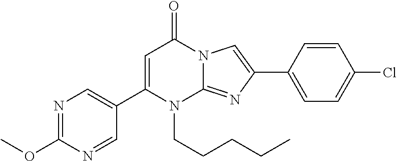 [SMILES: CCCCCn1c(-c2cnc(OC)nc2)cc(=O)n2cc(-c3ccc(Cl)cc3)nc12] | (δ ppm TMS/CDCl3) 8.64 (s, 2H), 7.93 (s, 1H), 7.84 (d, 2H, J = 8.3 Hz), 7.40 (d, 2H, J = 8.3 Hz), 5.76 (s, 1H), 4.19-4.15 (m, 5H), 1.78-1.74 (m, 2H), 1.28-1.22 (m, 4H), 0.86 (t, 3H, J = 6.9 Hz). |
| 374 | 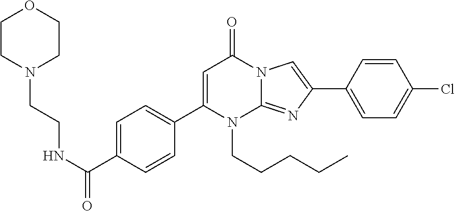 [SMILES: CCCCCn1c(-c2ccc(C(=O)NCCN3CCOCC3)cc2)cc(=O)n2cc(-c3ccc(Cl)cc3)nc12] | (δ ppm TMS/DMSO-d6) 8.62-8.65 (m, 1H), 8.33 (s, 1H), 8.00- 8.02 (m, 4H), 7.72 (d, 2H, J = 7.7 Hz), 7.51 (d, 2H, J = 7.9 Hz), 5.77 (s, 1H), 4.10-4.13 (m, 2H), 3.57-3.59 (m, 4H), 3.35-3.42 (m, 4H), 2.43 (s, 4H), 1.62-1.64 (m, 2H), 1.07- 1.09 (br m, 4H), 0.73 (t, 3H, J = 6.7 Hz). |
| 375 | 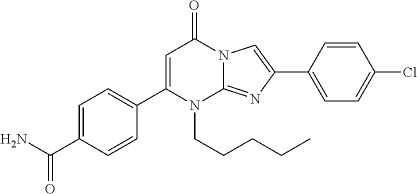 [SMILES: CCCCCn1c(-c2ccc(C(N)=O)cc2)cc(=O)n2cc(-c3ccc(Cl)cc3)nc12] | (δ ppm TMS/CDCl3) 8.01-8.03 (m, 5H), 7.69 (d, 2H, J = 7.8 Hz), 7.56 (d, 2H, J = 7.8 Hz), 6.20 (brs, 1H), 5.81 (s, 1H), 5.70 (brs, 1H), 4.23 (t, 2H, J = 6.8 Hz), 2.32 (t, 2H, J = 6.8 Hz), 1.88-1.91 (br m, 2H), 1.57 (br s, 2H). |

TABLE 77

| Compound | Structure | 1H-NMR |
|---|---|---|
| 376 | 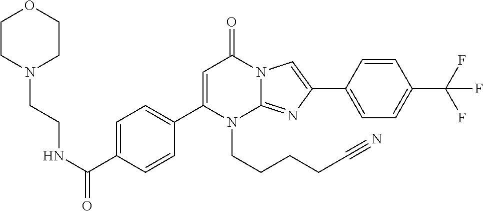 [SMILES: N#CCCCCn1c(-c2ccc(C(=O)NCCN3CCOCC3)cc2)cc(=O)n2cc(-c3ccc(C(F)(F)F)cc3)nc12] | (δ ppm TMS/CDCl3) 7.99-8.02 (m, 6H), 7.69 (d, 2H, J = 8.0 Hz), 7.54 (d, 2H, J = 7.5 Hz), 7.08 (s, 1H), 5.81 (s, 1H), 4.23 (t, 2H, J = 7.4 Hz), 3.76-3.79 (br m, 5H), 3.63- 3.64 (m, 2H), 2.69 (t, 2H, J = 6.9 Hz), 2.59 (s, 4H), 2.32 (t, 2H, J = 6.9 Hz), 1.89-1.92 (br m, 2H), 1.57-1.59 (m, 2H). |
| 377 | 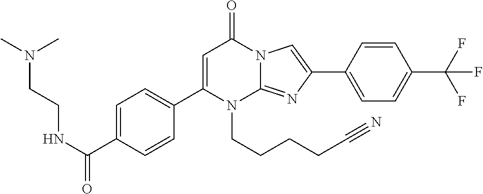 [SMILES: CN(C)CCNC(=O)c1ccc(-c2cc(=O)n3cc(-c4ccc(C(F)(F)F)cc4)nc3n2CCCCC#N)cc1] | (δ ppm TMS/CDCl3) 8.01-8.05 (br m, 5H), 7.78 (s, 1H), 7.69 (d, 2H, J = 8.0 Hz), 7.52 (d, 2H, J = 7.8 Hz), 5.80 (s, 1H), 4.23 (t, 2H, J = 7.4 Hz), 3.67-3.68 (m, 2H), 2.78 (t, 2H, J = 7.8 Hz), 2.46 (s, 6H), 2.32 (t, 2H, J = 6.9 Hz), 1.88-1.91 (br m, 2H), 1.57- 1.59 (m, 2H). |
| 378 | 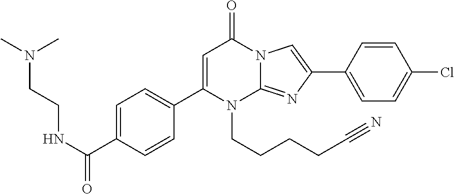 [SMILES: CN(C)CCNC(=O)c1ccc(-c2cc(=O)n3cc(-c4ccc(Cl)cc4)nc3n2CCCCC#N)cc1] | (δ ppm TMS/DMSO-d6) 8.57 (t, 1H, J = 5.3 Hz), 8.32 (s, 1H), 8.03-8.02 (m, 4H), 7.70 (d, 2H, J = 7.5 Hz), 7.50 (d, 2H, J = 7.3 Hz), 5.76 (s, 1H), 4.14 (t, 2H, J = 7.0 Hz), 3.40-3.39 (m, 2H), 2.41 (q, 4H, J = 7.4 Hz), 2.19 (s, 6H), 1.78-1.71 (m, 2H), 1.44-1.37 (m, 2H). |
| 379 | 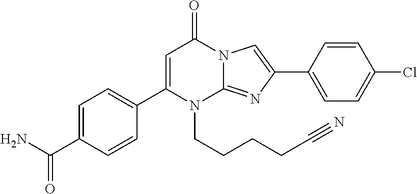 [SMILES: N#CCCCCn1c(-c2ccc(C(N)=O)cc2)cc(=O)n2cc(-c3ccc(Cl)cc3)nc12] | (δ ppm TMS/DMSO-d6) 8.31 (s, 1H), 8.15 (s, 1H), 8.05-8.03 (m, 4H), 7.69 (d, 2H, J = 7.8 Hz), 7.56 (s, 1H), 7.50 (d, 2H, J = 8.0 Hz), 5.77 (s, 1H), 4.14 (t, 2H, J = 6.9 Hz), 2.40 (t, 2H, J = 6.9 Hz), 1.78-1.71 (m, 2H), 1.44-1.37 (m, 2H). |
| 380 | 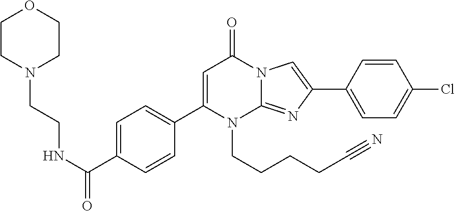 [SMILES: N#CCCCCn1c(-c2ccc(C(=O)NCCN3CCOCC3)cc2)cc(=O)n2cc(-c3ccc(Cl)cc3)nc12] | (δ ppm TMS/DMSO-d6) 8.60 (t, 1H, J = 5.8 Hz), 8.31 (s, 1H), 8.02 (t, 4H, J = 8.0 Hz), 7.70 (d, 2H, J = 7.8 Hz), 7.49 (d, 2H, J = 8.0 Hz), 5.76 (s, 1H), 4.14 (t, 2H, J = 7.2 Hz), 3.58 (t, 4H, J = 4.1 Hz), 2.41-2.39 (m, 6H), 1.76-1.73 (m, 2H), 1.44-1.36 (m, 2H). |

TABLE 78

| Compound | Structure | 1H-NMR |
|---|---|---|
| 381 | 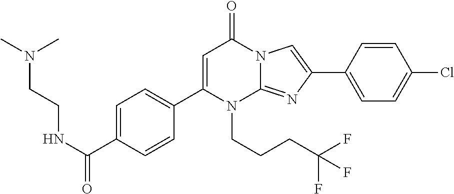 [SMILES: CN(C)CCNC(=O)c1ccc(-c2cc(=O)n3cc(-c4ccc(Cl)cc4)nc3n2CCCC(F)(F)F)cc1] | (δ ppm TMS/DMSO-d6) 8.57 (t, 1H, J = 6.1 Hz), 8.32 (s, 1H), 8.03-8.01 (m, 4H), 7.70 (d, 2H, J = 7.8 Hz), 7.51 (d, 2H, J = 7.8 Hz), 5.75 (s, 1H), 4.17 (t, 2H, J = 7.3 Hz), 3.40 (q, 2H, J = 6.3 Hz), 2.43 (t, 2H, J = 6.7 Hz), 2.19 (s, 8H), 1.94-1.90 (m, 2H). |
| 382 | 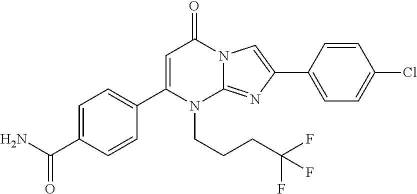 [SMILES: NC(=O)c1ccc(-c2cc(=O)n3cc(-c4ccc(Cl)cc4)nc3n2CCCC(F)(F)F)cc1] | (δ ppm TMS/DMSO-d6) 8.33 (s, 1H), 8.18 (s, 1H), 8.06-8.03 (m, 4H), 7.70 (d, 2H, J = 7.5 Hz), 7.60-7.50 (m, 4H), 5.77 (s, 1H), 4.16 (t, 2H, J = 7.2 Hz), 2.25-2.19 (m, 2H), 1.94- 1.90 (m, 2H). |
| 383 | 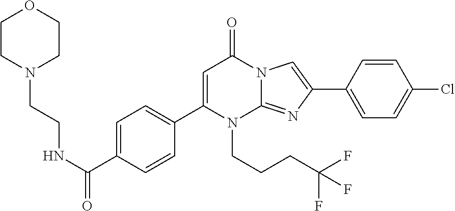 [SMILES: O=C(NCCN1CCOCC1)c1ccc(-c2cc(=O)n3cc(-c4ccc(Cl)cc4)nc3n2CCCC(F)(F)F)cc1] | (δ ppm TMS/DMSO-d6) 8.63 (t, 1H, J = 5.3 Hz), 8.33 (s, 1H), 8.04-8.02 (m, 4H), 7.71 (d, 2H, J = 7.5 Hz), 7.52 (d, 2H, J = 7.5 Hz), 5.77 (s, 1H), 4.17 (t, 2H, J = 6.3 Hz), 3.60-3.57 (m, 4H), 2.43 (br s, 4H), 2.25-2.20 (m, 2H), 1.94- 1.90 (m, 2H). |
| 384 | 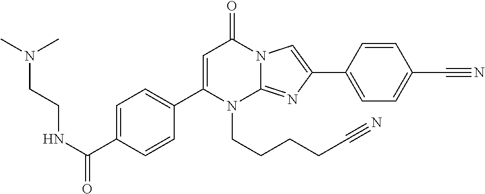 [SMILES: CN(C)CCNC(=O)c1ccc(-c2cc(=O)n3cc(-c4ccc(C#N)cc4)nc3n2CCCCC#N)cc1] | (δ ppm TMS/DMSO-d6) 8.57 (t, 1H, J = 5.5 Hz), 8.51 (s, 1H), 8.20 (d, 2H, J = 7.5 Hz), 8.02 (d, 2H, J = 7.8 Hz), 7.90 (d, 2H, J = 7.3 Hz), 7.70 (d, 2H, J = 7.5 Hz), 5.78 (br s, 1H), 4.15 (t, 2H, J = 7.0 Hz), 3.39 (q, 2H, J = 6.4 Hz), 2.44-2.35 (m, 4H), 2.19 (s, 6H), 1.79- 1.71 (m, 2H), 1.45-1.38 (m, 2H). |
| 385 | 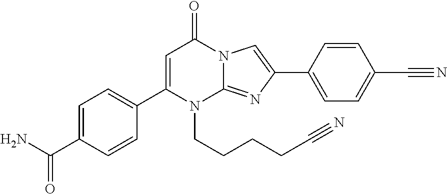 [SMILES: N#CCCCCn1c(-c2ccc(C(N)=O)cc2)cc(=O)n2cc(-c3ccc(C#N)cc3)nc12] | (δ ppm TMS/DMSO-d6) 8.51 (s, 1H), 8.21 (d, 2H, J = 7.8 Hz), 8.16 (s, 1H), 8.06 (d, 2H, J = 7.5 Hz), 7.90 (d, 2H, J = 7.8 Hz), 7.70 (d, 2H, J = 7.5 Hz), 7.57 (s, 1H), 5.79 (s, 1H), 4.15 (t, 2H, J = 6.9 Hz), 2.40 (t, 2H, J = 6.9 Hz), 1.78-1.73 (m, 2H), 1.45-1.37 (m, 2H). |

TABLE 79

| Compound | Structure | 1H-NMR |
|---|---|---|
| 386 | 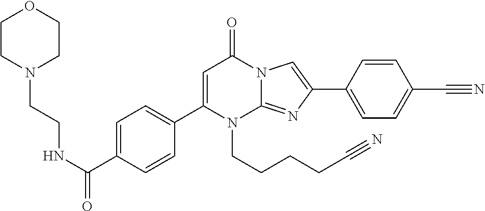 [SMILES: N#CCCCCn1c(-c2ccc(C(=O)NCCN3CCOCC3)cc2)cc(=O)n2cc(-c3ccc(C#N)cc3)nc12] | (δ ppm TMS/DMSO-d6) 8.60 (t, 1H, J = 5.4 Hz), 8.50 (s, 1H), 8.20 (d, 2H, J = 7.5 Hz), 8.01 (d, 2H, J = 7.5 Hz), 7.89 (d, 2H, J = 7.5 Hz), 7.70 (d, 2H, J = 7.5 Hz), 5.78 (s, 1H), 4.14 (t, 2H, J = 7.0 Hz), 3.58 (t, 4H, J = 4.3 Hz), 2.42-2.39 (m, 6H), 1.79-1.71 (m, 2H), 1.43-1.41 (m, 2H). |
| 387 | 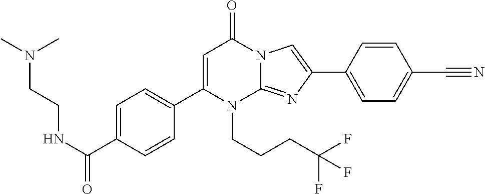 [SMILES: CN(C)CCNC(=O)c1ccc(-c2cc(=O)n3cc(-c4ccc(C#N)cc4)nc3n2CCCC(F)(F)F)cc1] | (δ ppm TMS/DMSO-d6) 8.60 (t, 1H, J = 5.2 Hz), 8.54 (br s, 1H), 8.20 (d, 2H, J = 8.3 Hz), 8.03 (d, 2H, J = 8.0 Hz), 7.92 (d, 2H, J = 8.2 Hz), 7.71 (d, 2H, J = 8.0 Hz), 5.79 (br s, 1H), 4.17 (t, 2H, J = 7.0 Hz), 3.41-3.40 (m, 2H), 2.42 (t, 2H, J = 6.7 Hz), 2.30-2.19 (m, 8H), 1.94-1.92 (m, 2H). |
| 388 | 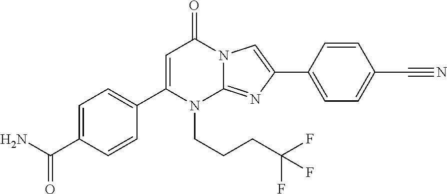 [SMILES: N#Cc1ccc(-c2cn3c(=O)cc(-c4ccc(C(N)=O)cc4)n(CCCC(F)(F)F)c3n2)cc1] | (δ ppm TMS/DMSO-d6) 8.53 (s, 1H), 8.21-8.19 (m, 3H), 8.06 (d, 2H, J = 7.7 Hz), 7.92 (d, 2H, J = 7.4 Hz), 7.70 (d, 2H, J = 7.5 Hz), 7.60 (s, 1H), 5.79 (s, 1H), 4.17 (t, 2H, J = 7.8 Hz), 2.29-2.16 (m, 2H), 1.94-1.90 (m, 2H). |
| 389 | 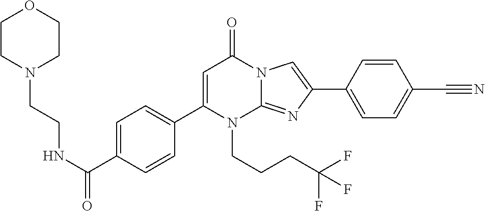 [SMILES: N#Cc1ccc(-c2cn3c(=O)cc(-c4ccc(C(=O)NCCN5CCOCC5)cc4)n(CCCC(F)(F)F)c3n2)cc1] | (δ ppm TMS/DMSO-d6) 8.62 (t, 1H, J = 5.8 Hz), 8.54 (s, 1H), 8.18 (d, 2H, J = 7.8 Hz), 8.03 (d, 2H, J = 7.8 Hz), 7.92 (d, 2H, J = 7.3 Hz), 7.71 (d, 2H, J = 7.7 Hz), 5.79 (s, 1H), 4.17 (t, 2H, J = 7.3 Hz), 3.58 (t, 4H, J = 4.3 Hz), 3.43 (q, 2H, J = 6.0 Hz), 2.45 (br s, 4H), 2.27-2.20 (m, 2H), 1.93- 1.91 (m, 2H). |
| 390 | 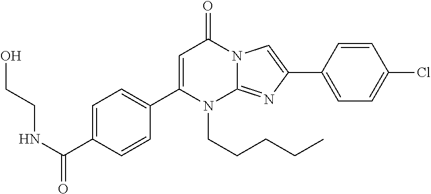 [SMILES: CCCCCn1c(-c2ccc(C(=O)NCCO)cc2)cc(=O)n2cc(-c3ccc(Cl)cc3)nc12] | (δ ppm TMS/DMSO-d6) 8.62 (t, 1H, J = 5.3 Hz), 8.29 (s, 1H), 8.01 (t, 4H, J = 7.7 Hz), 7.69 (d, 2H, J = 7.5 Hz), 7.50 (d, 2H, J = 7.8 Hz), 5.75 (s, 1H), 4.78 (t, 1H, J = 5.4 Hz), 4.11 (t, 2H, J = 7.5 Hz), 3.54 (q, 2H, J = 6.0 Hz), 1.64-1.61 (m, 2H), 1.13-1.09 (m, 5H), 0.73 (t, 3H, J = 6.8 Hz). |

TABLE 80

| | | | Retention | |
|---|---|---|---|---|
| | | | Time | Mass |
| Compound | Structure | LC/MS | [min] | [M + H] |
| 391 | 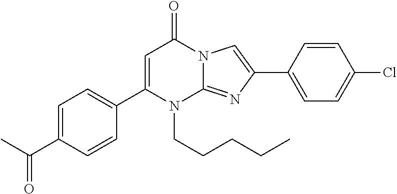 [SMILES: CCCCCn1c(-c2ccc(C(C)=O)cc2)cc(=O)n2cc(-c3ccc(Cl)cc3)nc12] | Method A | 3.00 | 434 |
| 392 | 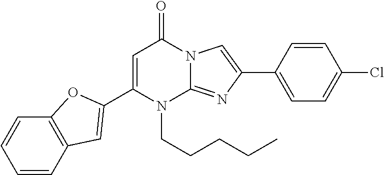 [SMILES: CCCCCn1c(-c2cc3ccccc3o2)cc(=O)n2cc(-c3ccc(Cl)cc3)nc12] | Method A | 3.42 | 432 |
| 393 | 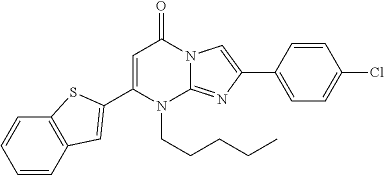 [SMILES: CCCCCn1c(-c2cc3ccccc3s2)cc(=O)n2cc(-c3ccc(Cl)cc3)nc12] | Method A | 3.48 | 448 |
| 394 | 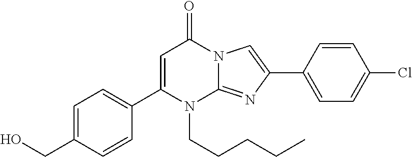 [SMILES: CCCCCn1c(-c2ccc(CO)cc2)cc(=O)n2cc(-c3ccc(Cl)cc3)nc12] | Method A | 2.76 | 422 |
| 395 | 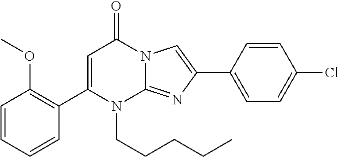 [SMILES: CCCCCn1c(-c2ccccc2OC)cc(=O)n2cc(-c3ccc(Cl)cc3)nc12] | Method A | 3.22 | 422 |

TABLE 81

| | | | Retention | |
|---|---|---|---|---|
| | | | Time | Mass |
| Compound | Structure | LC/MS | [min] | [M + H] |
| 396 | 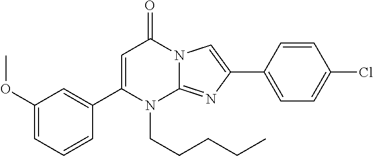 [SMILES: CCCCCn1c(-c2cccc(OC)c2)cc(=O)n2cc(-c3ccc(Cl)cc3)nc12] | Method A | 3.22 | 422 |
| 397 | 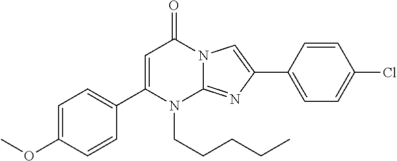 [SMILES: CCCCCn1c(-c2ccc(OC)cc2)cc(=O)n2cc(-c3ccc(Cl)cc3)nc12] | Method A | 3.20 | 422 |
| 398 | 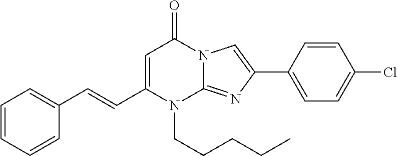 [SMILES: CCCCCn1c(/C=C/c2ccccc2)cc(=O)n2cc(-c3ccc(Cl)cc3)nc12] | Method A | 3.25 | 418 |
| 399 | 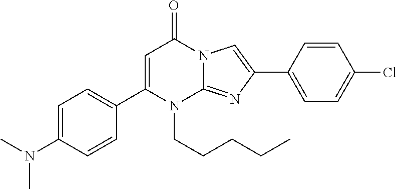 [SMILES: CCCCCn1c(-c2ccc(N(C)C)cc2)cc(=O)n2cc(-c3ccc(Cl)cc3)nc12] | Method A | 3.36 | 435 |
| 400 | 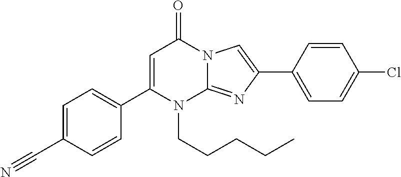 [SMILES: CCCCCn1c(-c2ccc(C#N)cc2)cc(=O)n2cc(-c3ccc(Cl)cc3)nc12] | Method A | 2.99 | 417 |

TABLE 82

| | | | Retention | |
|---|---|---|---|---|
| | | | Time | Mass |
| Compound | Structure | LC/MS | [min] | [M + H] |
| 401 | 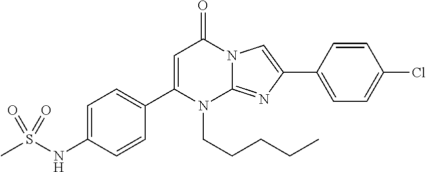 [SMILES: CCCCCn1c(-c2ccc(NS(C)(=O)=O)cc2)cc(=O)n2cc(-c3ccc(Cl)cc3)nc12] | Method A | 2.76 | 485 |
| 402 | 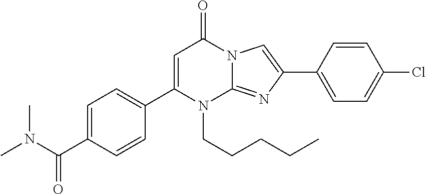 [SMILES: CCCCCn1c(-c2ccc(C(=O)N(C)C)cc2)cc(=O)n2cc(-c3ccc(Cl)cc3)nc12] | Method A | 2.73 | 463 |
| 403 | 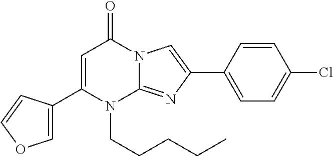 [SMILES: CCCCCn1c(-c2ccoc2)cc(=O)n2cc(-c3ccc(Cl)cc3)nc12] | Method A | 3.01 | 382 |
| 404 | 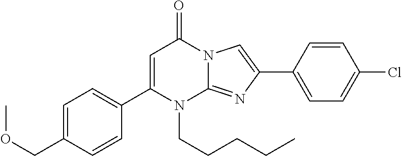 [SMILES: CCCCCn1c(-c2ccc(COC)cc2)cc(=O)n2cc(-c3ccc(Cl)cc3)nc12] | Method A | 3.18 | 436 |
| 405 | 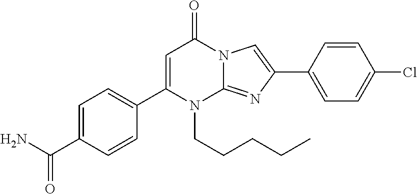 [SMILES: CCCCCn1c(-c2ccc(C(N)=O)cc2)cc(=O)n2cc(-c3ccc(Cl)cc3)nc12] | Method A | 2.52 | 435 |

TABLE 83

| | | | Retention | |
|---|---|---|---|---|
| | | | Time | Mass |
| Compound | Structure | LC/MS | [min] | [M + H] |
| 406 | 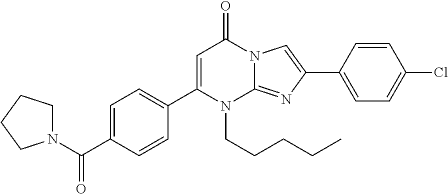 [SMILES: CCCCCn1c(-c2ccc(C(=O)N3CCCC3)cc2)cc(=O)n2cc(-c3ccc(Cl)cc3)nc12] | Method A | 2.84 | 489 |
| 407 | 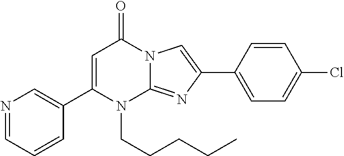 [SMILES: CCCCCn1c(-c2cccnc2)cc(=O)n2cc(-c3ccc(Cl)cc3)nc12] | Method A | 2.67 | 393 |
| 408 | 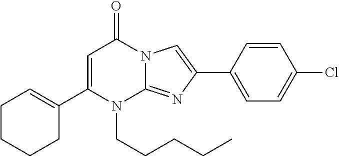 [SMILES: CCCCCn1c(C2=CCCCC2)cc(=O)n2cc(-c3ccc(Cl)cc3)nc12] | Method A | 3.42 | 396 |
| 409 | 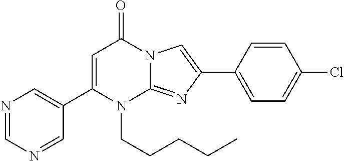 [SMILES: CCCCCn1c(-c2cncnc2)cc(=O)n2cc(-c3ccc(Cl)cc3)nc12] | Method A | 2.58 | 394 |
| 410 | 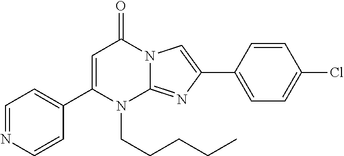 [SMILES: CCCCCn1c(-c2ccncc2)cc(=O)n2cc(-c3ccc(Cl)cc3)nc12] | Method A | 2.64 | 393 |

TABLE 84

| | | | Retention | |
|---|---|---|---|---|
| | | | Time | Mass |
| Compound | Structure | LC/MS | [min] | [M + H] |
| 411 | 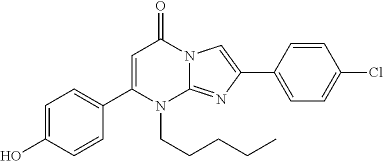 [SMILES: CCCCCn1c(-c2ccc(O)cc2)cc(=O)n2cc(-c3ccc(Cl)cc3)nc12] | Method A | 2.82 | 408 |
| 412 | 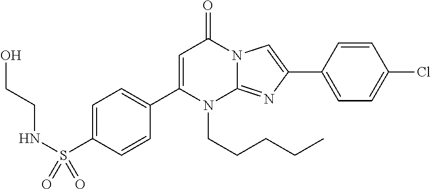 [SMILES: CCCCCn1c(-c2ccc(S(=O)(=O)NCCO)cc2)cc(=O)n2cc(-c3ccc(Cl)cc3)nc12] | Method A | 2.56 | 515 |
| 413 | 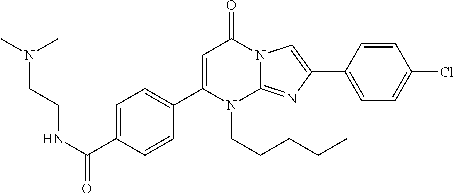 [SMILES: CCCCCn1c(-c2ccc(C(=O)NCCN(C)C)cc2)cc(=O)n2cc(-c3ccc(Cl)cc3)nc12] | Method A | 2.12 | 506 |
| 414 | 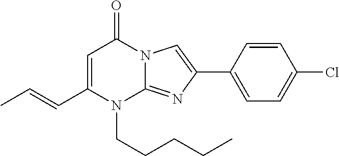 [SMILES: C/C=C/c1cc(=O)n2cc(-c3ccc(Cl)cc3)nc2n1CCCCC] | Method A | 3.05 | 356 |
| 415 | 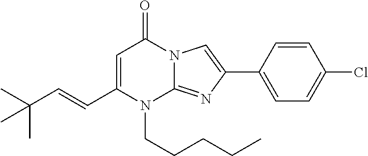 [SMILES: CCCCCn1c(/C=C/C(C)(C)C)cc(=O)n2cc(-c3ccc(Cl)cc3)nc12] | Method A | 3.46 | 398 |

TABLE 85

| | | | Retention | |
|---|---|---|---|---|
| | | | Time | Mass |
| Compound | Structure | LC/MS | [min] | [M + H] |
| 416 | 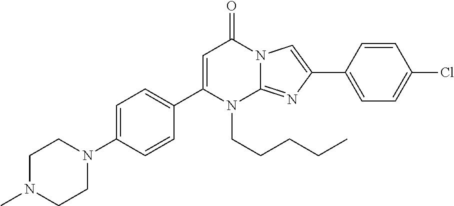 [SMILES: CCCCCn1c(-c2ccc(N3CCN(C)CC3)cc2)cc(=O)n2cc(-c3ccc(Cl)cc3)nc12] | Method A | 2.22 | 490 |
| 417 | 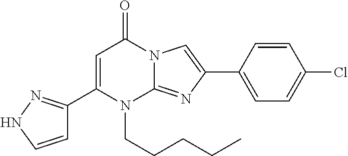 [SMILES: CCCCCn1c(-c2cc[nH]n2)cc(=O)n2cc(-c3ccc(Cl)cc3)nc12] | Method A | 2.66 | 382 |
| 418 | 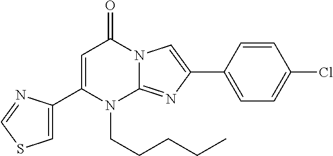 [SMILES: CCCCCn1c(-c2cscn2)cc(=O)n2cc(-c3ccc(Cl)cc3)nc12] | Method A | 2.93 | 399 |
| 419 | 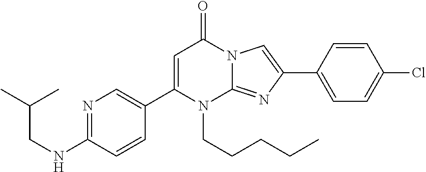 [SMILES: CCCCCn1c(-c2ccc(NCC(C)C)nc2)cc(=O)n2cc(-c3ccc(Cl)cc3)nc12] | Method A | 2.69 | 464 |
| 420 | 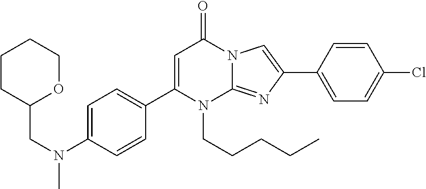 [SMILES: CCCCCn1c(-c2ccc(N(C)CC3CCCCO3)cc2)cc(=O)n2cc(-c3ccc(Cl)cc3)nc12] | Method A | 3.59 | 519 |

TABLE 86

| | | | Retention | |
|---|---|---|---|---|
| | | | Time | Mass |
| Compound | Structure | LC/MS | [min] | [M + H] |
| 421 | 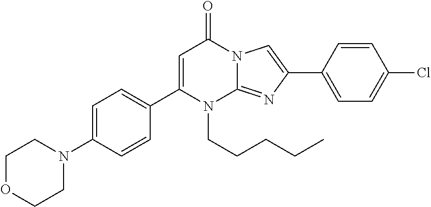 [SMILES: CCCCCn1c(-c2ccc(N3CCOCC3)cc2)cc(=O)n2cc(-c3ccc(Cl)cc3)nc12] | Method A | 3.14 | 477 |
| 422 | 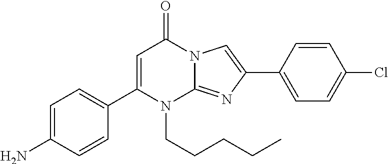 [SMILES: CCCCCn1c(-c2ccc(N)cc2)cc(=O)n2cc(-c3ccc(Cl)cc3)nc12] | Method A | 2.84 | 407 |
| 423 | 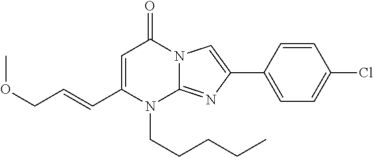 [SMILES: CCCCCn1c(/C=C/COC)cc(=O)n2cc(-c3ccc(Cl)cc3)nc12] | Method A | 2.93 | 386 |
| 424 | 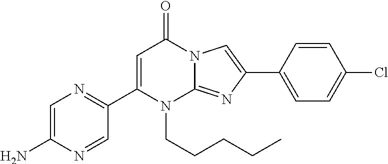 [SMILES: CCCCCn1c(-c2cnc(N)cn2)cc(=O)n2cc(-c3ccc(Cl)cc3)nc12] | Method A | 2.59 | 409 |
| 425 | 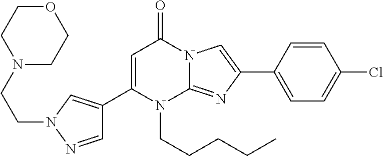 [SMILES: CCCCCn1c(-c2cnn(CCN3CCOCC3)c2)cc(=O)n2cc(-c3ccc(Cl)cc3)nc12] | Method A | 2.03 | 495 |

TABLE 87

| | | | Retention | |
|---|---|---|---|---|
| | | | Time | Mass |
| Compound | Structure | LC/MS | [min] | [M + H] |
| 426 | 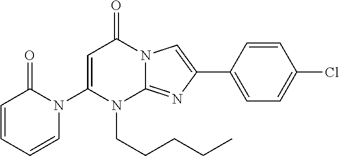 [SMILES: CCCCCn1c(-n2ccccc2=O)cc(=O)n2cc(-c3ccc(Cl)cc3)nc12] | Method A | 2.53 | 409 |
| 427 | 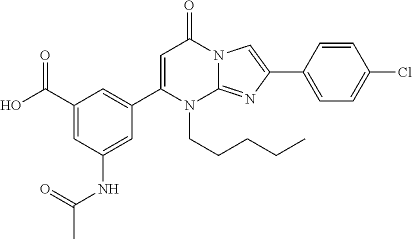 [SMILES: CCCCCn1c(-c2cc(NC(C)=O)cc(C(=O)O)c2)cc(=O)n2cc(-c3ccc(Cl)cc3)nc12] | Method A | 2.47 | 493 |
| 428 | 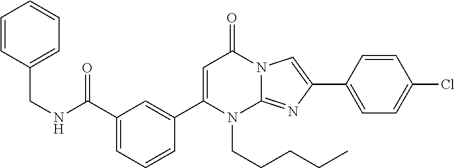 [SMILES: CCCCCn1c(-c2cccc(C(=O)NCc3ccccc3)c2)cc(=O)n2cc(-c3ccc(Cl)cc3)nc12] | Method A | 3.06 | 525 |
| 429 | 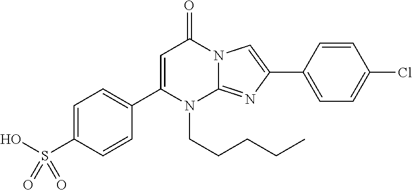 [SMILES: CCCCCn1c(-c2ccc(S(=O)(=O)O)cc2)cc(=O)n2cc(-c3ccc(Cl)cc3)nc12] | Method A | 2.18 | 472 |
| 430 | 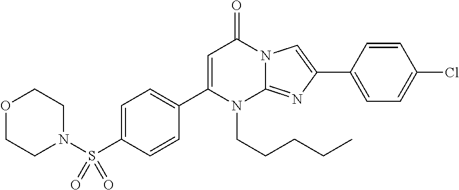 [SMILES: CCCCCn1c(-c2ccc(S(=O)(=O)N3CCOCC3)cc2)cc(=O)n2cc(-c3ccc(Cl)cc3)nc12] | Method A | 2.93 | 541 |

TABLE 88

| | | | Retention | |
|---|---|---|---|---|
| | | | Time | Mass |
| Compound | Structure | LC/MS | [min] | [M + H] |
| 431 | 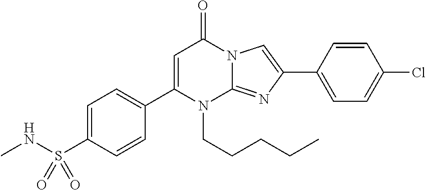 [SMILES: CCCCCn1c(-c2ccc(S(=O)(=O)NC)cc2)cc(=O)n2cc(-c3ccc(Cl)cc3)nc12] | Method A | 2.80 | 485 |
| 432 | 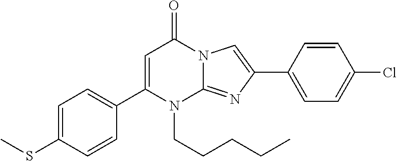 [SMILES: CCCCCn1c(-c2ccc(SC)cc2)cc(=O)n2cc(-c3ccc(Cl)cc3)nc12] | Method A | 3.35 | 438 |
| 433 | 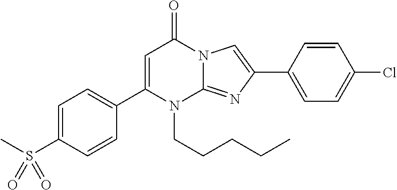 [SMILES: CCCCCn1c(-c2ccc(S(C)(=O)=O)cc2)cc(=O)n2cc(-c3ccc(Cl)cc3)nc12] | Method A | 2.79 | 470 |
| 434 | 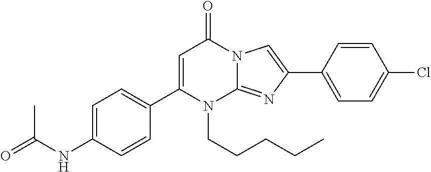 [SMILES: CCCCCn1c(-c2ccc(NC(C)=O)cc2)cc(=O)n2cc(-c3ccc(Cl)cc3)nc12] | Method A | 2.76 | 449 |
| 435 | 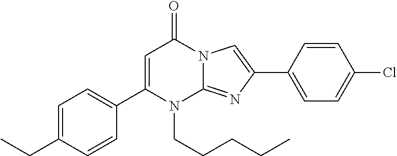 [SMILES: CCCCCn1c(-c2ccc(CC)cc2)cc(=O)n2cc(-c3ccc(Cl)cc3)nc12] | Method A | 3.52 | 420 |

TABLE 89

| | | | Retention | |
|---|---|---|---|---|
| | | | Time | |
| Compound | Structure | LC/MS | [min] | Mass [M + H] |
| 436 | 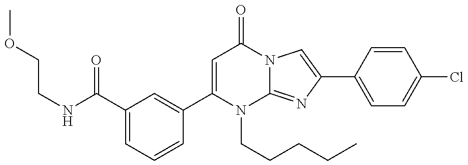 [SMILES: CCCCCn1c(-c2cccc(C(=O)NCCOC)c2)cc(=O)n2cc(-c3ccc(Cl)cc3)nc12] | Method A | 2.73 | 493 |
| 437 | 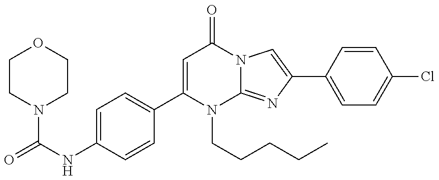 [SMILES: CCCCCn1c(-c2ccc(NC(=O)N3CCOCC3)cc2)cc(=O)n2cc(-c3ccc(Cl)cc3)nc12] | Method A | 2.77 | 520 |
| 438 | 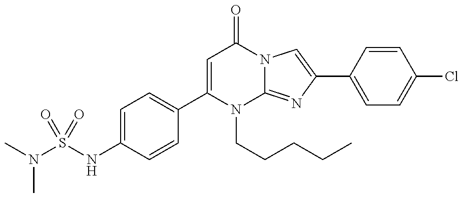 [SMILES: CCCCCn1c(-c2ccc(NS(=O)(=O)N(C)C)cc2)cc(=O)n2cc(-c3ccc(Cl)cc3)nc12] | Method A | 2.94 | 514 |
| 439 | 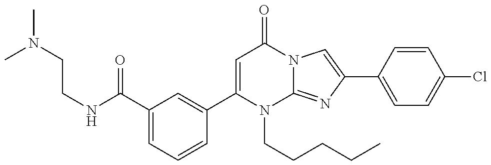 [SMILES: CCCCCn1c(-c2cccc(C(=O)NCCN(C)C)c2)cc(=O)n2cc(-c3ccc(Cl)cc3)nc12] | Method A | 2.12 | 506 |
| 440 | 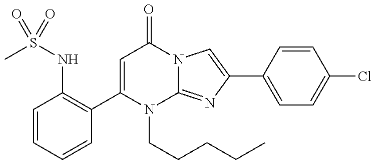 [SMILES: CCCCCn1c(-c2ccccc2NS(C)(=O)=O)cc(=O)n2cc(-c3ccc(Cl)cc3)nc12] | Method A | 2.86 | 485 |

TABLE 90

| | | | Retention | |
|---|---|---|---|---|
| | | | Time | |
| Compound | Structure | LC/MS | [min] | Mass [M + H] |
| 441 | 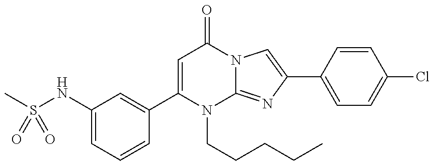 [SMILES: CCCCCn1c(-c2cccc(NS(C)(=O)=O)c2)cc(=O)n2cc(-c3ccc(Cl)cc3)nc12] | Method A | 2.80 | 485 |
| 442 | 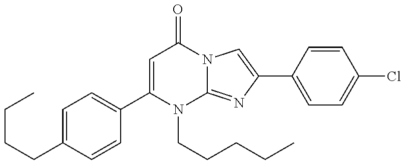 [SMILES: CCCCCn1c(-c2ccc(CCCC)cc2)cc(=O)n2cc(-c3ccc(Cl)cc3)nc12] | Method A | 3.77 | 448 |
| 443 | 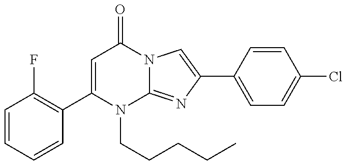 [SMILES: CCCCCn1c(-c2ccccc2F)cc(=O)n2cc(-c3ccc(Cl)cc3)nc12] | Method A | 3.25 | 410 |
| 444 | 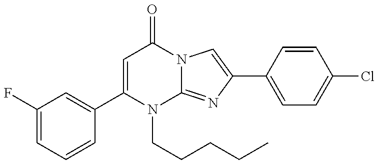 [SMILES: CCCCCn1c(-c2cccc(F)c2)cc(=O)n2cc(-c3ccc(Cl)cc3)nc12] | Method A | 3.23 | 410 |
| 445 | 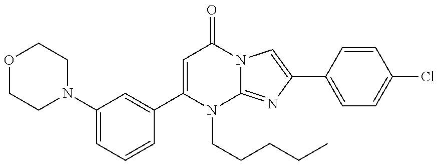 [SMILES: CCCCCn1c(-c2cccc(N3CCOCC3)c2)cc(=O)n2cc(-c3ccc(Cl)cc3)nc12] | Method A | 3.20 | 477 |

TABLE 91

| | | | Retention | |
|---|---|---|---|---|
| | | | Time | |
| Compound | Structure | LC/MS | [min] | Mass [M + H] |
| 446 | 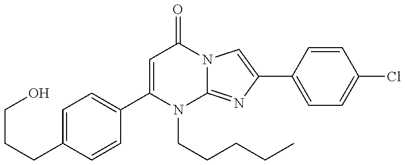 [SMILES: CCCCCn1c(-c2ccc(CCCO)cc2)cc(=O)n2cc(-c3ccc(Cl)cc3)nc12] | Method A | 2.93 | 450 |
| 447 | 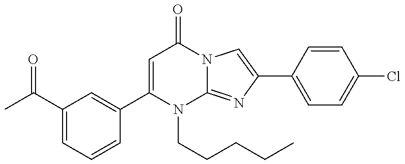 [SMILES: CCCCCn1c(-c2cccc(C(C)=O)c2)cc(=O)n2cc(-c3ccc(Cl)cc3)nc12] | Method A | 3.03 | 434 |
| 448 | 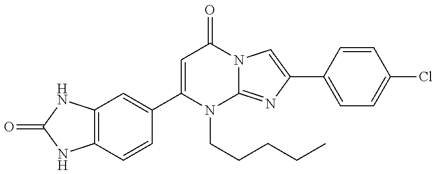 [SMILES: CCCCCn1c(-c2ccc3[nH]c(=O)[nH]c3c2)cc(=O)n2cc(-c3ccc(Cl)cc3)nc12] | Method A | 2.45 | 448 |
| 449 | 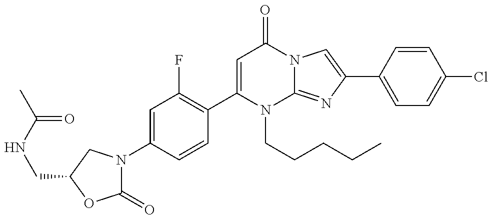 [SMILES: CCCCCn1c(-c2ccc(N3C[C@@H](CNC(C)=O)OC3=O)cc2F)cc(=O)n2cc(-c3ccc(Cl)cc3)nc12] | Method A | 2.69 | 566 |
| 450 | 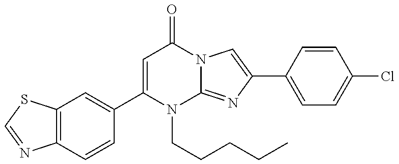 [SMILES: CCCCCn1c(-c2ccc3ncsc3c2)cc(=O)n2cc(-c3ccc(Cl)cc3)nc12] | Method A | 3.02 | 449 |

TABLE 92

| | | | Retention | |
|---|---|---|---|---|
| | | | Time | |
| Compound | Structure | LC/MS | [min] | Mass [M + H] |
| 451 | 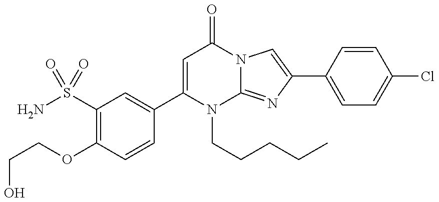 [SMILES: CCCCCn1c(-c2ccc(OCCO)c(S(N)(=O)=O)c2)cc(=O)n2cc(-c3ccc(Cl)cc3)nc12] | Method A | 2.50 | 531 |
| 452 | 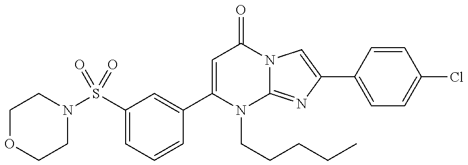 [SMILES: CCCCCn1c(-c2cccc(S(=O)(=O)N3CCOCC3)c2)cc(=O)n2cc(-c3ccc(Cl)cc3)nc12] | Method A | 2.94 | 541 |
| 453 | 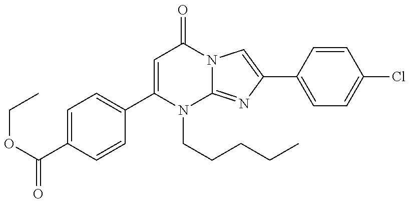 [SMILES: CCCCCn1c(-c2ccc(C(=O)OCC)cc2)cc(=O)n2cc(-c3ccc(Cl)cc3)nc12] | Method C | 2.97 | 464 |
| 454 | 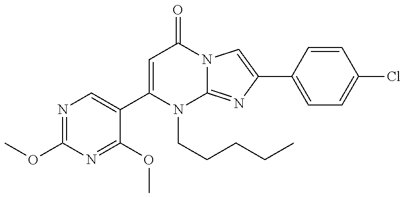 [SMILES: CCCCCn1c(-c2cnc(OC)nc2OC)cc(=O)n2cc(-c3ccc(Cl)cc3)nc12] | Method A | 2.94 | 454 |
| 455 | 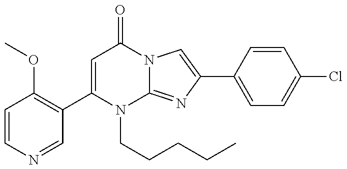 [SMILES: CCCCCn1c(-c2cnccc2OC)cc(=O)n2cc(-c3ccc(Cl)cc3)nc12] | Method A | 2.39 | 423 |

TABLE 93

| | | | Retention | |
|---|---|---|---|---|
| | | | Time | |
| Compound | Structure | LC/MS | [min] | Mass [M + H] |
| 456 | 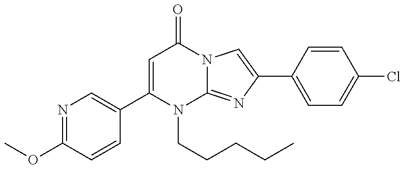 [SMILES: CCCCCn1c(-c2ccc(OC)nc2)cc(=O)n2cc(-c3ccc(Cl)cc3)nc12] | Method A | 3.05 | 423 |
| 457 | 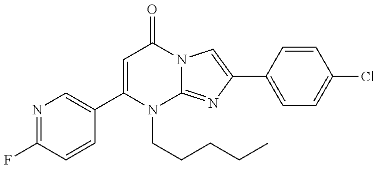 [SMILES: CCCCCn1c(-c2ccc(F)nc2)cc(=O)n2cc(-c3ccc(Cl)cc3)nc12] | Method A | 2.89 | 411 |
| 458 | 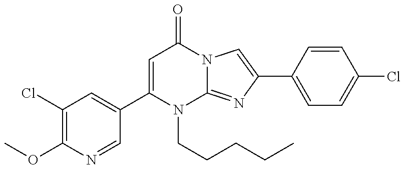 [SMILES: CCCCCn1c(-c2cnc(OC)c(Cl)c2)cc(=O)n2cc(-c3ccc(Cl)cc3)nc12] | Method A | 3.22 | 457 |
| 459 | 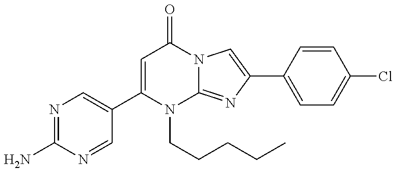 [SMILES: CCCCCn1c(-c2cnc(N)nc2)cc(=O)n2cc(-c3ccc(Cl)cc3)nc12] | Method A | 2.43 | 409 |
| 460 | 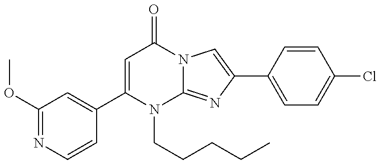 [SMILES: CCCCCn1c(-c2ccnc(OC)c2)cc(=O)n2cc(-c3ccc(Cl)cc3)nc12] | Method A | 3.05 | 423 |

TABLE 94

| | | | Retention | |
|---|---|---|---|---|
| Com- | | | Time | Mass |
| pound | Structure | LC/MS | [min] | [M + H] |
| 461 | 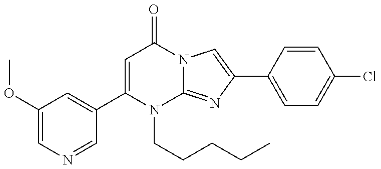 [SMILES: CCCCCn1c(-c2cncc(OC)c2)cc(=O)n2cc(-c3ccc(Cl)cc3)nc12] | Method A | 2.78 | 423 |
| 462 | 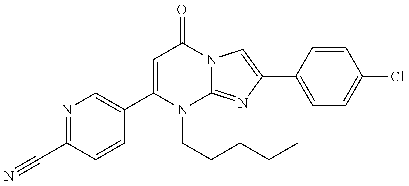 [SMILES: CCCCCn1c(-c2ccc(C#N)nc2)cc(=O)n2cc(-c3ccc(Cl)cc3)nc12] | Method A | 2.83 | 418 |
| 463 | 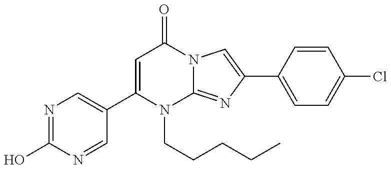 [SMILES: CCCCCn1c(-c2cnc(O)nc2)cc(=O)n2cc(-c3ccc(Cl)cc3)nc12] | Method A | 2.14 | 410 |
| 464 | 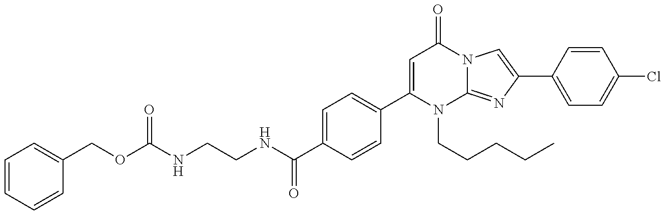 [SMILES: CCCCCn1c(-c2ccc(C(=O)NCCNC(=O)OCc3ccccc3)cc2)cc(=O)n2cc(-c3ccc(Cl)cc3)nc12] | Method C | 2.74 | 612 |

TABLE 95

| | | | Retention | |
|---|---|---|---|---|
| | | | Time | |
| Compound | Structure | LC/MS | [min] | Mass [M + H] |
| 465 | 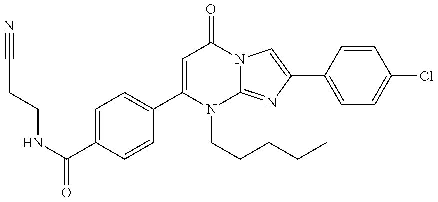 [SMILES: CCCCCn1c(-c2ccc(C(=O)NCCC#N)cc2)cc(=O)n2cc(-c3ccc(Cl)cc3)nc12] | Method A | 2.65 | 488 |
| 466 | 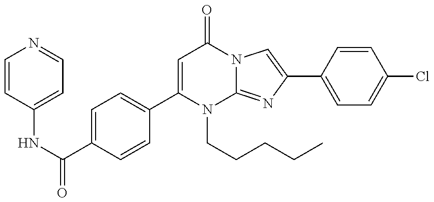 [SMILES: CCCCCn1c(-c2ccc(C(=O)Nc3ccncc3)cc2)cc(=O)n2cc(-c3ccc(Cl)cc3)nc12] | Method A | 2.26 | 512 |
| 467 | 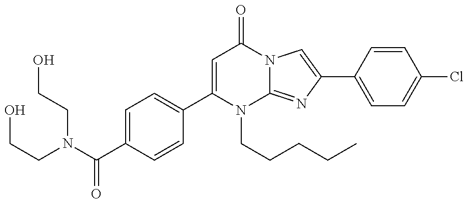 [SMILES: CCCCCn1c(-c2ccc(C(=O)N(CCO)CCO)cc2)cc(=O)n2cc(-c3ccc(Cl)cc3)nc12] | Method A | 2.29 | 523 |
| 468 | 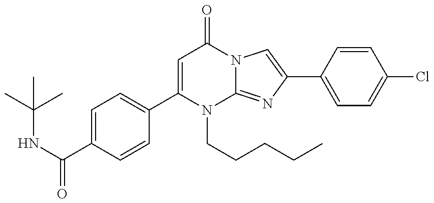 [SMILES: CCCCCn1c(-c2ccc(C(=O)NC(C)(C)C)cc2)cc(=O)n2cc(-c3ccc(Cl)cc3)nc12] | Method A | 3.07 | 491 |
| 469 | 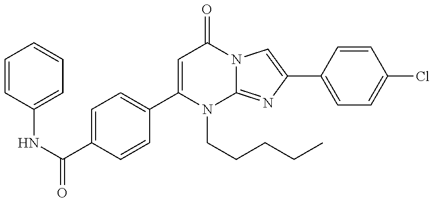 [SMILES: CCCCCn1c(-c2ccc(C(=O)Nc3ccccc3)cc2)cc(=O)n2cc(-c3ccc(Cl)cc3)nc12] | Method A | 3.08 | 511 |

TABLE 96

| | | | Retention | |
|---|---|---|---|---|
| | | | Time | |
| Compound | Structure | LC/MS | [min] | Mass [M + H] |
| 470 | 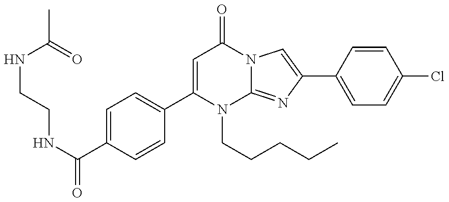 [SMILES: CCCCCn1c(-c2ccc(C(=O)NCCNC(C)=O)cc2)cc(=O)n2cc(-c3ccc(Cl)cc3)nc12] | Method A | 2.4 | 520 |
| 471 | 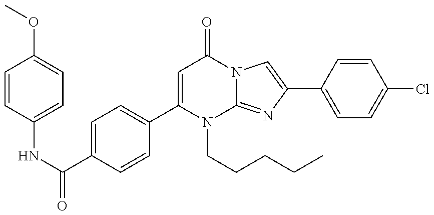 [SMILES: CCCCCn1c(-c2ccc(C(=O)Nc3ccc(OC)cc3)cc2)cc(=O)n2cc(-c3ccc(Cl)cc3)nc12] | Method A | 3.03 | 541 |
| 472 | 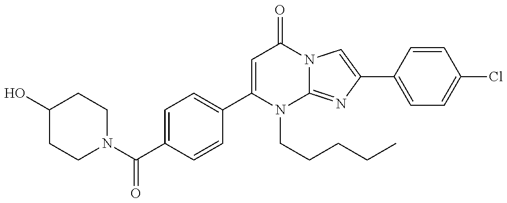 [SMILES: CCCCCn1c(-c2ccc(C(=O)N3CCC(O)CC3)cc2)cc(=O)n2cc(-c3ccc(Cl)cc3)nc12] | Method A | 2.47 | 519 |
| 473 | 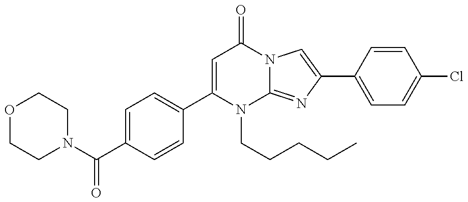 [SMILES: CCCCCn1c(-c2ccc(C(=O)N3CCOCC3)cc2)cc(=O)n2cc(-c3ccc(Cl)cc3)nc12] | Method A | 2.69 | 505 |

TABLE 97

| | | | Retention | |
|---|---|---|---|---|
| | | | Time | |
| Compound | Structure | LC/MS | [min] | Mass [M + H] |
| 474 | 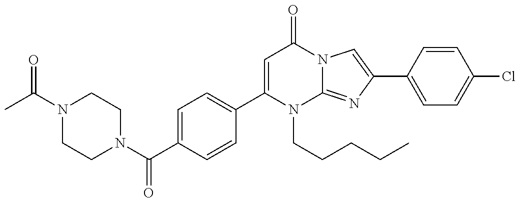 [SMILES: CCCCCn1c(-c2ccc(C(=O)N3CCN(C(C)=O)CC3)cc2)cc(=O)n2cc(-c3ccc(Cl)cc3)nc12] | Method A | 2.47 | 546 |
| 475 | 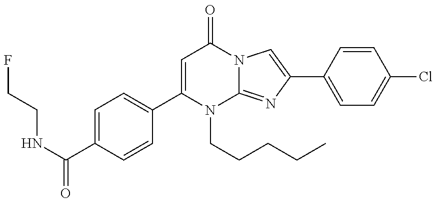 [SMILES: CCCCCn1c(-c2ccc(C(=O)NCCF)cc2)cc(=O)n2cc(-c3ccc(Cl)cc3)nc12] | Method A | 2.72 | 481 |
| 476 | 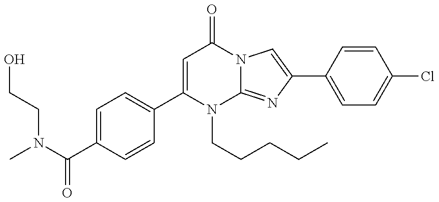 [SMILES: CCCCCn1c(-c2ccc(C(=O)N(C)CCO)cc2)cc(=O)n2cc(-c3ccc(Cl)cc3)nc12] | Method A | 2.46 | 493 |
| 477 | 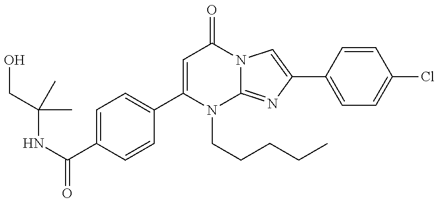 [SMILES: CCCCCn1c(-c2ccc(C(=O)NC(C)(C)CO)cc2)cc(=O)n2cc(-c3ccc(Cl)cc3)nc12] | Method A | 2.71 | 507 |

TABLE 98

| | | | Retention | |
|---|---|---|---|---|
| | | | Time | |
| Compound | Structure | LC/MS | [min] | Mass [M + H] |
| 478 | 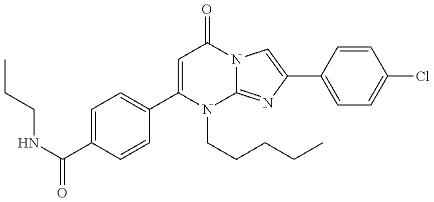 [SMILES: CCCCCn1c(-c2ccc(C(=O)NCCC)cc2)cc(=O)n2cc(-c3ccc(Cl)cc3)nc12] | Method A | 2.87 | 477 |
| 479 | 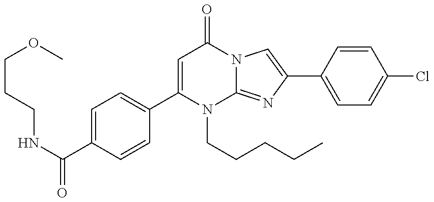 [SMILES: CCCCCn1c(-c2ccc(C(=O)NCCCOC)cc2)cc(=O)n2cc(-c3ccc(Cl)cc3)nc12] | Method A | 2.74 | 507 |
| 480 | 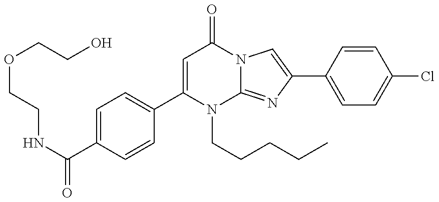 [SMILES: CCCCCn1c(-c2ccc(C(=O)NCCOCCO)cc2)cc(=O)n2cc(-c3ccc(Cl)cc3)nc12] | Method A | 2.43 | 523 |
| 481 | 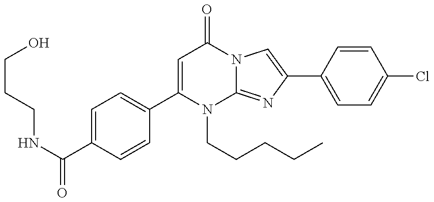 [SMILES: CCCCCn1c(-c2ccc(C(=O)NCCCO)cc2)cc(=O)n2cc(-c3ccc(Cl)cc3)nc12] | Method A | 2.47 | 493 |
| 482 | 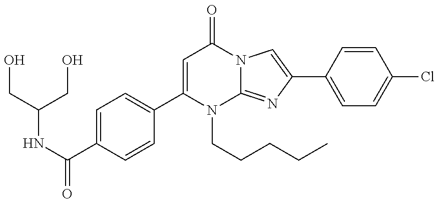 [SMILES: CCCCCn1c(-c2ccc(C(=O)NC(CO)CO)cc2)cc(=O)n2cc(-c3ccc(Cl)cc3)nc12] | Method A | 2.29 | 509 |

TABLE 99

| | | | Retention | |
|---|---|---|---|---|
| | | | Time | |
| Compound | Structure | LC/MS | [min] | Mass [M + H] |
| 483 | 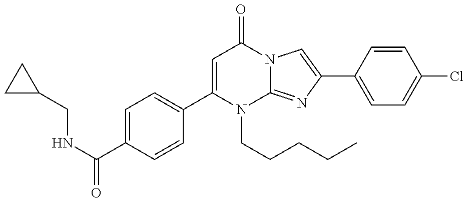 [SMILES: CCCCCn1c(-c2ccc(C(=O)NCC3CC3)cc2)cc(=O)n2cc(-c3ccc(Cl)cc3)nc12] | Method A | 2.9 | 489 |
| 484 | 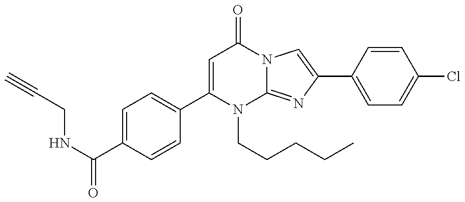 [SMILES: C#CCNC(=O)c1ccc(-c2cc(=O)n3cc(-c4ccc(Cl)cc4)nc3n2CCCCC)cc1] | Method A | 2.75 | 473 |
| 485 | 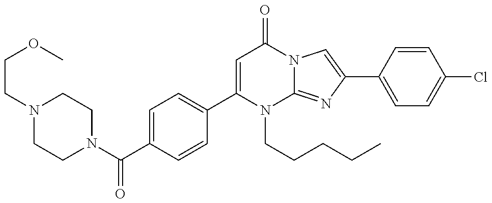 [SMILES: CCCCCn1c(-c2ccc(C(=O)N3CCN(CCOC)CC3)cc2)cc(=O)n2cc(-c3ccc(Cl)cc3)nc12] | Method A | 2.1 | 562 |
| 486 | 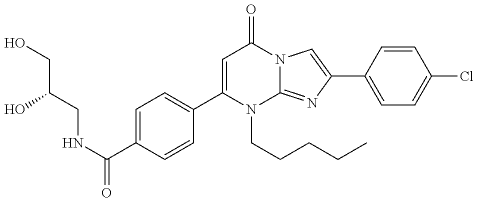 [SMILES: CCCCCn1c(-c2ccc(C(=O)NC[C@H](O)CO)cc2)cc(=O)n2cc(-c3ccc(Cl)cc3)nc12] | Method A | 2.31 | 509 |
| 487 | 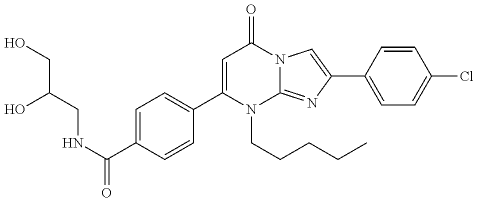 [SMILES: CCCCCn1c(-c2ccc(C(=O)NCC(O)CO)cc2)cc(=O)n2cc(-c3ccc(Cl)cc3)nc12] | Method A | 2.31 | 509 |

TABLE 100

| | | | Retention | |
|---|---|---|---|---|
| | | | Time | |
| Compound | Structure | LC/MS | [min] | Mass [M + H] |
| 488 | 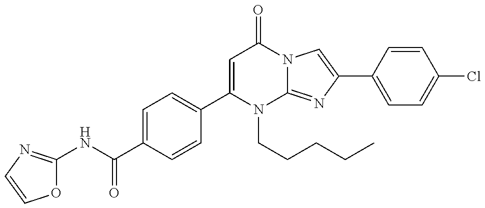 [SMILES: CCCCCn1c(-c2ccc(C(=O)Nc3ncco3)cc2)cc(=O)n2cc(-c3ccc(Cl)cc3)nc12] | Method A | 2.67 | 502 |
| 489 | 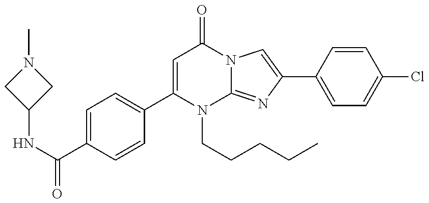 [SMILES: CCCCCn1c(-c2ccc(C(=O)NC3CN(C)C3)cc2)cc(=O)n2cc(-c3ccc(Cl)cc3)nc12] | Method A | 2.1 | 504 |
| 490 | 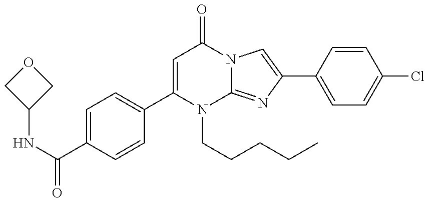 [SMILES: CCCCCn1c(-c2ccc(C(=O)NC3COC3)cc2)cc(=O)n2cc(-c3ccc(Cl)cc3)nc12] | Method A | 2.59 | 491 |
| 491 | 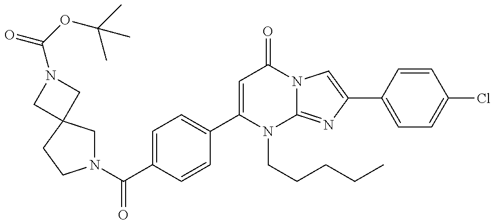 [SMILES: CCCCCn1c(-c2ccc(C(=O)N3CCC4(CN(C(=O)OC(C)(C)C)C4)C3)cc2)cc(=O)n2cc(-c3ccc(Cl)cc3)nc12] | Method A | 2.97 | 630 |

TABLE 101

| | | | Retention | |
|---|---|---|---|---|
| | | | Time | |
| Compound | Structure | LC/MS | [min] | Mass [M + H] |
| 492 | 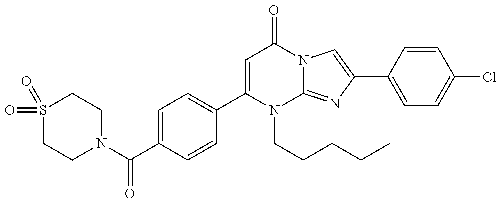 [SMILES: CCCCCn1c(-c2ccc(C(=O)N3CCS(=O)(=O)CC3)cc2)cc(=O)n2cc(-c3ccc(Cl)cc3)nc12] | Method A | 2.6 | 553 |
| 493 | 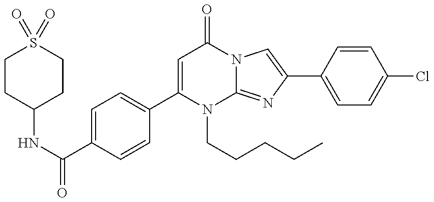 [SMILES: CCCCCn1c(-c2ccc(C(=O)NC3CCS(=O)(=O)CC3)cc2)cc(=O)n2cc(-c3ccc(Cl)cc3)nc12] | Method A | 2.59 | 567 |
| 494 | 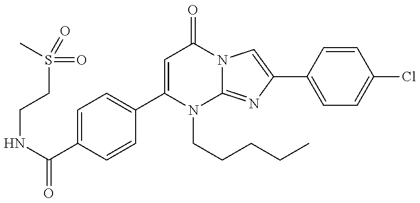 [SMILES: CCCCCn1c(-c2ccc(C(=O)NCCS(C)(=O)=O)cc2)cc(=O)n2cc(-c3ccc(Cl)cc3)nc12] | Method A | 2.54 | 541 |
| 495 | 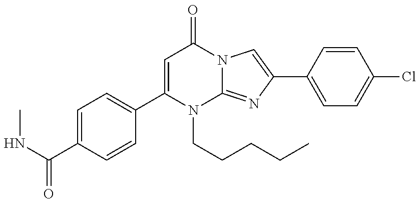 [SMILES: CCCCCn1c(-c2ccc(C(=O)NC)cc2)cc(=O)n2cc(-c3ccc(Cl)cc3)nc12] | Method A | 2.61 | 449 |

TABLE 102

| | | | Retention | |
|---|---|---|---|---|
| | | | Time | |
| Compound | Structure | LC/MS | [min] | Mass [M + H] |
| 496 | 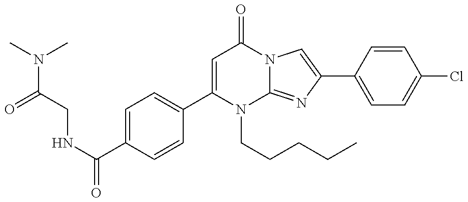 [SMILES: CCCCCn1c(-c2ccc(C(=O)NCC(=O)N(C)C)cc2)cc(=O)n2cc(-c3ccc(Cl)cc3)nc12] | Method A | 2.54 | 520 |
| 497 | 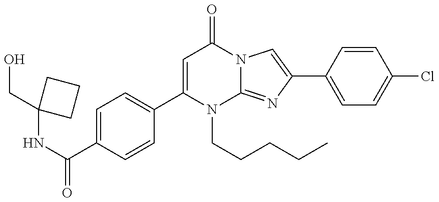 [SMILES: CCCCCn1c(-c2ccc(C(=O)NC3(CO)CCC3)cc2)cc(=O)n2cc(-c3ccc(Cl)cc3)nc12] | Method A | 2.72 | 519 |
| 498 | 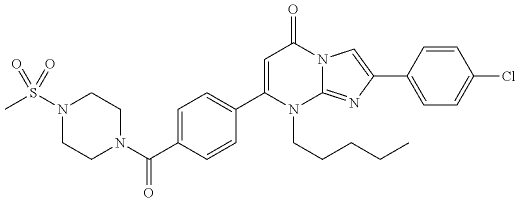 [SMILES: CCCCCn1c(-c2ccc(C(=O)N3CCN(S(C)(=O)=O)CC3)cc2)cc(=O)n2cc(-c3ccc(Cl)cc3)nc12] | Method A | 2.65 | 582 |
| 499 | 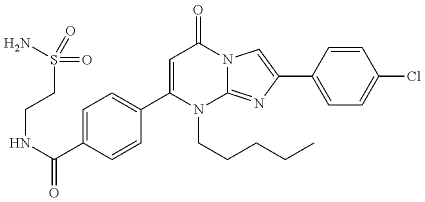 [SMILES: CCCCCn1c(-c2ccc(C(=O)NCCS(N)(=O)=O)cc2)cc(=O)n2cc(-c3ccc(Cl)cc3)nc12] | Method A | 2.46 | 542 |

TABLE 103

| | | | Retention | |
|---|---|---|---|---|
| | | | Time | |
| Compound | Structure | LC/MS | [min] | Mass [M + H] |
| 500 | 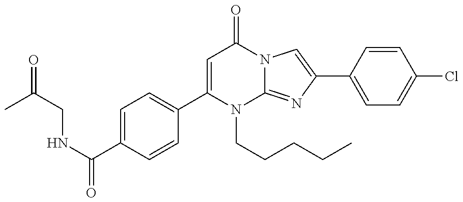 [SMILES: CCCCCn1c(-c2ccc(C(=O)NCC(C)=O)cc2)cc(=O)n2cc(-c3ccc(Cl)cc3)nc12] | Method A | 2.62 | 491 |
| 501 | 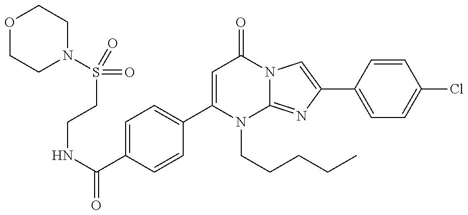 [SMILES: CCCCCn1c(-c2ccc(C(=O)NCCS(=O)(=O)N3CCOCC3)cc2)cc(=O)n2cc(-c3ccc(Cl)cc3)nc12] | Method A | 2.66 | 612 |
| 502 | 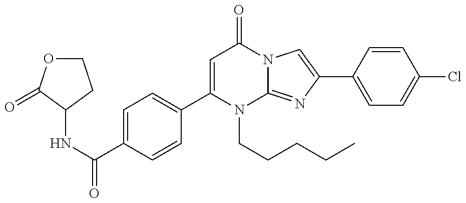 [SMILES: CCCCCn1c(-c2ccc(C(=O)NC3CCOC3=O)cc2)cc(=O)n2cc(-c3ccc(Cl)cc3)nc12] | Method A | 2.63 | 519 |
| 503 | 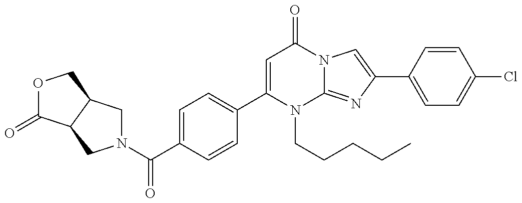 [SMILES: CCCCCn1c(-c2ccc(C(=O)N3C[C@@H]4COC(=O)[C@@H]4C3)cc2)cc(=O)n2cc(-c3ccc(Cl)cc3)nc12] | Method A | 2.58 | 545 |

TABLE 104

| | | | Retention | |
|---|---|---|---|---|
| | | | Time | |
| Compound | Structure | LC/MS | [min] | Mass [M + H] |
| 504 | 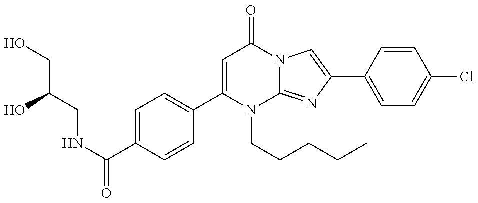 [SMILES: CCCCCn1c(-c2ccc(C(=O)NC[C@@H](O)CO)cc2)cc(=O)n2cc(-c3ccc(Cl)cc3)nc12] | Method A | 2.31 | 509 |
| 505 | 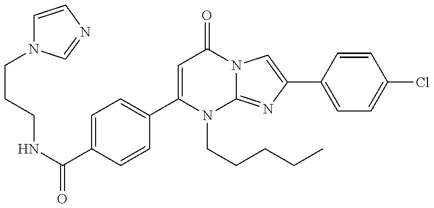 [SMILES: CCCCCn1c(-c2ccc(C(=O)NCCCn3ccnc3)cc2)cc(=O)n2cc(-c3ccc(Cl)cc3)nc12] | Method A | 2.09 | 543 |
| 506 | 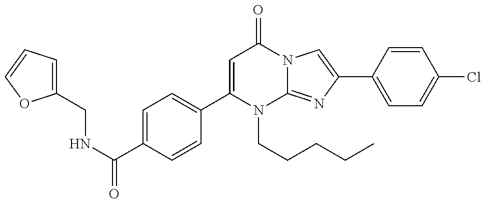 [SMILES: CCCCCn1c(-c2ccc(C(=O)NCc3ccco3)cc2)cc(=O)n2cc(-c3ccc(Cl)cc3)nc12] | Method A | 2.88 | 515 |
| 507 | 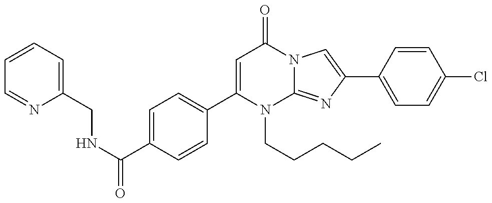 [SMILES: CCCCCn1c(-c2ccc(C(=O)NCc3ccccn3)cc2)cc(=O)n2cc(-c3ccc(Cl)cc3)nc12] | Method A | 2.43 | 526 |
| 508 | 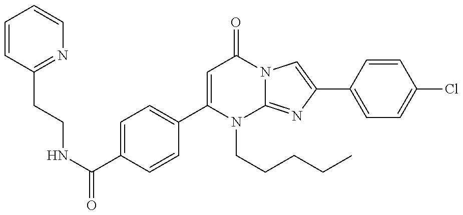 [SMILES: CCCCCn1c(-c2ccc(C(=O)NCCc3ccccn3)cc2)cc(=O)n2cc(-c3ccc(Cl)cc3)nc12] | Method A | 2.23 | 540 |

TABLE 105

| | | | Reten- | |
|---|---|---|---|---|
| | | | tion | Mass |
| Com- | | | Time | [M + |
| pound | Structure | LC/MS | [min] | H] |
| 509 | 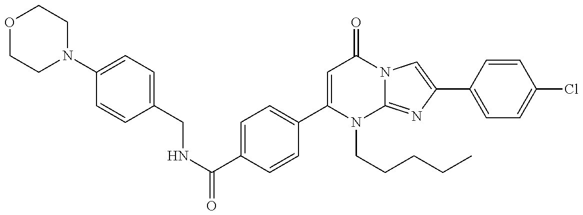 [SMILES: CCCCCn1c(-c2ccc(C(=O)NCc3ccc(N4CCOCC4)cc3)cc2)cc(=O)n2cc(-c3ccc(Cl)cc3)nc12] | Method A | 2.88 | 610 |
| 510 | 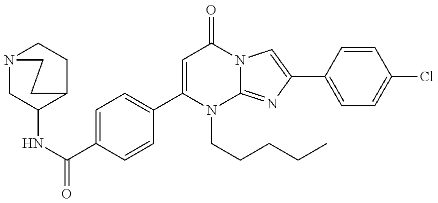 [SMILES: CCCCCn1c(-c2ccc(C(=O)NC3CN4CCC3CC4)cc2)cc(=O)n2cc(-c3ccc(Cl)cc3)nc12] | Method A | 2.17 | 544 |
| 511 | 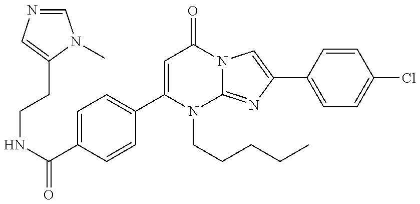 [SMILES: CCCCCn1c(-c2ccc(C(=O)NCCc3cncn3C)cc2)cc(=O)n2cc(-c3ccc(Cl)cc3)nc12] | Method A | 2.12 | 543 |
| 512 | 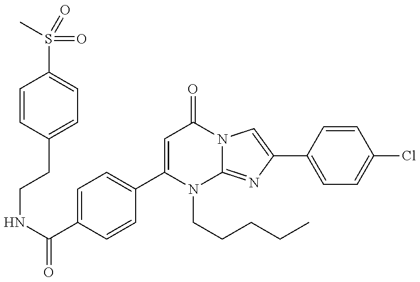 [SMILES: CCCCCn1c(-c2ccc(C(=O)NCCc3ccc(S(C)(=O)=O)cc3)cc2)cc(=O)n2cc(-c3ccc(Cl)cc3)nc12] | Method A | 2.63 | 618 |

TABLE 106

| | | | Retention | |
|---|---|---|---|---|
| | | | Time | |
| Compound | Structure | LC/MS | [min] | Mass [M + H] |
| 513 | 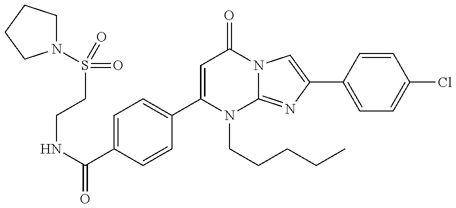 [SMILES: CCCCCn1c(-c2ccc(C(=O)NCCS(=O)(=O)N3CCCC3)cc2)cc(=O)n2cc(-c3ccc(Cl)cc3)nc12] | Method A | 2.78 | 596 |
| 514 | 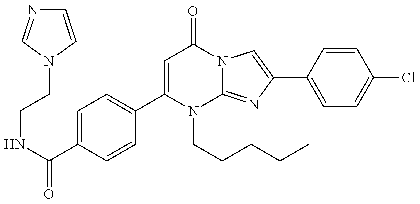 [SMILES: CCCCCn1c(-c2ccc(C(=O)NCCn3ccnc3)cc2)cc(=O)n2cc(-c3ccc(Cl)cc3)nc12] | Method A | 2.09 | 529 |
| 515 | 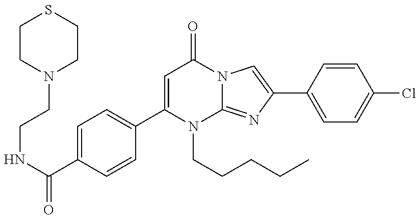 [SMILES: CCCCCn1c(-c2ccc(C(=O)NCCN3CCSCC3)cc2)cc(=O)n2cc(-c3ccc(Cl)cc3)nc12] | Method A | 2.21 | 564 |
| 516 | 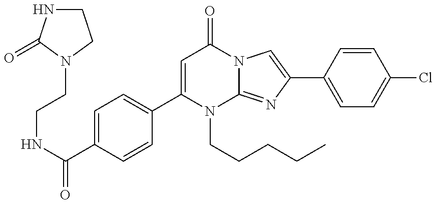 [SMILES: CCCCCn1c(-c2ccc(C(=O)NCCN3CCNC3=O)cc2)cc(=O)n2cc(-c3ccc(Cl)cc3)nc12] | Method A | 2.4 | 547 |
| 517 | 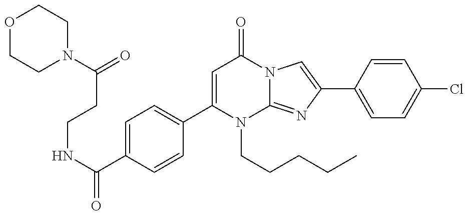 [SMILES: CCCCCn1c(-c2ccc(C(=O)NCCC(=O)N3CCOCC3)cc2)cc(=O)n2cc(-c3ccc(Cl)cc3)nc12] | Method A | 2.5 | 576 |

TABLE 107

| | | | Retention | |
|---|---|---|---|---|
| | | | Time | |
| Compound | Structure | LC/MS | [min] | Mass [M + H] |
| 518 | 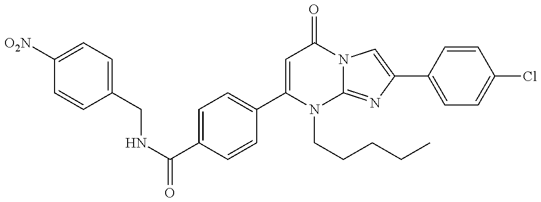 [SMILES: CCCCCn1c(-c2ccc(C(=O)NCc3ccc([N+](=O)[O-])cc3)cc2)cc(=O)n2cc(-c3ccc(Cl)cc3)nc12] | Method A | 2.97 | 570 |
| 519 | 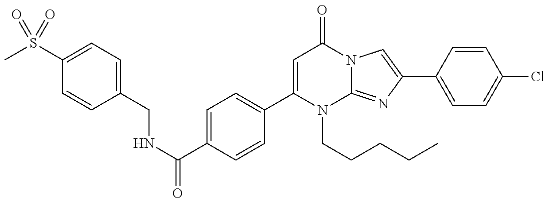 [SMILES: CCCCCn1c(-c2ccc(C(=O)NCc3ccc(S(C)(=O)=O)cc3)cc2)cc(=O)n2cc(-c3ccc(Cl)cc3)nc12] | Method A | 2.72 | 603 |
| 520 | 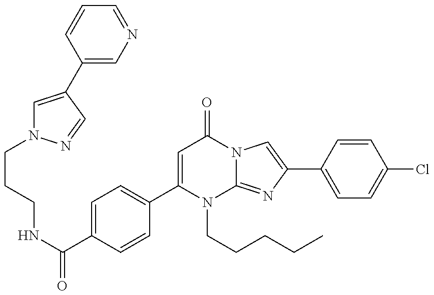 [SMILES: CCCCCn1c(-c2ccc(C(=O)NCCCn3cc(-c4cccnc4)cn3)cc2)cc(=O)n2cc(-c3ccc(Cl)cc3)nc12] | Method A | 2.28 | 620 |

TABLE 108

| | | | Retention | |
|---|---|---|---|---|
| Com- | | | Time | |
| pound | Structure | LC/MS | [min] | Mass [M + H] |
| 521 | 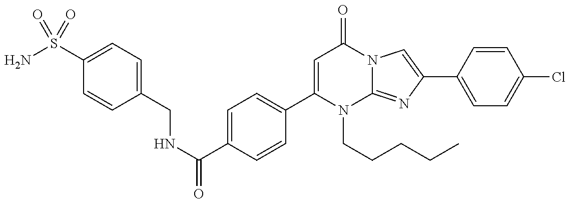 [SMILES: CCCCCn1c(-c2ccc(C(=O)NCc3ccc(S(N)(=O)=O)cc3)cc2)cc(=O)n2cc(-c3ccc(Cl)cc3)nc12] | Method A | 2.62 | 604 |
| 522 | 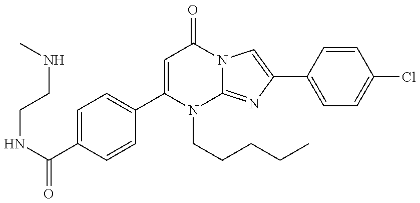 [SMILES: CCCCCn1c(-c2ccc(C(=O)NCCNC)cc2)cc(=O)n2cc(-c3ccc(Cl)cc3)nc12] | Method A | 2.07 | 492 |
| 523 | 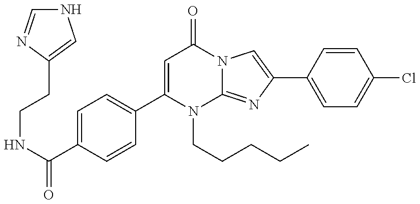 [SMILES: CCCCCn1c(-c2ccc(C(=O)NCCc3c[nH]cn3)cc2)cc(=O)n2cc(-c3ccc(Cl)cc3)nc12] | Method A | 2.09 | 529 |
| 524 | 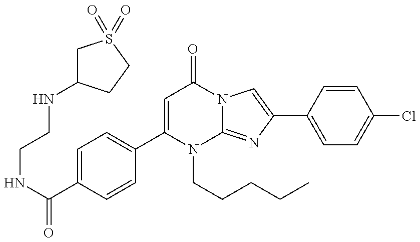 [SMILES: CCCCCn1c(-c2ccc(C(=O)NCCNC3CCS(=O)(=O)C3)cc2)cc(=O)n2cc(-c3ccc(Cl)cc3)nc12] | Method A | 2.1 | 596 |

TABLE 109

| | | | Retention | |
|---|---|---|---|---|
| | | | Time | Mass |
| Compound | Structure | LC/MS | [min] | [M + H] |
| 525 | 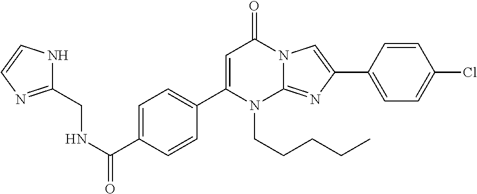 [SMILES: CCCCCn1c(-c2ccc(C(=O)NCc3ncc[nH]3)cc2)cc(=O)n2cc(-c3ccc(Cl)cc3)nc12] | Method A | 2.08 | 515 |
| 526 | 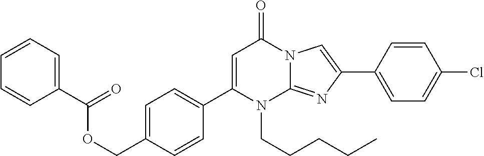 [SMILES: CCCCCn1c(-c2ccc(COC(=O)c3ccccc3)cc2)cc(=O)n2cc(-c3ccc(Cl)cc3)nc12] | Method A | 3.17 | 450 |
| 527 | 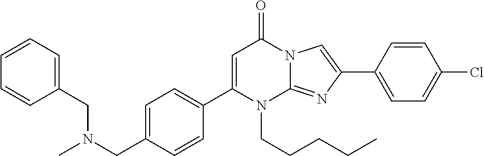 [SMILES: CCCCCn1c(-c2ccc(CN(C)Cc3ccccc3)cc2)cc(=O)n2cc(-c3ccc(Cl)cc3)nc12] | Method A | 3.32 | 559 |
| 528 | 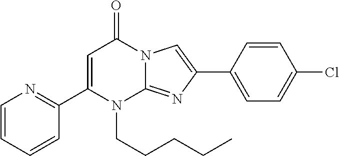 [SMILES: CCCCCn1c(-c2ccccn2)cc(=O)n2cc(-c3ccc(Cl)cc3)nc12] | Method B | 2.79 | 393 |

TABLE 110

| | | | Retention | |
|---|---|---|---|---|
| | | | Time | Mass |
| Compound | Structure | LC/MS | [min] | [M + H] |
| 529 | 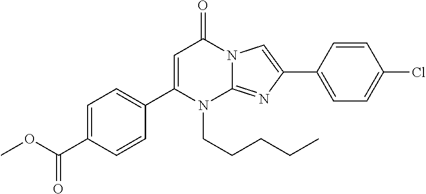 [SMILES: CCCCCn1c(-c2ccc(C(=O)OC)cc2)cc(=O)n2cc(-c3ccc(Cl)cc3)nc12] | B | 2.95 | 449 |
| 530 | 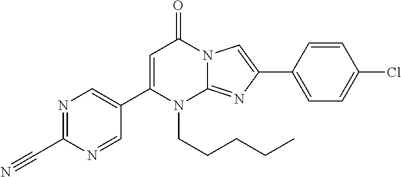 [SMILES: CCCCCn1c(-c2cnc(C#N)nc2)cc(=O)n2cc(-c3ccc(Cl)cc3)nc12] | B | 2.61 | 419 |
| 531 | 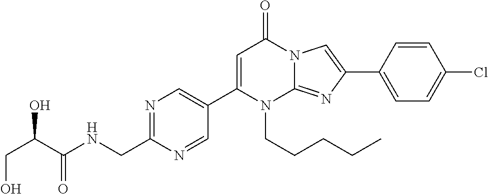 [SMILES: CCCCCn1c(-c2cnc(CNC(=O)[C@H](O)CO)nc2)cc(=O)n2cc(-c3ccc(Cl)cc3)nc12] | B | 2.09 | 511 |
| 532 | 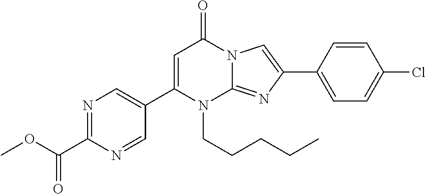 [SMILES: CCCCCn1c(-c2cnc(C(=O)OC)nc2)cc(=O)n2cc(-c3ccc(Cl)cc3)nc12] | B | 2.44 | 452 |
| 533 | 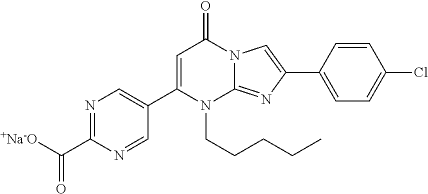 | B | 2.29 | 438 |

TABLE 111

| | | | Retention | |
|---|---|---|---|---|
| | | | Time | Mass |
| Compound | Structure | LC/MS | [min] | [M + H] |
| 534 | 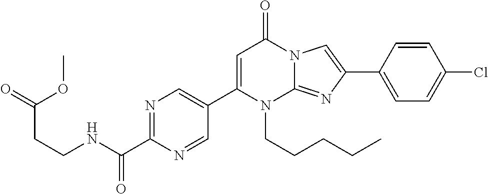 [SMILES: CCCCCn1c(-c2cnc(C(=O)NCCC(=O)OC)nc2)cc(=O)n2cc(-c3ccc(Cl)cc3)nc12] | B | 2.37 | 523 |
| 535 | 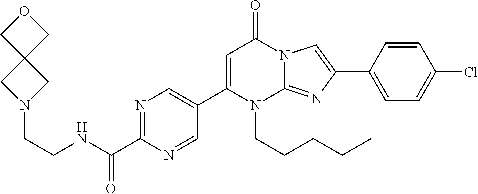 [SMILES: CCCCCn1c(-c2cnc(C(=O)NCCN3CC4(COC4)C3)nc2)cc(=O)n2cc(-c3ccc(Cl)cc3)nc12] | B | 1.74 | 562 |
| 536 | 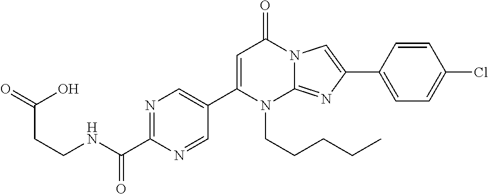 [SMILES: CCCCCn1c(-c2cnc(C(=O)NCCC(=O)O)nc2)cc(=O)n2cc(-c3ccc(Cl)cc3)nc12] | A | 2.20 | 509 |

TABLE 112

| | | | Retention | |
|---|---|---|---|---|
| | | | Time | Mass |
| Compound | Structure | LC/MS | [min] | [M + H] |
| 537 | 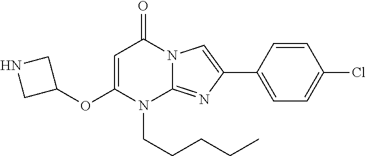 [SMILES: CCCCCn1c(OC2CNC2)cc(=O)n2cc(-c3ccc(Cl)cc3)nc12] | B | 1.56 | 387 |
| 538 | 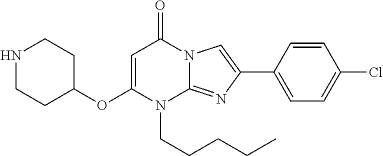 [SMILES: CCCCCn1c(OC2CCNCC2)cc(=O)n2cc(-c3ccc(Cl)cc3)nc12] | B | 1.61 | 415 |
| 539 | 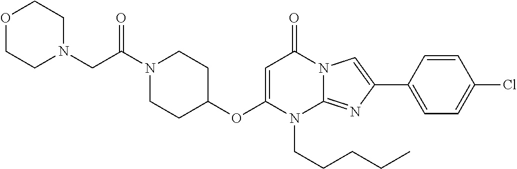 [SMILES: CCCCCn1c(OC2CCN(C(=O)CN3CCOCC3)CC2)cc(=O)n2cc(-c3ccc(Cl)cc3)nc12] | B | 1.68 | 542 |
| 540 | 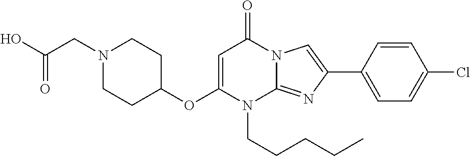 [SMILES: CCCCCn1c(OC2CCN(CC(=O)O)CC2)cc(=O)n2cc(-c3ccc(Cl)cc3)nc12] | B | 1.84 | 473 |
| 541 | 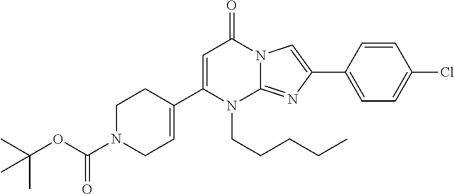 [SMILES: CCCCCn1c(C2=CCN(C(=O)OC(C)(C)C)CC2)cc(=O)n2cc(-c3ccc(Cl)cc3)nc12] | B | 3.07 | 497 |

TABLE 113

| | | | Retention | |
|---|---|---|---|---|
| | | | Time | Mass |
| Compound | Structure | LC/MS | [min] | [M + H] |
| 542 | 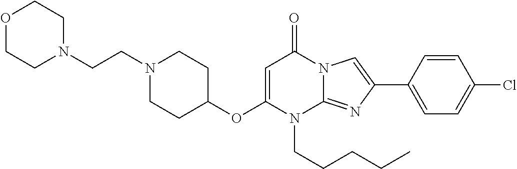 [SMILES: CCCCCn1c(OC2CCN(CCN3CCOCC3)CC2)cc(=O)n2cc(-c3ccc(Cl)cc3)nc12] | B | 1.64 | 528 |
| 543 | 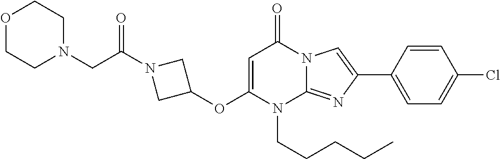 [SMILES: CCCCCn1c(OC2CN(C(=O)CN3CCOCC3)C2)cc(=O)n2cc(-c3ccc(Cl)cc3)nc12] | B | 1.68 | 514 |
| 544 | 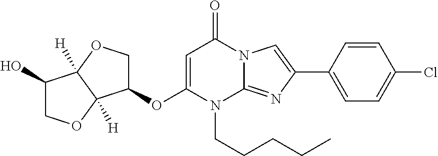 [SMILES: CCCCCn1c(O[C@@H]2CO[C@H]3[C@@H]2OC[C@H]3O)cc(=O)n2cc(-c3ccc(Cl)cc3)nc12] | B | 2.31 | 460 |
| 545 | 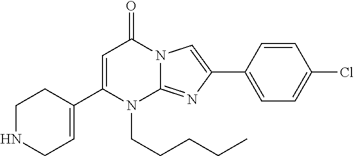 [SMILES: CCCCCn1c(C2=CCNCC2)cc(=O)n2cc(-c3ccc(Cl)cc3)nc12] | B | 1.57 | 438 |
| 546 | 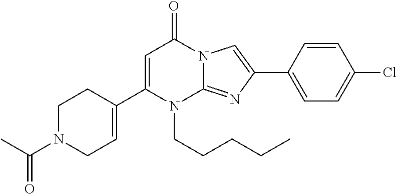 [SMILES: CCCCCn1c(C2=CCN(C(C)=O)CC2)cc(=O)n2cc(-c3ccc(Cl)cc3)nc12] | B | 2.43 | 439 |

TABLE 114

| | | | Retention | |
|---|---|---|---|---|
| | | | Time | Mass |
| Compound | Structure | LC/MS | [min] | [M + H] |
| 547 | 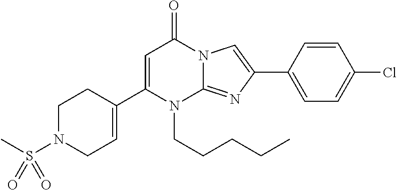 [SMILES: CCCCCn1c(C2=CCN(S(C)(=O)=O)CC2)cc(=O)n2cc(-c3ccc(Cl)cc3)nc12] | B | 2.55 | 475 |
| 548 | 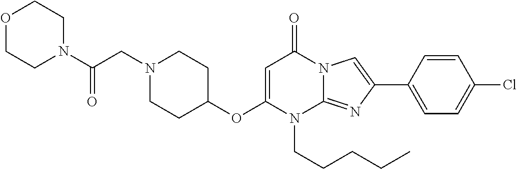 [SMILES: CCCCCn1c(OC2CCN(CC(=O)N3CCOCC3)CC2)cc(=O)n2cc(-c3ccc(Cl)cc3)nc12] | B | 1.64 | 542 |
| 549 | 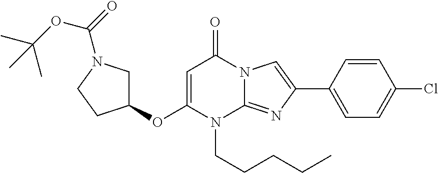 [SMILES: CCCCCn1c(O[C@H]2CCN(C(=O)OC(C)(C)C)C2)cc(=O)n2cc(-c3ccc(Cl)cc3)nc12] | B | 2.97 | 501 |
| 550 | 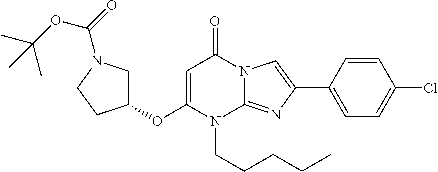 [SMILES: CCCCCn1c(O[C@@H]2CCN(C(=O)OC(C)(C)C)C2)cc(=O)n2cc(-c3ccc(Cl)cc3)nc12] | B | 2.98 | 501 |
| 551 | 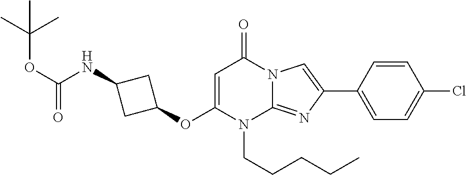 [SMILES: CCCCCn1c(O[C@H]2C[C@@H](NC(=O)OC(C)(C)C)C2)cc(=O)n2cc(-c3ccc(Cl)cc3)nc12] | B | 2.93 | 501 |

TABLE 115

| | | | Retention | |
|---|---|---|---|---|
| | | | Time | Mass |
| Compound | Structure | LC/MS | [min] | [M + H] |
| 552 | 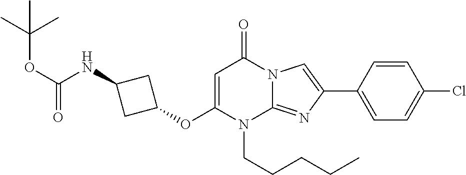 [SMILES: CCCCCn1c(O[C@H]2C[C@H](NC(=O)OC(C)(C)C)C2)cc(=O)n2cc(-c3ccc(Cl)cc3)nc12] | B | 2.98 | 501 |
| 553 | 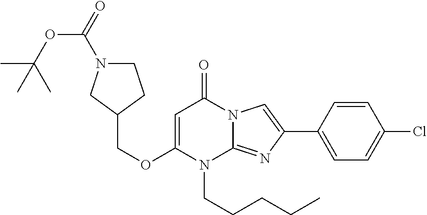 [SMILES: CCCCCn1c(OCC2CCN(C(=O)OC(C)(C)C)C2)cc(=O)n2cc(-c3ccc(Cl)cc3)nc12] | B | 3.05 | 515 |
| 554 | 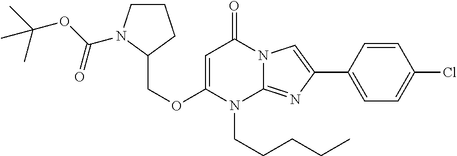 [SMILES: CCCCCn1c(OCC2CCCN2C(=O)OC(C)(C)C)cc(=O)n2cc(-c3ccc(Cl)cc3)nc12] | B | 3.13 | 515 |
| 555 | 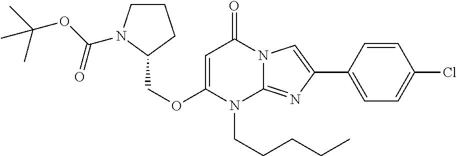 [SMILES: CCCCCn1c(OC[C@H]2CCCN2C(=O)OC(C)(C)C)cc(=O)n2cc(-c3ccc(Cl)cc3)nc12] | B | 3.13 | 515 |
| 556 | 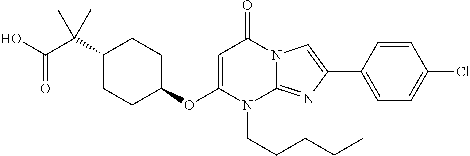 [SMILES: CCCCCn1c(O[C@H]2CC[C@H](C(C)(C)C(=O)O)CC2)cc(=O)n2cc(-c3ccc(Cl)cc3)nc12] | B | 2.95 | 500 |

TABLE 116

| | | | Retention | |
|---|---|---|---|---|
| | | | Time | Mass |
| Compound | Structure | LC/MS | [min] | [M + H] |
| 557 | 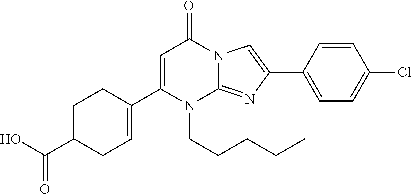 [SMILES: CCCCCn1c(C2=CCC(C(=O)O)CC2)cc(=O)n2cc(-c3ccc(Cl)cc3)nc12] | B | 2.57 | 440 |
| 558 | 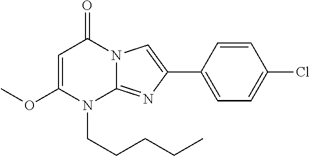 [SMILES: CCCCCn1c(OC)cc(=O)n2cc(-c3ccc(Cl)cc3)nc12] | B | 2.70 | 346 |
| 559 | 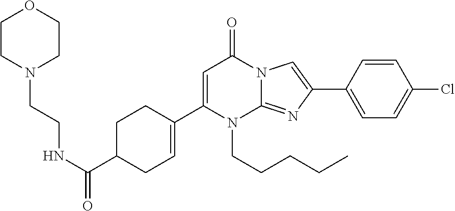 [SMILES: CCCCCn1c(C2=CCC(C(=O)NCCN3CCOCC3)CC2)cc(=O)n2cc(-c3ccc(Cl)cc3)nc12] | B | 2.95 | 500 |
| 560 | 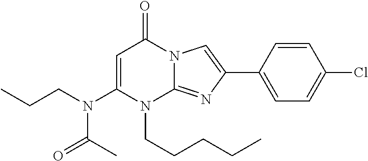 [SMILES: CCCCCn1c(N(CCC)C(C)=O)cc(=O)n2cc(-c3ccc(Cl)cc3)nc12] | B | 2.67 | 415 |
| 561 | 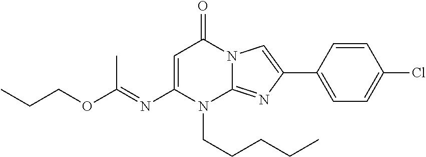 [SMILES: CCCCCn1c(/N=C(\C)OCCC)cc(=O)n2cc(-c3ccc(Cl)cc3)nc12] | B | 2.96 | 415 |

TABLE 117

| | | | Retention | |
|---|---|---|---|---|
| | | | Time | Mass |
| Compound | Structure | LC/MS | [min] | [M + H] |
| 562 | 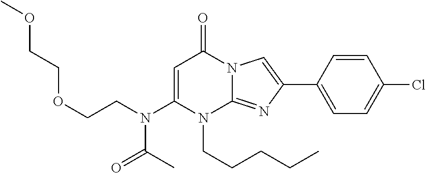 [SMILES: CCCCCn1c(N(CCOCCOC)C(C)=O)cc(=O)n2cc(-c3ccc(Cl)cc3)nc12] | B | 2.53 | 475 |
| 563 | 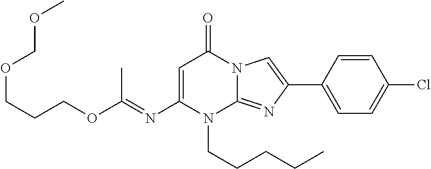 [SMILES: CCCCCn1c(/N=C(\C)OCCCOCOC)cc(=O)n2cc(-c3ccc(Cl)cc3)nc12] | B | 2.67 | 475 |
| 564 | 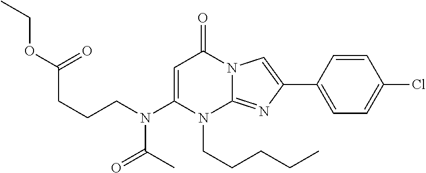 [SMILES: CCCCCn1c(N(CCCC(=O)OCC)C(C)=O)cc(=O)n2cc(-c3ccc(Cl)cc3)nc12] | B | 2.65 | 487 |
| 565 | 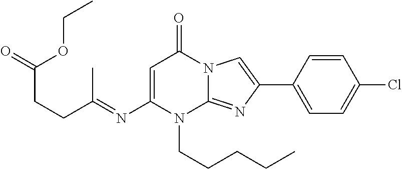 [SMILES: CCCCCn1c(/N=C(\C)CCC(=O)OCC)cc(=O)n2cc(-c3ccc(Cl)cc3)nc12] | B | 2.85 | 487 |
| 566 | 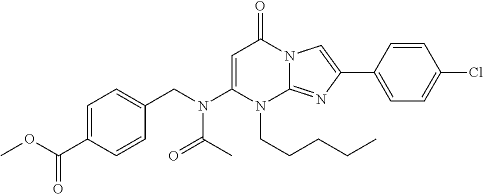 [SMILES: CCCCCn1c(N(Cc2ccc(C(=O)OC)cc2)C(C)=O)cc(=O)n2cc(-c3ccc(Cl)cc3)nc12] | B | 2.71 | 521 |

TABLE 118

| | | | Retention | |
|---|---|---|---|---|
| | | | Time | Mass |
| Compound | Structure | LC/MS | [min] | [M + H] |
| 567 | 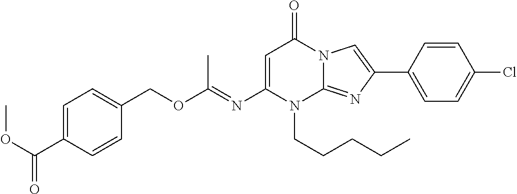 [SMILES: CCCCCn1c(/N=C(\C)OCc2ccc(C(=O)OC)cc2)cc(=O)n2cc(-c3ccc(Cl)cc3)nc12] | B | 2.88 | 521 |
| 568 | 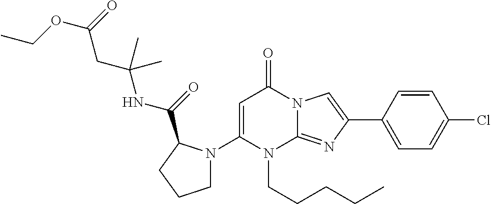 [SMILES: CCCCCn1c(N2CCC[C@H]2C(=O)NC(C)(C)CC(=O)OCC)cc(=O)n2cc(-c3ccc(Cl)cc3)nc12] | B | 2.75 | 557 |
| 569 | 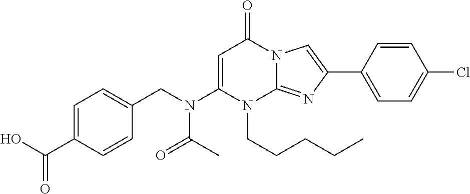 [SMILES: CCCCCn1c(N(Cc2ccc(C(=O)O)cc2)C(C)=O)cc(=O)n2cc(-c3ccc(Cl)cc3)nc12] | B | 2.47 | 507 |
| 570 | 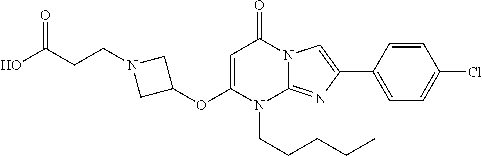 [SMILES: CCCCCn1c(OC2CN(CCC(=O)O)C2)cc(=O)n2cc(-c3ccc(Cl)cc3)nc12] | B | 1.63 | 459 |
| 571 | 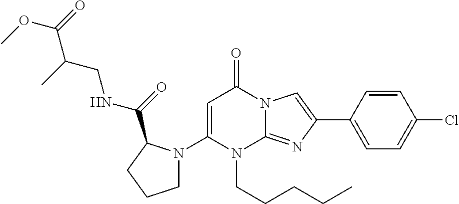 [SMILES: CCCCCn1c(N2CCC[C@H]2C(=O)NCC(C)C(=O)OC)cc(=O)n2cc(-c3ccc(Cl)cc3)nc12] | B | 2.50 | 528 |

TABLE 119

| | | | Retention | |
|---|---|---|---|---|
| | | | Time | Mass |
| Compound | Structure | LC/MS | min | [M + H] |
| 572 | 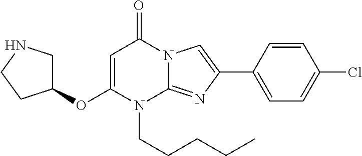 [SMILES: CCCCCn1c(O[C@H]2CCNC2)cc(=O)n2cc(-c3ccc(Cl)cc3)nc12] | B | 1.56 | 401 |
| 573 | 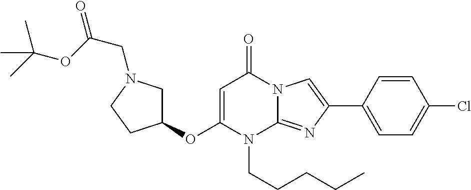 [SMILES: CCCCCn1c(O[C@H]2CCN(CC(=O)OC(C)(C)C)C2)cc(=O)n2cc(-c3ccc(Cl)cc3)nc12] | C | 2.16 | 515 |
| 574 | 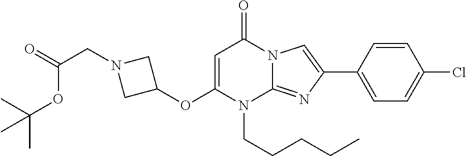 [SMILES: CCCCCn1c(OC2CN(CC(=O)OC(C)(C)C)C2)cc(=O)n2cc(-c3ccc(Cl)cc3)nc12] | C | 2.13 | 501 |
| 575 | 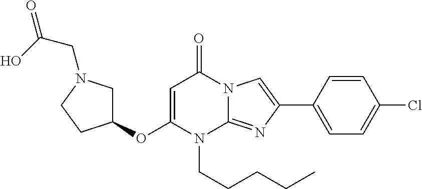 [SMILES: CCCCCn1c(O[C@H]2CCN(CC(=O)O)C2)cc(=O)n2cc(-c3ccc(Cl)cc3)nc12] | B | 1.79 | 459 |
| 576 | 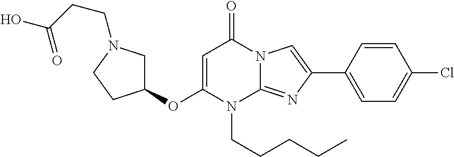 [SMILES: CCCCCn1c(O[C@H]2CCN(CCC(=O)O)C2)cc(=O)n2cc(-c3ccc(Cl)cc3)nc12] | C | 1.77 | 473 |

TABLE 120

| | | | Retention | |
|---|---|---|---|---|
| | | | Time | Mass |
| Compound | Structure | LC/MS | [min] | [M + H] |
| 577 | 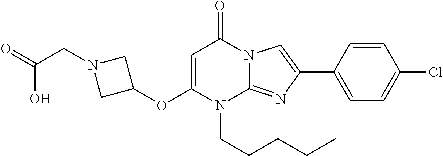 [SMILES: CCCCCn1c(OC2CN(CC(=O)O)C2)cc(=O)n2cc(-c3ccc(Cl)cc3)nc12] | C | 1.91 | 445 |
| 578 | 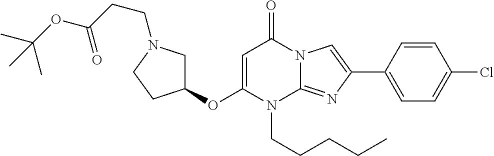 [SMILES: CCCCCn1c(O[C@H]2CCN(CCC(=O)OC(C)(C)C)C2)cc(=O)n2cc(-c3ccc(Cl)cc3)nc12] | B | 1.98 | 529 |
| 579 | 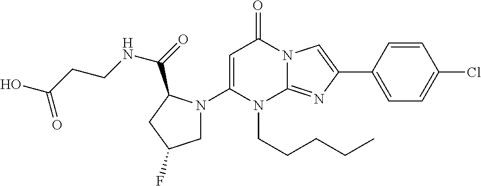 [SMILES: CCCCCn1c(N2C[C@H](F)C[C@H]2C(=O)NCCC(=O)O)cc(=O)n2cc(-c3ccc(Cl)cc3)nc12] | A | 2.26 | 518 |
| 580 | 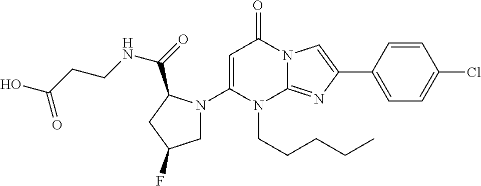 [SMILES: CCCCCn1c(N2C[C@@H](F)C[C@H]2C(=O)NCCC(=O)O)cc(=O)n2cc(-c3ccc(Cl)cc3)nc12] | A | 2.29 | 518 |
| 581 | 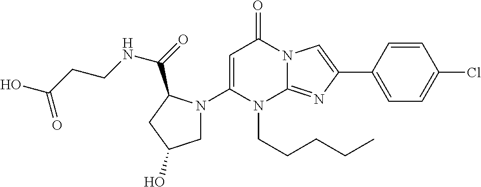 [SMILES: CCCCCn1c(N2C[C@H](O)C[C@H]2C(=O)NCCC(=O)O)cc(=O)n2cc(-c3ccc(Cl)cc3)nc12] | A | 2.00 | 516 |

TABLE 121

| | | | Retention | |
|---|---|---|---|---|
| | | | Time | Mass |
| Compound | Structure | LC/MS | [min] | [M + H] |
| 582 | 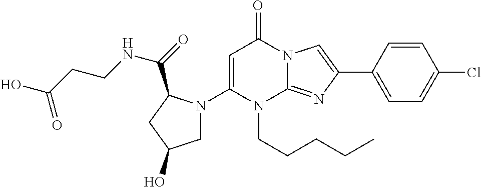 [SMILES: CCCCCn1c(N2C[C@@H](O)C[C@H]2C(=O)NCCC(=O)O)cc(=O)n2cc(-c3ccc(Cl)cc3)nc12] | A | 2.03 | 516 |
| 583 | 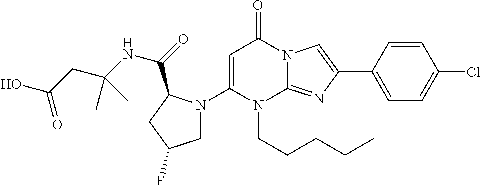 [SMILES: CCCCCn1c(N2C[C@H](F)C[C@H]2C(=O)NC(C)(C)CC(=O)O)cc(=O)n2cc(-c3ccc(Cl)cc3)nc12] | A | 2.46 | 546 |
| 584 | 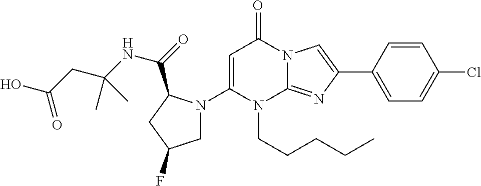 [SMILES: CCCCCn1c(N2C[C@@H](F)C[C@H]2C(=O)NC(C)(C)CC(=O)O)cc(=O)n2cc(-c3ccc(Cl)cc3)nc12] | A | 2.52 | 546 |
| 585 | 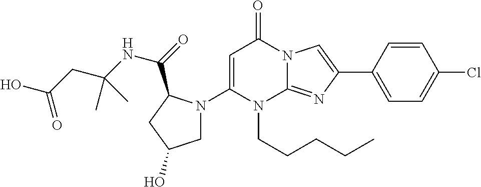 [SMILES: CCCCCn1c(N2C[C@H](O)C[C@H]2C(=O)NC(C)(C)CC(=O)O)cc(=O)n2cc(-c3ccc(Cl)cc3)nc12] | A | 2.19 | 544 |
| 586 | 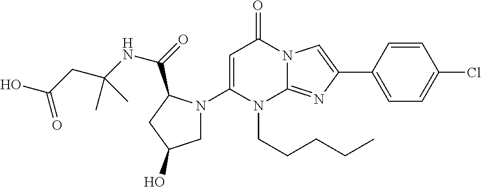 [SMILES: CCCCCn1c(N2C[C@@H](O)C[C@H]2C(=O)NC(C)(C)CC(=O)O)cc(=O)n2cc(-c3ccc(Cl)cc3)nc12] | A | 2.24 | 544 |

TABLE 122

| | | | Retention | |
|---|---|---|---|---|
| Com- | | | Time | Mass |
| pound | Structure | LC/MS | [min] | [M + H] |
| 587 | 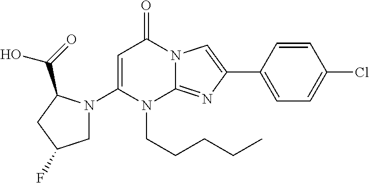 [SMILES: CCCCCn1c(N2C[C@H](F)C[C@H]2C(=O)O)cc(=O)n2cc(-c3ccc(Cl)cc3)nc12] | A | 2.43 | 447 |
| 588 | 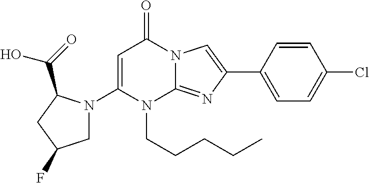 [SMILES: CCCCCn1c(N2C[C@@H](F)C[C@H]2C(=O)O)cc(=O)n2cc(-c3ccc(Cl)cc3)nc12] | A | 2.47 | 447 |
| 589 | 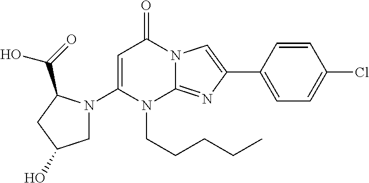 [SMILES: CCCCCn1c(N2C[C@H](O)C[C@H]2C(=O)O)cc(=O)n2cc(-c3ccc(Cl)cc3)nc12] | A | 2.11 | 445 |
| 590 | 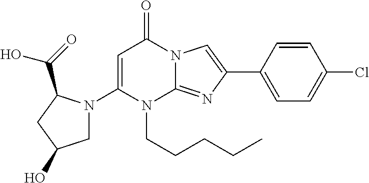 [SMILES: CCCCCn1c(N2C[C@@H](O)C[C@H]2C(=O)O)cc(=O)n2cc(-c3ccc(Cl)cc3)nc12] | A | 2.13 | 445 |
| 591 | 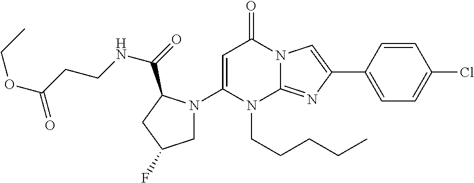 [SMILES: CCCCCn1c(N2C[C@H](F)C[C@H]2C(=O)NCCC(=O)OCC)cc(=O)n2cc(-c3ccc(Cl)cc3)nc12] | A | 2.59 | 546 |

TABLE 123

| | | | Retention | |
|---|---|---|---|---|
| Com- | | | Time | Mass |
| pound | Structure | LC/MS | [min] | [M + H] |
| 592 | 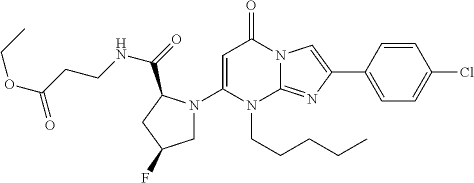 [SMILES: CCCCCn1c(N2C[C@@H](F)C[C@H]2C(=O)NCCC(=O)OCC)cc(=O)n2cc(-c3ccc(Cl)cc3)nc12] | A | 2.69 | 546 |
| 593 | 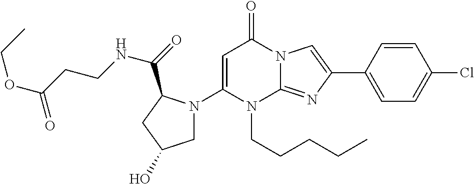 [SMILES: CCCCCn1c(N2C[C@H](O)C[C@H]2C(=O)NCCC(=O)OCC)cc(=O)n2cc(-c3ccc(Cl)cc3)nc12] | A | 2.32 | 544 |
| 594 | 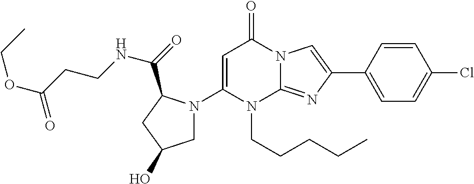 [SMILES: CCCCCn1c(N2C[C@@H](O)C[C@H]2C(=O)NCCC(=O)OCC)cc(=O)n2cc(-c3ccc(Cl)cc3)nc12] | A | 2.36 | 544 |
| 595 | 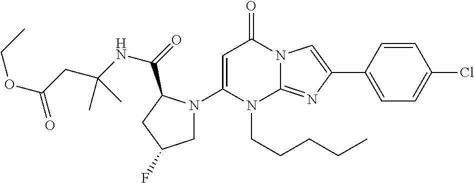 [SMILES: CCCCCn1c(N2C[C@H](F)C[C@H]2C(=O)NC(C)(C)CC(=O)OCC)cc(=O)n2cc(-c3ccc(Cl)cc3)nc12] | A | 2.83 | 574 |
| 596 | 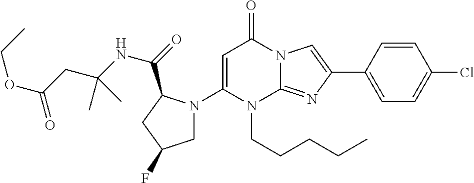 [SMILES: CCCCCn1c(N2C[C@@H](F)C[C@H]2C(=O)NC(C)(C)CC(=O)OCC)cc(=O)n2cc(-c3ccc(Cl)cc3)nc12] | A | 2.96 | 574 |

TABLE 124

| | | | Retention | |
|---|---|---|---|---|
| Com- | | | Time | Mass |
| pound | Structure | LC/MS | [min] | [M + H] |
| 597 | 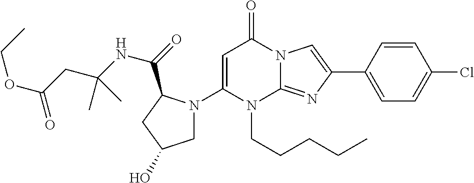 [SMILES: CCCCCn1c(N2C[C@H](O)C[C@H]2C(=O)NC(C)(C)CC(=O)OCC)cc(=O)n2cc(-c3ccc(Cl)cc3)nc12] | A | 2.57 | 572 |
| 598 | 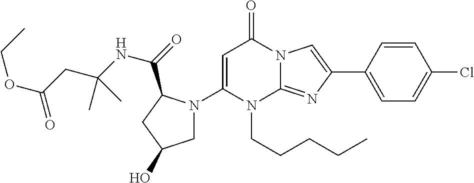 [SMILES: CCCCCn1c(N2C[C@@H](O)C[C@H]2C(=O)NC(C)(C)CC(=O)OCC)cc(=O)n2cc(-c3ccc(Cl)cc3)nc12] | A | 2.61 | 572 |
| 599 | 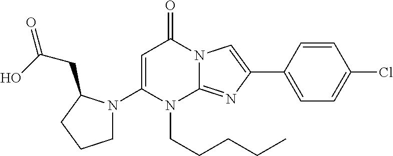 [SMILES: CCCCCn1c(N2CCC[C@H]2CC(=O)O)cc(=O)n2cc(-c3ccc(Cl)cc3)nc12] | A | 2.60 | 443 |
| 600 | 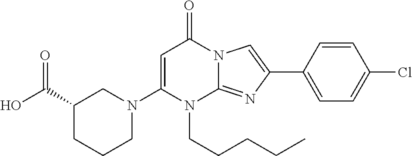 [SMILES: CCCCCn1c(N2CCC[C@H](C(=O)O)C2)cc(=O)n2cc(-c3ccc(Cl)cc3)nc12] | A | 2.65 | 443 |
| 601 | 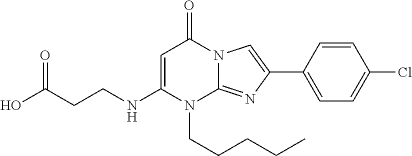 [SMILES: CCCCCn1c(NCCC(=O)O)cc(=O)n2cc(-c3ccc(Cl)cc3)nc12] | A | 2.27 | 403 |

TABLE 125

| | | | Retention | |
|---|---|---|---|---|
| Com- | | | Time | Mass |
| pound | Structure | LC/MS | [min] | [M + H] |
| 602 | 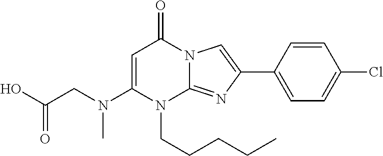 [SMILES: CCCCCn1c(N(C)CC(=O)O)cc(=O)n2cc(-c3ccc(Cl)cc3)nc12] | A | 2.43 | 403 |
| 603 | 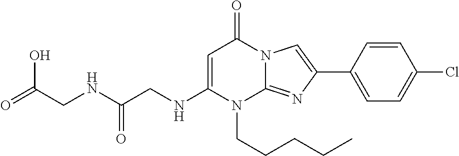 [SMILES: CCCCCn1c(NCC(=O)NCC(=O)O)cc(=O)n2cc(-c3ccc(Cl)cc3)nc12] | A | 2.12 | 446 |
| 604 | 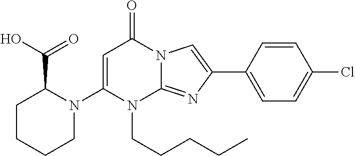 [SMILES: CCCCCn1c(N2CCCC[C@H]2C(=O)O)cc(=O)n2cc(-c3ccc(Cl)cc3)nc12] | A | 2.79 | 443 |
| 605 | 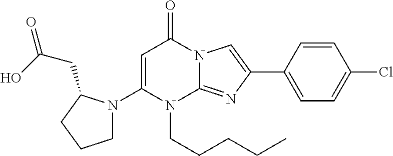 [SMILES: CCCCCn1c(N2CCC[C@@H]2CC(=O)O)cc(=O)n2cc(-c3ccc(Cl)cc3)nc12] | A | 2.60 | 443 |
| 606 | 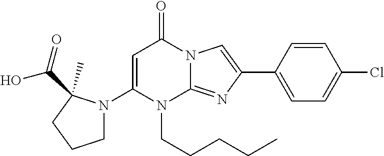 [SMILES: CCCCCn1c(N2CCC[C@@]2(C)C(=O)O)cc(=O)n2cc(-c3ccc(Cl)cc3)nc12] | A | 2.73 | 443 |

TABLE 126

| | | | Retention | |
|---|---|---|---|---|
| Com- | | | Time | Mass |
| pound | Structure | LC/MS | [min] | [M + H] |
| 607 | 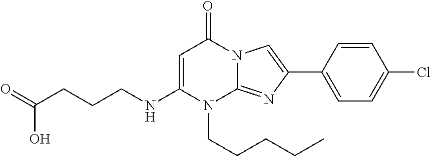 [SMILES: CCCCCn1c(NCCCC(=O)O)cc(=O)n2cc(-c3ccc(Cl)cc3)nc12] | A | 2.34 | 417 |
| 608 | 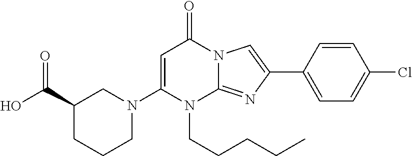 [SMILES: CCCCCn1c(N2CCC[C@@H](C(=O)O)C2)cc(=O)n2cc(-c3ccc(Cl)cc3)nc12] | A | 2.65 | 443 |
| 609 | 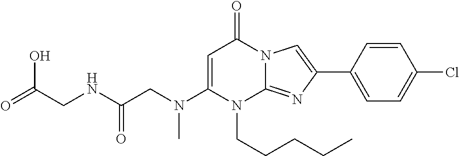 [SMILES: CCCCCn1c(N(C)CC(=O)NCC(=O)O)cc(=O)n2cc(-c3ccc(Cl)cc3)nc12] | A | 2.24 | 460 |
| 610 | 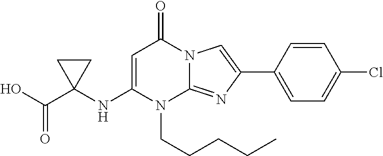 [SMILES: CCCCCn1c(NC2(C(=O)O)CC2)cc(=O)n2cc(-c3ccc(Cl)cc3)nc12] | A | 2.37 | 415 |
| 611 | 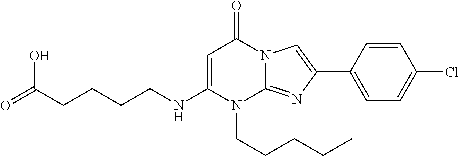 [SMILES: CCCCCn1c(NCCCCC(=O)O)cc(=O)n2cc(-c3ccc(Cl)cc3)nc12] | A | 2.39 | 431 |

TABLE 127

| | | | Retention | |
|---|---|---|---|---|
| Com- | | | Time | Mass |
| pound | Structure | LC/MS | [min] | [M + H] |
| 612 | 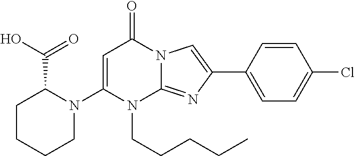 [SMILES: CCCCCn1c(N2CCCC[C@@H]2C(=O)O)cc(=O)n2cc(-c3ccc(Cl)cc3)nc12] | A | 2.79 | 443 |
| 613 | 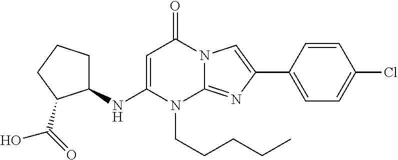 [SMILES: CCCCCn1c(N[C@@H]2CCC[C@H]2C(=O)O)cc(=O)n2cc(-c3ccc(Cl)cc3)nc12] | A | 2.50 | 443 |
| 614 | 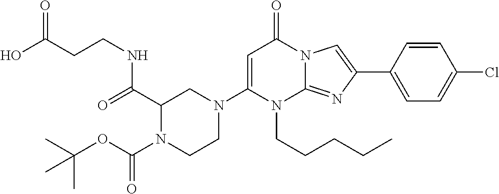 [SMILES: CCCCCn1c(N2CCN(C(=O)OC(C)(C)C)C(C(=O)NCCC(=O)O)C2)cc(=O)n2cc(-c3ccc(Cl)cc3)nc12] | A | 2.50 | 615 |
| 615 | 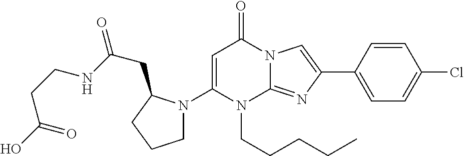 [SMILES: CCCCCn1c(N2CCC[C@H]2CC(=O)NCCC(=O)O)cc(=O)n2cc(-c3ccc(Cl)cc3)nc12] | A | 2.35 | 514 |
| 616 | 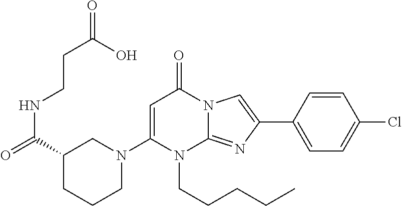 [SMILES: CCCCCn1c(N2CCC[C@H](C(=O)NCCC(=O)O)C2)cc(=O)n2cc(-c3ccc(Cl)cc3)nc12] | A | 2.37 | 514 |

TABLE 128

| | | | Retention | |
|---|---|---|---|---|
| Com- | | | Time | Mass |
| pound | Structure | LC/MS | [min] | [M + H] |
| 617 | 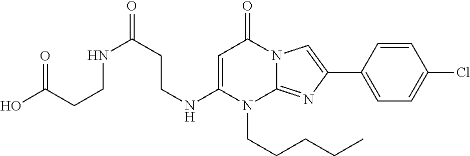 [SMILES: CCCCCn1c(NCCC(=O)NCCC(=O)O)cc(=O)n2cc(-c3ccc(Cl)cc3)nc12] | A | 2.11 | 474 |
| 618 | 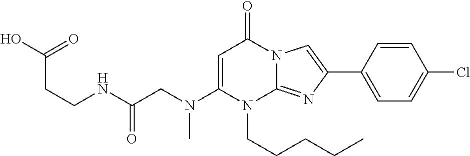 [SMILES: CCCCCn1c(N(C)CC(=O)NCCC(=O)O)cc(=O)n2cc(-c3ccc(Cl)cc3)nc12] | A | 2.20 | 474 |
| 619 | 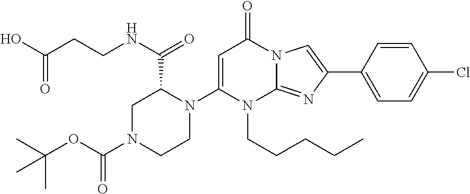 [SMILES: CCCCCn1c(N2CCN(C(=O)OC(C)(C)C)C[C@@H]2C(=O)NCCC(=O)O)cc(=O)n2cc(-c3ccc(Cl)cc3)nc12] | A | 2.71 | 615 |
| 620 | 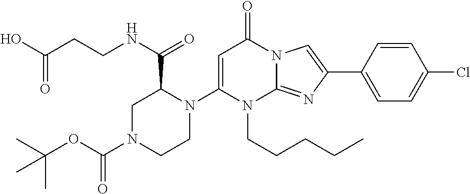 [SMILES: CCCCCn1c(N2CCN(C(=O)OC(C)(C)C)C[C@H]2C(=O)NCCC(=O)O)cc(=O)n2cc(-c3ccc(Cl)cc3)nc12] | A | 2.71 | 615 |
| 621 | 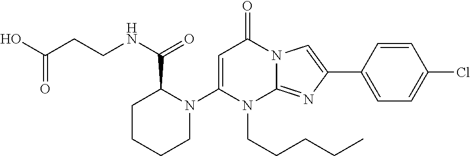 [SMILES: CCCCCn1c(N2CCCC[C@H]2C(=O)NCCC(=O)O)cc(=O)n2cc(-c3ccc(Cl)cc3)nc12] | A | 2.55 | 514 |

TABLE 129

| | | | Retention | |
|---|---|---|---|---|
| Com- | | | Time | Mass |
| pound | Structure | LC/MS | [min] | [M + H] |
| 622 | 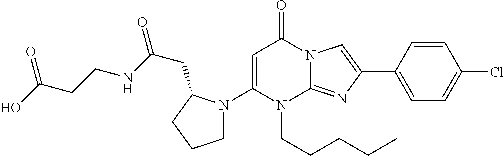 [SMILES: CCCCCn1c(N2CCC[C@@H]2CC(=O)NCCC(=O)O)cc(=O)n2cc(-c3ccc(Cl)cc3)nc12] | A | 2.35 | 514 |
| 623 | 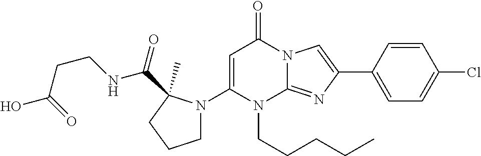 [SMILES: CCCCCn1c(N2CCC[C@@]2(C)C(=O)NCCC(=O)O)cc(=O)n2cc(-c3ccc(Cl)cc3)nc12] | A | 2.40 | 514 |
| 624 | 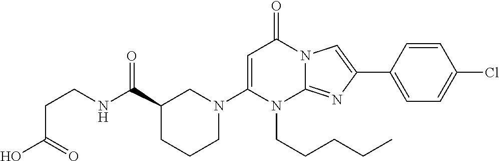 [SMILES: CCCCCn1c(N2CCC[C@@H](C(=O)NCCC(=O)O)C2)cc(=O)n2cc(-c3ccc(Cl)cc3)nc12] | A | 2.37 | 514 |
| 625 | 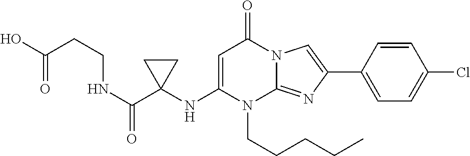 [SMILES: CCCCCn1c(NC2(C(=O)NCCC(=O)O)CC2)cc(=O)n2cc(-c3ccc(Cl)cc3)nc12] | A | 2.23 | 486 |
| 626 | 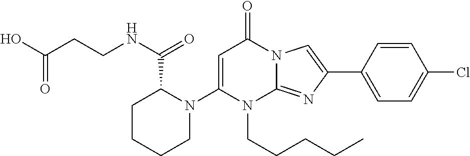 [SMILES: CCCCCn1c(N2CCCC[C@@H]2C(=O)NCCC(=O)O)cc(=O)n2cc(-c3ccc(Cl)cc3)nc12] | A | 2.55 | 514 |

TABLE 130

| | | | Retention | |
|---|---|---|---|---|
| Com- | | | Time | Mass |
| pound | Structure | LC/MS | [min] | [M + H] |
| 627 | 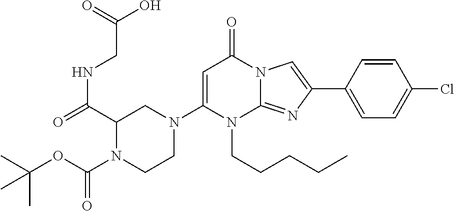 [SMILES: CCCCCn1c(N2CCN(C(=O)OC(C)(C)C)C(C(=O)NCC(=O)O)C2)cc(=O)n2cc(-c3ccc(Cl)cc3)nc12] | A | 2.49 | 601 |
| 628 | 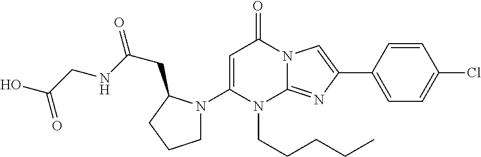 [SMILES: CCCCCn1c(N2CCC[C@H]2CC(=O)NCC(=O)O)cc(=O)n2cc(-c3ccc(Cl)cc3)nc12] | A | 2.35 | 500 |
| 629 | 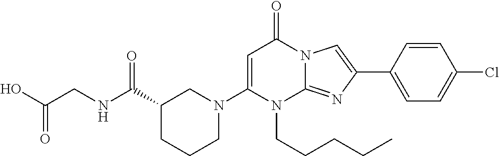 [SMILES: CCCCCn1c(N2CCC[C@H](C(=O)NCC(=O)O)C2)cc(=O)n2cc(-c3ccc(Cl)cc3)nc12] | A | 2.37 | 500 |
| 630 | 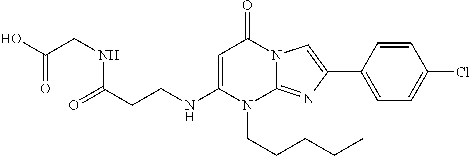 [SMILES: CCCCCn1c(NCCC(=O)NCC(=O)O)cc(=O)n2cc(-c3ccc(Cl)cc3)nc12] | A | 2.09 | 460 |
| 631 | 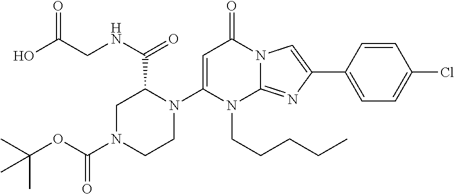 [SMILES: CCCCCn1c(N2CCN(C(=O)OC(C)(C)C)C[C@@H]2C(=O)NCC(=O)O)cc(=O)n2cc(-c3ccc(Cl)cc3)nc12] | A | 2.70 | 601 |

TABLE 131

| | | | Retention | |
|---|---|---|---|---|
| Com- | | | Time | Mass |
| pound | Structure | LC/MS | [min] | [M + H] |
| 632 | 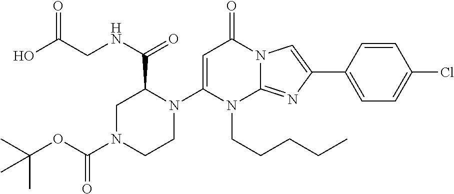 [SMILES: CCCCCn1c(N2CCN(C(=O)OC(C)(C)C)C[C@H]2C(=O)NCC(=O)O)cc(=O)n2cc(-c3ccc(Cl)cc3)nc12] | A | 2.70 | 601 |
| 633 | 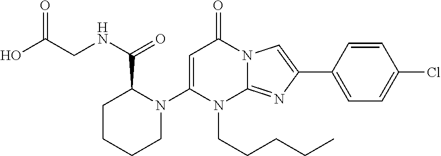 [SMILES: CCCCCn1c(N2CCCC[C@H]2C(=O)NCC(=O)O)cc(=O)n2cc(-c3ccc(Cl)cc3)nc12] | A | 2.55 | 500 |
| 634 | 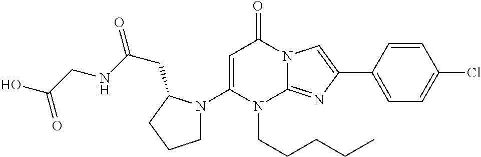 [SMILES: CCCCCn1c(N2CCC[C@@H]2CC(=O)NCC(=O)O)cc(=O)n2cc(-c3ccc(Cl)cc3)nc12] | A | 2.35 | 500 |
| 635 | 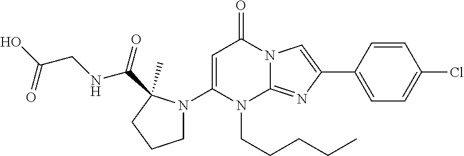 [SMILES: CCCCCn1c(N2CCC[C@@]2(C)C(=O)NCC(=O)O)cc(=O)n2cc(-c3ccc(Cl)cc3)nc12] | A | 2.41 | 500 |
| 636 | 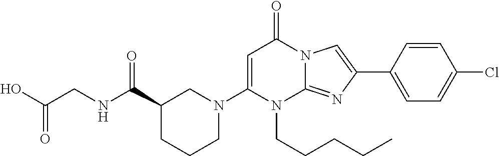 [SMILES: CCCCCn1c(N2CCC[C@@H](C(=O)NCC(=O)O)C2)cc(=O)n2cc(-c3ccc(Cl)cc3)nc12] | A | 2.37 | 500 |

TABLE 132

| | | | Retention | |
|---|---|---|---|---|
| Com- | | | Time | Mass |
| pound | Structure | LC/MS | [min] | [M + H] |
| 637 | 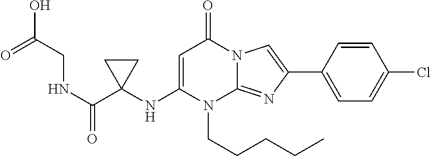 [SMILES: CCCCCn1c(NC2(C(=O)NCC(=O)O)CC2)cc(=O)n2cc(-c3ccc(Cl)cc3)nc12] | A | 2.23 | 472 |
| 638 | 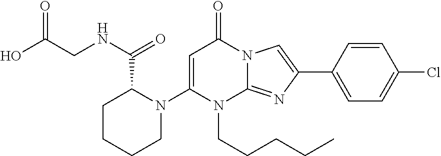 [SMILES: CCCCCn1c(N2CCCC[C@@H]2C(=O)NCC(=O)O)cc(=O)n2cc(-c3ccc(Cl)cc3)nc12] | A | 2.55 | 500 |
| 639 | 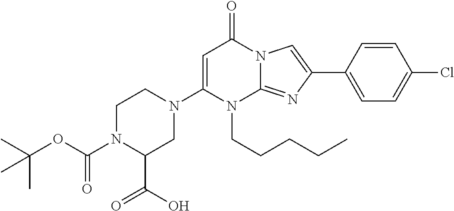 [SMILES: CCCCCn1c(N2CCN(C(=O)OC(C)(C)C)C(C(=O)O)C2)cc(=O)n2cc(-c3ccc(Cl)cc3)nc12] | A | 2.68 | 544 |
| 640 | 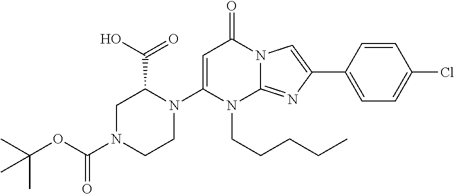 [SMILES: CCCCCn1c(N2CCN(C(=O)OC(C)(C)C)C[C@@H]2C(=O)O)cc(=O)n2cc(-c3ccc(Cl)cc3)nc12] | A | 2.86 | 544 |
| 641 | 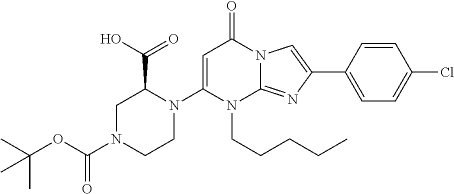 [SMILES: CCCCCn1c(N2CCN(C(=O)OC(C)(C)C)C[C@H]2C(=O)O)cc(=O)n2cc(-c3ccc(Cl)cc3)nc12] | A | 2.86 | 544 |

TABLE 133

| | | | Retention | |
|---|---|---|---|---|
| Com- | | | Time | Mass |
| pound | Structure | LC/MS | [min] | [M + H] |
| 642 | 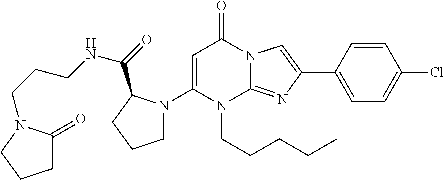 [SMILES: CCCCCn1c(N2CCC[C@H]2C(=O)NCCCN2CCCC2=O)cc(=O)n2cc(-c3ccc(Cl)cc3)nc12] | A | 2.34 | 553 |
| 643 | 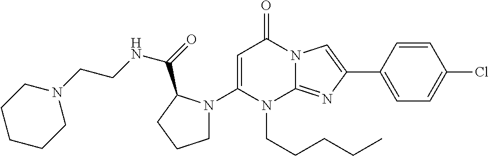 [SMILES: CCCCCn1c(N2CCC[C@H]2C(=O)NCCN2CCCCC2)cc(=O)n2cc(-c3ccc(Cl)cc3)nc12] | A | 1.98 | 539 |
| 644 | 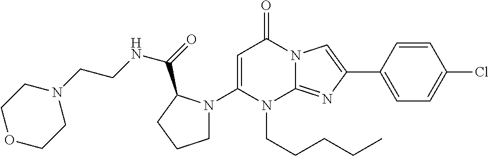 [SMILES: CCCCCn1c(N2CCC[C@H]2C(=O)NCCN2CCOCC2)cc(=O)n2cc(-c3ccc(Cl)cc3)nc12] | A | 1.94 | 541 |
| 645 | 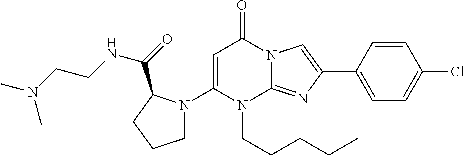 [SMILES: CCCCCn1c(N2CCC[C@H]2C(=O)NCCN(C)C)cc(=O)n2cc(-c3ccc(Cl)cc3)nc12] | A | 1.92 | 499 |
| 646 | 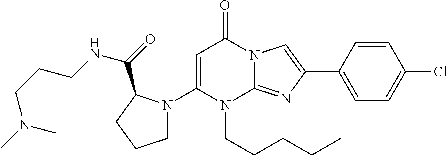 [SMILES: CCCCCn1c(N2CCC[C@H]2C(=O)NCCCN(C)C)cc(=O)n2cc(-c3ccc(Cl)cc3)nc12] | A | 1.92 | 513 |

TABLE 134

| | | | Retention | |
|---|---|---|---|---|
| | | | Time | |
| Compound | Structure | LC/MS | [min] | Mass [M + H] |
| 647 | 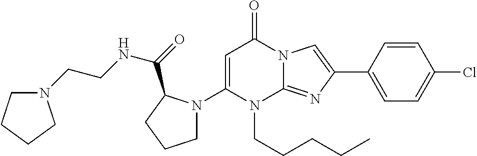 [SMILES: CCCCCn1c(N2CCC[C@H]2C(=O)NCCN2CCCC2)cc(=O)n2cc(-c3ccc(Cl)cc3)nc12] | A | 1.97 | 525 |
| 648 | 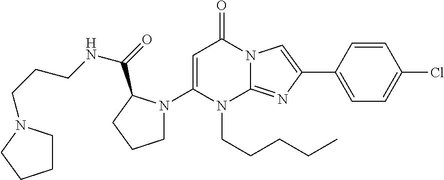 [SMILES: CCCCCn1c(N2CCC[C@H]2C(=O)NCCCN2CCCC2)cc(=O)n2cc(-c3ccc(Cl)cc3)nc12] | A | 1.97 | 539 |
| 649 | 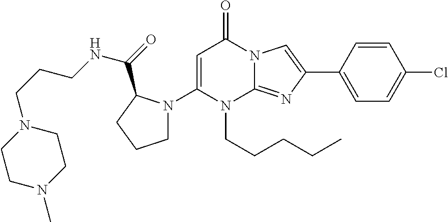 [SMILES: CCCCCn1c(N2CCC[C@H]2C(=O)NCCCN2CCN(C)CC2)cc(=O)n2cc(-c3ccc(Cl)cc3)nc12] | A | 1.75 | 568 |
| 650 | 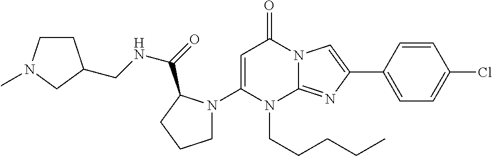 [SMILES: CCCCCn1c(N2CCC[C@H]2C(=O)NCC2CCN(C)C2)cc(=O)n2cc(-c3ccc(Cl)cc3)nc12] | A | 1.92 | 525 |
| 651 | 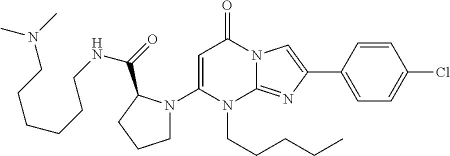 [SMILES: CCCCCn1c(N2CCC[C@H]2C(=O)NCCCCCCN(C)C)cc(=O)n2cc(-c3ccc(Cl)cc3)nc12] | A | 2.03 | 555 |

TABLE 135

| | | | Retention | |
|---|---|---|---|---|
| | | | Time | |
| Compound | Structure | LC/MS | [min] | Mass [M + H] |
| 652 | 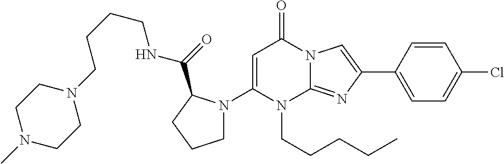 [SMILES: CCCCCn1c(N2CCC[C@H]2C(=O)NCCCCN2CCN(C)CC2)cc(=O)n2cc(-c3ccc(Cl)cc3)nc12] | A | 1.74 | 582 |
| 653 | 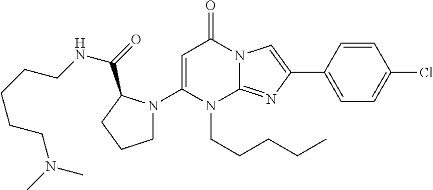 [SMILES: CCCCCn1c(N2CCC[C@H]2C(=O)NCCCCCN(C)C)cc(=O)n2cc(-c3ccc(Cl)cc3)nc12] | A | 1.99 | 541 |
| 654 | 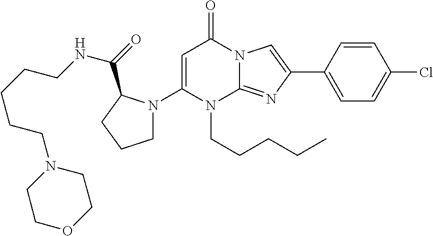 [SMILES: CCCCCn1c(N2CCC[C@H]2C(=O)NCCCCCN2CCOCC2)cc(=O)n2cc(-c3ccc(Cl)cc3)nc12] | A | 1.96 | 583 |
| 655 | 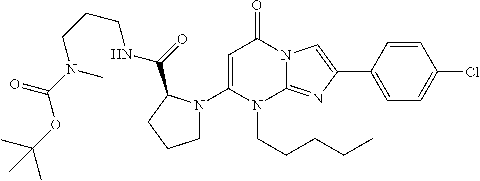 [SMILES: CCCCCn1c(N2CCC[C@H]2C(=O)NCCCN(C)C(=O)OC(C)(C)C)cc(=O)n2cc(-c3ccc(Cl)cc3)nc12] | A | 2.94 | 599 |
| 656 | 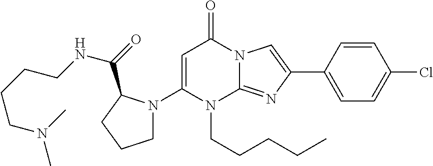 [SMILES: CCCCCn1c(N2CCC[C@H]2C(=O)NCCCCN(C)C)cc(=O)n2cc(-c3ccc(Cl)cc3)nc12] | A | 1.94 | 527 |

TABLE 136

| | | | Retention | |
|---|---|---|---|---|
| | | | Time | |
| Compound | Structure | LC/MS | [min] | Mass [M + H] |
| 657 | 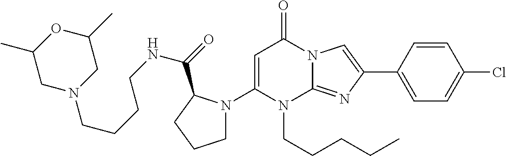 [SMILES: CCCCCn1c(N2CCC[C@H]2C(=O)NCCCCN2CC(C)OC(C)C2)cc(=O)n2cc(-c3ccc(Cl)cc3)nc12] | A | 2.07 | 597 |
| 658 | 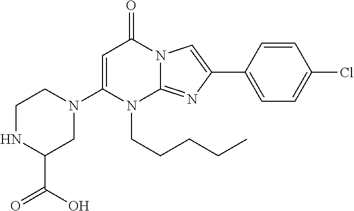 [SMILES: CCCCCn1c(N2CCNC(C(=O)O)C2)cc(=O)n2cc(-c3ccc(Cl)cc3)nc12] | A | 1.87 | 444 |
| 659 | 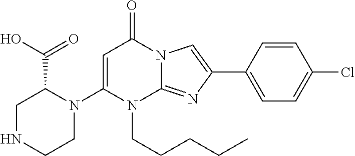 [SMILES: CCCCCn1c(N2CCNC[C@@H]2C(=O)O)cc(=O)n2cc(-c3ccc(Cl)cc3)nc12] | A | 1.85 | 444 |
| 660 | 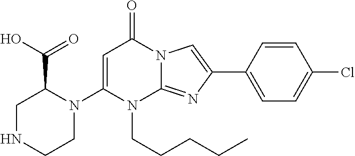 [SMILES: CCCCCn1c(N2CCNC[C@H]2C(=O)O)cc(=O)n2cc(-c3ccc(Cl)cc3)nc12] | A | 1.85 | 444 |
| 661 | 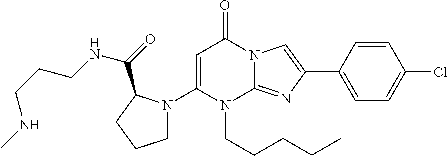 [SMILES: CCCCCn1c(N2CCC[C@H]2C(=O)NCCCNC)cc(=O)n2cc(-c3ccc(Cl)cc3)nc12] | A | 1.99 | 499 |

TABLE 137

| | | | Retention | |
|---|---|---|---|---|
| | | | Time | |
| Compound | Structure | LC/MS | [min] | Mass [M + H] |
| 662 | 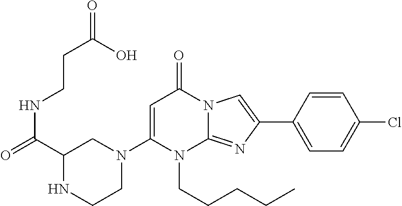 [SMILES: CCCCCn1c(N2CCNC(C(=O)NCCC(=O)O)C2)cc(=O)n2cc(-c3ccc(Cl)cc3)nc12] | A | 1.87 | 515 |
| 663 | 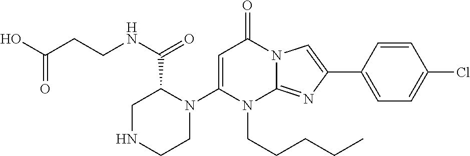 [SMILES: CCCCCn1c(N2CCNC[C@@H]2C(=O)NCCC(=O)O)cc(=O)n2cc(-c3ccc(Cl)cc3)nc12] | A | 1.86 | 515 |
| 664 | 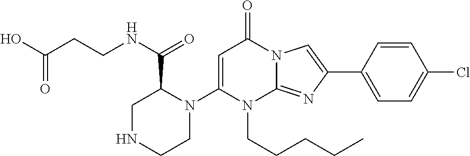 [SMILES: CCCCCn1c(N2CCNC[C@H]2C(=O)NCCC(=O)O)cc(=O)n2cc(-c3ccc(Cl)cc3)nc12] | A | 1.85 | 515 |
| 665 | 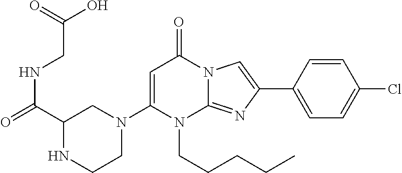 [SMILES: CCCCCn1c(N2CCNC(C(=O)NCC(=O)O)C2)cc(=O)n2cc(-c3ccc(Cl)cc3)nc12] | A | 1.87 | 501 |
| 666 | 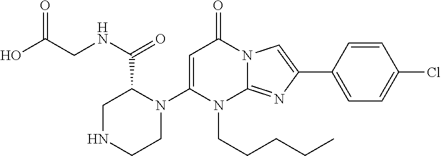 [SMILES: CCCCCn1c(N2CCNC[C@@H]2C(=O)NCC(=O)O)cc(=O)n2cc(-c3ccc(Cl)cc3)nc12] | A | 1.85 | 501 |

TABLE 138

| | | | Retention | |
|---|---|---|---|---|
| | | | Time | |
| Compound | Structure | LC/MS | [min] | Mass [M + H] |
| 667 | 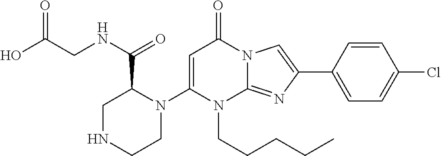 [SMILES: CCCCCn1c(N2CCNC[C@H]2C(=O)NCC(=O)O)cc(=O)n2cc(-c3ccc(Cl)cc3)nc12] | A | 1.85 | 501 |
| 668 | 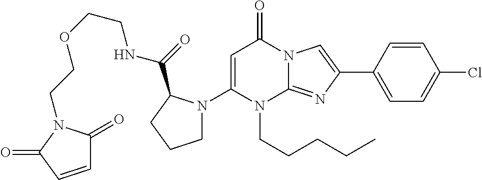 [SMILES: CCCCCn1c(N2CCC[C@H]2C(=O)NCCOCCN2C(=O)C=CC2=O)cc(=O)n2cc(-c3ccc(Cl)cc3)nc12] | A | 2.48 | 595 |
| 669 | 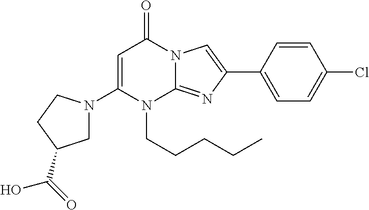 [SMILES: CCCCCn1c(N2CC[C@@H](C(=O)O)C2)cc(=O)n2cc(-c3ccc(Cl)cc3)nc12] | A | 1.68 | 429 |
| 670 | 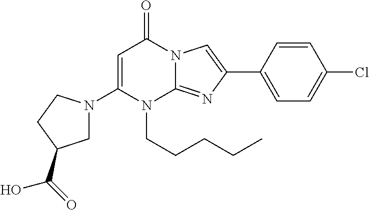 [SMILES: CCCCCn1c(N2CC[C@H](C(=O)O)C2)cc(=O)n2cc(-c3ccc(Cl)cc3)nc12] | A | 1.69 | 429 |
| 671 | 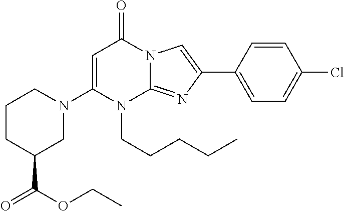 [SMILES: CCCCCn1c(N2CCC[C@H](C(=O)OCC)C2)cc(=O)n2cc(-c3ccc(Cl)cc3)nc12] | A | 3.14 | 471 |

TABLE 139

| | | | Retention | |
|---|---|---|---|---|
| | | | Time | |
| Compound | Structure | LC/MS | [min] | Mass [M + H] |
| 672 | 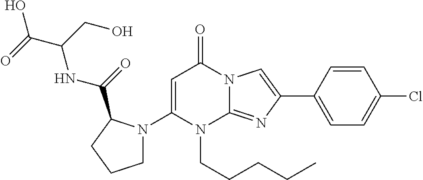 [SMILES: CCCCCn1c(N2CCC[C@H]2C(=O)NC(CO)C(=O)O)cc(=O)n2cc(-c3ccc(Cl)cc3)nc12] | A | 2.18 | 516 |
| 673 | 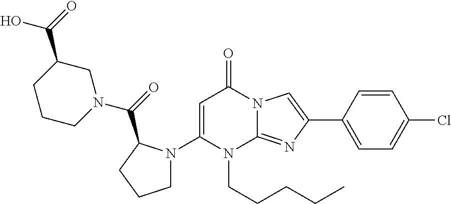 [SMILES: CCCCCn1c(N2CCC[C@H]2C(=O)N2CCC[C@@H](C(=O)O)C2)cc(=O)n2cc(-c3ccc(Cl)cc3)nc12] | A | 2.50 | 540 |
| 674 | 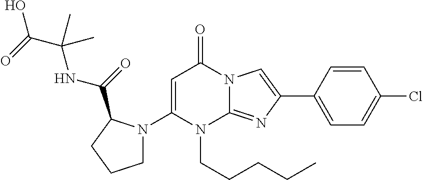 [SMILES: CCCCCn1c(N2CCC[C@H]2C(=O)NC(C)(C)C(=O)O)cc(=O)n2cc(-c3ccc(Cl)cc3)nc12] | A | 2.39 | 514 |
| 675 | 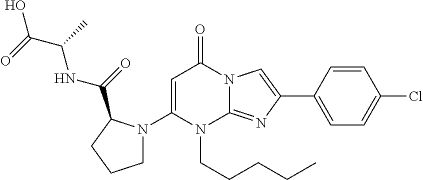 [SMILES: CCCCCn1c(N2CCC[C@H]2C(=O)N[C@@H](C)C(=O)O)cc(=O)n2cc(-c3ccc(Cl)cc3)nc12] | A | 2.33 | 500 |
| 676 | 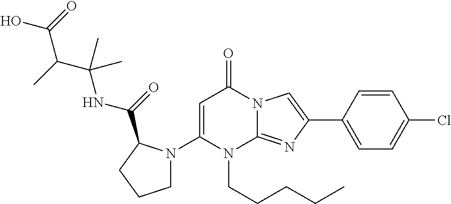 [SMILES: CCCCCn1c(N2CCC[C@H]2C(=O)NC(C)(C)C(C)C(=O)O)cc(=O)n2cc(-c3ccc(Cl)cc3)nc12] | A | 2.55 | 542 |

TABLE 140

| | | | Retention | |
|---|---|---|---|---|
| | | | Time | Mass |
| Compound | Structure | LC/MS | [min] | [M + H] |
| 677 | 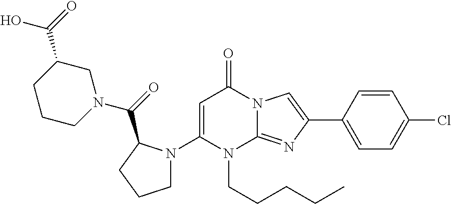 [SMILES: CCCCCn1c(N2CCC[C@H]2C(=O)N2CCC[C@H](C(=O)O)C2)cc(=O)n2cc(-c3ccc(Cl)cc3)nc12] | A | 2.44 | 540 |
| 678 | 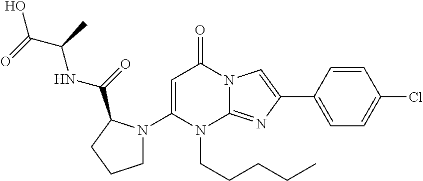 [SMILES: CCCCCn1c(N2CCC[C@H]2C(=O)N[C@H](C)C(=O)O)cc(=O)n2cc(-c3ccc(Cl)cc3)nc12] | A | 1.35 | 500 |
| 679 | 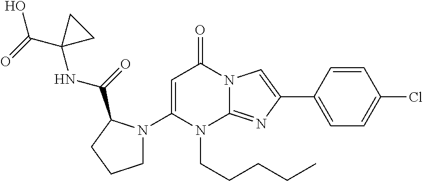 [SMILES: CCCCCn1c(N2CCC[C@H]2C(=O)NC2(C(=O)O)CC2)cc(=O)n2cc(-c3ccc(Cl)cc3)nc12] | A | 2.34 | 512 |
| 680 | 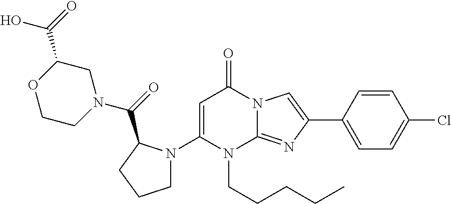 [SMILES: CCCCCn1c(N2CCC[C@H]2C(=O)N2CCO[C@H](C(=O)O)C2)cc(=O)n2cc(-c3ccc(Cl)cc3)nc12] | A | 2.31 | 542 |
| 681 | 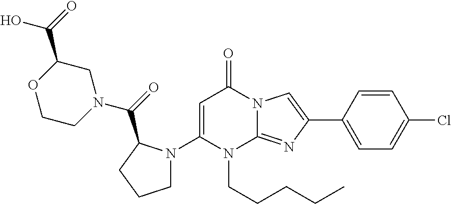 [SMILES: CCCCCn1c(N2CCC[C@H]2C(=O)N2CCO[C@@H](C(=O)O)C2)cc(=O)n2cc(-c3ccc(Cl)cc3)nc12] | A | 2.38 | 542 |

TABLE 141

| | | | Retention | |
|---|---|---|---|---|
| | | | Time | Mass |
| Compound | Structure | LC/MS | [min] | [M + H] |
| 682 | 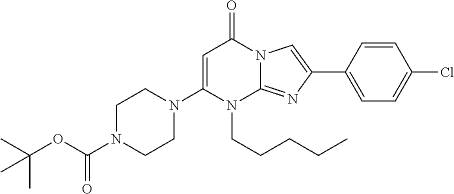 [SMILES: CCCCCn1c(N2CCN(C(=O)OC(C)(C)C)CC2)cc(=O)n2cc(-c3ccc(Cl)cc3)nc12] | B | 3.01 | 500 |
| 683 | 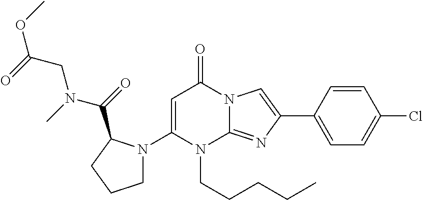 [SMILES: CCCCCn1c(N2CCC[C@H]2C(=O)N(C)CC(=O)OC)cc(=O)n2cc(-c3ccc(Cl)cc3)nc12] | A | 2.56 | 514 |
| 684 | 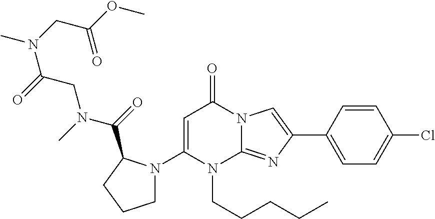 [SMILES: CCCCCn1c(N2CCC[C@H]2C(=O)N(C)CC(=O)N(C)CC(=O)OC)cc(=O)n2cc(-c3ccc(Cl)cc3)nc12] | A | 2.43 | 585 |
| 685 | 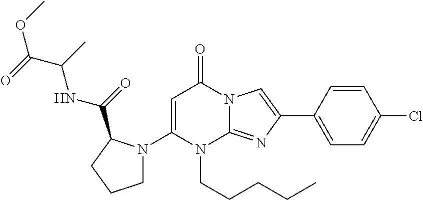 [SMILES: CCCCCn1c(N2CCC[C@H]2C(=O)NC(C)C(=O)OC)cc(=O)n2cc(-c3ccc(Cl)cc3)nc12] | A | 2.57 | 514 |
| 686 | 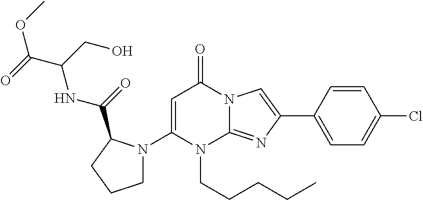 [SMILES: CCCCCn1c(N2CCC[C@H]2C(=O)NC(CO)C(=O)OC)cc(=O)n2cc(-c3ccc(Cl)cc3)nc12] | A | 2.34 | 530 |

TABLE 142

| | | | Retention | |
|---|---|---|---|---|
| Com- | | | Time | Mass |
| pound | Structure | LC/MS | [min] | [M + H] |
| 687 | 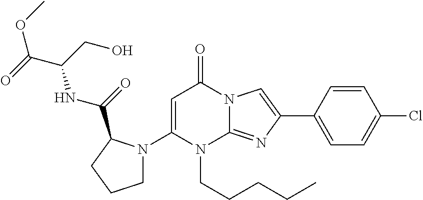 [SMILES: CCCCCn1c(N2CCC[C@H]2C(=O)N[C@@H](CO)C(=O)OC)cc(=O)n2cc(-c3ccc(Cl)cc3)nc12] | A | 2.36 | 530 |
| 688 | 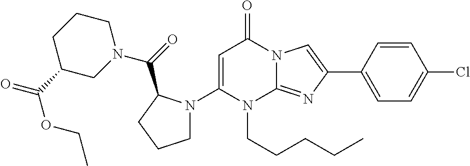 [SMILES: CCCCCn1c(N2CCC[C@H]2C(=O)N2CCC[C@@H](C(=O)OCC)C2)cc(=O)n2cc(-c3ccc(Cl)cc3)nc12] | A | 2.83 | 568 |
| 689 | 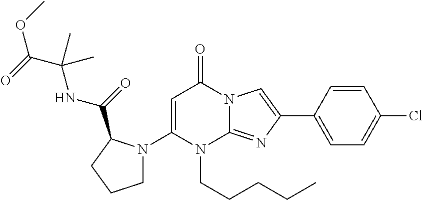 [SMILES: CCCCCn1c(N2CCC[C@H]2C(=O)NC(C)(C)C(=O)OC)cc(=O)n2cc(-c3ccc(Cl)cc3)nc12] | A | 2.62 | 528 |
| 690 | 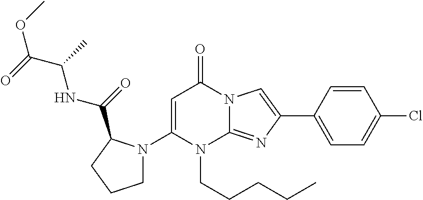 [SMILES: CCCCCn1c(N2CCC[C@H]2C(=O)N[C@@H](C)C(=O)OC)cc(=O)n2cc(-c3ccc(Cl)cc3)nc12] | A | 2.57 | 514 |
| 691 | 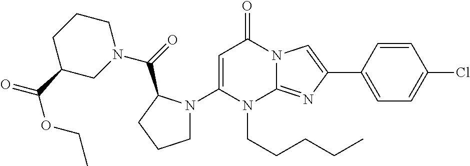 [SMILES: CCCCCn1c(N2CCC[C@H]2C(=O)N2CCC[C@H](C(=O)OCC)C2)cc(=O)n2cc(-c3ccc(Cl)cc3)nc12] | A | 2.83 | 568 |

TABLE 143

| | | | Retention | |
|---|---|---|---|---|
| Com- | | | Time | Mass |
| pound | Structure | LC/MS | [min] | [M + H] |
| 692 | 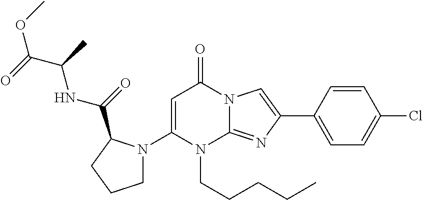 [SMILES: CCCCCn1c(N2CCC[C@H]2C(=O)N[C@H](C)C(=O)OC)cc(=O)n2cc(-c3ccc(Cl)cc3)nc12] | A | 2.56 | 514 |
| 693 | 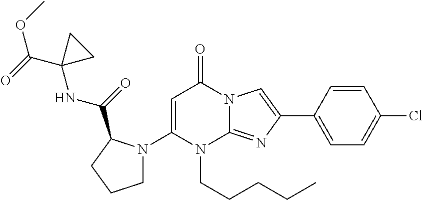 [SMILES: CCCCCn1c(N2CCC[C@H]2C(=O)NC2(C(=O)OC)CC2)cc(=O)n2cc(-c3ccc(Cl)cc3)nc12] | A | 2.52 | 526 |
| 694 | 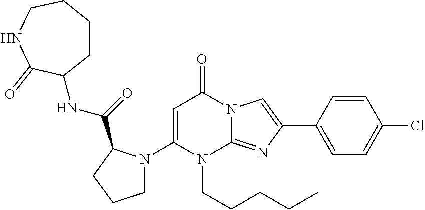 [SMILES: CCCCCn1c(N2CCC[C@H]2C(=O)NC2CCCCNC2=O)cc(=O)n2cc(-c3ccc(Cl)cc3)nc12] | A | 2.43 | 539 |
| 695 | 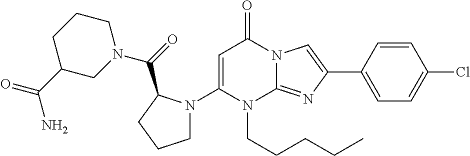 [SMILES: CCCCCn1c(N2CCC[C@H]2C(=O)N2CCCC(C(N)=O)C2)cc(=O)n2cc(-c3ccc(Cl)cc3)nc12] | A | 2.30 | 539 |
| 696 | 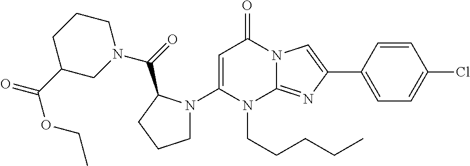 [SMILES: CCCCCn1c(N2CCC[C@H]2C(=O)N2CCCC(C(=O)OCC)C2)cc(=O)n2cc(-c3ccc(Cl)cc3)nc12] | A | 2.83 | 568 |

TABLE 144

| | | | Retention | |
|---|---|---|---|---|
| | | | Time | |
| Compound | Structure | LC/MS | [min] | Mass [M + H] |
| 697 | 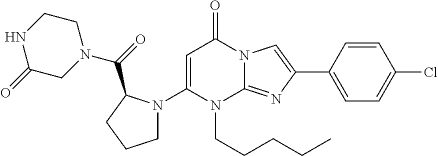 [SMILES: CCCCCn1c(N2CCC[C@H]2C(=O)N2CCNC(=O)C2)cc(=O)n2cc(-c3ccc(Cl)cc3)nc12] | A | 2.21 | 511 |
| 698 | 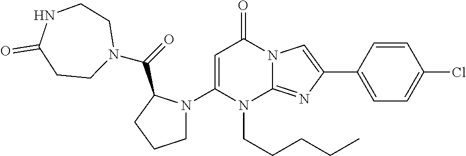 [SMILES: CCCCCn1c(N2CCC[C@H]2C(=O)N2CCNC(=O)CC2)cc(=O)n2cc(-c3ccc(Cl)cc3)nc12] | A | 2.22 | 525 |
| 699 | 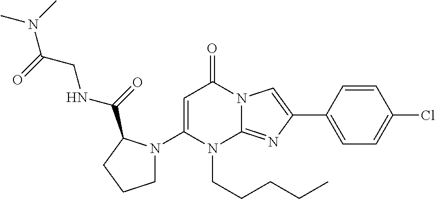 [SMILES: CCCCCn1c(N2CCC[C@H]2C(=O)NCC(=O)N(C)C)cc(=O)n2cc(-c3ccc(Cl)cc3)nc12] | A | 2.37 | 513 |
| 700 | 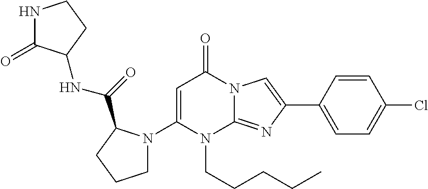 [SMILES: CCCCCn1c(N2CCC[C@H]2C(=O)NC2CCNC2=O)cc(=O)n2cc(-c3ccc(Cl)cc3)nc12] | A | 2.21 | 511 |
| 701 | 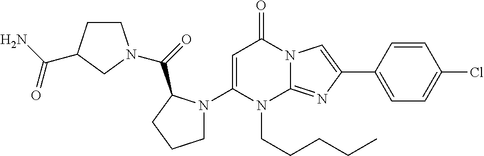 [SMILES: CCCCCn1c(N2CCC[C@H]2C(=O)N2CCC(C(N)=O)C2)cc(=O)n2cc(-c3ccc(Cl)cc3)nc12] | A | 2.18 | 525 |

TABLE 145

| | | | Retention | |
|---|---|---|---|---|
| | | | Time | |
| Compound | Structure | LC/MS | [min] | Mass [M + H] |
| 702 | 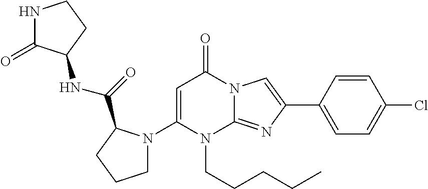 [SMILES: CCCCCn1c(N2CCC[C@H]2C(=O)N[C@@H]2CCNC2=O)cc(=O)n2cc(-c3ccc(Cl)cc3)nc12] | A | 2.21 | 511 |
| 703 | 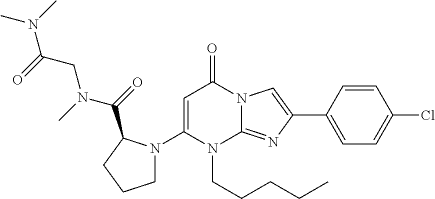 [SMILES: CCCCCn1c(N2CCC[C@H]2C(=O)N(C)CC(=O)N(C)C)cc(=O)n2cc(-c3ccc(Cl)cc3)nc12] | A | 2.36 | 527 |
| 704 | 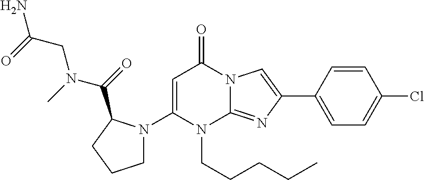 [SMILES: CCCCCn1c(N2CCC[C@H]2C(=O)N(C)CC(N)=O)cc(=O)n2cc(-c3ccc(Cl)cc3)nc12] | A | 2.21 | 499 |
| 705 | 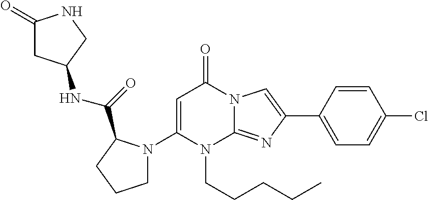 [SMILES: CCCCCn1c(N2CCC[C@H]2C(=O)N[C@@H]2CNC(=O)C2)cc(=O)n2cc(-c3ccc(Cl)cc3)nc12] | A | 2.15 | 511 |
| 706 | 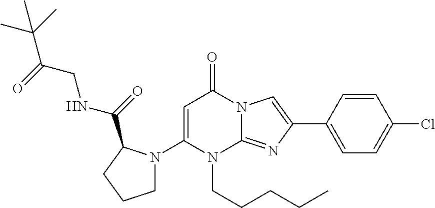 [SMILES: CCCCCn1c(N2CCC[C@H]2C(=O)NCC(=O)C(C)(C)C)cc(=O)n2cc(-c3ccc(Cl)cc3)nc12] | A | 2.81 | 526 |

TABLE 146

| | | | Retention | |
|---|---|---|---|---|
| | | | Time | |
| Compound | Structure | LC/MS | [min] | Mass [M + H] |
| 707 | 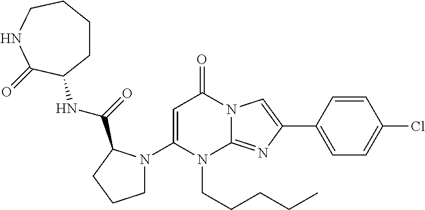 [SMILES: CCCCCn1c(N2CCC[C@H]2C(=O)N[C@H]2CCCCNC2=O)cc(=O)n2cc(-c3ccc(Cl)cc3)nc12] | A | 2.43 | 539 |
| 708 | 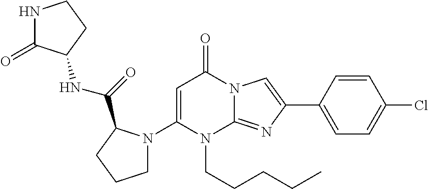 [SMILES: CCCCCn1c(N2CCC[C@H]2C(=O)N[C@H]2CCNC2=O)cc(=O)n2cc(-c3ccc(Cl)cc3)nc12] | A | 2.21 | 511 |
| 709 | 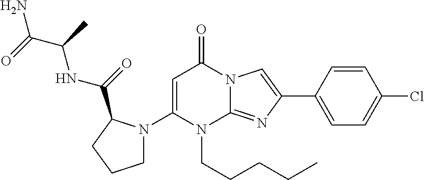 [SMILES: CCCCCn1c(N2CCC[C@H]2C(=O)N[C@H](C)C(N)=O)cc(=O)n2cc(-c3ccc(Cl)cc3)nc12] | A | 2.23 | 499 |
| 710 | 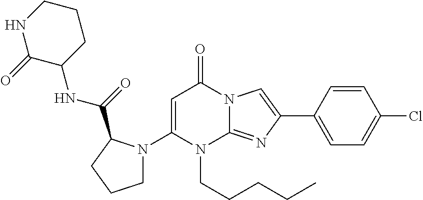 [SMILES: CCCCCn1c(N2CCC[C@H]2C(=O)NC2CCCNC2=O)cc(=O)n2cc(-c3ccc(Cl)cc3)nc12] | A | 2.26 | 525 |
| 711 | 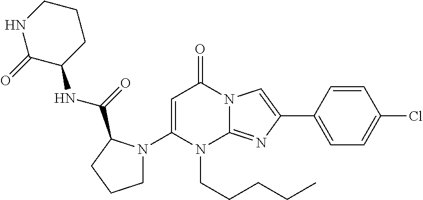 [SMILES: CCCCCn1c(N2CCC[C@H]2C(=O)N[C@@H]2CCCNC2=O)cc(=O)n2cc(-c3ccc(Cl)cc3)nc12] | A | 2.26 | 525 |

TABLE 147

| | | | Retention | |
|---|---|---|---|---|
| | | | Time | |
| Compound | Structure | LC/MS | [min] | Mass [M + H] |
| 712 | 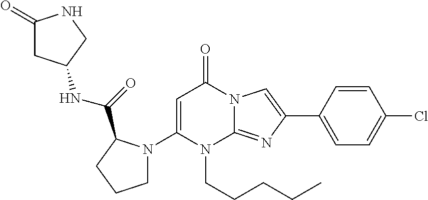 [SMILES: CCCCCn1c(N2CCC[C@H]2C(=O)N[C@H]2CNC(=O)C2)cc(=O)n2cc(-c3ccc(Cl)cc3)nc12] | A | 2.16 | 511 |
| 713 | 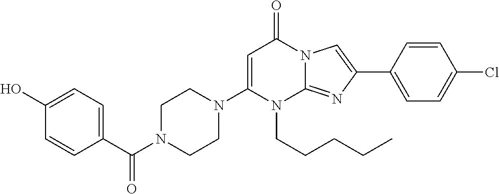 [SMILES: CCCCCn1c(N2CCN(C(=O)c3ccc(O)cc3)CC2)cc(=O)n2cc(-c3ccc(Cl)cc3)nc12] | B | 2.45 | 520 |
| 714 | 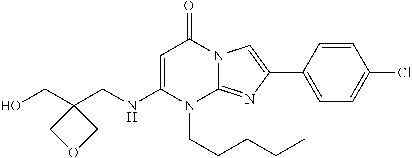 [SMILES: CCCCCn1c(NCC2(CO)COC2)cc(=O)n2cc(-c3ccc(Cl)cc3)nc12] | A | 2.17 | 431 |
| 715 | 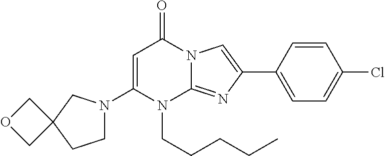 [SMILES: CCCCCn1c(N2CCC3(COC3)C2)cc(=O)n2cc(-c3ccc(Cl)cc3)nc12] | A | 2.48 | 427 |
| 716 | 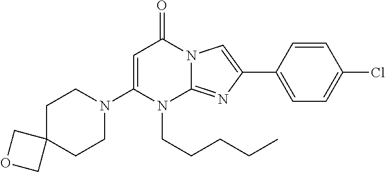 [SMILES: CCCCCn1c(N2CCC3(CC2)COC3)cc(=O)n2cc(-c3ccc(Cl)cc3)nc12] | A | 2.71 | 441 |

TABLE 148

| | | | Retention | |
|---|---|---|---|---|
| | | | Time | |
| Compound | Structure | LC/MS | [min] | Mass [M + H] |
| 717 | 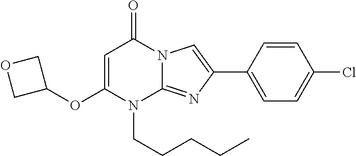 [SMILES: CCCCCn1c(OC2COC2)cc(=O)n2cc(-c3ccc(Cl)cc3)nc12] | A | 2.61 | 338 |
| 718 | 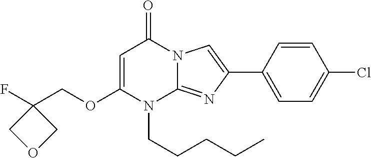 [SMILES: CCCCCn1c(OCC2(F)COC2)cc(=O)n2cc(-c3ccc(Cl)cc3)nc12] | A | 2.69 | 420 |
| 719 | 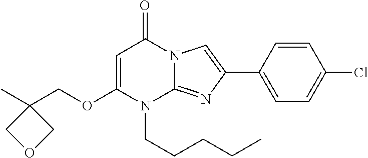 [SMILES: CCCCCn1c(OCC2(C)COC2)cc(=O)n2cc(-c3ccc(Cl)cc3)nc12] | A | 2.69 | 416 |
| 720 | 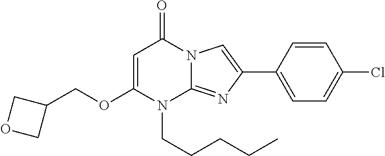 [SMILES: CCCCCn1c(OCC2COC2)cc(=O)n2cc(-c3ccc(Cl)cc3)nc12] | A | 2.6 | 402 |
| 721 | 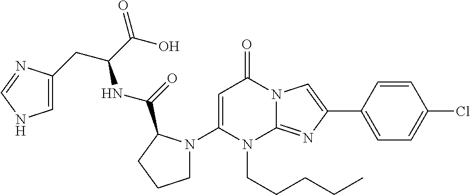 [SMILES: CCCCCn1c(N2CCC[C@H]2C(=O)N[C@@H](Cc2c[nH]cn2)C(=O)O)cc(=O)n2cc(-c3ccc(Cl)cc3)nc12] | A | 1.89 | 566 |

TABLE 149

| | | | Retention | |
|---|---|---|---|---|
| | | | Time | |
| Compound | Structure | LC/MS | [min] | Mass [M + H] |
| 722 | 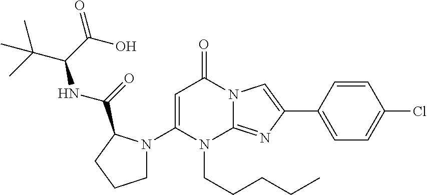 [SMILES: CCCCCn1c(N2CCC[C@H]2C(=O)N[C@H](C(=O)O)C(C)(C)C)cc(=O)n2cc(-c3ccc(Cl)cc3)nc12] | A | 2.60 | 542 |
| 723 | 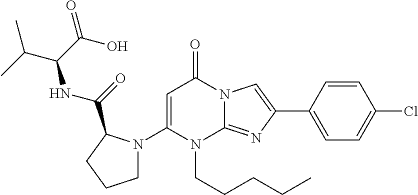 [SMILES: CCCCCn1c(N2CCC[C@H]2C(=O)N[C@H](C(=O)O)C(C)C)cc(=O)n2cc(-c3ccc(Cl)cc3)nc12] | A | 2.50 | 528 |
| 724 | 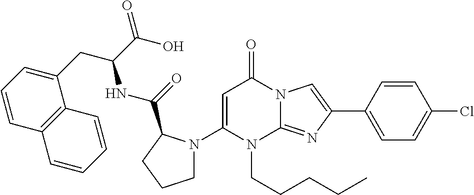 [SMILES: CCCCCn1c(N2CCC[C@H]2C(=O)N[C@@H](Cc2cccc3ccccc23)C(=O)O)cc(=O)n2cc(-c3ccc(Cl)cc3)nc12] | A | 2.81 | 626 |
| 725 | 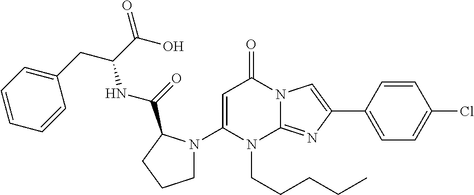 [SMILES: CCCCCn1c(N2CCC[C@H]2C(=O)N[C@H](Cc2ccccc2)C(=O)O)cc(=O)n2cc(-c3ccc(Cl)cc3)nc12] | A | 2.63 | 576 |
| 726 | 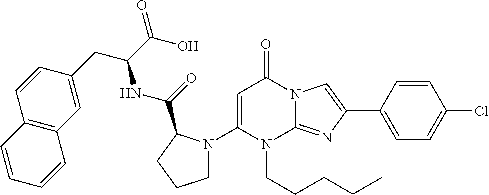 [SMILES: CCCCCn1c(N2CCC[C@H]2C(=O)N[C@@H](Cc2ccc3ccccc3c2)C(=O)O)cc(=O)n2cc(-c3ccc(Cl)cc3)nc12] | A | 2.80 | 626 |

TABLE 150

| | | | Retention | |
|---|---|---|---|---|
| | | | Time | |
| Compound | Structure | LC/MS | [min] | Mass [M + H] |
| 727 | 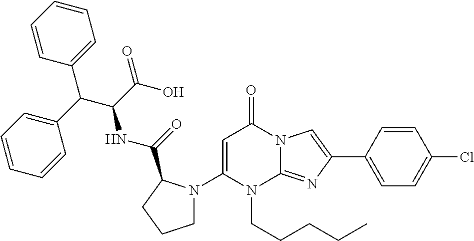 [SMILES: CCCCCn1c(N2CCC[C@H]2C(=O)N[C@H](C(=O)O)C(c2ccccc2)c2ccccc2)cc(=O)n2cc(-c3ccc(Cl)cc3)nc12] | A | 2.86 | 652 |
| 728 | 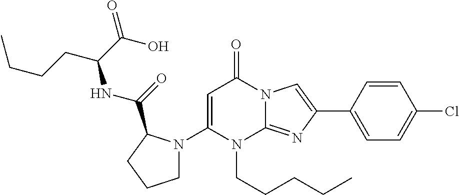 [SMILES: CCCCCn1c(N2CCC[C@H]2C(=O)N[C@@H](CCCC)C(=O)O)cc(=O)n2cc(-c3ccc(Cl)cc3)nc12] | A | 2.62 | 542 |
| 729 | 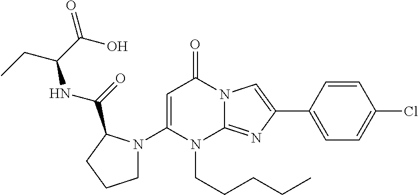 [SMILES: CCCCCn1c(N2CCC[C@H]2C(=O)N[C@@H](CC)C(=O)O)cc(=O)n2cc(-c3ccc(Cl)cc3)nc12] | A | 2.41 | 514 |
| 730 | 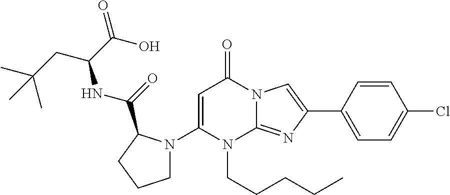 [SMILES: CCCCCn1c(N2CCC[C@H]2C(=O)N[C@@H](CC(C)(C)C)C(=O)O)cc(=O)n2cc(-c3ccc(Cl)cc3)nc12] | A | 2.68 | 556 |
| 731 | 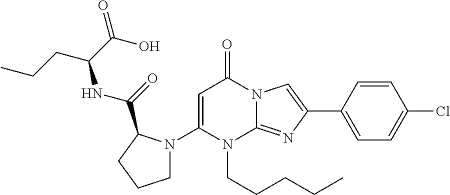 [SMILES: CCCCCn1c(N2CCC[C@H]2C(=O)N[C@@H](CCC)C(=O)O)cc(=O)n2cc(-c3ccc(Cl)cc3)nc12] | A | 2.51 | 528 |

TABLE 151

| | | | Retention | |
|---|---|---|---|---|
| | | | Time | Mass |
| Compound | Structure | LC/MS | [min] | [M + H] |
| 732 | 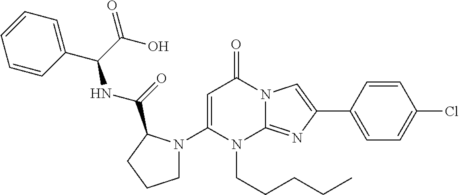 [SMILES: CCCCCn1c(N2CCC[C@H]2C(=O)N[C@H](C(=O)O)c2ccccc2)cc(=O)n2cc(-c3ccc(Cl)cc3)nc12] | A | 2.56 | 562 |
| 733 | 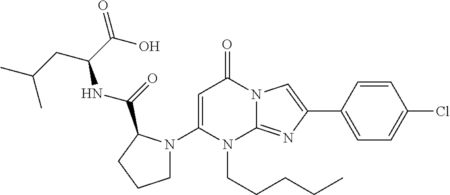 [SMILES: CCCCCn1c(N2CCC[C@H]2C(=O)N[C@@H](CC(C)C)C(=O)O)cc(=O)n2cc(-c3ccc(Cl)cc3)nc12] | A | 2.60 | 542 |
| 734 | 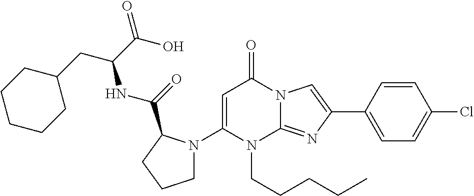 [SMILES: CCCCCn1c(N2CCC[C@H]2C(=O)N[C@@H](CC2CCCCC2)C(=O)O)cc(=O)n2cc(-c3ccc(Cl)cc3)nc12] | A | 2.83 | 582 |
| 735 | 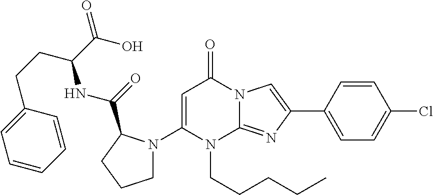 [SMILES: CCCCCn1c(N2CCC[C@H]2C(=O)N[C@@H](CCc2ccccc2)C(=O)O)cc(=O)n2cc(-c3ccc(Cl)cc3)nc12] | A | 2.69 | 590 |
| 736 | 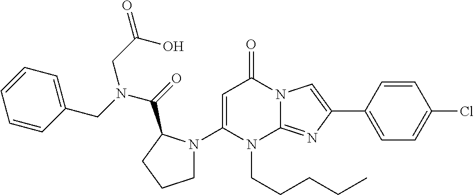 [SMILES: CCCCCn1c(N2CCC[C@H]2C(=O)N(CC(=O)O)Cc2ccccc2)cc(=O)n2cc(-c3ccc(Cl)cc3)nc12] | A | 2.73 | 576 |

TABLE 152

| | | | Retention | |
|---|---|---|---|---|
| | | | Time | Mass |
| Compound | Structure | LC/MS | [min] | [M + H] |
| 737 | 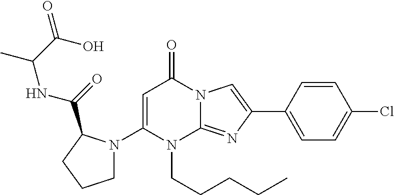 [SMILES: CCCCCn1c(N2CCC[C@H]2C(=O)NC(C)C(=O)O)cc(=O)n2cc(-c3ccc(Cl)cc3)nc12] | A | 2.34 | 500 |
| 738 | 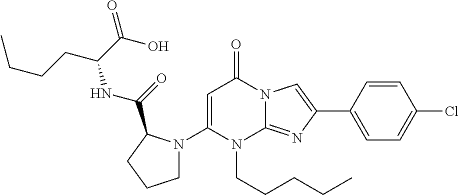 [SMILES: CCCCCn1c(N2CCC[C@H]2C(=O)N[C@H](CCCC)C(=O)O)cc(=O)n2cc(-c3ccc(Cl)cc3)nc12] | A | 2.63 | 542 |
| 739 | 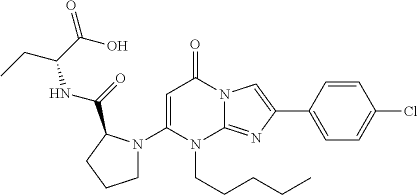 [SMILES: CCCCCn1c(N2CCC[C@H]2C(=O)N[C@H](CC)C(=O)O)cc(=O)n2cc(-c3ccc(Cl)cc3)nc12] | A | 2.43 | 514 |
| 740 | 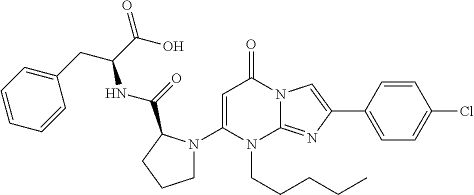 [SMILES: CCCCCn1c(N2CCC[C@H]2C(=O)N[C@@H](Cc2ccccc2)C(=O)O)cc(=O)n2cc(-c3ccc(Cl)cc3)nc12] | A | 2.64 | 576 |
| 741 | 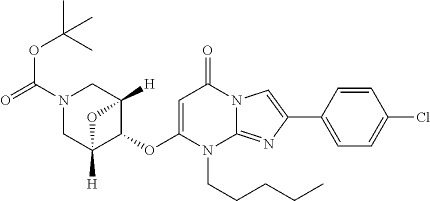 | C | 2.85 | 557 |

TABLE 153

| | | | Retention | |
|---|---|---|---|---|
| | | | Time | Mass |
| Compound | Structure | LC/MS | [min] | [M + H] |
| 742 | 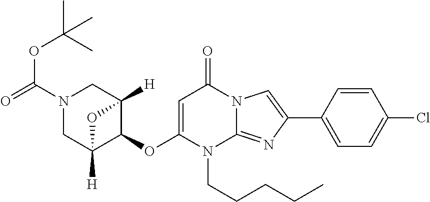 | C | 2.85 | 557 |
| 743 | 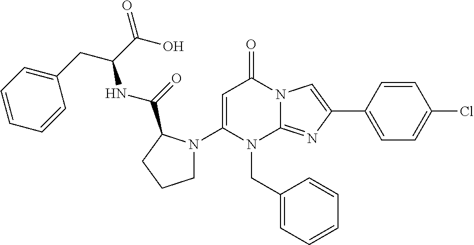 [SMILES: O=C(O)[C@H](Cc1ccccc1)NC(=O)[C@@H]1CCCN1c1cc(=O)n2cc(-c3ccc(Cl)cc3)nc2n1Cc1ccccc1] | A | 2.44 | 596 |
| 744 | 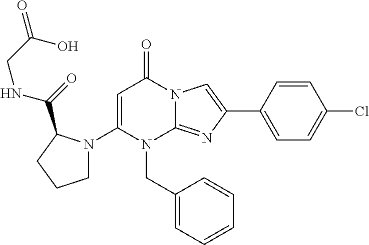 [SMILES: O=C(O)CNC(=O)[C@@H]1CCCN1c1cc(=O)n2cc(-c3ccc(Cl)cc3)nc2n1Cc1ccccc1] | A | 2.08 | 506 |
| 745 | 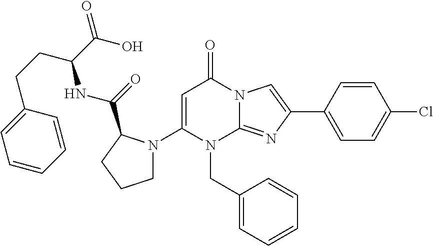 [SMILES: O=C(O)[C@H](CCc1ccccc1)NC(=O)[C@@H]1CCCN1c1cc(=O)n2cc(-c3ccc(Cl)cc3)nc2n1Cc1ccccc1] | A | 2.51 | 610 |
| 746 | 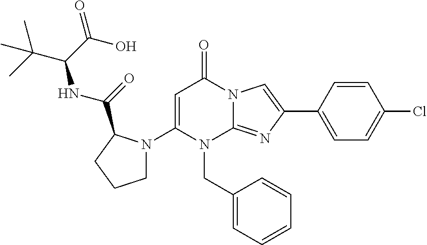 [SMILES: CC(C)(C)[C@H](NC(=O)[C@@H]1CCCN1c1cc(=O)n2cc(-c3ccc(Cl)cc3)nc2n1Cc1ccccc1)C(=O)O] | A | 2.39 | 562 |

TABLE 154

| | | | Retention | |
|---|---|---|---|---|
| | | | Time | Mass |
| Compound | Structure | LC/MS | [min] | [M + H] |
| 747 | 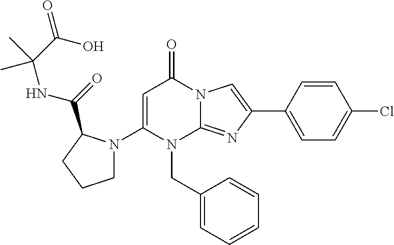 [SMILES: CC(C)(NC(=O)[C@@H]1CCCN1c1cc(=O)n2cc(-c3ccc(Cl)cc3)nc2n1Cc1ccccc1)C(=O)O] | A | 2.20 | 534 |
| 748 | 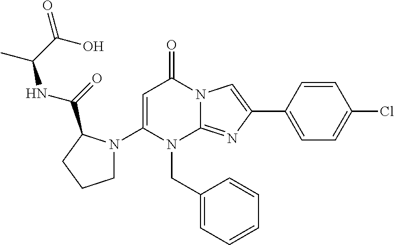 [SMILES: C[C@H](NC(=O)[C@@H]1CCCN1c1cc(=O)n2cc(-c3ccc(Cl)cc3)nc2n1Cc1ccccc1)C(=O)O] | A | 2.14 | 520 |
| 749 | 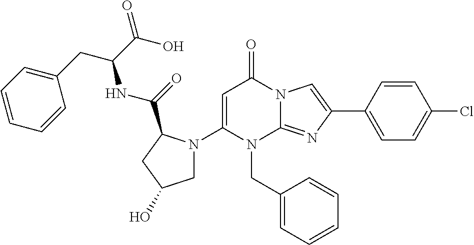 [SMILES: O=C(O)[C@H](Cc1ccccc1)NC(=O)[C@@H]1C[C@@H](O)CN1c1cc(=O)n2cc(-c3ccc(Cl)cc3)nc2n1Cc1ccccc1] | A | 2.20 | 612 |
| 750 | 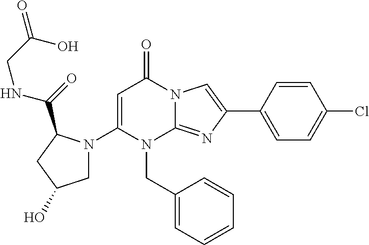 [SMILES: O=C(O)CNC(=O)[C@@H]1C[C@@H](O)CN1c1cc(=O)n2cc(-c3ccc(Cl)cc3)nc2n1Cc1ccccc1] | A | 1.86 | 522 |
| 751 | 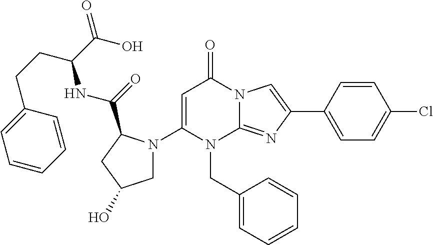 [SMILES: O=C(O)[C@H](CCc1ccccc1)NC(=O)[C@@H]1C[C@@H](O)CN1c1cc(=O)n2cc(-c3ccc(Cl)cc3)nc2n1Cc1ccccc1] | A | 2.27 | 626 |

TABLE 155

| | | | Retention | |
|---|---|---|---|---|
| | | | Time | Mass |
| Compound | Structure | LC/MS | [min] | [M+H] |
| 752 | 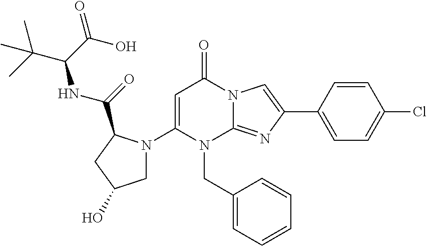 [SMILES: CC(C)(C)[C@H](NC(=O)[C@@H]1C[C@@H](O)CN1c1cc(=O)n2cc(-c3ccc(Cl)cc3)nc2n1Cc1ccccc1)C(=O)O] | A | 2.15 | 578 |
| 753 | 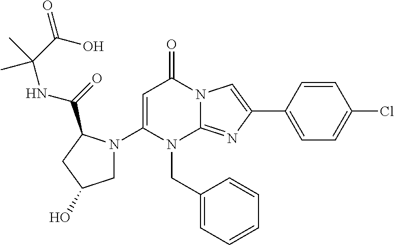 [SMILES: CC(C)(NC(=O)[C@@H]1C[C@@H](O)CN1c1cc(=O)n2cc(-c3ccc(Cl)cc3)nc2n1Cc1ccccc1)C(=O)O] | A | 1.96 | 550 |
| 754 | 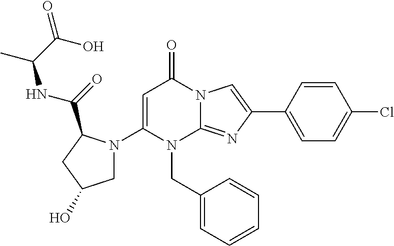 [SMILES: C[C@H](NC(=O)[C@@H]1C[C@@H](O)CN1c1cc(=O)n2cc(-c3ccc(Cl)cc3)nc2n1Cc1ccccc1)C(=O)O] | A | 1.91 | 536 |
| 755 | 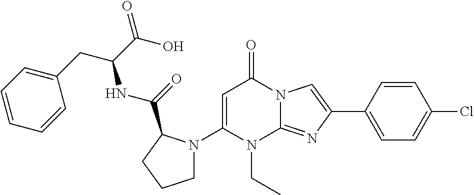 [SMILES: CCn1c(N2CCC[C@H]2C(=O)N[C@@H](Cc2ccccc2)C(=O)O)cc(=O)n2cc(-c3ccc(Cl)cc3)nc12] | A | 2.18 | 534 |
| 756 | 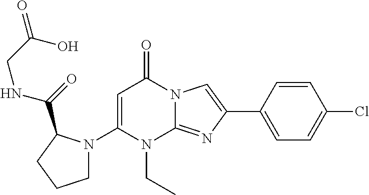 [SMILES: CCn1c(N2CCC[C@H]2C(=O)NCC(=O)O)cc(=O)n2cc(-c3ccc(Cl)cc3)nc12] | A | 1.81 | 444 |

TABLE 156

| | | | Retention | |
|---|---|---|---|---|
| | | | Time | Mass |
| Compound | Structure | LC/MS | [min] | [M+H] |
| 757 | 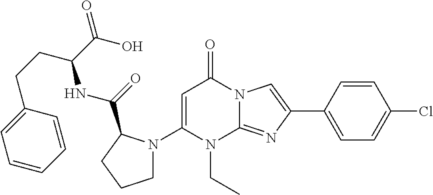 [SMILES: CCn1c(N2CCC[C@H]2C(=O)N[C@@H](CCc2ccccc2)C(=O)O)cc(=O)n2cc(-c3ccc(Cl)cc3)nc12] | A | 2.27 | 548 |
| 758 | 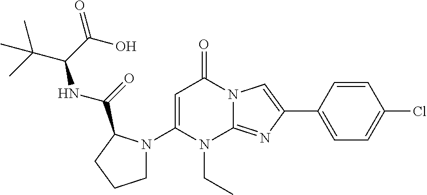 [SMILES: CCn1c(N2CCC[C@H]2C(=O)N[C@H](C(=O)O)C(C)(C)C)cc(=O)n2cc(-c3ccc(Cl)cc3)nc12] | A | 2.14 | 500 |
| 759 | 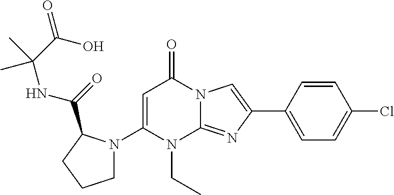 [SMILES: CCn1c(N2CCC[C@H]2C(=O)NC(C)(C)C(=O)O)cc(=O)n2cc(-c3ccc(Cl)cc3)nc12] | A | 1.93 | 472 |
| 760 | 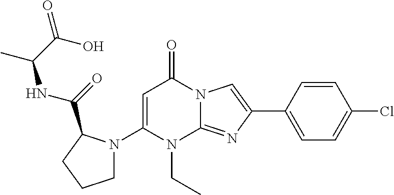 [SMILES: CCn1c(N2CCC[C@H]2C(=O)N[C@@H](C)C(=O)O)cc(=O)n2cc(-c3ccc(Cl)cc3)nc12] | A | 1.86 | 458 |
| 761 | 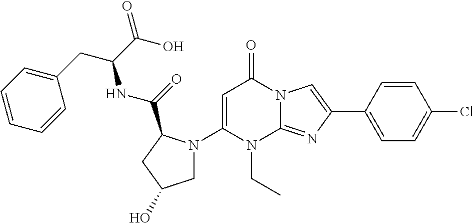 [SMILES: CCn1c(N2C[C@H](O)C[C@H]2C(=O)N[C@@H](Cc2ccccc2)C(=O)O)cc(=O)n2cc(-c3ccc(Cl)cc3)nc12] | A | 1.95 | 550 |

TABLE 157

| | | | Retention | |
|---|---|---|---|---|
| | | | Time | Mass |
| Compound | Structure | LC/MS | [min] | [M+H] |
| 762 | 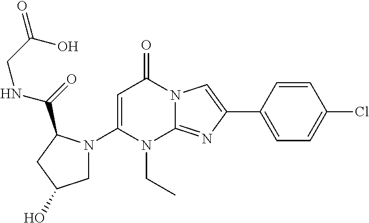 [SMILES: CCn1c(N2C[C@H](O)C[C@H]2C(=O)NCC(=O)O)cc(=O)n2cc(-c3ccc(Cl)cc3)nc12] | A | 1.57 | 460 |
| 763 | 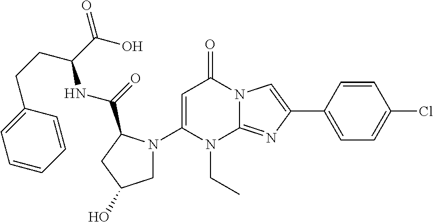 [SMILES: CCn1c(N2C[C@H](O)C[C@H]2C(=O)N[C@@H](CCc2ccccc2)C(=O)O)cc(=O)n2cc(-c3ccc(Cl)cc3)nc12] | A | 2.05 | 564 |
| 764 | 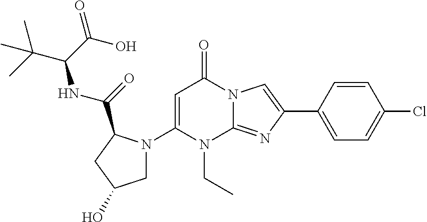 [SMILES: CCn1c(N2C[C@H](O)C[C@H]2C(=O)N[C@H](C(=O)O)C(C)(C)C)cc(=O)n2cc(-c3ccc(Cl)cc3)nc12] | A | 1.90 | 516 |
| 765 | 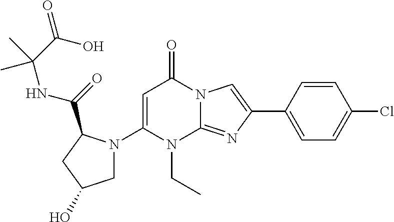 [SMILES: CCn1c(N2C[C@H](O)C[C@H]2C(=O)NC(C)(C)C(=O)O)cc(=O)n2cc(-c3ccc(Cl)cc3)nc12] | A | 1.69 | 488 |
| 766 | 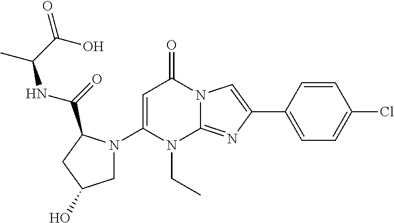 [SMILES: CCn1c(N2C[C@H](O)C[C@H]2C(=O)N[C@@H](C)C(=O)O)cc(=O)n2cc(-c3ccc(Cl)cc3)nc12] | A | 1.64 | 474 |

TABLE 158

| | | | Retention | |
|---|---|---|---|---|
| | | | Time | Mass |
| Compound | Structure | LC/MS | [min] | [M+H] |
| 767 | 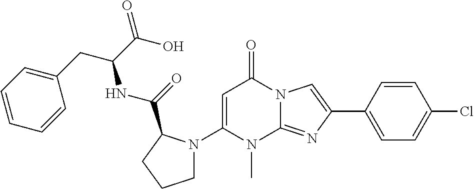 [SMILES: Cn1c(N2CCC[C@H]2C(=O)N[C@@H](Cc2ccccc2)C(=O)O)cc(=O)n2cc(-c3ccc(Cl)cc3)nc12] | A | 1.98 | 520 |
| 768 | 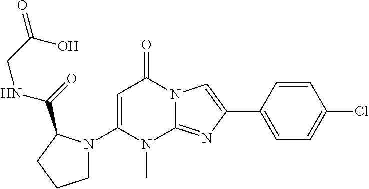 [SMILES: Cn1c(N2CCC[C@H]2C(=O)NCC(=O)O)cc(=O)n2cc(-c3ccc(Cl)cc3)nc12] | A | 1.58 | 430 |
| 769 | 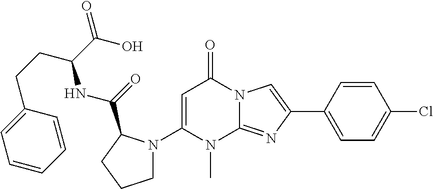 [SMILES: Cn1c(N2CCC[C@H]2C(=O)N[C@@H](CCc2ccccc2)C(=O)O)cc(=O)n2cc(-c3ccc(Cl)cc3)nc12] | A | 2.07 | 534 |
| 770 | 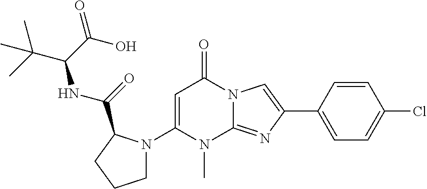 [SMILES: Cn1c(N2CCC[C@H]2C(=O)N[C@H](C(=O)O)C(C)(C)C)cc(=O)n2cc(-c3ccc(Cl)cc3)nc12] | A | 1.90 | 486 |
| 771 | 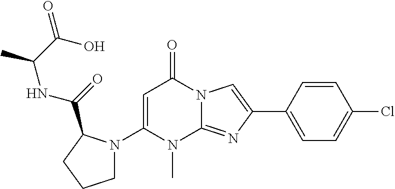 [SMILES: C[C@H](NC(=O)[C@@H]1CCCN1c1cc(=O)n2cc(-c3ccc(Cl)cc3)nc2n1C)C(=O)O] | A | 1.64 | 444 |

TABLE 159

| | | | Retention | |
|---|---|---|---|---|
| | | | Time | Mass |
| Compound | Structure | LC/MS | [min] | [M+H] |
| 772 | 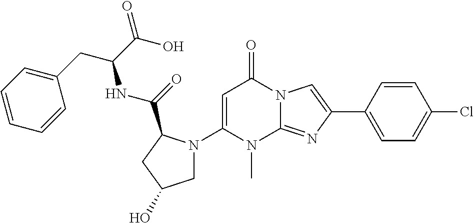 [SMILES: Cn1c(N2C[C@H](O)C[C@H]2C(=O)N[C@@H](Cc2ccccc2)C(=O)O)cc(=O)n2cc(-c3ccc(Cl)cc3)nc12] | A | 1.78 | 536 |
| 773 | 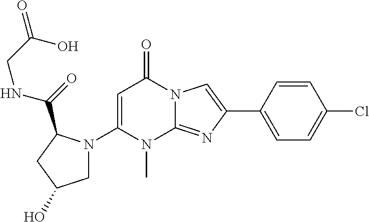 [SMILES: Cn1c(N2C[C@H](O)C[C@H]2C(=O)NCC(=O)O)cc(=O)n2cc(-c3ccc(Cl)cc3)nc12] | A | 1.40 | 446 |
| 774 | 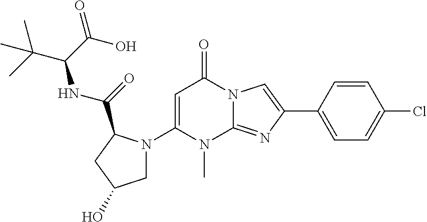 [SMILES: Cn1c(N2C[C@H](O)C[C@H]2C(=O)N[C@H](C(=O)O)C(C)(C)C)cc(=O)n2cc(-c3ccc(Cl)cc3)nc12] | A | 1.72 | 502 |
| 775 | 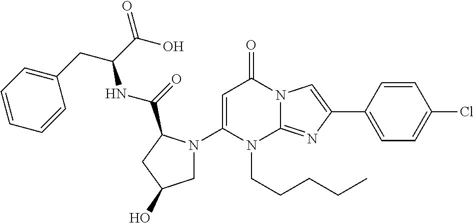 [SMILES: CCCCCn1c(N2C[C@@H](O)C[C@H]2C(=O)N[C@@H](Cc2ccccc2)C(=O)O)cc(=O)n2cc(-c3ccc(Cl)cc3)nc12] | A | 2.41 | 592 |
| 776 | 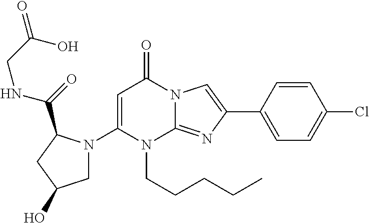 [SMILES: CCCCCn1c(N2C[C@@H](O)C[C@H]2C(=O)NCC(=O)O)cc(=O)n2cc(-c3ccc(Cl)cc3)nc12] | A | 2.03 | 502 |

TABLE 160

| | | | Retention | |
|---|---|---|---|---|
| | | | Time | Mass |
| Compound | Structure | LC/MS | [min] | [M+H] |
| 777 | 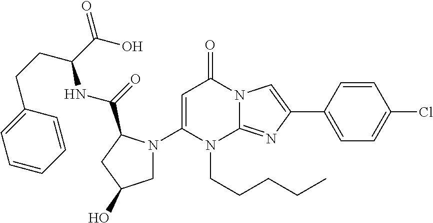 [SMILES: CCCCCn1c(N2C[C@@H](O)C[C@H]2C(=O)N[C@@H](CCc2ccccc2)C(=O)O)cc(=O)n2cc(-c3ccc(Cl)cc3)nc12] | A | 2.48 | 606 |
| 778 | 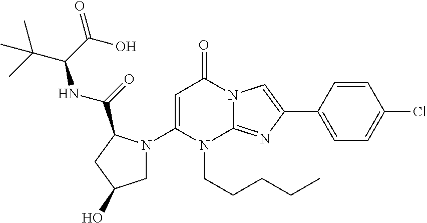 [SMILES: CCCCCn1c(N2C[C@@H](O)C[C@H]2C(=O)N[C@H](C(=O)O)C(C)(C)C)cc(=O)n2cc(-c3ccc(Cl)cc3)nc12] | A | 2.37 | 558 |
| 779 | 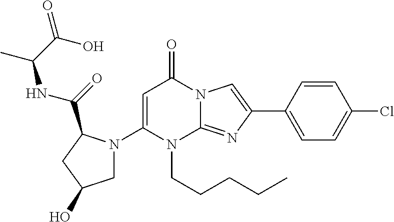 [SMILES: CCCCCn1c(N2C[C@@H](O)C[C@H]2C(=O)N[C@@H](C)C(=O)O)cc(=O)n2cc(-c3ccc(Cl)cc3)nc12] | A | 2.10 | 516 |

TABLE 161

| | | | Retention | |
|---|---|---|---|---|
| | | | Time | Mass |
| Compound | Structure | LC/MS | [min] | [M+H] |
| 780 | 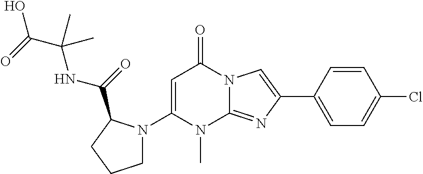 [SMILES: Cn1c(N2CCC[C@H]2C(=O)NC(C)(C)C(=O)O)cc(=O)n2cc(-c3ccc(Cl)cc3)nc12] | A | 1.69 | 458 |
| 781 | 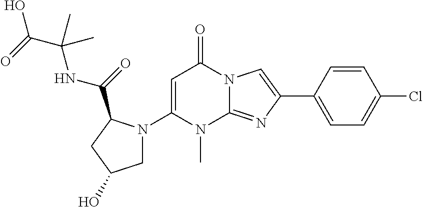 [SMILES: Cn1c(N2C[C@H](O)C[C@H]2C(=O)NC(C)(C)C(=O)O)cc(=O)n2cc(-c3ccc(Cl)cc3)nc12] | A | 1.51 | 474 |
| 782 | 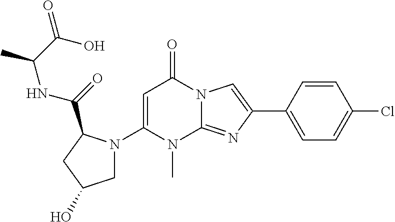 [SMILES: C[C@H](NC(=O)[C@@H]1C[C@@H](O)CN1c1cc(=O)n2cc(-c3ccc(Cl)cc3)nc2n1C)C(=O)O] | A | 1.46 | 460 |
| 783 | 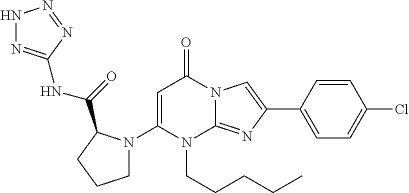 [SMILES: CCCCCn1c(N2CCC[C@H]2C(=O)Nc2nn[nH]n2)cc(=O)n2cc(-c3ccc(Cl)cc3)nc12] | A | 2.38 | 496 |
| 784 | 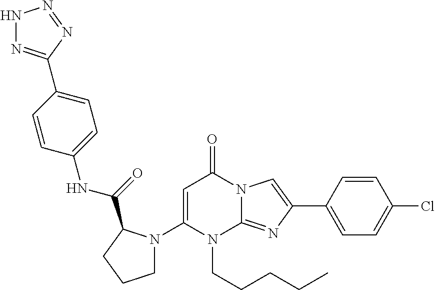 [SMILES: CCCCCn1c(N2CCC[C@H]2C(=O)Nc2ccc(-c3nn[nH]n3)cc2)cc(=O)n2cc(-c3ccc(Cl)cc3)nc12] | A | 2.54 | 572 |

TABLE 162

| | | | Retention | |
|---|---|---|---|---|
| | | | Time | Mass |
| Compound | Structure | LC/MS | [min] | [M+H] |
| 785 | 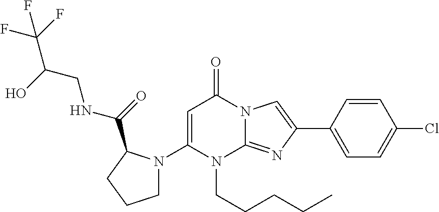 [SMILES: CCCCCn1c(N2CCC[C@H]2C(=O)NCC(O)C(F)(F)F)cc(=O)n2cc(-c3ccc(Cl)cc3)nc12] | A | 2.57 | 540 |
| 786 | 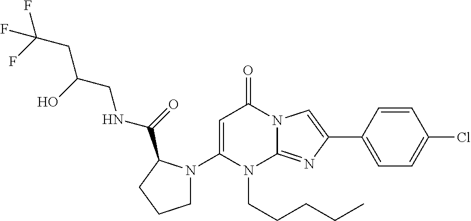 [SMILES: CCCCCn1c(N2CCC[C@H]2C(=O)NCC(O)CC(F)(F)F)cc(=O)n2cc(-c3ccc(Cl)cc3)nc12] | A | 2.53 | 554 |
| 787 | 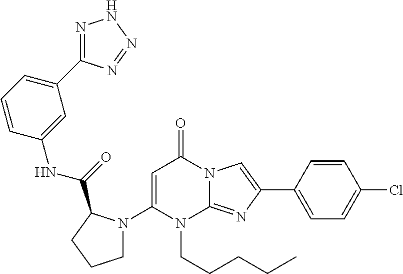 [SMILES: CCCCCn1c(N2CCC[C@H]2C(=O)Nc2cccc(-c3nn[nH]n3)c2)cc(=O)n2cc(-c3ccc(Cl)cc3)nc12] | A | 2.56 | 572 |
| 788 | 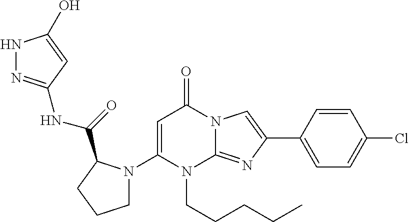 [SMILES: CCCCCn1c(N2CCC[C@H]2C(=O)Nc2cc(O)[nH]n2)cc(=O)n2cc(-c3ccc(Cl)cc3)nc12] | A | 2.34 | 510 |
| 789 | 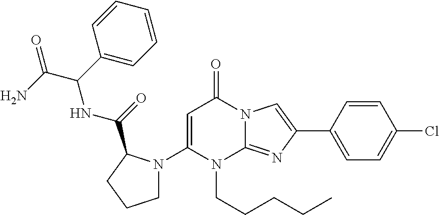 [SMILES: CCCCCn1c(N2CCC[C@H]2C(=O)NC(C(N)=O)c2ccccc2)cc(=O)n2cc(-c3ccc(Cl)cc3)nc12] | A | 2.50 | 561 |

TABLE 163

| | | | Retention | |
|---|---|---|---|---|
| | | | Time | Mass |
| Compound | Structure | LC/MS | [min] | [M+H] |
| 790 | 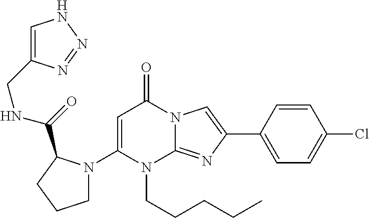 [SMILES: CCCCCn1c(N2CCC[C@H]2C(=O)NCc2c[nH]nn2)cc(=O)n2cc(-c3ccc(Cl)cc3)nc12] | A | 2.25 | 509 |
| 791 | 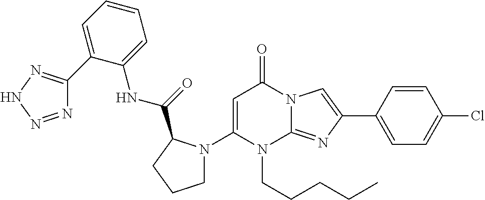 [SMILES: CCCCCn1c(N2CCC[C@H]2C(=O)Nc2ccccc2-c2nn[nH]n2)cc(=O)n2cc(-c3ccc(Cl)cc3)nc12] | A | 2.68 | 572 |
| 792 | 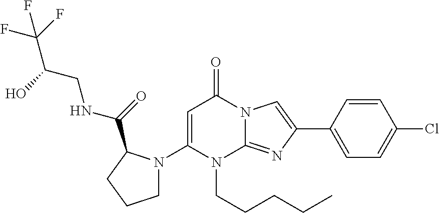 [SMILES: CCCCCn1c(N2CCC[C@H]2C(=O)NC[C@H](O)C(F)(F)F)cc(=O)n2cc(-c3ccc(Cl)cc3)nc12] | A | 2.56 | 540 |
| 793 | 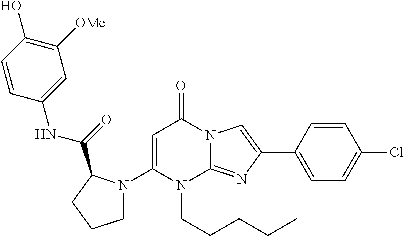 [SMILES: CCCCCn1c(N2CCC[C@H]2C(=O)Nc2ccc(O)c(OC)c2)cc(=O)n2cc(-c3ccc(Cl)cc3)nc12] | A | 2.55 | 550 |
| 794 | 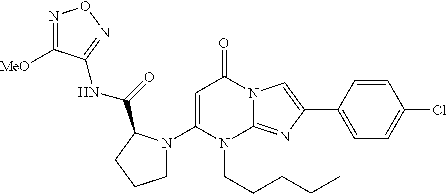 [SMILES: CCCCCn1c(N2CCC[C@H]2C(=O)Nc2nonc2OC)cc(=O)n2cc(-c3ccc(Cl)cc3)nc12] | A | 2.40 | 526 |

TABLE 164

| | | | Retention | |
|---|---|---|---|---|
| | | | Time | Mass |
| Compound | Structure | LC/MS | [min] | [M+H] |
| 795 | 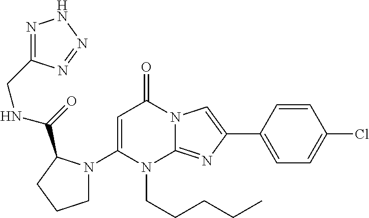 [SMILES: CCCCCn1c(N2CCC[C@H]2C(=O)NCc2nn[nH]n2)cc(=O)n2cc(-c3ccc(Cl)cc3)nc12] | A | 2.28 | 510 |
| 796 | 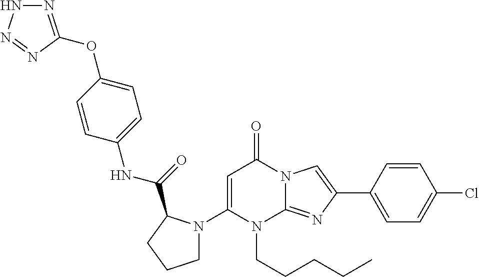 [SMILES: CCCCCn1c(N2CCC[C@H]2C(=O)Nc2ccc(Oc3nn[nH]n3)cc2)cc(=O)n2cc(-c3ccc(Cl)cc3)nc12] | A | 2.53 | 588 |
| 797 | 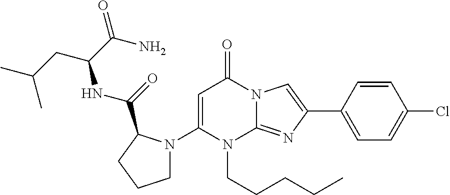 [SMILES: CCCCCn1c(N2CCC[C@H]2C(=O)N[C@@H](CC(C)C)C(N)=O)cc(=O)n2cc(-c3ccc(Cl)cc3)nc12] | A | 2.51 | 541 |
| 798 | 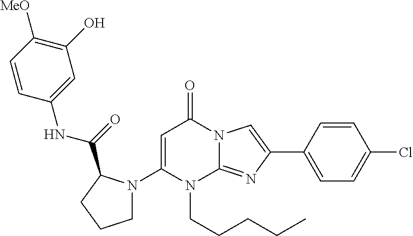 [SMILES: CCCCCn1c(N2CCC[C@H]2C(=O)Nc2ccc(OC)c(O)c2)cc(=O)n2cc(-c3ccc(Cl)cc3)nc12] | A | 2.57 | 550 |
| 799 | 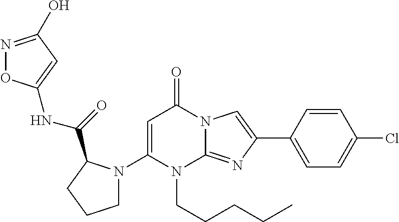 [SMILES: CCCCCn1c(N2CCC[C@H]2C(=O)Nc2cc(O)no2)cc(=O)n2cc(-c3ccc(Cl)cc3)nc12] | A | 2.44 | 511 |

TABLE 165

| | | | Retention | |
|---|---|---|---|---|
| | | | Time | Mass |
| Compound | Structure | LC/MS | [min] | [M+H] |
| 800 | 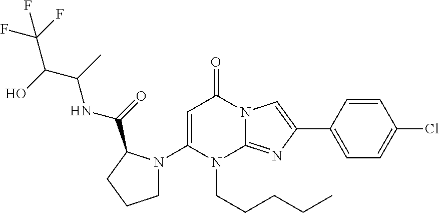 [SMILES: CCCCCn1c(N2CCC[C@H]2C(=O)NC(C)C(O)C(F)(F)F)cc(=O)n2cc(-c3ccc(Cl)cc3)nc12] | A | 2.62 | 554 |
| 801 | 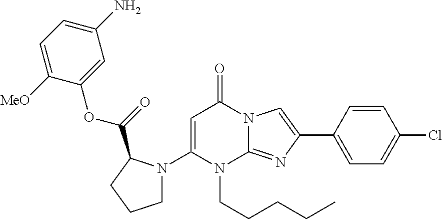 [SMILES: CCCCCn1c(N2CCC[C@H]2C(=O)Oc2cc(N)ccc2OC)cc(=O)n2cc(-c3ccc(Cl)cc3)nc12] | A | 2.48 | 550 |
| 802 | 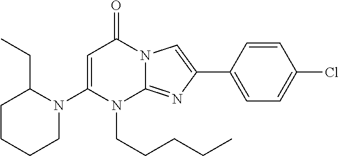 [SMILES: CCCCCn1c(N2CCCCC2CC)cc(=O)n2cc(-c3ccc(Cl)cc3)nc12] | A | 3.47 | 427 |
| 803 | 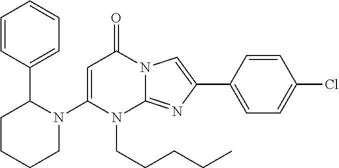 [SMILES: CCCCCn1c(N2CCCCC2c2ccccc2)cc(=O)n2cc(-c3ccc(Cl)cc3)nc12] | A | 3.49 | 475 |
| 804 | 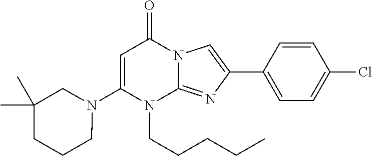 [SMILES: CCCCCn1c(N2CCCC(C)(C)C2)cc(=O)n2cc(-c3ccc(Cl)cc3)nc12] | A | 3.49 | 427 |

TABLE 166

| | | | Retention | |
|---|---|---|---|---|
| | | | Time | Mass |
| Compound | Structure | LC/MS | [min] | [M+H] |
| 805 | 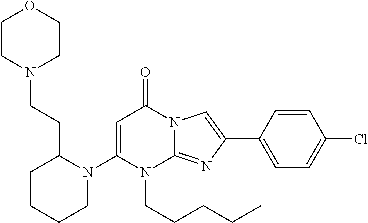 [SMILES: CCCCCn1c(N2CCCCC2CCN2CCOCC2)cc(=O)n2cc(-c3ccc(Cl)cc3)nc12] | A | 2.94 | 512 |
| 806 | 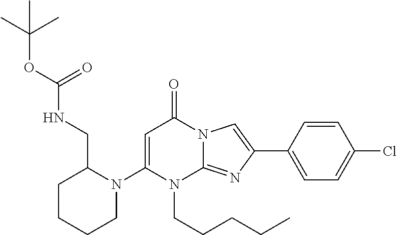 [SMILES: CCCCCn1c(N2CCCCC2CNC(=O)OC(C)(C)C)cc(=O)n2cc(-c3ccc(Cl)cc3)nc12] | A | 3.21 | 528 |
| 807 | 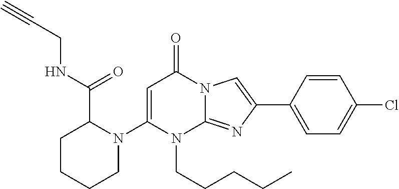 [SMILES: C#CCNC(=O)C1CCCCN1c1cc(=O)n2cc(-c3ccc(Cl)cc3)nc2n1CCCCC] | A | 2.65 | 480 |
| 808 | 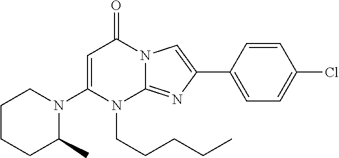 [SMILES: CCCCCn1c(N2CCCC[C@@H]2C)cc(=O)n2cc(-c3ccc(Cl)cc3)nc12] | A | 3.40 | 413 |
| 809 | 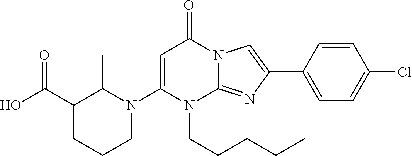 [SMILES: CCCCCn1c(N2CCCC(C(=O)O)C2C)cc(=O)n2cc(-c3ccc(Cl)cc3)nc12] | A | 2.67 | 457 |

TABLE 167

| | | | Retention | |
|---|---|---|---|---|
| | | | Time | Mass |
| Compound | Structure | LC/MS | [min] | [M+H] |
| 810 | 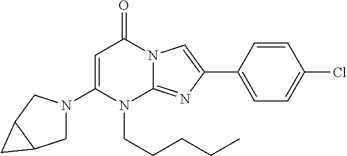 [SMILES: CCCCCn1c(N2CC3CC3C2)cc(=O)n2cc(-c3ccc(Cl)cc3)nc12] | A | 3.07 | 397 |
| 811 | 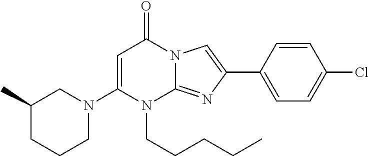 [SMILES: CCCCCn1c(N2CCC[C@@H](C)C2)cc(=O)n2cc(-c3ccc(Cl)cc3)nc12] | A | 3.39 | 413 |
| 812 | 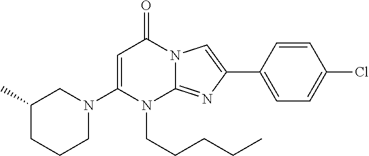 [SMILES: CCCCCn1c(N2CCC[C@H](C)C2)cc(=O)n2cc(-c3ccc(Cl)cc3)nc12] | A | 3.40 | 413 |
| 813 | 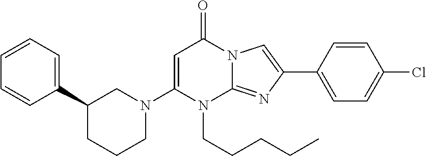 [SMILES: CCCCCn1c(N2CCC[C@@H](c3ccccc3)C2)cc(=O)n2cc(-c3ccc(Cl)cc3)nc12] | A | 3.49 | 476 |
| 814 | 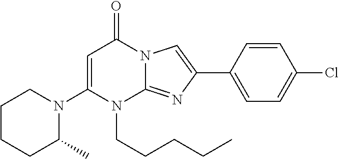 [SMILES: CCCCCn1c(N2CCCC[C@H]2C)cc(=O)n2cc(-c3ccc(Cl)cc3)nc12] | A | 3.40 | 413 |

TABLE 168

| | | | Retention | |
|---|---|---|---|---|
| | | | Time | Mass |
| Compound | Structure | LC/MS | [min] | [M+H] |
| 815 | 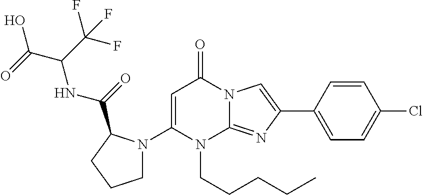 [SMILES: CCCCCn1c(N2CCC[C@H]2C(=O)NC(C(=O)O)C(F)(F)F)cc(=O)n2cc(-c3ccc(Cl)cc3)nc12] | B | 2.69 | 554 |
| 816 | 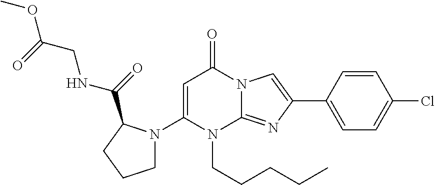 [SMILES: CCCCCn1c(N2CCC[C@H]2C(=O)NCC(=O)OC)cc(=O)n2cc(-c3ccc(Cl)cc3)nc12] | B | 2.44 | 500 |
| 817 | 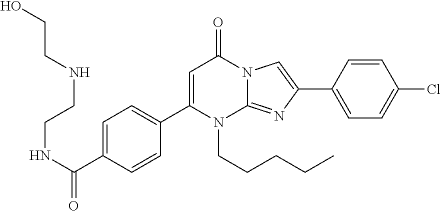 [SMILES: CCCCCn1c(-c2ccc(C(=O)NCCNCCO)cc2)cc(=O)n2cc(-c3ccc(Cl)cc3)nc12] | B | 1.97 | 522 |
| 818 | 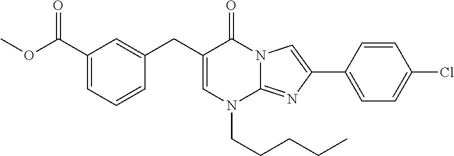 [SMILES: CCCCCn1cc(Cc2cccc(C(=O)OC)c2)c(=O)n2cc(-c3ccc(Cl)cc3)nc12] | B | 3.00 | 478 |
| 819 | 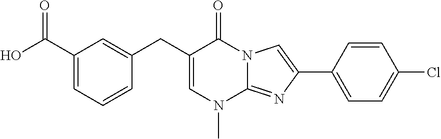 [SMILES: Cn1cc(Cc2cccc(C(=O)O)c2)c(=O)n2cc(-c3ccc(Cl)cc3)nc12] | B | 2.04 | 394 |

TABLE 169

| | | | Retention | |
|---|---|---|---|---|
| | | | Time | Mass |
| Compound | Structure | LC/MS | [min] | [M+H] |
| 820 | 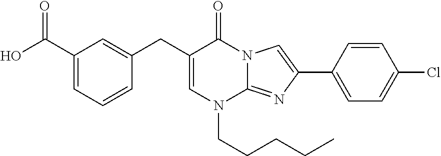 [SMILES: CCCCCn1cc(Cc2cccc(C(=O)O)c2)c(=O)n2cc(-c3ccc(Cl)cc3)nc12] | B | 2.66 | 450 |
| 821 | 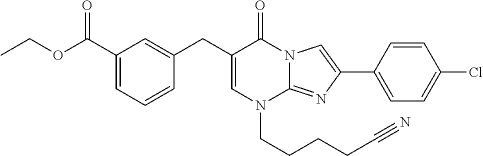 [SMILES: CCOC(=O)c1cccc(Cc2cn(CCCCC#N)c3nc(-c4ccc(Cl)cc4)cn3c2=O)c1] | B | 2.54 | 489 |
| 822 | 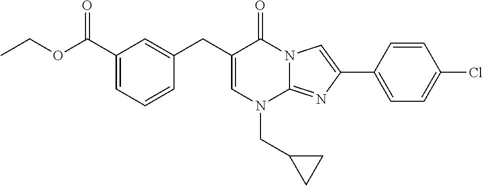 [SMILES: CCOC(=O)c1cccc(Cc2cn(CC3CC3)c3nc(-c4ccc(Cl)cc4)cn3c2=O)c1] | B | 2.81 | 462 |
| 823 | 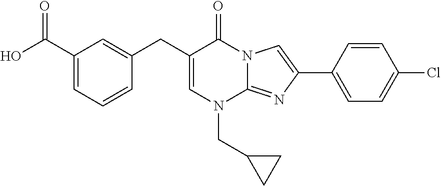 [SMILES: O=C(O)c1cccc(Cc2cn(CC3CC3)c3nc(-c4ccc(Cl)cc4)cn3c2=O)c1] | B | 2.44 | 434 |
| 824 | 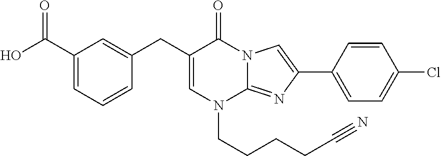 [SMILES: N#CCCCCn1cc(Cc2cccc(C(=O)O)c2)c(=O)n2cc(-c3ccc(Cl)cc3)nc12] | B | 2.19 | 461 |

TABLE 170

| | | | Retention | |
|---|---|---|---|---|
| | | | Time | Mass |
| Compound | Structure | LC/MS | [min] | [M+H] |
| 825 | 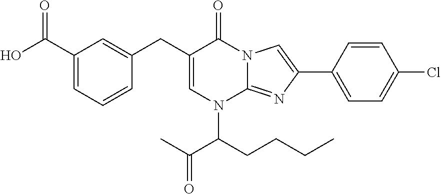 [SMILES: CCCCC(C(C)=O)n1cc(Cc2cccc(C(=O)O)c2)c(=O)n2cc(-c3ccc(Cl)cc3)nc12] | B | 2.54 | 492 |
| 826 | 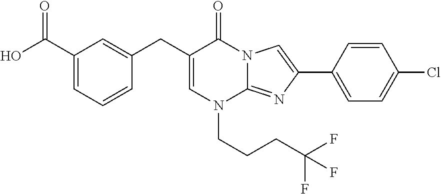 [SMILES: O=C(O)c1cccc(Cc2cn(CCCC(F)(F)F)c3nc(-c4ccc(Cl)cc4)cn3c2=O)c1] | B | 2.46 | 490 |
| 827 | 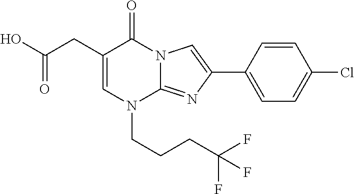 [SMILES: O=C(O)Cc1cn(CCCC(F)(F)F)c2nc(-c3ccc(Cl)cc3)cn2c1=O] | B | 2.21 | 414 |
| 828 | 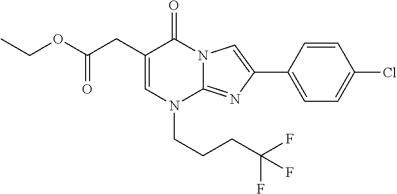 [SMILES: CCOC(=O)Cc1cn(CCCC(F)(F)F)c2nc(-c3ccc(Cl)cc3)cn2c1=O] | B | 2.51 | 442 |
| 829 | 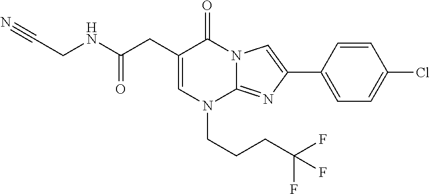 [SMILES: N#CCNC(=O)Cc1cn(CCCC(F)(F)F)c2nc(-c3ccc(Cl)cc3)cn2c1=O] | B | 2.21 | 452 |

TABLE 171

| | | | Retention | |
|---|---|---|---|---|
| | | | Time | Mass |
| Compound | Structure | LC/MS | [min] | [M+H] |
| 830 | 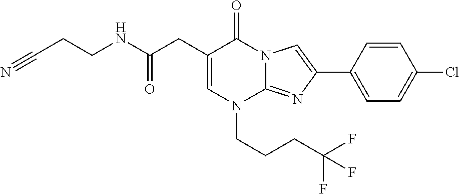 [SMILES: N#CCCNC(=O)Cc1cn(CCCC(F)(F)F)c2nc(-c3ccc(Cl)cc3)cn2c1=O] | B | 2.19 | 466 |
| 831 | 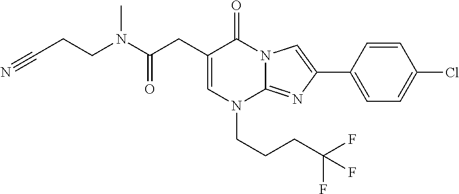 [SMILES: CN(CCC#N)C(=O)Cc1cn(CCCC(F)(F)F)c2nc(-c3ccc(Cl)cc3)cn2c1=O] | B | 2.26 | 480 |
| 832 | 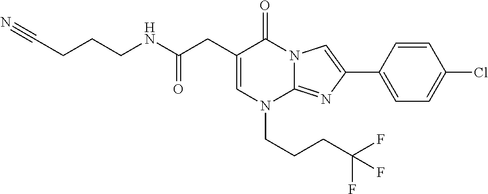 [SMILES: N#CCCCNC(=O)Cc1cn(CCCC(F)(F)F)c2nc(-c3ccc(Cl)cc3)cn2c1=O] | B | 2.22 | 480 |
| 833 | 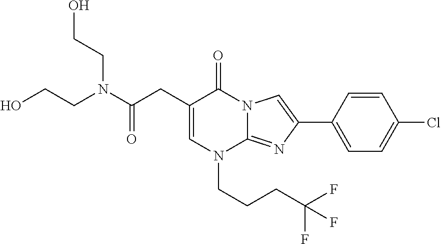 [SMILES: O=C(Cc1cn(CCCC(F)(F)F)c2nc(-c3ccc(Cl)cc3)cn2c1=O)N(CCO)CCO] | A | 2.02 | 501 |
| 834 | 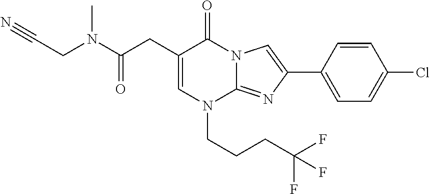 [SMILES: CN(CC#N)C(=O)Cc1cn(CCCC(F)(F)F)c2nc(-c3ccc(Cl)cc3)cn2c1=O] | A | 2.39 | 466 |

TABLE 172

| | | | Retention | |
|---|---|---|---|---|
| | | | Time | Mass |
| Compound | Structure | LC/MS | [min] | [M+H] |
| 835 | 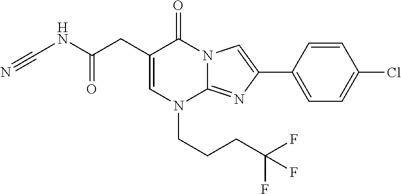 [SMILES: N#CNC(=O)Cc1cn(CCCC(F)(F)F)c2nc(-c3ccc(Cl)cc3)cn2c1=O] | A | 2.35 | 438 |
| 836 | 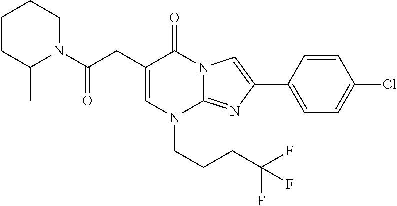 [SMILES: CC1CCCCN1C(=O)Cc1cn(CCCC(F)(F)F)c2nc(-c3ccc(Cl)cc3)cn2c1=O] | A | 2.68 | 495 |
| 837 | 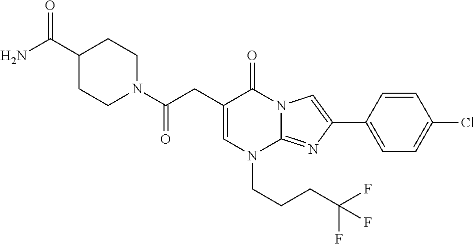 [SMILES: NC(=O)C1CCN(C(=O)Cc2cn(CCCC(F)(F)F)c3nc(-c4ccc(Cl)cc4)cn3c2=O)CC1] | A | 2.08 | 524 |
| 838 | 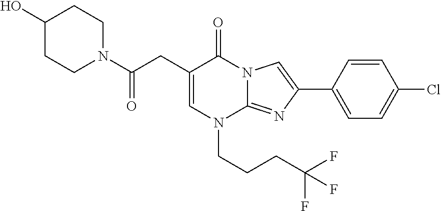 [SMILES: O=C(Cc1cn(CCCC(F)(F)F)c2nc(-c3ccc(Cl)cc3)cn2c1=O)N1CCC(O)CC1] | A | 2.14 | 497 |
| 839 | 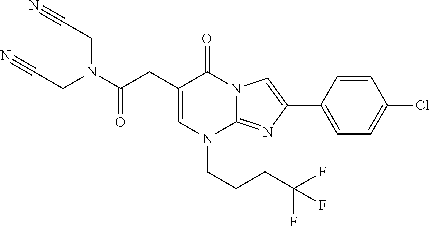 [SMILES: N#CCN(CC#N)C(=O)Cc1cn(CCCC(F)(F)F)c2nc(-c3ccc(Cl)cc3)cn2c1=O] | A | 2.44 | 491 |

TABLE 173

| | | | Retention | |
|---|---|---|---|---|
| | | | Time | Mass |
| Compound | Structure | LC/MS | [min] | [M+H] |
| 840 | 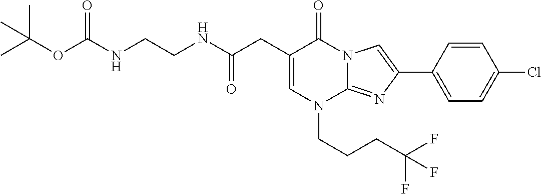 [SMILES: CC(C)(C)OC(=O)NCCNC(=O)Cc1cn(CCCC(F)(F)F)c2nc(-c3ccc(Cl)cc3)cn2c1=O] | A | 2.5 | 556 |
| 841 | 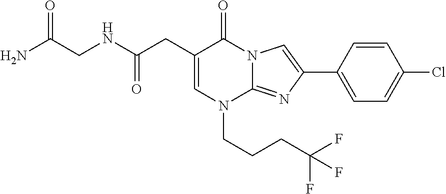 [SMILES: NC(=O)CNC(=O)Cc1cn(CCCC(F)(F)F)c2nc(-c3ccc(Cl)cc3)cn2c1=O] | A | 2.01 | 470 |
| 842 | 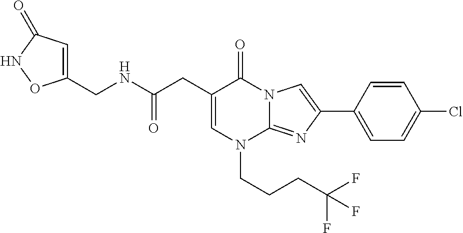 [SMILES: O=C(Cc1cn(CCCC(F)(F)F)c2nc(-c3ccc(Cl)cc3)cn2c1=O)NCc1cc(=O)[nH]o1] | A | 2.19 | 510 |
| 843 | 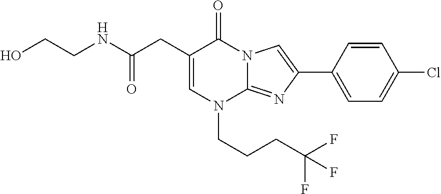 [SMILES: O=C(Cc1cn(CCCC(F)(F)F)c2nc(-c3ccc(Cl)cc3)cn2c1=O)NCCO] | A | 2.07 | 457 |
| 844 | 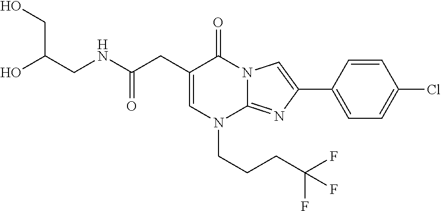 [SMILES: O=C(Cc1cn(CCCC(F)(F)F)c2nc(-c3ccc(Cl)cc3)cn2c1=O)NCC(O)CO] | A | 1.99 | 487 |

TABLE 174

| | | | Retention | |
|---|---|---|---|---|
| | | | Time | Mass |
| Compound | Structure | LC/MS | [min] | [M+H] |
| 845 | 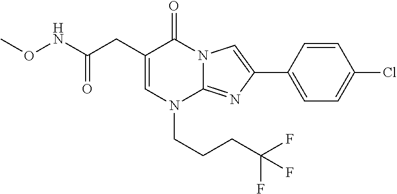 [SMILES: CONC(=O)Cc1cn(CCCC(F)(F)F)c2nc(-c3ccc(Cl)cc3)cn2c1=O] | A | 2.2 | 443 |
| 846 | 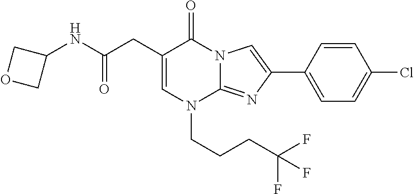 [SMILES: O=C(Cc1cn(CCCC(F)(F)F)c2nc(-c3ccc(Cl)cc3)cn2c1=O)NC1COC1] | A | 2.17 | 469 |
| 847 | 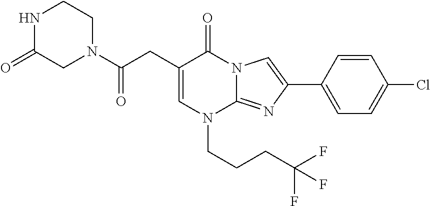 [SMILES: O=C1CN(C(=O)Cc2cn(CCCC(F)(F)F)c3nc(-c4ccc(Cl)cc4)cn3c2=O)CCN1] | A | 2.06 | 496 |
| 848 | 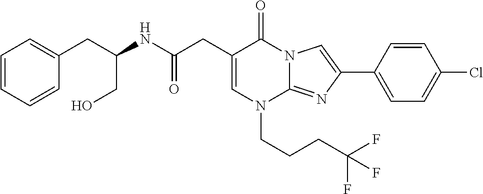 [SMILES: O=C(Cc1cn(CCCC(F)(F)F)c2nc(-c3ccc(Cl)cc3)cn2c1=O)N[C@@H](CO)Cc1ccccc1] | A | 2.45 | 547 |
| 849 | 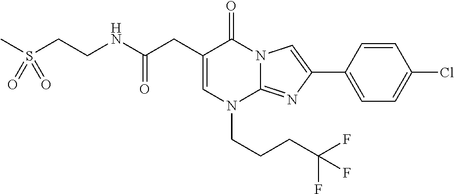 [SMILES: CS(=O)(=O)CCNC(=O)Cc1cn(CCCC(F)(F)F)c2nc(-c3ccc(Cl)cc3)cn2c1=O] | A | 2.18 | 519 |

TABLE 175

| | | | Retention | |
|---|---|---|---|---|
| | | | Time | Mass |
| Compound | Structure | LC/MS | [min] | [M+H] |
| 850 | 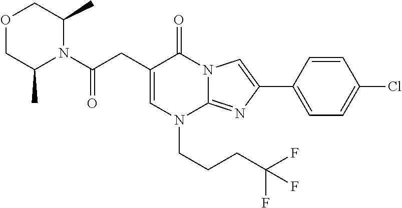 [SMILES: C[C@@H]1COC[C@H](C)N1C(=O)Cc1cn(CCCC(F)(F)F)c2nc(-c3ccc(Cl)cc3)cn2c1=O] | A | 2.49 | 511 |
| 851 | 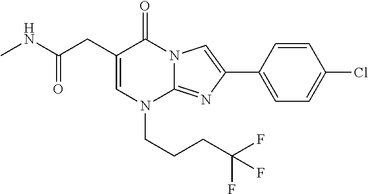 [SMILES: CNC(=O)Cc1cn(CCCC(F)(F)F)c2nc(-c3ccc(Cl)cc3)cn2c1=O] | A | 2.21 | 427 |
| 852 | 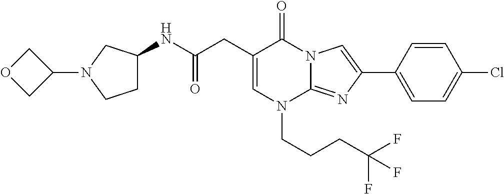 [SMILES: O=C(Cc1cn(CCCC(F)(F)F)c2nc(-c3ccc(Cl)cc3)cn2c1=O)N[C@H]1CCN(C2COC2)C1] | A | 1.79 | 538 |
| 853 | 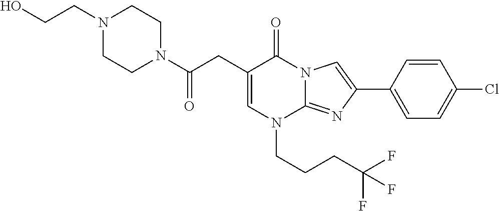 [SMILES: O=C(Cc1cn(CCCC(F)(F)F)c2nc(-c3ccc(Cl)cc3)cn2c1=O)N1CCN(CCO)CC1] | A | 1.82 | 526 |
| 854 | 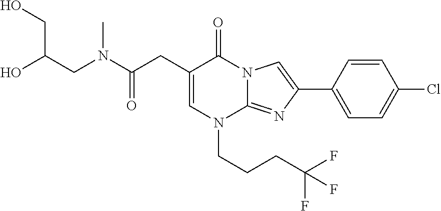 [SMILES: CN(CC(O)CO)C(=O)Cc1cn(CCCC(F)(F)F)c2nc(-c3ccc(Cl)cc3)cn2c1=O] | A | 2.03 | 501 |

TABLE 176

| | | | Retention | |
|---|---|---|---|---|
| | | | Time | Mass |
| Compound | Structure | LC/MS | [min] | [M+H] |
| 855 | 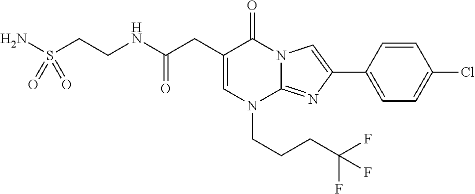 [SMILES: NS(=O)(=O)CCNC(=O)Cc1cn(CCCC(F)(F)F)c2nc(-c3ccc(Cl)cc3)cn2c1=O] | A | 2.12 | 520 |
| 856 | 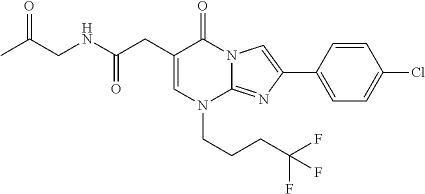 [SMILES: CC(=O)CNC(=O)Cc1cn(CCCC(F)(F)F)c2nc(-c3ccc(Cl)cc3)cn2c1=O] | A | 2.22 | 469 |
| 857 | 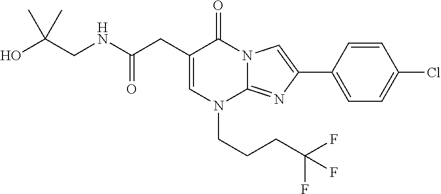 [SMILES: CC(C)(O)CNC(=O)Cc1cn(CCCC(F)(F)F)c2nc(-c3ccc(Cl)cc3)cn2c1=O] | A | 2.18 | 485 |
| 858 | 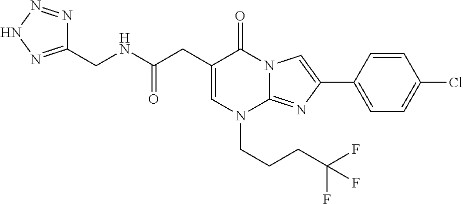 [SMILES: O=C(Cc1cn(CCCC(F)(F)F)c2nc(-c3ccc(Cl)cc3)cn2c1=O)NCc1nn[nH]n1] | A | 2.13 | 495 |
| 859 | 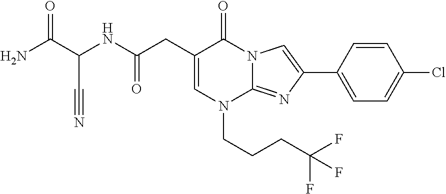 [SMILES: N#CC(NC(=O)Cc1cn(CCCC(F)(F)F)c2nc(-c3ccc(Cl)cc3)cn2c1=O)C(N)=O] | A | 2.18 | 495 |

TABLE 177

| | | | Retention | |
|---|---|---|---|---|
| | | | Time | Mass |
| Compound | Structure | LC/MS | [min] | [M+H] |
| 860 | 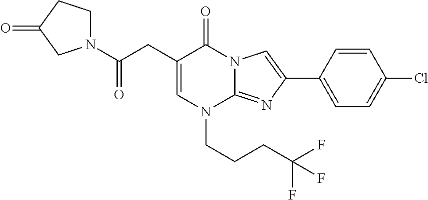 [SMILES: O=C1CCN(C(=O)Cc2cn(CCCC(F)(F)F)c3nc(-c4ccc(Cl)cc4)cn3c2=O)C1] | A | 2.25 | 481 |
| 861 | 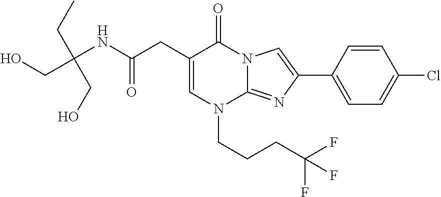 [SMILES: CCC(CO)(CO)NC(=O)Cc1cn(CCCC(F)(F)F)c2nc(-c3ccc(Cl)cc3)cn2c1=O] | A | 2.19 | 515 |
| 862 | 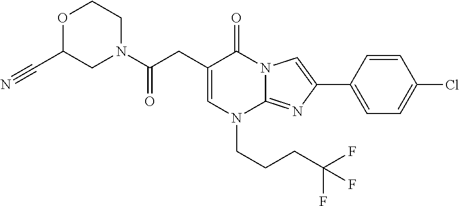 [SMILES: N#CC1CN(C(=O)Cc2cn(CCCC(F)(F)F)c3nc(-c4ccc(Cl)cc4)cn3c2=O)CCO1] | A | 2.41 | 508 |
| 863 | 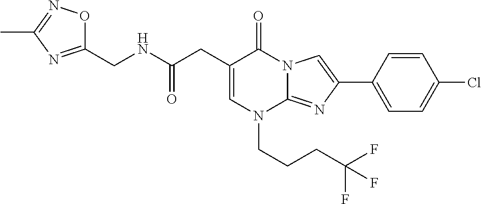 [SMILES: Cc1noc(CNC(=O)Cc2cn(CCCC(F)(F)F)c3nc(-c4ccc(Cl)cc4)cn3c2=O)n1] | A | 2.3 | 509 |
| 864 | 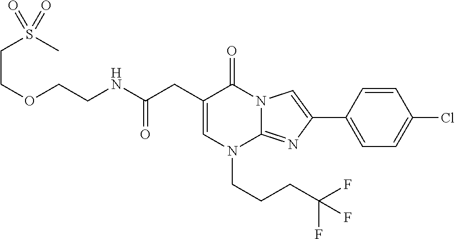 [SMILES: CS(=O)(=O)CCOCCNC(=O)Cc1cn(CCCC(F)(F)F)c2nc(-c3ccc(Cl)cc3)cn2c1=O] | A | 2.17 | 563 |

TABLE 178

| | | | Retention | |
|---|---|---|---|---|
| | | | Time | Mass |
| Compound | Structure | LC/MS | [min] | [M+H] |
| 865 | 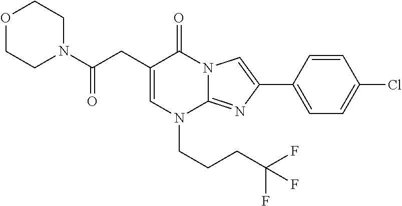 [SMILES: O=C(Cc1cn(CCCC(F)(F)F)c2nc(-c3ccc(Cl)cc3)cn2c1=O)N1CCOCC1] | A | 2.28 | 483 |
| 866 | 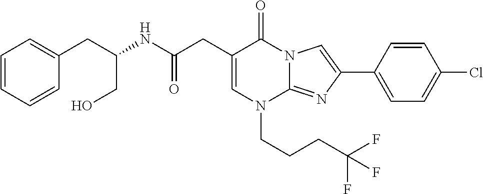 [SMILES: O=C(Cc1cn(CCCC(F)(F)F)c2nc(-c3ccc(Cl)cc3)cn2c1=O)N[C@H](CO)Cc1ccccc1] | A | 2.45 | 547 |
| 867 | 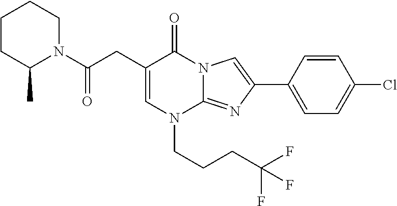 [SMILES: C[C@H]1CCCCN1C(=O)Cc1cn(CCCC(F)(F)F)c2nc(-c3ccc(Cl)cc3)cn2c1=O] | A | 2.68 | 495 |

### Test Example 1

### (Method A) Evaluation of Autotaxin Inhibitor

Solution A containing 25 mM Tris-HCl buffer (pH7.5), 100 mM NaCl, 5 mM MgCl₂ and 0.1% BSA was prepared. Mouse autotaxin enzyme (purchased from R&D System) was diluted with Solution A, and 5 μL of which was added to a solution of test compound in DMSO. Furthermore, 5 μl of 0.5 μM TG-mTMP in Solution A was added and allowed to react at room temperature for 2 hours. 5 μl of 150 mM EDTA in Solution A was added to quench the reaction, and a fluorescent dye TokyoGreen, which was produced by the reaction, was detected. The fluorescence was detected using ViewLux (PerkinElmer, Inc.) with an excitation wavelength of 480 nm and a fluorescence wavelength of 540 nm.

The percent inhibition of the test compound was calculated by assuming the sample with no test compound as 0% inhibition and the sample with no enzyme as 100% inhibition, and the percent inhibitions at different concentrations of the test compound were plotted to obtain a concentration-dependent curve. The IC50 value, which is the concentration of the test compound that resulted in 50% inhibition, was determined from the curve.

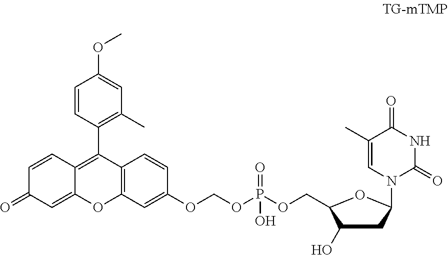 [SMILES: COc1ccc(-c2c3ccc(=O)cc-3oc3cc(OCOP(=O)(O)OC[C@H]4O[C@@H](n5cc(C)c(=O)[nH]c5=O)C[C@H]4O)ccc23)c(C)c1]

### Test Example 2

### (Method B) Evaluation of Autotaxin Inhibitor

Solution A containing 25 mM Tris-HCl buffer (pH7.5), 100 mM NaCl, 5 mM MgCl₂ and 0.1% BSA was prepared. Human autotaxin enzyme (purchased from R&D System) was diluted with Solution A, and 5 μL of which was added to a solution of test compound in DMSO. Furthermore, 5 μl of 0.5 μM TG-mTMP in Solution A was added and allowed to react at room temperature for 2 hours. 5 μl of 150 mM EDTA in Solution A was added to quench the reaction, and the fluorescent dye TokyoGreen, which was produced by the reaction, was detected. The fluorescence was detected using ViewLux (PerkinElmer, Inc.) with an excitation wavelength of 480 nm and a fluorescence wavelength of 540 nm.

The percent inhibition of the test compound was calculated by assuming the sample with no test compound as 0% inhibition and the sample with no enzyme as 100% inhibition, and the percent inhibitions at different concentrations of the test compound were plotted to obtain a concentration-dependent curve. The IC50 value, which is the concentration of the test compound that resulted in 50% inhibition, was determined from the curve.

### Test Example 3

### (Method C) Evaluation of Autotaxin Inhibitor

Solution B containing 100 mM Tris-HCl buffer (pH7.5), 150 mM NaCl, 5 mM MgCl₂ and 0.05% Triton X-100 was prepared. Human autotaxin enzyme (purchased from R&D System) was diluted with Solution B, and 2.5 μL of which was added to a solution of test compound in DMSO. Furthermore, 2.5 μl of 200 μM 18:0 Lyso PC (purchased from Avanti Polar Lipids) in Solution B was added and allowed to react at room temperature for 2 hours. After completion of the reaction, 15 μL of the coline assay reagent (100 mM Tris-HCl buffer (pH7.5), 5 mM MgCl2, 77 μg/mL choline oxidase, 10 μg/mL peroxidase, 25 μM 10-acetyl-3,7-dihydroxyphenoxazine and excess autotaxin inhibitor) was added and allowed to react at room temperature for 20 minutes. The fluorescent dye Resorufin, which was produced by the reaction, was detected. The fluorescence was detected using ViewLux (PerkinElmer, Inc.) with an excitation wavelength of 531 nm and a fluorescence wavelength of 598 nm.

The percent inhibition of the test compound was calculated by assuming the sample with no test compound as 0% inhibition and the sample with no enzyme as 100% inhibition, and the percent inhibitions at different concentrations of the test compound were plotted to obtain a concentration-dependent curve. The IC50 value, which is the concentration of the test compound that resulted in 50% inhibition, was determined from the curve.

The results obtained by the test methods described above are shown in the following tables.

### Test Method:

### Method A: Test Example 1; Method B: Test Example 2; Method C: Test Example 3

### Enzyme Inhibitory Activity:

A: IC50≦10 nM; B: 10 nM≦IC50≦100 nM; C: 100 nM≦IC50≦1000 nM; D: 1000 nM≦IC50

TABLE 179

| | | |
|---|---|---|
| | | Inhibitory |
| Compound | Assay Method | Activity |

| | |
|---|---|
| 1 | no data |
| 2 | no data |

| | | |
|---|---|---|
| 3 | Method B | A |
| 4 | Method A | C |
| 5 | Method A | B |
| 6 | Method A | B |
| 7 | Method B | B |
| 8 | Method B | B |
| 9 | Method A | B |
| 10 | Method A | B |
| 11 | Method A | C |
| 12 | Method A | C |
| 13 | Method B | B |
| 14 | Method A | B |
| 15 | Method A | B |
| 16 | Method A | B |
| 17 | Method A | B |
| 18 | Method B | A |
| 19 | Method A | B |
| 20 | Method B | A |
| 21 | Method A | B |
| 22 | Method A | B |
| 23 | Method A | B |
| 24 | Method A | C |
| 25 | Method A | C |
| 26 | Method B | B |
| 27 | Method A | D |
| 28 | Method B | B |
| 29 | Method B | B |
| 30 | Method A | B |
| 31 | Method A | B |
| 32 | Method A | B |
| 33 | Method A | D |
| 34 | Method A | B |
| 35 | Method A | B |
| 36 | Method A | B |
| 37 | Method A | C |
| 38 | Method A | D |
| 39 | Method A | D |
| 40 | Method A | B |
| 41 | Method C | C |
| 42 | Method B | B |
| 43 | Method B | B |
| 44 | Method B | B |
| 45 | Method B | C |
| 46 | Method C | D |
| 47 | Method C | D |
| 48 | Method C | D |
| 49 | Method C | C |
| 50 | Method B | C |
| 51 | Method B | B |
| 52 | Method B | D |
| 53 | Method B | B |
| 54 | Method B | B |
| 55 | Method B | B |
| 56 | Method B | B |
| 57 | Method B | B |
| 58 | Method B | B |
| 59 | Method B | B |
| 60 | Method B | C |
| 61 | Method B | C |
| 62 | Method B | C |
| 63 | Method C | C |
| 64 | Method B | C |
| 65 | Method B | C |
| 66 | Method B | B |
| 67 | Method B | B |
| 68 | Method B | D |
| 69 | Method B | D |
| 70 | Method B | C |
| 71 | Method B | B |
| 72 | Method B | B |
| 73 | Method B | B |
| 74 | Method B | C |
| 75 | Method B | C |
| 76 | Method B | C |
| 77 | Method B | B |
| 78 | Method B | B |
| 79 | Method B | B |
| 80 | Method B | B |
| 81 | Method B | A |
| 82 | Method B | B |
| 83 | Method B | B |
| 84 | Method B | A |
| 85 | Method B | A |
| 86 | Method B | C |

TABLE 180

| | | Inhibitory |
|---|---|---|
| Compound | Assay Method | Activity |
| 87 | Method B | B |
| 88 | Method B | C |
| 89 | Method B | A |
| 90 | Method B | B |
| 91 | Method B | B |
| 92 | Method B | B |
| 93 | Method B | D |
| 94 | Method B | B |
| 95 | Method B | B |
| 96 | Method B | C |
| 97 | Method B | B |
| 98 | Method C | B |
| 99 | Method B | C |
| 100 | Method B | B |
| 101 | Method B | B |
| 102 | Method B | C |
| 103 | Method B | B |
| 104 | Method B | B |
| 105 | Method B | B |
| 106 | Method B | B |
| 107 | Method B | C |
| 108 | Method B | B |
| 109 | Method B | C |
| 110 | Method B | B |
| 111 | Method B | C |
| 112 | Method B | B |
| 113 | Method B | B |
| 114 | Method B | B |
| 115 | Method B | B |
| 116 | Method B | C |
| 117 | Method B | B |
| 118 | Method B | C |
| 119 | Method C | C |
| 120 | Method B | D |
| 121 | Method B | B |
| 122 | Method B | C |
| 123 | Method B | C |
| 124 | Method B | B |

| | |
|---|---|
| 125 | no data |
| 126 | no data |

| | | |
|---|---|---|
| 127 | Method B | D |
| 128 | Method B | D |
| 129 | Method B | D |

| | |
|---|---|
| 130 | no data |
| 131 | no data |
| 132 | no data |
| 133 | no data |
| 134 | no data |

| | | |
|---|---|---|
| 135 | Method C | D |
| 136 | Method C | C |
| 137 | Method C | C |
| 138 | Method B | C |
| 139 | Method B | C |
| 140 | Method B | C |
| 141 | Method B | B |
| 142 | Method B | B |
| 143 | Method B | C |
| 144 | Method B | C |
| 145 | Method B | D |
| 146 | Method B | C |
| 147 | Method B | D |
| 148 | Method B | D |
| 149 | Method B | C |
| 150 | Method B | C |
| 151 | Method B | D |
| 152 | Method B | B |
| 153 | Method B | D |
| 154 | Method B | B |
| 155 | Method B | B |
| 156 | Method B | A |
| 157 | Method B | A |
| 158 | Method B | B |
| 159 | Method B | D |
| 160 | Method B | C |
| 161 | Method B | A |
| 162 | Method B | C |
| 163 | Method B | C |
| 164 | Method C | B |
| 165 | Method C | B |
| 166 | Method C | D |
| 167 | Method B | C |
| 168 | Method B | C |
| 169 | Method B | B |
| 170 | Method B | B |
| 171 | Method B | B |
| 172 | Method B | D |

TABLE 181

| | | Inhibitory |
|---|---|---|
| Compound | Assay Method | Activity |
| 173 | Method B | D |

| | |
|---|---|
| 174 | no data |
| 175 | no data |

| | | |
|---|---|---|
| 176 | Method B | B |
| 177 | Method B | B |
| 178 | Method B | A |
| 179 | Method B | B |
| 180 | Method B | A |
| 181 | Method B | A |
| 182 | Method B | D |
| 183 | Method B | B |
| 184 | Method B | D |
| 185 | Method B | C |
| 186 | Method B | B |
| 187 | Method B | B |
| 188 | Method B | B |
| 189 | Method B | B |
| 190 | Method C | B |
| 191 | Method B | A |
| 192 | Method C | B |
| 193 | Method B | A |
| 194 | Method C | B |
| 195 | Method B | A |
| 196 | Method B | A |
| 197 | Method C | B |
| 198 | Method C | B |
| 199 | Method B | B |
| 200 | Method B | A |
| 201 | Method B | A |
| 202 | Method C | B |
| 203 | Method B | B |
| 204 | Method B | C |
| 205 | Method B | B |
| 206 | Method B | B |
| 207 | Method C | B |
| 208 | Method B | B |
| 209 | Method B | C |
| 210 | Method B | C |
| 211 | Method B | D |
| 212 | Method B | D |
| 213 | Method B | C |
| 214 | Method B | A |
| 215 | Method B | A |
| 216 | Method B | D |
| 217 | Method B | C |
| 218 | Method B | B |
| 219 | Method C | B |
| 220 | Method B | B |
| 221 | Method B | B |
| 222 | Method B | B |
| 223 | Method B | B |
| 224 | Method B | B |
| 225 | Method B | A |
| 226 | Method B | B |
| 227 | Method B | A |
| 228 | Method B | B |
| 229 | Method B | B |
| 230 | Method B | A |
| 231 | Method B | A |
| 232 | Method B | A |
| 233 | Method B | B |
| 234 | Method B | A |
| 235 | Method B | B |
| 236 | Method B | B |
| 237 | Method B | B |
| 238 | Method B | A |
| 239 | Method B | B |
| 240 | Method B | A |
| 241 | Method B | A |
| 242 | Method C | B |
| 243 | Method B | A |
| 244 | Method B | B |
| 245 | Method B | B |
| 246 | Method B | B |
| 247 | Method B | B |
| 248 | Method B | B |
| 249 | Method B | B |
| 250 | Method B | B |
| 251 | Method B | B |
| 252 | Method B | B |
| 253 | Method B | B |
| 254 | Method B | A |
| 255 | Method B | A |
| 256 | Method B | A |
| 257 | Method B | A |
| 258 | Method B | B |

TABLE 182

| | | Inhibitory |
|---|---|---|
| Compound | Assay Method | Activity |
| 259 | Method B | B |
| 260 | Method B | B |
| 261 | Method B | C |
| 262 | Method B | C |
| 263 | Method B | C |
| 264 | Method B | C |
| 265 | Method B | D |
| 266 | Method B | C |
| 267 | Method B | C |
| 268 | Method B | A |
| 269 | Method B | A |
| 270 | Method B | C |
| 271 | Method B | D |
| 272 | Method B | B |
| 273 | Method B | C |
| 274 | Method B | A |
| 275 | Method B | B |
| 276 | Method B | B |
| 277 | Method B | B |
| 278 | Method B | B |
| 279 | Method B | B |
| 280 | Method B | B |
| 281 | Method B | B |
| 282 | Method C | B |
| 283 | Method B | A |
| 284 | Method C | B |
| 285 | Method B | A |
| 286 | Method B | A |
| 287 | Method B | A |
| 288 | Method C | B |
| 289 | Method B | A |
| 290 | Method C | C |
| 291 | Method C | C |
| 292 | Method C | C |
| 293 | Method C | C |
| 294 | Method C | C |
| 295 | Method C | C |
| 296 | Method C | C |
| 297 | Method C | C |
| 298 | Method C | C |
| 299 | Method C | B |
| 300 | Method C | C |
| 301 | Method C | C |
| 302 | Method C | B |
| 303 | Method C | D |
| 304 | Method C | C |
| 305 | Method C | D |
| 306 | Method C | B |
| 307 | Method C | D |
| 308 | Method C | B |
| 309 | Method C | B |
| 310 | Method C | C |
| 311 | Method C | C |
| 312 | Method C | B |
| 313 | Method C | C |
| 314 | Method C | D |
| 315 | Method C | C |
| 316 | Method C | B |
| 317 | Method C | D |
| 318 | Method C | C |
| 319 | Method C | B |
| 320 | Method C | C |
| 321 | Method C | C |
| 322 | Method C | D |
| 323 | Method C | C |
| 324 | Method C | C |
| 325 | Method C | C |
| 326 | Method C | A |
| 327 | Method C | C |
| 328 | Method C | C |
| 329 | Method C | C |
| 330 | Method C | C |
| 331 | Method C | C |
| 332 | Method C | C |
| 333 | Method C | C |
| 334 | Method C | D |
| 335 | Method C | C |
| 336 | Method C | B |
| 337 | Method C | B |
| 338 | Method C | B |
| 339 | Method C | B |
| 340 | Method C | B |
| 341 | Method C | B |
| 342 | Method C | C |
| 343 | Method C | B |
| 344 | Method C | C |

TABLE 183

| | | Inhibitory |
|---|---|---|
| Compound | Assay Method | Activity |
| 345 | Method C | A |
| 346 | Method C | B |
| 347 | Method C | B |
| 348 | Method C | A |
| 349 | Method C | A |
| 350 | Method C | A |
| 351 | Method C | B |
| 352 | Method C | D |
| 353 | Method C | A |
| 354 | Method C | A |
| 355 | Method C | A |
| 356 | Method C | A |
| 357 | Method C | C |
| 358 | Method C | C |
| 359 | Method C | C |
| 360 | Method C | D |
| 361 | Method C | D |
| 362 | Method C | B |
| 363 | Method C | B |
| 364 | Method C | B |
| 365 | Method C | B |
| 366 | Method B | B |
| 367 | Method B | A |
| 368 | Method B | A |
| 369 | Method B | A |
| 370 | Method B | A |
| 371 | Method B | B |
| 372 | Method B | B |
| 373 | Method C | B |
| 374 | Method B | A |
| 375 | Method C | B |
| 376 | Method C | B |
| 377 | Method C | B |
| 378 | Method C | B |
| 379 | Method B | A |
| 380 | Method B | A |
| 381 | Method C | B |
| 382 | Method C | B |
| 383 | Method C | B |
| 384 | Method B | A |
| 385 | Method B | A |
| 386 | Method B | A |
| 387 | Method C | B |
| 388 | Method C | B |
| 389 | Method B | A |
| 390 | Method C | B |
| 391 | Method C | B |
| 392 | Method B | C |
| 393 | Method B | B |
| 394 | Method C | B |
| 395 | Method B | B |
| 396 | Method B | B |
| 397 | Method B | A |
| 398 | Method B | B |
| 399 | Method B | B |
| 400 | Method B | A |
| 401 | Method C | B |
| 402 | Method B | A |
| 403 | Method B | B |
| 404 | Method B | A |
| 405 | Method C | B |
| 406 | Method C | C |
| 407 | Method C | B |
| 408 | Method B | B |
| 409 | Method C | B |
| 410 | Method B | A |
| 411 | Method B | A |
| 412 | Method C | B |
| 413 | Method C | B |
| 414 | Method B | B |
| 415 | Method B | B |
| 416 | Method B | A |
| 417 | Method B | B |
| 418 | Method B | B |
| 419 | Method B | A |
| 420 | Method B | B |
| 421 | Method B | B |
| 422 | Method B | B |
| 423 | Method B | B |
| 424 | Method B | A |
| 425 | Method C | B |
| 426 | Method B | A |
| 427 | Method C | B |
| 428 | Method C | C |
| 429 | Method C | B |
| 430 | Method C | B |

TABLE 184

| | | Inhibitory |
|---|---|---|
| Compound | Assay Method | Activity |
| 431 | Method C | B |
| 432 | Method C | C |
| 433 | Method C | B |
| 434 | Method C | C |
| 435 | Method C | C |
| 436 | Method C | B |
| 437 | Method C | B |
| 438 | Method C | B |
| 439 | Method C | B |
| 440 | Method C | C |
| 441 | Method C | C |
| 442 | Method C | D |
| 443 | Method C | C |
| 444 | Method C | C |
| 445 | Method C | C |
| 446 | Method C | C |
| 447 | Method C | B |
| 448 | Method C | B |
| 449 | Method C | B |
| 450 | Method C | B |
| 451 | Method C | B |
| 452 | Method C | B |
| 453 | Method C | C |
| 454 | Method C | B |
| 455 | Method C | C |
| 456 | Method C | B |
| 457 | Method C | B |
| 458 | Method C | C |
| 459 | Method C | B |
| 460 | Method C | C |
| 461 | Method C | B |
| 462 | Method C | B |
| 463 | Method C | B |
| 464 | Method C | B |
| 465 | Method C | B |
| 466 | Method C | B |
| 467 | Method C | B |
| 468 | Method C | C |
| 469 | Method C | B |
| 470 | Method C | B |
| 471 | Method C | C |
| 472 | Method C | B |
| 473 | Method C | B |
| 474 | Method C | B |
| 475 | Method C | B |
| 476 | Method C | B |
| 477 | Method C | B |
| 478 | Method C | B |
| 479 | Method C | B |
| 480 | Method C | B |
| 481 | Method C | B |
| 482 | Method C | A |
| 483 | Method C | B |
| 484 | Method C | B |
| 485 | Method C | B |
| 486 | Method C | A |
| 487 | Method C | A |
| 488 | Method C | B |
| 489 | Method C | B |
| 490 | Method C | A |
| 491 | Method C | C |
| 492 | Method C | B |
| 493 | Method C | B |
| 494 | Method C | B |
| 495 | Method C | B |
| 496 | Method C | B |
| 497 | Method C | B |
| 498 | Method C | B |
| 499 | Method C | B |
| 500 | Method C | B |
| 501 | Method C | B |
| 502 | Method C | B |
| 503 | Method C | B |
| 504 | Method C | B |
| 505 | Method C | A |
| 506 | Method C | B |
| 507 | Method C | B |
| 508 | Method C | B |
| 509 | Method C | B |
| 510 | Method C | A |
| 511 | Method C | B |
| 512 | Method C | A |
| 513 | Method C | B |
| 514 | Method C | A |
| 515 | Method C | B |
| 516 | Method C | B |

TABLE 185

| | | Inhibitory |
|---|---|---|
| Compound | Assay Method | Activity |
| 517 | Method C | B |
| 518 | Method C | C |
| 519 | Method C | B |
| 520 | Method C | B |
| 521 | Method C | B |
| 522 | Method C | B |
| 523 | Method C | B |
| 524 | Method C | B |
| 525 | Method C | B |
| 526 | Method C | C |
| 527 | Method C | D |
| 528 | Method C | C |

TABLE 186

| | | Inhibitory |
|---|---|---|
| Compound | Assay Method | Activity |
| 529 | Method C | C |
| 530 | Method C | B |
| 531 | Method C | A |
| 532 | Method C | B |
| 533 | Method C | A |
| 534 | Method C | A |
| 535 | Method C | A |
| 536 | Method C | A |
| 537 | Method C | B |
| 538 | Method C | C |
| 539 | Method C | B |
| 540 | Method C | B |
| 541 | Method C | C |
| 542 | Method C | C |
| 543 | Method C | B |
| 544 | Method C | C |
| 545 | Method C | C |
| 546 | Method C | B |
| 547 | Method C | B |
| 548 | Method C | C |
| 549 | Method C | B |
| 550 | Method C | C |
| 551 | Method C | C |
| 552 | Method C | C |
| 553 | Method C | C |
| 554 | Method C | C |
| 555 | Method C | B |
| 556 | Method C | B |
| 557 | Method C | B |
| 558 | Method C | C |
| 559 | Method C | B |
| 560 | Method C | B |
| 561 | Method C | D |
| 562 | Method C | C |
| 563 | Method C | C |
| 564 | Method C | B |
| 565 | Method C | C |
| 566 | Method C | B |
| 567 | Method C | D |
| 568 | Method C | B |
| 569 | Method C | A |
| 570 | Method C | A |
| 571 | Method C | C |
| 572 | Method C | B |
| 573 | Method C | B |
| 574 | Method C | B |
| 575 | Method C | B |
| 576 | Method C | B |
| 577 | Method C | A |
| 578 | Method C | B |
| 579 | Method C | B |
| 580 | Method C | A |
| 581 | Method C | A |
| 582 | Method C | A |
| 583 | Method C | A |
| 584 | Method C | A |
| 585 | Method C | A |
| 586 | Method C | A |
| 587 | Method C | D |
| 588 | Method C | C |
| 589 | Method C | D |
| 590 | Method C | C |
| 591 | Method C | C |
| 592 | Method C | B |
| 593 | Method C | B |
| 594 | Method C | B |
| 595 | Method C | C |
| 596 | Method C | B |
| 597 | Method C | B |
| 598 | Method C | B |
| 599 | Method C | B |
| 600 | Method C | A |
| 601 | Method C | C |
| 602 | Method C | C |
| 603 | Method C | C |
| 604 | Method C | C |
| 605 | Method C | C |
| 606 | Method C | D |
| 607 | Method C | B |
| 608 | Method C | B |
| 609 | Method C | B |
| 610 | Method C | D |
| 611 | Method C | B |
| 612 | Method C | C |
| 613 | Method C | B |
| 614 | Method C | C |

TABLE 187

| | Assay | Inhibitory |
|---|---|---|
| Compound | Method | Activity |
| 615 | Method C | A |
| 616 | Method C | A |
| 617 | Method C | C |
| 618 | Method C | B |
| 619 | Method C | B |
| 620 | Method C | A |
| 621 | Method C | A |
| 622 | Method C | C |
| 623 | Method C | C |
| 624 | Method C | B |
| 625 | Method C | C |
| 626 | Method C | B |
| 627 | Method C | C |
| 628 | Method C | A |
| 629 | Method C | A |
| 630 | Method C | C |
| 631 | Method C | C |
| 632 | Method C | B |
| 633 | Method C | B |
| 634 | Method C | C |
| 635 | Method C | D |
| 636 | Method C | B |
| 637 | Method C | D |
| 638 | Method C | C |
| 639 | Method C | C |
| 640 | Method C | D |
| 641 | Method C | D |
| 642 | Method C | B |
| 643 | Method C | C |
| 644 | Method C | C |
| 645 | Method C | C |
| 646 | Method C | D |
| 647 | Method C | D |
| 648 | Method C | D |
| 649 | Method C | B |
| 650 | Method C | D |
| 651 | Method C | B |
| 652 | Method C | B |
| 653 | Method C | C |
| 654 | Method C | A |
| 655 | Method C | C |
| 656 | Method C | C |
| 657 | Method C | A |
| 658 | Method C | B |
| 659 | Method C | D |
| 660 | Method C | D |
| 661 | Method C | D |
| 662 | Method C | B |
| 663 | Method C | C |
| 664 | Method C | B |
| 665 | Method C | B |
| 666 | Method C | B |
| 667 | Method C | B |
| 668 | Method C | C |
| 669 | Method C | B |
| 670 | Method C | B |
| 671 | Method C | C |
| 672 | Method C | B |
| 673 | Method C | A |
| 674 | Method C | A |
| 675 | Method C | A |
| 676 | Method C | A |
| 677 | Method C | A |
| 678 | Method C | B |
| 679 | Method C | A |
| 680 | Method C | B |
| 681 | Method C | A |
| 682 | Method C | C |
| 683 | Method C | B |
| 684 | Method C | B |
| 685 | Method C | A |
| 686 | Method C | B |
| 687 | Method C | C |
| 688 | Method C | B |
| 689 | Method C | A |
| 690 | Method C | B |
| 691 | Method C | C |
| 692 | Method C | B |
| 693 | Method C | B |
| 694 | Method C | B |
| 695 | Method C | C |
| 696 | Method C | C |
| 697 | Method C | B |
| 698 | Method C | B |
| 699 | Method C | C |
| 700 | Method C | B |

TABLE 188

| | Assay | Inhibitory |
|---|---|---|
| Compound | Method | Activity |
| 701 | Method C | C |
| 702 | Method C | B |
| 703 | Method C | B |
| 704 | Method C | B |
| 705 | Method C | C |
| 706 | Method C | B |
| 707 | Method C | C |
| 708 | Method C | B |
| 709 | Method C | C |
| 710 | Method C | C |
| 711 | Method C | C |
| 712 | Method C | C |
| 713 | Method C | B |
| 714 | Method C | C |
| 715 | Method C | B |
| 716 | Method C | C |
| 717 | Method C | C |
| 718 | Method C | B |
| 719 | Method C | C |
| 720 | Method C | C |
| 721 | Method C | B |
| 722 | Method C | A |
| 723 | Method C | A |
| 724 | Method C | A |
| 725 | Method C | B |
| 726 | Method C | A |
| 727 | Method C | C |
| 728 | Method C | A |
| 729 | Method C | A |
| 730 | Method C | A |
| 731 | Method C | A |
| 732 | Method C | A |
| 733 | Method C | A |
| 734 | Method C | A |
| 735 | Method C | A |
| 736 | Method C | A |
| 737 | Method C | A |
| 738 | Method C | A |
| 739 | Method C | A |
| 740 | Method C | A |
| 741 | Method C | B |
| 742 | Method C | B |
| 743 | Method C | A |
| 744 | Method C | B |
| 745 | Method C | A |
| 746 | Method C | A |
| 747 | Method C | B |
| 748 | Method C | A |
| 749 | Method C | A |
| 750 | Method C | B |
| 751 | Method C | A |
| 752 | Method C | A |
| 753 | Method C | A |
| 754 | Method C | A |
| 755 | Method C | C |
| 756 | Method C | C |
| 757 | Method C | B |
| 758 | Method C | B |
| 759 | Method C | B |
| 760 | Method C | B |
| 761 | Method C | C |
| 762 | Method C | D |
| 763 | Method C | B |
| 764 | Method C | B |
| 765 | Method C | C |
| 766 | Method C | B |
| 767 | Method C | C |
| 768 | Method C | D |
| 769 | Method C | B |
| 770 | Method C | C |
| 771 | Method C | C |
| 772 | Method C | C |
| 773 | Method C | D |
| 774 | Method C | B |
| 775 | Method C | A |
| 776 | Method C | A |
| 777 | Method C | A |
| 778 | Method C | A |
| 779 | Method C | A |

TABLE 189

| | Assay | Inhibitory |
|---|---|---|
| Compound | Method | Activity |
| 780 | Method C | C |
| 781 | Method C | D |
| 782 | Method C | C |
| 783 | Method C | B |
| 784 | Method C | C |
| 785 | Method C | C |
| 786 | Method C | C |
| 787 | Method C | C |
| 788 | Method C | D |
| 789 | Method C | B |
| 790 | Method C | A |
| 791 | Method C | C |
| 792 | Method C | C |
| 793 | Method C | C |
| 794 | Method C | B |
| 795 | Method C | A |
| 796 | Method C | C |
| 797 | Method C | B |
| 798 | Method C | C |
| 799 | Method C | B |
| 800 | Method C | B |
| 801 | Method C | C |
| 802 | Method C | D |
| 803 | Method C | D |
| 804 | Method C | D |
| 805 | Method C | C |
| 806 | Method C | D |
| 807 | Method C | B |
| 808 | Method C | D |
| 809 | Method C | C |
| 810 | Method C | C |
| 811 | Method C | D |
| 812 | Method C | C |
| 813 | Method C | D |
| 823 | Method C | B |
| 824 | Method C | A |
| 825 | Method C | D |
| 826 | Method C | B |
| 827 | Method C | D |
| 828 | Method C | D |
| 829 | Method C | C |
| 830 | Method C | C |
| 831 | Method C | C |
| 832 | Method C | D |
| 833 | Method C | D |
| 834 | Method C | C |
| 835 | Method C | D |
| 836 | Method C | B |
| 837 | Method C | D |
| 838 | Method C | D |
| 839 | Method C | C |
| 840 | Method C | C |
| 841 | Method C | D |
| 842 | Method C | C |
| 843 | Method C | D |
| 844 | Method C | D |
| 845 | Method C | C |
| 846 | Method C | D |
| 847 | Method C | D |
| 848 | Method C | D |
| 849 | Method C | D |
| 850 | Method C | C |
| 851 | Method C | C |
| 852 | Method C | D |
| 853 | Method C | D |
| 854 | Method C | D |
| 855 | Method C | D |
| 856 | Method C | D |

TABLE 190

| | Assay | Inhibitory |
|---|---|---|
| Compound | Method | Activity |
| 814 | Method C | C |
| 815 | Method C | A |
| 816 | Method C | B |
| 817 | Method C | A |
| 818 | Method C | D |
| 819 | Method C | C |
| 820 | Method C | B |
| 821 | Method C | C |
| 822 | Method C | D |
| 857 | Method C | D |
| 858 | Method C | B |
| 859 | Method C | D |
| 860 | Method C | C |
| 861 | Method C | B |
| 862 | Method C | B |
| 863 | Method C | C |
| 864 | Method C | D |
| 865 | Method C | C |

TABLE 191

| | Assay | Inhibitory |
|---|---|---|
| Compound | Method | Activity |
| 866 | Method C | D |
| 867 | Method C | B |

### Test Example 4

### CYP Inhibition Test

Using commercially available pooled human hepatic microsome, the compound was tested to assess inhibitory effect on the typical substrate metabolism reactions of human main five CYP enzyme forms (CYP1A2, 2C9, 2C19, 2D6, 3A4), specifically, 7-ethoxyresorufin O-deethylation (CYP1A2), tolbutamide methyl-hydroxylation (CYP2C9), mephenyloin 4′-hydroxylation (CYP2C19), dextromethorphan O-demethylation (CYP2D6) and terfenedine hydroxylation (CYP3A4).

The reaction conditions were as follows.

### Substrates:

0.5 μmol/L ethoxyresorufin (CYP1A2),
100 μmol/L tolbutamide (CYP2C9),
50 μmol/L S-mephenitoin (CYP2C19),
5 μmol/L dextromethorphan (CYP2D6),
1 μmol/L terfenedine (CYP3A4);
   Reaction Time: 15 minutes;

### Reaction Temperature: 37° C.;

Enzyme: pooled human hepatic microsome 0.2 mg protein/mL;
Concentration of Test Compound: 1, 5, 10, 20 μmol/L (four points).

A test sample, which contains the substrate, human hepatic microsome and test compound at the amounts as described above in 50 mM Hepes buffer, was added to a 96-well plate.

The cofactor NADPH was added to initiate metabolism reaction. After the incubation at 37° C. for 15 minutes, a methanol/acetonitrile=1/1 (v/v) solution was added to stop the reaction. After centrifugation at 3000 rpm for 15 minutes, resorufin (CYP1A2 metabolite) in the supernatant was quantified by fluorescent multilabel counter. Tributamide hydroxide (CYP2C9 metabolite), mephenyloin 4′ hydroxide (CYP2C19 metabolite), dextromethorphan (CYP2D6 metabolite) and terfenadine alcohol (CYP3A4 metabolite) were determined by LC/MS/MS.

Only DMSO, which was the solvent for the test compound, was added to the reaction system as the control (100%). For each concentration of the test compound, the remaining activity (%) was calculated, and the IC50 was calculated by reverse presumption by a logistic model using the concentration and the inhibition rate.

### Test Example 5

### Metabolic Stability

Assessment of metabolic stability in hepatic microsomes:

To tris-hydrochloric acid buffer (pH 7.4), were added NADPH (the final concentration was 1 mM in case of oxidative metabolism), hepatic microsomes (the final concentration was 0.5 mg protein/mL) and test compound (the final concentration was 2 μM), and the mixture was reacted at 37° C. for 0 and 30 minutes. In case of conjugated glucuronic acid, UDPGA (the final concentration was 5 mM) was added instead of NADPH. The reaction was stopped by addition of acetonitrile/methanol=1/1 (v/v) (2 parts by volume of the reaction solution). After the centrifugation, the supernatant was measured by HPLC. By comparing the results obtained from the reactions for 0 and 30 minutes, the loss of the compound by metabolic reaction was calculated to assess the metabolic stability of the compound of the invention.

### Test Example 6

### Powder Solubility Test

Appropriate amount of test sample was put into appropriate container. To the container was added 200 μL each of JP-1 solution (sodium chloride 2.0 g, hydrochloric acid 7.0 mL and water to reach 1000 mL), JP-2 solution (phosphate buffer (pH 6.8) 500 mL and water 500 mL) and 20 mmol/L TCA (sodium taurocholate)/JP-2 solution (TCA 1.08 g and water to reach 100 mL). In the case that the test compound was dissolved after the addition of the test solution, bulk powder was added as appropriate. The container was sealed and shaken for 1 hour at 37° C. The mixture was filtered, and 100 μL of methanol was added to each 100-μL aliquot of the filtrate to make the filtrates two-fold diluted. The dilution ratio was changed if necessary. After checking if any bubble or precipitate occurred, the container was sealed and shaken. Quantification was performed by absolute calibration method using HPLC.

### Formulation Examples

The following Formulation Examples are only exemplified and not intended to limit the scope of the invention.

### Formulation Example 1

### Tablets

| | |
|---|---|
| Compound of formula (I) | 15 mg |
| Starch | 15 mg |
| Lactose | 15 mg |
| Crystalline cellulose | 19 mg |
| Polyvinyl alcohol | 3 mg |
| Distilled water | 30 ml |
| Calcium stearate | 3 mg |

The above ingredients except calcium stearate are uniformly mixed and milled to granulate, and dried to obtain a suitable size of granules. Then, the granules are added with calcium stearate and compressed to form a tablet.

### Formulation Example 2

### Capsules

| | |
|---|---|
| Compound of formula (I) | 10 mg |
| Magnesium stearate | 10 mg |
| Lactose | 80 mg |

The above ingredients are mixed uniformly to obtain powders or fine granules, which are then filled in a capsule.

### Formulation Example 3

### Granules

| | |
|---|---|
| Compound of formula (I) | 30 g |
| Lactose | 265 g |
| Magnesium Stearate | 5 g |

The above ingredients are mixed uniformly and compressed. The compressed mixture is milled, granulated and sieved to obtain the desired size of granules.

### INDUSTRIAL APPLICABILITY

The present invention is applicable in the pharmaceutical field, for example, in the development and production of medicaments for the treatment of fibrotic diseases.

1. An autotaxin inhibitor comprising a compound of formula (I): 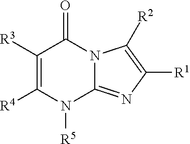
wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
R², R³ and R⁴ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino,
—N═C(R^{4a})(OR^{4b}) wherein R^{4a} is substituted or unsubstituted alkyl and R^{4b} is substituted or unsubstituted alkyl,
substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group,
or a pharmaceutically acceptable salt thereof.

2. A compound of formula (I) or a pharmaceutically acceptable salt thereof: 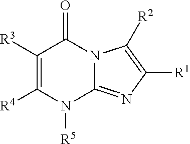
wherein
R¹ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group;
R², R³ and R⁴ are each independently hydrogen, halogen, hydroxy, cyano, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted amino,
—N═C(R^{4a})(OR^{4b}) wherein R^{4a} is substituted or unsubstituted alkyl and R^{4b} is substituted or unsubstituted alkyl,
substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl or substituted or unsubstituted aromatic heterocyclylsulfonyl;
R⁵ is substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group, provided that
(a) a compound
wherein R⁵ is a group of formula: 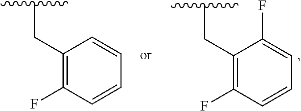 [SMILES: *Cc1ccccc1F ; *Cc1c(F)cccc1F]
and
which conforms to one of provisions (i) to (v):
(i) R² is substituted or unsubstituted amino-(C1-C2)alkyl or substituted or unsubstituted bromomethyl,
(ii) R¹ is phenyl substituted with a group other than halogen, haloalkyl or haloalkyloxy or unsubstituted phenyl, R² is methyl, and R⁴ is hydrogen or methyl,
(iii) R¹ is substituted phenyl, R² is hydrogen, R³ is substituted phenyl, and R⁴ is methyl,
(iv) R³ is bromo or alkyloxycarbonyl, and R⁴ is hydrogen, or
(v) R¹ is alkyl substituted with alkyloxycarbonyl or unsubstituted alkyl, R² is alkyl substituted with substituted or unsubstituted nitrogen-containing aromatic heterocyclic group, and R³ is substituted phenyl and R⁴ is methyl;
(b) a compound wherein R¹ is substituted or unsubstituted aromatic carbocyclic group or unsubstituted furyl, and R² is substituted or unsubstituted phenyl; and
aromatic carbocyclic group or unsubstituted furyl, and R² is substituted or unsubstituted phenyl; and
(c) the compounds of the formula: 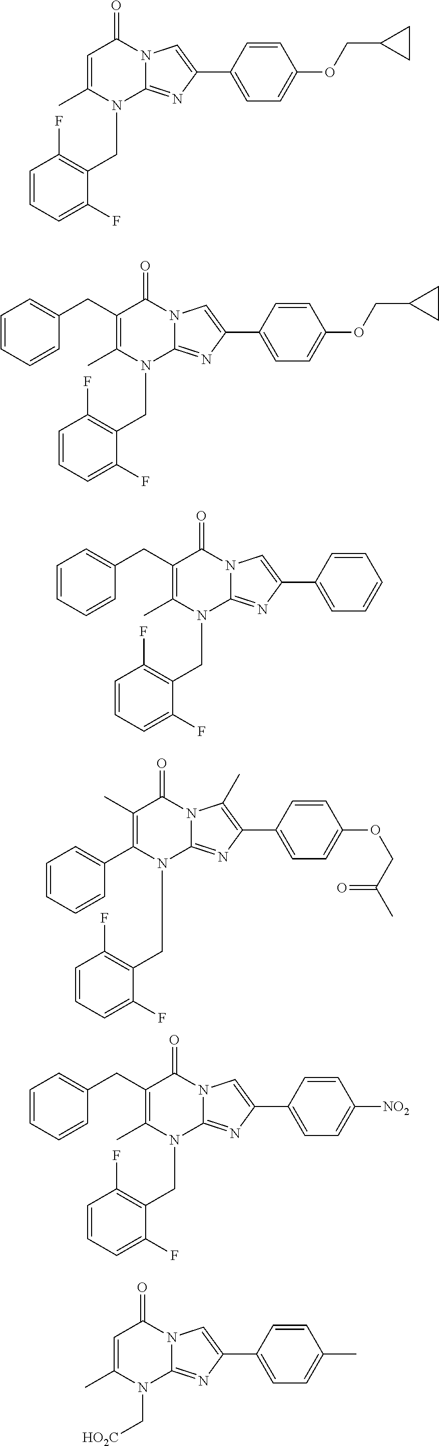 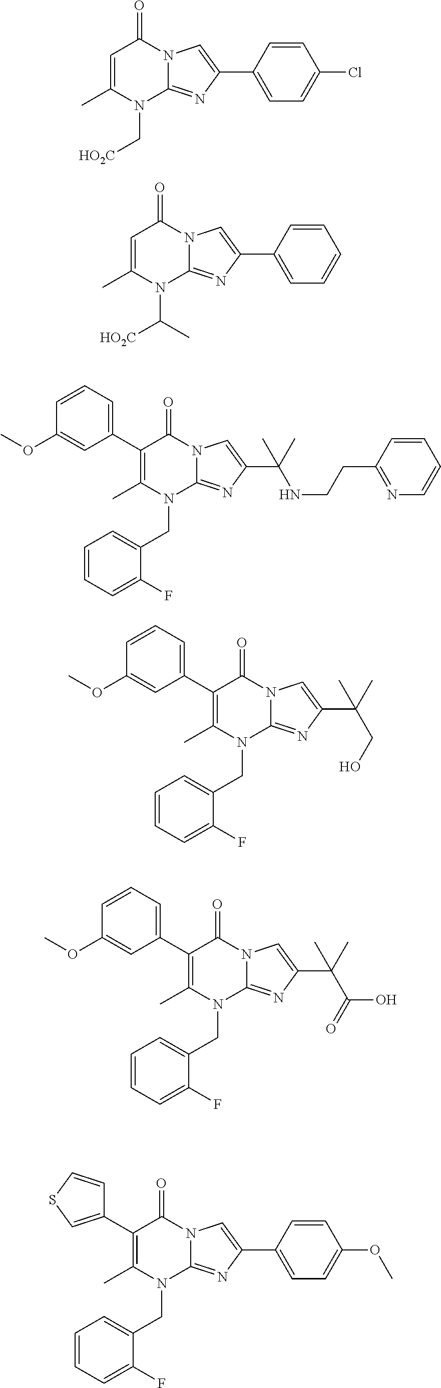 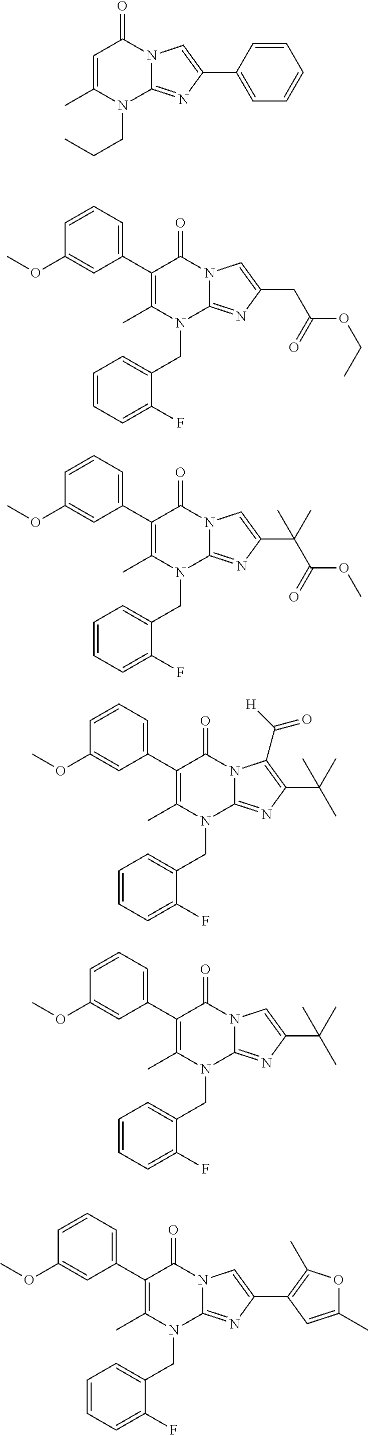 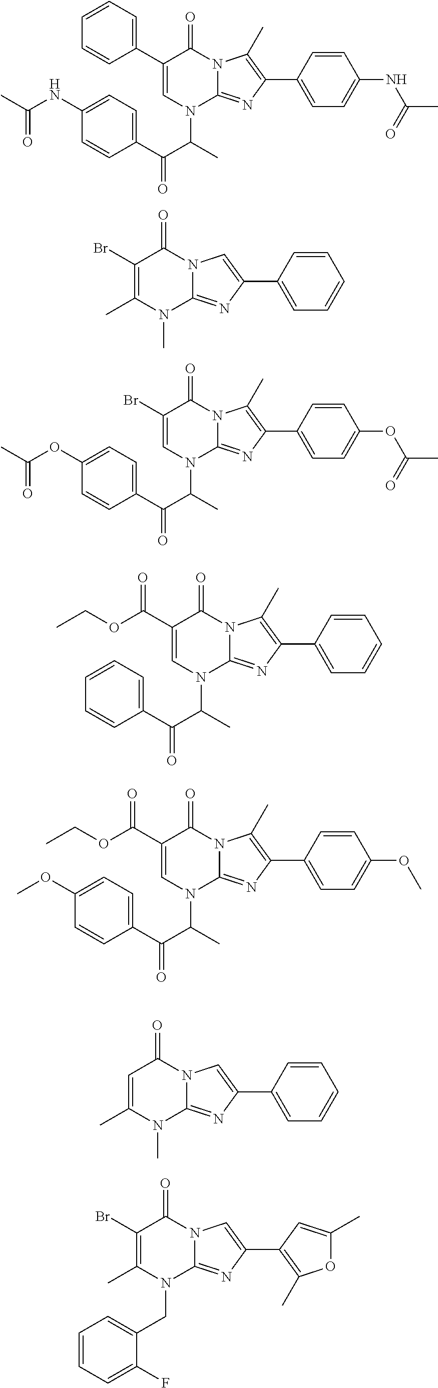 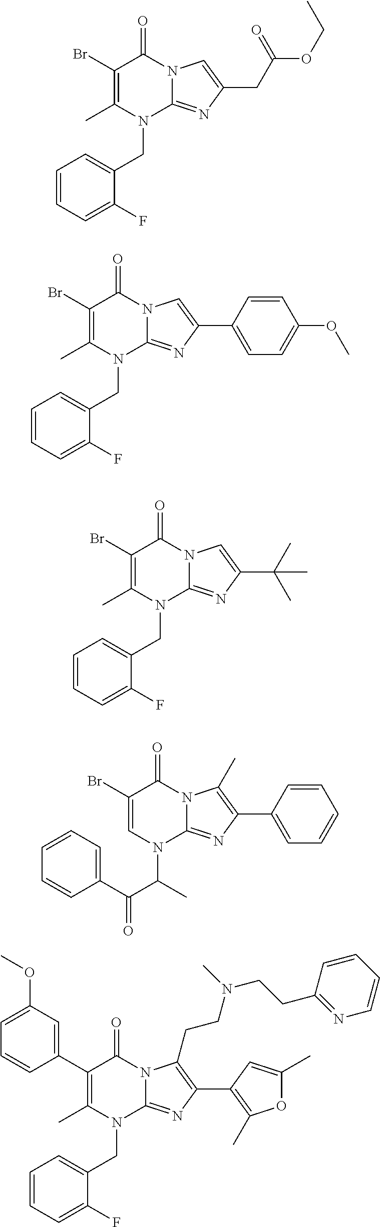 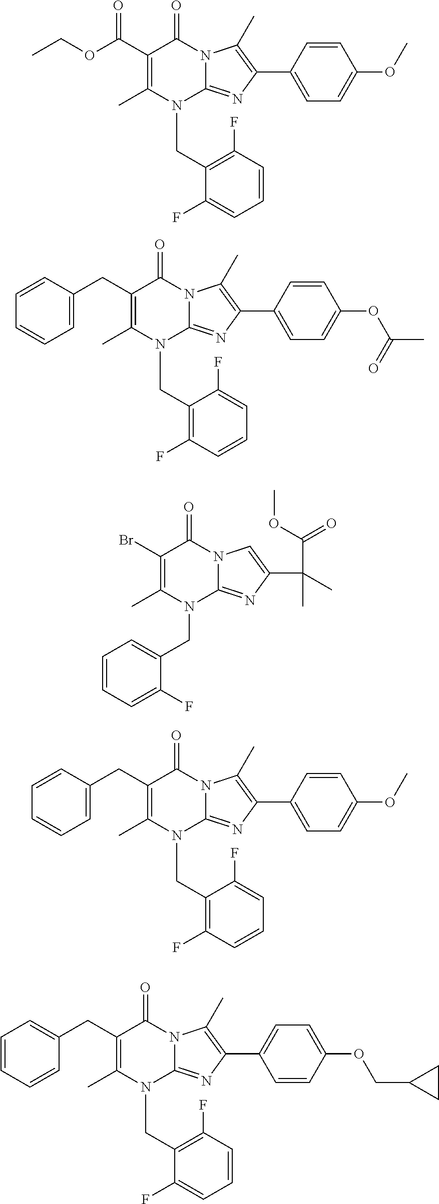 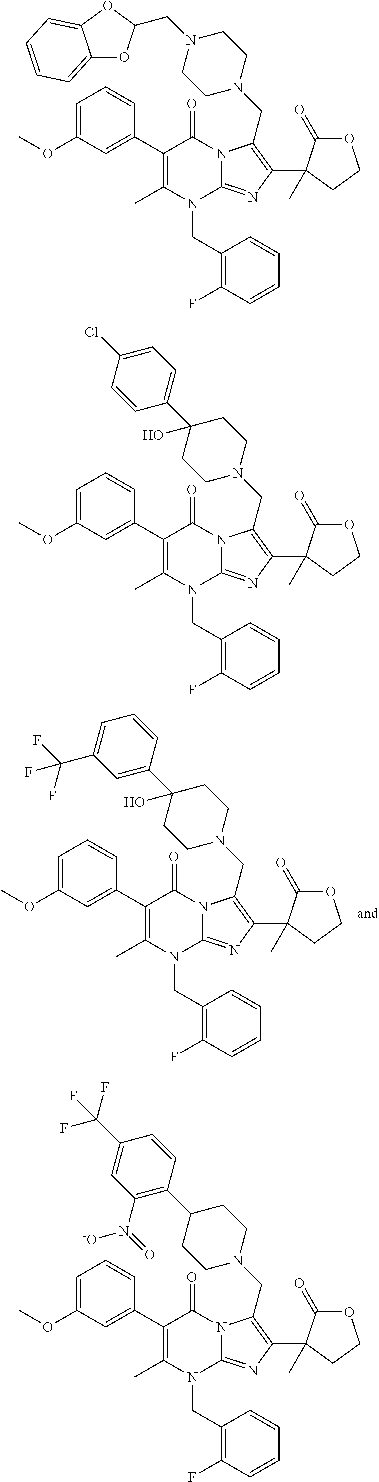
are excluded.

3. The compound according to claim 2 wherein R⁵ is substituted or unsubstituted C4-C8 alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group or substituted or unsubstituted aromatic heterocyclic group, or a pharmaceutically acceptable salt thereof.

4. The compound according to claim 2 wherein R⁵ is substituted or unsubstituted C4-C8 alkyl, substituted or unsubstituted alkenyl or substituted or unsubstituted alkynyl, or a pharmaceutically acceptable salt thereof.

5. The compound according to claim 2 wherein R⁵ is alkyl substituted with one or more substituents selected from the Substituent Group A consisting of halogen, cyano, hydroxy, formyl, substituted or unsubstituted non-aromatic carbocyclic group, substituted or unsubstituted non-aromatic heterocyclic group, substituted or unsubstituted aromatic heterocyclic group, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkylcarbonyl, substituted or unsubstituted alkenylcarbonyl, substituted or unsubstituted alkynylcarbonyl, substituted or unsubstituted non-aromatic carbocyclylcarbonyl, substituted or unsubstituted non-aromatic heterocyclylcarbonyl, substituted or unsubstituted aromatic heterocyclylcarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl, substituted or unsubstituted aromatic heterocyclylsulfonyl and substituted or unsubstituted amino, or a pharmaceutically acceptable salt thereof.

6. The compound according to claim 2 wherein R⁵ is a group of formula: 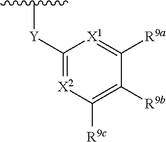
wherein
X¹ and X² are each independently N or CH,
Y is substituted or unsubstituted alkylene, substituted or unsubstituted alkenylene or substituted or unsubstituted alkynylene,
R^{9a}, R^{9b} and R^{9c} are each independently hydrogen, halogen, cyano, hydroxy, formyl, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted alkyloxy, substituted or unsubstituted alkenyloxy, substituted or unsubstituted alkynyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted alkylthio, substituted or unsubstituted alkenylthio, substituted or unsubstituted alkynylthio, substituted or unsubstituted non-aromatic carbocyclylthio, substituted or unsubstituted aromatic carbocyclylthio, substituted or unsubstituted non-aromatic heterocyclylthio, substituted or unsubstituted aromatic heterocyclylthio, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted alkyloxycarbonyl, substituted or unsubstituted alkenyloxycarbonyl, substituted or unsubstituted alkynyloxycarbonyl, substituted or unsubstituted non-aromatic carbocyclyloxycarbonyl, substituted or unsubstituted aromatic carbocyclyloxycarbonyl, substituted or unsubstituted non-aromatic heterocyclyloxycarbonyl, substituted or unsubstituted aromatic heterocyclyloxycarbonyl, substituted or unsubstituted carbamoyl, substituted or unsubstituted sulfamoyl, substituted or unsubstituted alkylsulfinyl, substituted or unsubstituted alkenylsulfinyl, substituted or unsubstituted alkynylsulfinyl, substituted or unsubstituted non-aromatic carbocyclylsulfinyl, substituted or unsubstituted aromatic carbocyclylsulfinyl, substituted or unsubstituted non-aromatic heterocyclylsulfinyl, substituted or unsubstituted aromatic heterocyclylsulfinyl, substituted or unsubstituted alkylsulfonyl, substituted or unsubstituted alkenylsulfonyl, substituted or unsubstituted alkynylsulfonyl, substituted or unsubstituted non-aromatic carbocyclylsulfonyl, substituted or unsubstituted aromatic carbocyclylsulfonyl, substituted or unsubstituted non-aromatic heterocyclylsulfonyl, substituted or unsubstituted aromatic heterocyclylsulfonyl or substituted or unsubstituted amino, or a pharmaceutically acceptable salt thereof.

7. The compound according to claim 2 wherein R² is hydrogen, halogen, formyl or substituted or unsubstituted alkyl, or a pharmaceutically acceptable salt thereof.

8. The compound according to claim 2 wherein R³ is hydrogen, halogen, cyano, carboxy, substituted or unsubstituted alkyl, substituted or unsubstituted alkenyl, substituted or unsubstituted alkynyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group or substituted or unsubstituted amino, or a pharmaceutically acceptable salt thereof.

9. The compound according to claim 2 wherein R⁴ is hydrogen, halogen, formyl, carboxy, substituted or unsubstituted alkyl, substituted or unsubstituted alkenyl, substituted or unsubstituted non-aromatic carbocyclic group, substituted or unsubstituted aromatic carbocyclic group, substituted or unsubstituted non-aromatic heterocyclic group, substituted or unsubstituted aromatic heterocyclic group, substituted or unsubstituted alkyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted carbamoyl or substituted or unsubstituted amino, or a pharmaceutically acceptable salt thereof.

10. The compound according to claim 2 wherein R⁴ is halogen, formyl, substituted methyl, substituted or unsubstituted C2-C8 alkyl, substituted or unsubstituted alkenyl, substituted or unsubstituted non-aromatic carbocyclic group, substituted aromatic carbocyclic group, substituted or unsubstituted non-aromatic heterocyclic group, substituted or unsubstituted aromatic heterocyclic group, substituted or unsubstituted alkyloxy, substituted or unsubstituted non-aromatic carbocyclyloxy, substituted or unsubstituted aromatic carbocyclyloxy, substituted or unsubstituted non-aromatic heterocyclyloxy, substituted or unsubstituted aromatic heterocyclyloxy, substituted or unsubstituted carbamoyl or substituted or unsubstituted amino, or a pharmaceutically acceptable salt thereof.

11. A pharmaceutical composition comprising the compound according to claim 2 or a pharmaceutically acceptable salt thereof as an active ingredient.

12. The pharmaceutical composition according to claim 11 that has autotaxin inhibitory effect.

13. The pharmaceutical composition according to claim 11 for the prevention or treatment of a disease involving autotaxin.

14. Use of a compound according to claim 2 or a pharmaceutically acceptable salt thereof for the manufacture of a medicament for the prevention or treatment of a disease involving autotaxin.

15. A method for the prevention or treatment of a disease involving autotaxin comprising administering a compound according to claim 2 or a pharmaceutically acceptable salt thereof.

16. The compound according to claim 2 or a pharmaceutically acceptable salt thereof for the prevention or treatment of a disease involving autotaxin.

17. The pharmaceutical composition according to claim 11 which is a chronic kidney disease therapeutic agent.
